(19) United States
(12) Patent Application Publication
(10) Pub. No.: US 2025/0122546 A1
(43) Pub. Date: Apr. 17, 2025

(54) RNA CONSTRUCTS AND USES THEREOF

(71) Applicant: BioNTech SE, Mainz (DE)

(72) Inventors: Gábor Boros, Mainz (DE); Katalin Karikó, Rydal, PA (US)

(21) Appl. No.: 18/705,383

(22) PCT Filed: Oct. 28, 2022
(86) PCT No.: PCT/EP2022/080237

§ 371 Date: Apr. 26, 2024

Related U.S. Application Data
(60) Provisional application No. 63/273,102, filed on Oct. 28, 2021.

(51) Int. Cl. C12P 19/34

(52) U.S. Cl. CPC C12P 19/34

(57) ABSTRACT

Disclosed herein are RNA polynucleotides comprising a 5′ Cap, a 5′ UTR comprising a cap proximal sequence disclosed herein, and a sequence encoding a payload. Also disclosed herein are compositions and medical preparations comprising the same, and compositions and methods of making and using the same.

### BACKGROUND

Use of RNA polynucleotides as therapeutics is a new and emerging field.

### SUMMARY

The present disclosure identifies certain challenges that can be associated with in vitro production of RNA, for example of RNA therapeutics.

For example, in some embodiments, the present disclosure identifies the source of certain problems that can be encountered with expression of polypeptides encoded by RNA therapeutics. Among other things, the present disclosure provides technologies for improving capping efficiency (e.g., percentage of capped transcripts in an in vitro transcription reaction), quality of an RNA preparation (e.g., of an in vitro transcribed RNA, e.g., the amount of short polynucleotide byproducts produced), translation efficiency of an RNA encoding a payload, and/or expression of a polypeptide payload encoded by an RNA. In some embodiments, translation efficiency and/or expression of an RNA-encoded payload can be improved with an RNA polynucleotide comprising: a 5′ cap as defined and described herein; a 5′ UTR comprising a cap proximal sequence as defined and described herein, and a sequence encoding a payload.

Without wishing to be bound by a particular theory, the present disclosure proposes that improved RNA transcription, capping efficiency, translation efficiency, and/or polypeptide payload expression and/or reduced transcription byproduct formation can be achieved through use of a 5′ cap structure as described herein in combination with certain transcription start site sequences of a template DNA.

In some embodiments, the present disclosure recognizes that certain transcription start sites provide improved RNA transcription, capping efficiency, translation efficiency, and/or polypeptide payload expression and/or reduced byproduct formation, e.g., when utilized with particular caps.

T7 RNA polymerase most commonly utilizes a GGG transcriptional start site (e.g., generating an RNA whose first three residues, N1, N2, and N3, are each “G”), and, moreover, has been reported to prefer “G” as an initiating residue (e.g., generating an RNA whose first residue, N1, is “G”). Conrad, et al. (2020) Communications Biology 3:439. Studies comparing T7 transcription of templates with different initiating residues report levels of transcripts beginning with “A” are only 25% of those observed for transcripts beginning with “G”. Milligan, et al. (1987) Nucleic Acids Research 15:8783-8798.

The 3′ end of commonly used dinucleotide cap also employ “G” (e.g., m₂^{7,2′-O}GppSpG “β-S-ARCA” or “D1”). Grudzien-Nogalska, et al. RNA 13:1745-1755. Indeed, certain such caps, e.g., β-S-ARCA, provide advantages including, e.g., being more resistant to human decapping enzymes (Kowalska et al. (2008) RNA 14:1119-1131) and interferon-induced proteins with tetratricopeptide repeats (IFITs), which inhibit Cap0-dependent translation (Diamond et al. (2014) Cytokine & Growth Factor Reviews 25:543-550; and Miedziak et al. (2019) RNA 25:58-68). However, poor capping efficiency is sometimes observed. Without wishing to be bound by any particular theory, the present disclosure proposes that competition with GTP in the transcription reaction may contribute to such poor capping efficiency.

Furthermore, the present disclosure provides a surprising finding that DNA template sequence, and particularly sequence of a transcriptional start site in a DNA template, may impact the usefulness of certain caps (e.g., 3-terminal-G-caps) in in vitro transcription reactions as described herein. Among other things, for example, the present disclosure demonstrates that DNA template including a GGG transcriptional start sequence can promote production of undesired short poly(G) byproducts, e.g., when 3′-terminal caps are utilized. The present disclosure thus identifies the source of a problem with certain in vitro transcription strategies, and furthermore provides surprising insights regarding in vitro transcription, including solutions to such problem(s).

For example, the present disclosure provides an insight that RNA transcripts comprising certain start sequences (e.g., those comprising a pyrimidine base (C or U) at the +2 position, such as GCG, GUG, or GCA) show certain benefits as compared to a purine base (A or G) at the same position. For example, in some embodiments, the present disclosure provides an insight that an RNA transcript having a pyrimidine base (C or U) at the +2 position improves transcription efficiency and/or translation, higher capping efficiency, less immunogenicity, and/or improved and/or prolonged expression, as compared to a purine base (A or G) at the same position, such as GGG as the initial sequence.

Additionally or alternatively, in some embodiments, the present disclosure recognizes that certain 5′ cap structures, when paired with certain transcription start sites, provide improved RNA transcription, translation efficiency, and/or polypeptide payload expression. In some embodiments, the present disclosure provides that certain 5′ cap structures (e.g., m₂^{(7,3′O)}Gppp^{(m2′O)}ApG), when paired with certain transcription start sites (e.g., AGN, such as AGA) result in higher capping efficiency, less immunogenicity, and much improved and prolonged expression as compared to transcripts comprising other 5′ cap structures combined with other transcription start sequences (such as, e.g., a β-S-ARCA cap used in combination with a GGG transcription start sequence). In some embodiments, the present disclosure also provides that certain trinucleotide 5′ cap structures (e.g., m₂^{(7,3′O)}Gppp^{(m2′O)}ApG) can be used in combination with a transcription start site that is not completely complementary to the 5′cap (e.g., in some embodiments, the present disclosure provides that m₂^{(7,3′)} Gppp^{(m2′O)}ApG can be used in combination with a GGG or GCG transcription start site). This can be advantageous, as it allows for the incorporation of a 5′ cap that has certain desired properties (such as, e.g, reduced immunogenicity), without having to create a new DNA template that is complementary to the 5′ cap of choice.

In some embodiments, the present disclosure provides an insight that RNAs generated with certain ARCA cap structures, when paired with certain transcription start sites other than a GGG start sequence, which has been thought to be the preferred start site for the ARCA caps, can surprisingly produce higher protein expression as compared to RNAs generated with the same cap and a GGG start sequence. For example, in some embodiments, the present disclosure has demonstrated that RNA generated with β-S-ARCA D1 cap (the “D1 cap”) and a GCG start sequence surprisingly produced higher protein expression as compared to the D1 cap with a GGG start sequence.

Still further, additionally or alternatively, in some embodiments, the present disclosure recognizes that identity of particular sequence(s) proximal to a 5′ cap can influence RNA transcription and/or translation efficiency of an associated payload. Without wishing to be bound by any particular theory, the present disclosure proposes that eIF4E competes with IFIT1 for binding to an RNA polynucleotide based on the identity of one or more nucleotides downstream of a 5′ cap, e.g., a cap proximal sequence as disclosed herein.

Accordingly, in some embodiments, the present disclosure provides, inter alia, a composition or medical preparation comprising an RNA polynucleotide, comprising: (i) a 5′ cap; (ii) a cap proximal sequence, e.g., as disclosed herein; and (iii) a sequence encoding a payload. Also disclosed herein are methods of making and using the same to, e.g., induce an immune response in a subject.

In some embodiments, the present disclosure recognizes that a GGG transcription start site, when paired with certain 5′ caps as defined and described herein, provide improved RNA transcription, translation efficiency, and/or polypeptide payload expression. For example, in some embodiments, the present disclosure provides a composition or medical preparation comprising an RNA polynucleotide comprising:
a 5′ cap; a cap proximal sequence comprising positions +1, +2, +3, +4, and +5 of the RNA polynucleotide; and a sequence encoding a payload, wherein:
   (i) the 5′ cap is a trinucleotide cap structure comprising N₁pN₂, wherein N₁ is position +1 of the RNA polynucleotide, and N2 is position +2 of the RNA polynucleotide, and wherein N₁ and N₂ are selected from one of the following combinations: (a) N₁ is C and N₂ is G; (b) N₁ is U and N₂ is G; or (c) N₁ is A and N₂ is G; and
   (ii) the cap proximal sequence comprises:
N₁ and N₂ of the trinucleotide cap structure and a sequence comprising N₃N₄N₅ at positions +3, +4, and +5 respectively of the RNA polynucleotide, wherein N₃ and N₄ are G, and N₅ is selected from: A, C, G, and U.

In some embodiments, the present disclosure provides a composition or medical preparation comprising an RNA polynucleotide comprising:
a 5′ cap; a cap proximal sequence comprising positions +1, +2, +3, +4, and +5 of the RNA polynucleotide; and a sequence encoding a payload, wherein:
   (i) the 5′ cap is a trinucleotide cap structure comprising N₁pN₂, wherein N₁ is position +1 of the RNA polynucleotide, and N₂ is position +2 of the RNA polynucleotide, and wherein N₁ and N₂ are each G; and
   (ii) the cap proximal sequence comprises:
N₁ and N₂ of the trinucleotide cap structure and a sequence comprising N₃N₄N₅ at positions +3, +4, and +5 respectively of the RNA polynucleotide, wherein N₃ is G, and each N₄ and N₅ is selected from: A, C, G, and U.

In some embodiments, the present disclosure provides a composition or medical preparation comprising an RNA polynucleotide comprising:
a 5′ cap; a cap proximal sequence comprising positions +1, +2, +3, +4, and +5 of the RNA polynucleotide; and a sequence encoding a payload, wherein:
   (i) the 5′ cap is a dinucleotide cap structure comprising N₁, wherein N₁ is position +1 of the RNA polynucleotide, and wherein N₁ is G; and
   (ii) the cap proximal sequence comprises:
      N₁ of the dinucleotide cap structure and a sequence comprising N₂N₃N₄N₅ at positions +2, +3, +4, and +5 respectively of the RNA polynucleotide, wherein each N₂ and N₃ is G, and each N₄ and N₅ is selected from: A, C, G, and U.

In some embodiments, the present disclosure provides a composition or medical preparation comprising an RNA polynucleotide comprising:
a 5′ cap; a cap proximal sequence comprising positions +1, +2, +3, +4, and +5 of the RNA polynucleotide; and a sequence encoding a payload, wherein:
   (i) the 5′ cap is a tetranucleotide cap structure comprising N₁pN₂pN₃, wherein N₁ is position +1 of the RNA polynucleotide, N₂ is position +2 of the RNA polynucleotide, and N₃ is position +3 of the polynucleotide, and wherein N₁, N₂, and N₃ are selected from one of the following combinations: (a) N₁ is C, N₂ is G, and N₃ is G; (b) N₁ is U, N₂ is G, and N₃ is G; or (c) N₁ is A, N₂ is G, and N₃ is G; and
   (ii) the cap proximal sequence comprises:
      N₁, N₂, and N₃ of the tetranucleotide cap structure and a sequence comprising N₄N₅ at positions +4 and +5 respectively of the RNA polynucleotide, wherein N₄ is G, and N₅ is selected from: A, C, G, and U.

In some embodiments, the present disclosure provides a composition or medical preparation comprising an RNA polynucleotide comprising:
a 5′ cap; a cap proximal sequence comprising positions +1, +2, +3, +4, and +5 of the RNA polynucleotide; and a sequence encoding a payload, wherein:
   (i) the 5′ cap is a tetranucleotide cap structure comprising N₁pN₂pN₃, wherein N₁ is position +1 of the RNA polynucleotide, N₂ is position +2 of the RNA polynucleotide, and N₃ is position +3 of the polynucleotide, and wherein N₁ is G, N₂ is G, and N₃ is G; and
   (ii) the cap proximal sequence comprises:
      N₁, N₂, and N₃ of the tetranucleotide cap structure and a sequence comprising N₄N₅ at positions +4 and +5 respectively of the RNA polynucleotide, wherein each N₄ and N₅ is selected from: A, C, G, and U.

In some embodiments, the present disclosure recognizes that a pyrimidine at +2 position of a transcription start site can improve capping efficiency (e.g., percentage of capped transcripts in an in vitro transcription reaction), quality of an RNA preparation (e.g., of an in vitro transcribed RNA, e.g., the amount of short polynucleotide byproducts produced), translation efficiency of an RNA encoding a payload, and/or expression of a polypeptide payload encoded by an RNA. In some embodiments, such technical effects can be observed independent of the identity of a 5′ UTR, capping method (e.g., enzymatic capping vs. co-transcriptional capping), cap structures (e.g., Cap0, Cap1, or Cap2), coding sequences, types of ribonucleotides (e.g., modified nucleotides vs. non-modified nucleotides), or combinations thereof.

For example, in some embodiments, the present disclosure recognizes that a GCG transcription start site, when paired with certain 5′ caps as defined and described herein, provide improved RNA transcription, translation efficiency, and/or polypeptide payload expression. For example, in some embodiments, the present disclosure provides a composition or medical preparation comprising an RNA polynucleotide comprising:
a 5′ cap; a cap proximal sequence comprising positions +1, +2, +3, +4, and +5 of the RNA polynucleotide; and a sequence encoding a payload, wherein:
   (i) the 5′ cap is a trinucleotide cap structure comprising N₁pN₂, wherein N₁ is position +1 of the RNA polynucleotide, and N₂ is position +2 of the RNA polynucleotide, and wherein N₁ and N₂ are selected from one of the following combinations: (a) N₁ is G and N₂ is G; (b) N₁ is U and N₂ is G; (c) N₁ is A and N₂ is G; or (d) N₁ is C and N₂ is G; and
   (ii) the cap proximal sequence comprises:
N₁ and N₂ of the trinucleotide cap structure and a sequence comprising N₃N₄N₅ at positions +3, +4, and +5 respectively of the RNA polynucleotide, wherein N₃ is C, N₄ is G, and N₅ is selected from: A, C, G, and U.

In some embodiments, the present disclosure provides a composition or medical preparation comprising an RNA polynucleotide comprising:
a 5′ cap; a cap proximal sequence comprising positions +1, +2, +3, +4, and +5 of the RNA polynucleotide; and a sequence encoding a payload, wherein:
   (i) the 5′ cap is a trinucleotide cap structure comprising N₁pN₂, wherein N₁ is position +1 of the RNA polynucleotide, and N₂ is position +2 of the RNA polynucleotide, and wherein N₁ is G and N₂ is C; and
   (ii) the cap proximal sequence comprises:
      N₁ and N₂ of the trinucleotide cap structure and a sequence comprising N₃N₄N₅ at positions +3, +4, and +5 respectively of the RNA polynucleotide, wherein N₃ is G, and each N₄ and N₅ is selected from: A, C, G, and U.

In some embodiments, the present disclosure provides a composition or medical preparation comprising an RNA polynucleotide comprising:
a 5′ cap; a cap proximal sequence comprising positions +1, +2, +3, +4, and +5 of the RNA polynucleotide; and a sequence encoding a payload, wherein:
(i) the 5′ cap is a dinucleotide cap structure comprising N₁, wherein N₁ is position +1 of the RNA polynucleotide, and wherein N₁ is G; and
(ii) the cap proximal sequence comprises: N₁ of the dinucleotide cap structure and a sequence comprising N₂N₃N₄N₅ at positions +2, +3, +4, and +5 respectively of the RNA polynucleotide, wherein N₂ is a pyrimidine (e.g., C or U), and each of N₃, N₄ and N₅ is selected from: A, C, G, and U. In some embodiments N₃ is G or A, and N₄ and N₅ are each separately and independently selected from: A, C, G, and U.

In some embodiments, the present disclosure provides a composition or medical preparation comprising an RNA polynucleotide comprising:
a 5′ cap; a cap proximal sequence comprising positions +1, +2, +3, +4, and +5 of the RNA polynucleotide; and a sequence encoding a payload, wherein:
   (i) the 5′ cap is a dinucleotide cap structure comprising N₁, wherein N₁ is position +1 of the RNA polynucleotide, and wherein N₁ is G; and
   (ii) the cap proximal sequence comprises:
      N₁ of the dinucleotide cap structure and a sequence comprising N₂N₃N₄N₅ at positions +2, +3, +4, and +5 respectively of the RNA polynucleotide, wherein N₂ is C, N₃ is G, and each N₄ and N₅ is selected from: A, C, G, and U.

In some embodiments, the present disclosure provides a composition or medical preparation comprising an RNA polynucleotide comprising:
a 5′ cap; a cap proximal sequence comprising positions +1, +2, +3, +4, and +5 of the RNA polynucleotide; and a sequence encoding a payload, wherein:
   (i) the 5′ cap is a tetranucleotide cap structure comprising N₁pN₂pN₃, wherein N₁ is position +1 of the RNA polynucleotide, N₂ is position +2 of the RNA polynucleotide, and N₃ is position +3 of the polynucleotide, and wherein N₁, N₂, and N₃ are selected from one of the following combinations: (a) N₁ is C, N₂ is G, and N₃ is C; (b) N₁ is U, N₂ is G, and N₃ is C; or (c) N₁ is A, N₂ is G, and N₃ is C; and
   (ii) the cap proximal sequence comprises:
      N₁, N₂, and N₃ of the tetranucleotide cap structure and a sequence comprising N₄N₅ at positions +4 and +5 respectively of the RNA polynucleotide, wherein N₄ is G, and N₅ is selected from: A, C, G, and U.

In some embodiments, the present disclosure provides a composition or medical preparation comprising an RNA polynucleotide comprising:
a 5′ cap; a cap proximal sequence comprising positions +1, +2, +3, +4, and +5 of the RNA polynucleotide; and a sequence encoding a payload, wherein:
   (i) the 5′ cap is a tetranucleotide cap structure comprising N₁pN₂pN₃, wherein N₁ is position +1 of the RNA polynucleotide, N₂ is position +2 of the RNA polynucleotide, and N₃ is position +3 of the polynucleotide, and wherein N₁ is G, N₂ is C, and N₃ is G; and
   (ii) the cap proximal sequence comprises:
      N₁, N₂, and N₃ of the tetranucleotide cap structure and a sequence comprising N₄N₅ at positions +4 and +5 respectively of the RNA polynucleotide, wherein each N₄ and N₅ is selected from: A, C, G, and U.

In some embodiments, the present disclosure recognizes that a CGC transcription start site, when paired with certain 5′ caps as defined and described herein, provide improved RNA transcription, translation efficiency, and/or polypeptide payload expression. For example, in some embodiments, the present disclosure provides a composition or medical preparation comprising an RNA polynucleotide comprising:
a 5′ cap; a cap proximal sequence comprising positions +1, +2, +3, +4, and +5 of the RNA polynucleotide; and a sequence encoding a payload, wherein:
   (i) the 5′ cap is a trinucleotide cap structure comprising N₁pN₂, wherein N₁ is position +1 of the RNA polynucleotide, and N₂ is position +2 of the RNA polynucleotide, and wherein N₁ and N₂ are selected from one of the following combinations: (a) N₁ is G and N₂ is C; (b) N₁ is U and N₂ is C; (c) N₁ is A and N₂ is C; or (d) N₁ is C and N₂ is C; and
   (ii) the cap proximal sequence comprises:
      N₁ and N₂ of the trinucleotide cap structure and a sequence comprising N₃N₄N₅ at positions +3, +4, and +5 respectively of the RNA polynucleotide, wherein N₃ is G, N₄ is C, and N₅ is selected from: A, C, G, and U.

In some embodiments, the present disclosure provides a composition or medical preparation comprising an RNA polynucleotide comprising:
a 5′ cap; a cap proximal sequence comprising positions +1, +2, +3, +4, and +5 of the RNA polynucleotide; and a sequence encoding a payload, wherein:
   (i) the 5′ cap is a trinucleotide cap structure comprising N₁pN₂, wherein N₁ is position +1 of the RNA polynucleotide, and N₂ is position +2 of the RNA polynucleotide, and wherein N₁ is C and N₂ is G; and
   (ii) the cap proximal sequence comprises:
      N₁ and N₂ of the trinucleotide cap structure and a sequence comprising N₃N₄N₅ at positions +3, +4, and +5 respectively of the RNA polynucleotide, wherein N₃ is C, and each N₄ and N₅ is selected from: A, C, G, and U.

In some embodiments, the present disclosure provides a composition or medical preparation comprising an RNA polynucleotide comprising:
a 5′ cap; a cap proximal sequence comprising positions +1, +2, +3, +4, and +5 of the RNA polynucleotide; and a sequence encoding a payload, wherein:
   (i) the 5′ cap is a tetranucleotide cap structure comprising N₁pN₂pN₃, wherein N₁ is position +1 of the RNA polynucleotide, N₂ is position +2 of the RNA polynucleotide, and N₃ is position +3 of the polynucleotide, and wherein N₁, N₂, and N₃ are selected from one of the following combinations: (a) N₁ is G, N₂ is C, and N₃ is G; (b) N₁ is U, N₂ is C, and N₃ is G; or (c) N₁ is A, N₂ is C, and N₃ is G; and
   (ii) the cap proximal sequence comprises:
      N₁, N₂, and N₃ of the tetranucleotide cap structure and a sequence comprising N₄N₅ at positions +4 and +5 respectively of the RNA polynucleotide, wherein N₄ is C, and N₅ is selected from: A, C, G, and U.

In some embodiments, the present disclosure provides a composition or medical preparation comprising an RNA polynucleotide comprising:
a 5′ cap; a cap proximal sequence comprising positions +1, +2, +3, +4, and +5 of the RNA polynucleotide; and a sequence encoding a payload, wherein:
   (i) the 5′ cap is a tetranucleotide cap structure comprising N₁pN₂pN₃, wherein N₁ is position +1 of the RNA polynucleotide, N₂ is position +2 of the RNA polynucleotide, and N₃ is position +3 of the polynucleotide, and wherein N₁ is C, N₂ is G, and N₃ is C; and
   (ii) the cap proximal sequence comprises:
      N₁, N₂, and N₃ of the tetranucleotide cap structure and a sequence comprising N₄N₅ at positions +4 and +5 respectively of the RNA polynucleotide, wherein each N₄ and N₅ is selected from: A, C, G, and U.

In some embodiments, the present disclosure provides a composition or medical preparation comprising an RNA polynucleotide comprising:
a 5′ cap; a cap proximal sequence comprising positions +1, +2, +3, +4, and +5 of the RNA polynucleotide; and a sequence encoding a payload, wherein:
   (i) the 5′ cap is a trinucleotide cap structure comprising N₁pN₂, wherein N₁ is position +1 of the RNA polynucleotide, and N₂ is position +2 of the RNA polynucleotide, and wherein N₁ is A and N₂ is U; and
   (ii) the cap proximal sequence comprises:
      N₁ and N₂ of the trinucleotide cap structure and a sequence comprising N₃N₄N₅ at positions +3, +4, and +5 respectively of the RNA polynucleotide, wherein N₃ is A, and each N₄ and N₅ is selected from: A, C, G, and U.

In some embodiments, the present disclosure provides a composition or medical preparation comprising an RNA polynucleotide comprising:
a 5′ cap; a cap proximal sequence comprising positions +1, +2, +3, +4, and +5 of the RNA polynucleotide; and a sequence encoding a payload, wherein:
   (i) the 5′ cap is a tetranucleotide cap structure comprising N₁pN₂pN₃, wherein N₁ is position +1 of the RNA polynucleotide, N₂ is position +2 of the RNA polynucleotide, and N₃ is position +3 of the polynucleotide, and wherein N₁ is A, N₂ is U, and N₃ is A; and
   (ii) the cap proximal sequence comprises:
      N₁, N₂, and N₃ of the tetranucleotide cap structure and a sequence comprising N₄N₅ at positions +4 and +5 respectively of the RNA polynucleotide, wherein each N₄ and N₅ is selected from: A, C, G, and U.

Additionally or alternatively, in some embodiments, the present disclosure recognizes that certain 5′ cap structures (e.g., as defined and described herein), when paired with certain transcription start sites, provide improved RNA transcription, translation efficiency, and/or polypeptide payload expression. In some embodiments, a 5′ cap is a dinucleotide cap structure (e.g., comprising N₁, wherein N₁ is a defined and described herein), a trinucleotide cap structure (e.g., comprising N₁pN₂, wherein N₁ and N₂ are as defined and described herein), or a tetranucleotide cap structure (e.g., comprising N₁pN₂pN₃, wherein N₁, N₂, and N₃ are as defined and described herein). In some embodiments, a 5′ cap comprises G*, wherein:

G* comprises a structure of formula (I):

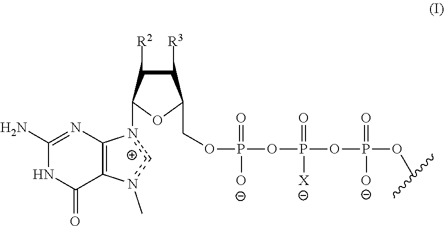

or a salt thereof, wherein R², R³, and X are as defined an described herein.

In some embodiments, the present disclosure recognizes that a 5′ cap having a dinucleotide cap structure comprising G*N¹, wherein N¹ is G, when combined with a GCG transcription start site, exhibit improved RNA transcription, translation efficiency, and/or polypeptide payload expression. In some embodiments, the present disclosure recognizes that a 5′ cap having a dinucleotide cap structure comprising G*N¹, wherein N¹ is C, when combined with a CGC transcription start site, exhibit improved RNA transcription, translation efficiency, and/or polypeptide payload expression.

In some embodiments, the present disclosure recognizes that a 5′ cap having a trinucleotide cap structure comprising G*N¹pN₂, wherein N₁ and N₂ are selected from one of the following combinations: (a) N₁ is C and N₂ is G; (b) N₁ is U and N₂ is G; (c) N₁ is A and N₂ is G; or (d) N₁ and N₂ are each G, when combined with a GGG transcription start site exhibit improved RNA transcription, translation efficiency, and/or polypeptide payload expression.

In some embodiments, the present disclosure recognizes that a 5′ cap having a trinucleotide cap structure comprising G*N¹pN₂, wherein N₁ and N₂ are selected from one of the following combinations (a) N₁ is G and N₂ is G; (b) N₁ is U and N₂ is G; (c) N₁ is A and N₂ is G; (d) N₁ is C and N₂ is G; or (e) N₁ is G and N₂ is C; when combined with a GCG transcription start site exhibit improved RNA transcription, translation efficiency, and/or polypeptide payload expression.

In some embodiments, the present disclosure recognizes that a 5′ cap having a trinucleotide cap structure comprising G*N¹pN₂, wherein N₁ and N₂ are selected from one of the following combinations: (a) N₁ is G and N₂ is C; (b) N₁ is U and N₂ is C; (c) N₁ is A and N₂ is C; (d) N₁ is C and N₂ is C; or (e) N₁ is C and N₂ is G; when combined with a CGC transcription start site exhibit improved RNA transcription, translation efficiency, and/or polypeptide payload expression.

In some embodiments, the present disclosure recognizes that a 5′ cap having a tetranucleotide cap structure comprising G*N¹pN₂pN₃, wherein N₁ and N₂ are selected from one of the following combinations (a) N₁ is C, N₂ is G, and N₃ is G; (b) N₁ is U, N₂ is G, and N₃ is G; (c) N₁ is A, N₂ is G, and N₃ is G; or (d) N₁ is G, N₂ is G, and N₃ is G, when combined with a GGG transcription start site exhibit improved RNA transcription, translation efficiency, and/or polypeptide payload expression.

In some embodiments, the present disclosure recognizes that a 5′ cap having a tetranucleotide cap structure comprising G*N¹pN₂pN₃, wherein N₁ and N₂ are selected from one of the following combinations (a) N₁ is C, N₂ is G, and N₃ is C; (b) N₁ is U, N₂ is G, and N₃ is C; (c) N₁ is A, N₂ is G, and N₃ is C or (d) N₁ is G, N₂ is C, and N₃ is G; when combined with a GCG transcription start site exhibit improved RNA transcription, translation efficiency, and/or polypeptide payload expression.

In some embodiments, the present disclosure recognizes that a 5′ cap having a tetranucleotide cap structure comprising G*N¹pN₂pN₃, wherein N₁ and N₂ are selected from one of the following (a) N₁ is G, N₂ is C, and N₃ is G; (b) N₁ is U, N₂ is C, and N₃ is G; (c) N₁ is A, N₂ is C, and N₃ is G; (d) N₁ is C, N₂ is G, and N₃ is C; when combined with a CGC transcription start site exhibit improved RNA transcription, translation efficiency, and/or polypeptide payload expression.

In some embodiments, the present disclosure recognizes that a 5′ cap having a trinucleotide cap structure comprising G*ApG, e.g., m₂^{(7′3′o)}Gppp^{(m2′O)}ApG, when combined with an AGN (e.g., AGA or AGC) transcription start site exhibit improved RNA transcription, translation efficiency, and/or polypeptide payload expression, e.g., as compared to a GGG transcription start site. For example, in some embodiments, the present disclosure provides composition or medical preparation comprising an RNA polynucleotide comprising:
a 5′ cap; a cap proximal sequence comprising positions +1, +2, +3, +4, and +5 of the RNA polynucleotide; and a sequence encoding a payload, wherein:
   (i) the 5′ cap is m₂^{(7,3′O)}Gppp^{(m2′O)}A₁pG₂, wherein A₁ is position +1 of the RNA polynucleotide, and G₂ is position +2 of the RNA polynucleotide; and
   (ii) the cap proximal sequence comprises:
      A₁ and G₂ of the 5′ cap and a sequence comprising N₃N₄N₅ at positions +3, +4, and +5 respectively of the RNA polynucleotide, wherein N₃-N₅ is selected from: A, C, G, and U.

In some embodiments, the present disclosure provides composition or medical preparation comprising an RNA polynucleotide comprising:
a 5′ cap; a cap proximal sequence comprising positions +1, +2, +3, +4, and +5 of the RNA polynucleotide; and a sequence encoding a payload, wherein:
   (i) the 5′ cap is m₂^{(7,3′O)}Gppp^{(m2′O)}A₁pG₂, wherein A₁ is position +1 of the RNA polynucleotide, and G₂ is position +2 of the RNA polynucleotide; and
   (ii) the cap proximal sequence comprises:
      A₁ and G₂ of the 5′ cap and a sequence comprising N₃N₄N₅ at positions +3, +4, and +5 respectively of the RNA polynucleotide, wherein N₃ is A, and N₄ and N₅ are selected from: A, C, G, and U.

In some embodiments, the present disclosure provides composition or medical preparation comprising an RNA polynucleotide comprising:
a 5′ cap; a cap proximal sequence comprising positions +1, +2, +3, +4, and +5 of the RNA polynucleotide; and a sequence encoding a payload, wherein:
   (i) the 5′ cap is m₂^{(7,3′O)}Gppp^{(m2′O)}A₁pG₂, wherein A₁ is position +1 of the RNA polynucleotide, and G₂ is position +2 of the RNA polynucleotide; and
   (ii) the cap proximal sequence comprises:
      A₁ and G₂ of the 5′ cap and a sequence comprising N₃N₄N₅ at positions +3, +4, and +5 respectively of the RNA polynucleotide, wherein N₃ and N₄ are G, and N₅ is selected from: A, C, G, and U.

In some embodiments, the present disclosure provides composition or medical preparation comprising an RNA polynucleotide comprising:
a 5′ cap; a cap proximal sequence comprising positions +1, +2, +3, +4, and +5 of the RNA polynucleotide; and a sequence encoding a payload, wherein:
   (i) the 5′ cap is m₂^{(7,3′O)}Gppp^{(m2′O)}A₁pG₂, wherein A₁ is position +1 of the RNA polynucleotide, and G₂ is position +2 of the RNA polynucleotide; and
   (ii) the cap proximal sequence comprises:
      A₁ and G₂ of the 5′ cap and a sequence comprising N₃N₄N₅ at positions +3, +4, and +5 respectively of the RNA polynucleotide, wherein N₃ is C, N₄ is G, and N₅ is selected from: A, C, G, and U.

This disclosure provides a composition or medical preparation comprising a capped RNA polynucleotide encoding a gene product, which RNA polynucleotide comprises the formula:

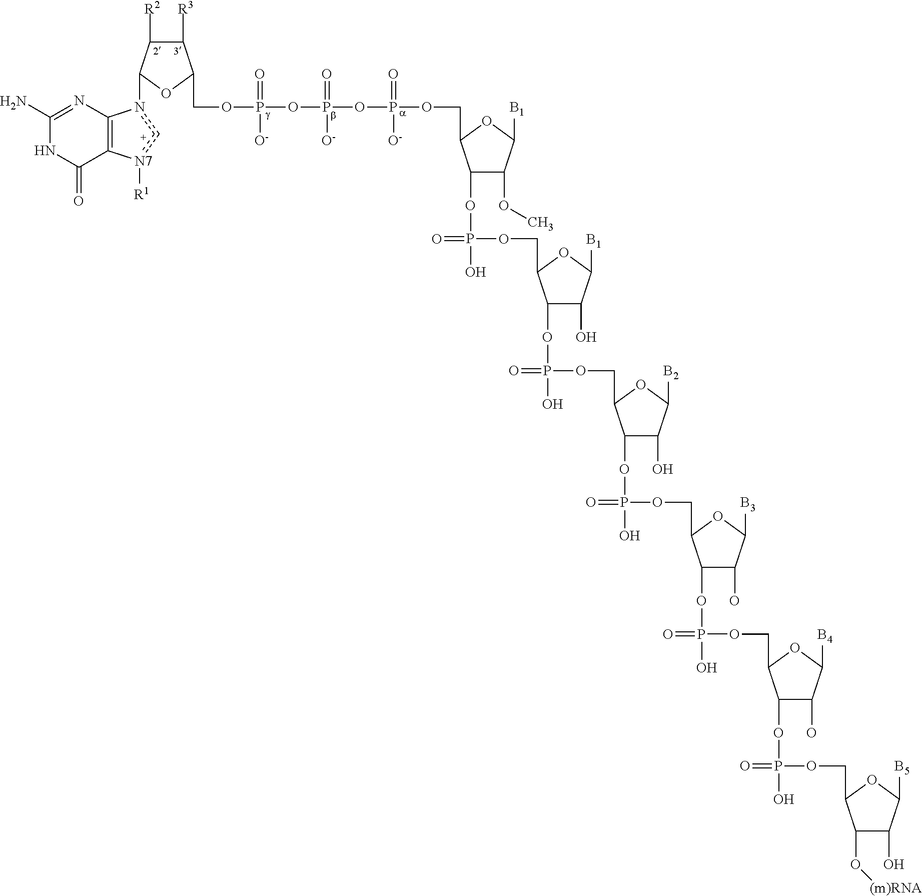

wherein R¹ is CH₃, R² and R³ are as defined above and herein,
wherein B₁ is any nucleobase, preferably A; B₂ is any nucleobase, preferably G; B₃ is any nucleobase, preferably A or C; B₄ is any nucleobase; and B₅ is any nucleobase, and
wherein, when the RNA polynucleotide is administered to a subject, the levels of expression of the encoded gene product at about 6 hours after administration and at about 48 hours after administration do not differ by more than 5-fold.

Provided herein is a pharmaceutical composition comprising an RNA polynucleotide disclosed herein. In some embodiments, a pharmaceutical composition comprises a composition or a medical preparation disclosed herein.

Also provided herein is a method of manufacturing a pharmaceutical composition, e.g., comprising an RNA polynucleotide disclosed herein, by combining an RNA polynucleotide with lipids to form lipid nanoparticles that encapsulate said RNA.

This disclosure provides a nucleic acid template suitable to produce a capped RNA, in which the first five nucleotides transcribed from the template strand of the nucleic acid template comprise the sequence N₁pN₂pN₃pN₄pN₅, wherein N₁ is any nucleotide, preferably T; N₂ is any nucleotide, preferably C; N₃ is any nucleotide, preferably T or G; N₄ is any nucleotide; and N₅ is any nucleotide. In some embodiments, a DNA template comprises: a sequence encoding a 5′ UTR, a sequence encoding a payload, a sequence encoding a 3′ UTR and a sequence encoding polyA sequence.

Provided herein is an vitro transcription reaction comprising:
(i) a template DNA comprising a polynucleotide sequence complementary to an RNA polynucleotide sequence disclosed herein;
(ii) a polymerase; and
(iii) an RNA polynucleotide.

Also provided herein is an RNA polynucleotide isolated from an in vitro transcription reaction provided.

Also provided herein is a composition comprising a DNA polynucleotide comprising a sequence complementary to an RNA polynucleotide sequence provided. In some embodiments, a DNA polynucleotide disclosed herein can be used to transcribe an RNA polynucleotide disclosed herein.

This disclosure provides, a method comprising: administering to a subject, a pharmaceutical composition comprising an RNA polynucleotide disclosed herein formulated in a lipid nanoparticle (LNP) or a lipoplex (LPX) particle, e.g., as disclosed herein. In some embodiments, the provided compositions, medical preparation, and therapeutics described herein increase expression of RNA when administered in an LNP formulation.

Also provided herein is a method of inducing an immune response in a subject, comprising administering to a subject, a pharmaceutical composition comprising an RNA polynucleotide disclosed herein formulated in a lipid nanoparticle (LNP) or a lipoplex (LPX) particle, e.g., as disclosed herein.

Provided herein is a method of vaccination of a subject by administering a pharmaceutical composition comprising an RNA polynucleotide disclosed herein formulated in a lipid nanoparticle (LNP) or a lipoplex (LPX) particle, e.g., as disclosed herein.

This disclosure provides, a method of decreasing interaction with IFIT1 of an RNA polynucleotide that comprises a 5′ cap and a cap proximal sequence comprising positions +1, +2, +3, +4, and +5 of the RNA polynucleotide, the method comprising a step of: providing a variant of an RNA polynucleotide that differs from a parental RNA polynucleotide by substitution of one or more residues within a cap proximal sequence, and determining that interaction of a variant with IFIT1 is decreased relative to that of a parental RNA polynucleotide.

Also provided herein is a method of producing a polypeptide comprising a step of: providing an RNA polynucleotide that comprises a 5′ cap, a cap proximal sequence that comprises positions +1, +2, +3, +4, and +5 of the RNA polynucleotide, and a sequence encoding a payload; wherein an RNA polynucleotide is characterized in that when assessed in an organism administered an RNA polynucleotide or a composition comprising the same, elevated expression and/or increased duration of expression of a payload is observed relative to an appropriate reference comparator.

Provided herein is a method of increasing translatability of an RNA polynucleotide that comprises a 5′ cap, a cap proximal sequence that comprises positions +1, +2, +3, +4, and +5 of the RNA polynucleotide and a sequence encoding a payload, the method comprising a step of: providing a variant of an RNA polynucleotide that differs from a parental RNA polynucleotide by substitution of one or more residues within a cap proximal sequence; and determining that expression of a variant is increased relative to that of a parental RNA polynucleotide.

Also provided herein is a method of improving capping efficiency (e.g., percentage of capped transcripts in an in vitro transcription reaction) of RNA transcripts, the improvement that comprises including a pyrimidine at +2 position of a transcription start site in a DNA template for in vitro transcription. Exemplary pyrimidines include, e.g., C or U. In some embodiments, the +1 position of a transcription start site is G. In some embodiments, the +3 position of a transcription start site is a pyrimidine or a purine. In some embodiments, a transcription start site may be GCG, GUG, or GCA. In some embodiments, such improvements can be observed independent of the identity of a 5′ UTR, capping method (e.g., enzymatic capping vs. co-transcriptional capping), cap structures (e.g., Cap0, Cap1, or Cap2), coding sequences, types of ribonucleotides (e.g., modified nucleotides vs. non-modified nucleotides), formulation (e.g., lipoplex vs. lipid nanoparticles) or combinations thereof.

Also provided herein is a method of improving quality of an RNA preparation (e.g., of an in vitro transcribed RNA, e.g., the amount of short polynucleotide byproducts produced), the improvement that comprises including a pyrimidine at +2 position of a transcription start site in a DNA template for in vitro transcription. Exemplary pyrimidines include, e.g., C or U. In some embodiments, the +1 position of a transcription start site is G. In some embodiments, the +3 position of a transcription start site is a pyrimidine or a purine. In some embodiments, a transcription start site may be GCG, GUG, or GCA. In some embodiments, such improvements can be observed independent of the identity of a 5′ UTR, capping method (e.g., enzymatic capping vs. co-transcriptional capping), cap structures (e.g., Cap0, Cap1, or Cap2), coding sequences, types of ribonucleotides (e.g., modified nucleotides vs. non-modified nucleotides), formulation (e.g., lipoplex vs. lipid nanoparticles), or combinations thereof.

Also provided herein is a method of improving translation efficiency of an RNA encoding a payload, and/or expression of a polypeptide payload encoded by an RNA, the improvement that comprises including a pyrimidine at +2 position of a transcription start site in a DNA template for in vitro transcription. Exemplary pyrimidines include, e.g., C or U. In some embodiments, the +1 position of a transcription start site is G. In some embodiments, the +3 position of a transcription start site is a pyrimidine or a purine. In some embodiments, a transcription start site may be GCG, GUG, or GCA. In some embodiments, such improvements can be observed independent of the identity of a 5′ UTR, capping method (e.g., enzymatic capping vs. co-transcriptional capping), cap structures (e.g., Cap0, Cap1, or Cap2), coding sequences, types of ribonucleotides (e.g., modified nucleotides vs. non-modified nucleotides), formulation (e.g., lipoplex vs. lipid nanoparticles), or combinations thereof.

Also provided herein is a method of providing a framework for an RNA polynucleotide that comprises a 5′ cap, a cap proximal sequence, and a payload sequence, the method comprising a step of:
assessing at least two variants of an RNA polynucleotide, wherein:
each variant includes a same 5′ cap and payload sequence; and
the variants differ from one another at one or more specific residues of a cap proximal sequence;
wherein the assessing comprises determining expression levels and/or duration of expression of a payload sequence; and
selecting at least one combination of 5′ cap and a cap proximal sequence that displays elevated expression relative to at least one other combination.

### BRIEF DESCRIPTION OF THE DRAWING

FIG. 1. Chemical structure of certain functional caps characterized herein. Red circle indicates modification (—CH₃) at the C2′ or C3′ position of 7-methylguanosine of each anti-reverse cap analog (ARCA) to prevent reverse orientation. β-S-ARCA dinucleotide cap (m₂^{7,2′O}GppspG) bears a single phosphorothioate moiety at the β position of the 5′,5′-triphosphate bridge (blue circle). This cap exists in two diastereomers designated D1 and D2 according to the fractions of HPLC run (Kowalska, et al. (2008) RNA 14:1119-1131). CleanCap AG 3′OMe trinucleotide cap (CC413—m₂^{(7,3′O)}Gppp^{(m2′O)}ApG) contains another methyl group (—CH₃) highlighted with orange circle at the 2′OH position of the first ribose sugar of the first nucleotide in opposite to dinucleotide caps. The non-ARCA version of CC413 corresponds to the standard CleanCap AG (CC113-m₂⁽⁷⁾Gppp^{(m2′O)}ApG which is not modified at C3′ position of 7-methylguanosine.

FIG. 2. Characteristics of in vitro transcribed mRNA disclosed herein. (A) The yield and the corresponding fractions of mRNA encoding murine erythropoietin (EPO mRNA) or firefly luciferase (LUC mRNA) were analyzed by a spectrophotometer and electrophoresis on a 1.4% agarose gel, respectively. All mRNAs contain N1-methylpseudouridine (m1Ψ) nucleoside modifications. In order to examine the capping efficiency of mRNAs, a ribozyme assay followed by self-made urea polyacrylamide gel electrophoresis (Urea PAGE) was performed. For quantification of capping reactions, the percentage of capped transcripts in the total pool of capped and uncapped mRNA was determined. (B) Quantification of the luminescent signal obtained by LUC mRNAs uncapped (none) or capped with anti-reverse cap analog (ARCA-G), β-S-ARCA (D1), enzymatic cap (Ecap1) and CleanCap AG 3′OMe (CC413) using Rabbit Reticulocyte Lysate translation system. All data are represented as mean±standard deviation of the values (SEM) obtained from quadruplicate data points. RLU=relative light unit. G=guanosine; A=adenosine.

FIG. 3. Durability and biodistribution of luciferase translated from m1Ψ-modified LUC mRNAs in mice. (A) Representative IVIS images of groups of four BALB/c mice injected IV with 3.0 μg TransIT-complexed LUC mRNAs containing different 5′cap structures (ARCA-G, D1, Ecap1 and CC413). LUC activity was measured at the indicated time points. Relative luminescence images are shown, and the scale of average radiance is indicated. (B) Quantification of the bioluminescent signal measured in mice at 6, 24 and 48 h after injection of 3.0 μg TransIT-complexed mRNA encoding firefly luciferase. All data are represented as mean±standard deviation of the values obtained from 4 animals per group. A p value of equal to or less than 0.05 was considered statistically significant (asterisks indicate p<0.05). RLU=relative light unit: A=adenosine; G=guanosine. hAg=5′UTR derived from human α-globin mRNA. ARCA-G=anti-reverse cap analog; D1=β-S-ARCA; Ecap1=enzymatic cap; CC413=CleanCap AG 3′OMe.

FIG. 4. Biological activity of murine EPO-encoding mRNA prepared with different 5′cap structures. Mice received a single intravenous injection of 3.0 μg of ARCA-G, β-S-ARCA (D1) enzymatically (Ecap1) or CleanCap AG 3′OMe (CC413)-capped mRNA complexed with TransIT mRNA reagent. (A) Plasma EPO levels were determined by ELISA 6, 24, 48 and 72 hours post injection. (B) Hematocrits were measured at the indicated time points using 20 μl of blood. Three animals per group were analyzed. Error bars are standard error of the mean (SEM) and less than 0.05 was considered statistically significant (asterisks indicate p<0.05). mock=TransIT reagent without RNA sample; A=adenosine; G=guanosine.

FIG. 5. Reduced immunogenicity of in vitro transcribed mRNA capped with CleanCap AG. (A) Heatmap representing changes in level of the symbolized proinflammatory cytokines and chemokines was derived from MSD (Meso Scale Discovery) immunoassay of human peripheral blood mononuclear cells (PBMCs) treated for 24 hours with cationic lipid-complexed mRNA (RNA-LPX) carrying various 5′cap structures ((anti-reverse cap analog (ARCA), 3-S-ARCA (D1) enzymatic cap (Ecap1) or CleanCap AG 3′OMe (CC413)). Each capped mRNA was used at three different final concentrations, as indicated. Values obtained from PBS-treated cells were used as the baseline for comparison. The results are expressed as mean±standard deviation of values from three independent experiments in triplicate performed in three donors. (B) Short abortive byproducts produced during in vitro transcription of capped mRNAs starting with GGG or AGA were separated by self-made denaturing urea polyacrylamide gel electrophoresis. Single-stranded RNA (ssRNA) ladder was used as a marker for size approximation of small transcripts. G=guanosine; A=adenosine; TNF-α=Tumor necrosis factor alpha; IFN-γ=Interferon gamma; IL-6=Interleukin 6; IL-1β=Interleukin 1 beta; MIP-1β=Macrophage inflammatory protein 1 beta.

FIG. 6. Physiological responses to injection of EPO mRNA capped with conventional or anti-reverse cap1 analog in mice. Plasma EPO levels and hematocrits were determined in mice following intravenous injection of 3.0 μg TransIT-complexed CC113 (CleanCap AG) or CC413 (CleanCap AG 3′OMe) EPO mRNA at the indicated days. Error bars are referred to as the standard error of the mean of a data set obtained from 3 mice per group.

FIG. 7. Schematic representation of each mRNA utilized in Example 1. In vitro transcribed mRNAs contain 5′cap (anti-reverse cap analog (ARCA-G); phosphorothioate group-containing cap analog (β-S-ARCA); Ecap1 (enzymatic cap) or CC413 (CleanCap AG 3′OMe)); GGG and AGA as two different start site (S); 5′UTR of human alpha globin (hAg) mRNA, the coding sequence (CDS) of murine erythropoietin (EPO—582 nt) or firefly luciferase (Luc—1,653 nt), FI element as 3′UTR and an encoded poly(A)tail (AAA₁₀₀, 100 nt) interrupted by a linker (L, 10 nt) (A30LA70). All mRNAs used in this study contain N1-methylpseudouridine (m1Ψ) nucleoside modification. UTR=untranslated region, G=guanosine, A=adenosine.

FIG. 8. Comparison of cytokine and chemokine levels in human PBMC transfected with CleanCap AG 3′OMe-capped mRNA with or without nucleoside modification. CleanCap AG 3′OMe (CC413)-capped mRNA containing 1-methylpseudouridine-(m1Ψ) or uridine-(U) was synthesized by in vitro transcription and then purified with cellulose. Human peripheral blood mononuclear cells (PBMC) were transfected with cationic lipid-complexed mRNA (RNA-LPX) at a final concentration of 0.2, 0.5 and 1.5 μg/ml. Supernatants were collected at 24 h post-transfection and level of the indicated proinflammatory cytokines and chemokines were determined by MSD. Data presented in the heatmap were obtained from three independent experiment performed in three donors. TNF-α=Tumor necrosis factor alpha; IFN-γ=Interferon gamma; IL-6=Interleukin 6; IL-1β=Interleukin 1 beta; MIP-1β=Macrophage inflammatory protein 1 beta.

FIG. 9. D1-capped mRNA starting with GGA provides improved expression as compared to D1-capped mRNA starting with AGA. (A) Quantification of plasma concentrations of murine EPO (mEPO) in mice 6, 24, 48, and 72 hours after IV injection of 3 μg of TransIT-formulated RNA with modified nucleotides (m1Ψ), encoding mEPO and comprising a cap structure (D1 cap or CC413 cap) with a start sequence (e.g., GGA or AGA); and a TEV 5′UTR. (B) Quantification of luciferase expression in mice 6 and 24 hours after injection of 3 μg of TransIT-formulated mRNA transcripts, where the transcripts with modified nucleotides (m1Ψ) encode firefly luciferase and comprise a cap structure (D1 cap or CC413 cap) with a start sequence (GGA or AGA); and a TEV 5′UTR.

FIG. 10. Beneficial effects of pyrimidine base at +2 position of an IVT mRNA described herein on the performance of the IVT mRNA. (A) Quantification of plasma concentrations of murine EPO (mEPO) in mice 6, 24, and 48 hours after IV injection of TransIT-formulated mRNA with modified nucleotides (m1Ψ) encoding mEPO and comprising a cap structure (D1 cap) with a start sequence (GGG, GAG, GGA, GGU, GGC, GUG, GCA, or GCG); and a TEV 5′UTR. In the figure, R stands for purine nucleotide and Y stands for pyrimidine nucleotide. (B) Hematocrit levels in the same mice 0, 7 and 14 days after injection of RNA.

FIG. 11. Impact of pyrimidine base at +2 position of an IVT mRNA on the performance of IVT mRNA is independent of 5′ cap. (A) Quantification of murine EPO (mEPO) in the plasma of mice 6, 24, 48, and 72 hours after injection of 3 μg of TransIT-formulated mRNA with modified nucleotides (m1Ψ) encoding mEPO and comprising a cap structure (a D1 cap, an enzymatically incorporated cap (Ecap1) or a CC413 cap) with a start sequence (GGG, GGA, GUG, or GCG) and a TEV 5′UTR. An mRNA comprising a CC413 cap with a start sequence of AGC was used as a control for comparison. (B) Hematocrit levels measured in the same mice 0, 7 and 14 days after injection.

FIG. 12. Pyrimidine base effect is independent of nucleoside modification and/or 5′UTR of IVT mRNA. (A) Plasma concentrations of murine EPO (mEPO) in mice 6, 24, 48, and 72 hours after injection of 3 μg of TransIT-formulated mRNA transcripts encoding mEPO and comprising a cap structure (an enzymatically incorporated cap0 (Ecap0), an enzymatically incorporated cap1 (Ecap1), ARCA-G cap, or D1 cap) with a start sequence (GGG or GCG) and a hAg 5′UTR. The mRNA transcripts utilized in this experiment contained non-modified uracil residues. An mRNA comprising a CC413 cap with a start sequence of AGA was used as a control for comparison. (B) Hematocrit levels measured in the same mice 0 and 7 days after injection of mRNA (each dot represents one moust).

FIG. 13. Pyrimidine base effect is independent of coding sequence and/or formulation. (A) Representative IVIS images of mice 24, 48, and 72 hours after injection with 10 μg of F-12 (lipoplex)-formulated mRNA or no mRNA, where the mRNA encodes firefly luciferase and comprise a cap structure (a D1 cap or a CC413 cap) with a start sequence (GGG, GAG, GGA, GGU, GGC, GUG, GCA, or GCG) and an hAg 5′ UTR. The mRNA transcripts utilized in this experiment contained non-modified uracil residues. In the figure, R stands for a pyrimidine nucleotide and Y stands for a purine nucleotide. (B) Quantification of luciferase expression for the mice depicted in (A).

FIG. 14. Shown is a schematic comparison of DNA templates with transcription start site GGG or GCG and their resulting RNA transcripts. RNA transcripts synthesized from DNA templates with or without Lig3 self-hybridization sequence in 3′ UTR are compared. In the construct having Lig3 self-hybridization sequence in 3′ UTR, the coding strand of the DNA template with transcription start site GGG or GCG has CG or AA, respectively, at its positions +4 and +5. In the construct without Lig3 self-hybridization sequence in 3′ UTR, the coding strand of the DNA template with transcription start site GGG or GCG has the same AT at its positions +4 and +5. In such a construct, the only difference between the two templates having transcription start site GGG or GCG is the nucleotide in the second position (+2).

FIG. 15. Start sequence GGG results in higher capping efficiency for D1-capped mRNAs. Capping efficiency for D1-capped mRNA encoding EPO or firefly luciferase and comprising a GGG or GCG start sequence was compared. Following in vitro transcription, reaction mixtures were run on a Urea-PAGE gel and an agarose gel, and capping efficiency was determined by comparing the intensity of the top band (capped) with that of the lower band (uncapped) in the Urea-PAGE gel.

FIG. 16. Impact of changing the nucleotide from a purine to a pyrimidine at the second position of an RNA construct on translation. (A) Plasma concentrations of murine EPO (mEPO) in mice 6, 24, 48, and 72 hours after injection of 3 μg of TransIT-formulated m1Ψ-RNA transcripts encoding mEPO and comprising a cap structure (an ARCA-G, D1, Ecap1 or CC413 cap) with a start sequence (GGG or GCG; or AGA for CC413 only) and a hAg 5′UTR. (B) Hematocrit levels measured in the same mice characterized in (A), 0, 7, and 14 days after injection of RNA.

FIG. 17. Changing the nucleotide from a purine to a pyrimidine at the second position of an RNA construct can eliminate short byproducts. In vitro transcription reactions were performed to produce m1Ψ-mRNA transcripts encoding mEPO and comprising a cap (an Ecap1, ARCA-G, D1, or CC413 cap) with a start sequence (GGG or GCG; or AGA for CC413 only) and a hAg 5′UTR). Following in vitro transcription, reaction mixtures were run on a Urea-PAGE gel. Short byproducts correspond to the bands towards the bottom of the gel.

FIG. 18. Changing the nucleotide from a purine to a pyrimidine at the second position of an RNA construct can result in a less immunogenic mRNA. Secretion of various proinflammatory cytokines by human PBMCs was analyzed after incubation with 1.5 or 5.0 μg of D1-capped m1Ψ-mRNA comprising a GGG or GCG start sequence. TNF-α=Tumor necrosis factor alpha; IFN-γ=Interferon gamma; IL-6=Interleukin 6; IL-1β=Interleukin 1 beta; MIP-1β=Macrophage inflammatory protein 1 beta.

### CERTAIN DEFINITIONS

Although the present disclosure is described in detail below, it is to be understood that this disclosure is not limited to the particular methodologies, protocols and reagents described herein as these may vary. It is also to be understood that the terminology used herein is for the purpose of describing particular embodiments only, and is not intended to limit the scope of the present disclosure which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, H. G. W. Leuenberger, B. Nagel, and H. Köbl, Eds., Helvetica Chimica Acta, CH-4010 Basel, Switzerland, (1995).

The practice of the present disclosure will employ, unless otherwise indicated, conventional methods of chemistry, biochemistry, cell biology, immunology, and recombinant DNA techniques which are explained in the literature in the field (cf., e.g., Molecular Cloning: A Laboratory Manual, 2nd Edition, J. Sambrook et al. eds., Cold Spring Harbor Laboratory Press, Cold Spring Harbor 1989).

In the following, the elements of the present disclosure will be described. These elements are listed with specific embodiments, however, it should be understood that they may be combined in any manner and in any number to create additional embodiments. The variously described examples and embodiments should not be construed to limit the present disclosure to only the explicitly described embodiments. This description should be understood to disclose and encompass embodiments which combine the explicitly described embodiments with any number of the disclosed elements. Furthermore, any permutations and combinations of all described elements should be considered disclosed by this description unless the context indicates otherwise. The term “about” means approximately or nearly, and in the context of a numerical value or range set forth herein in some embodiments means ±20%, ±10%, ±5%, or ±3% of the numerical value or range recited or claimed.

The terms “a” and “an” and “the” and similar reference used in the context of describing the disclosure (especially in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it was individually recited herein. All methods described herein can be performed in any suitable order unless otherwise indicated herein or otherwise clearly contradicted by context. The use of any and all examples, or exemplary language (e.g., “such as”), provided herein is intended merely to better illustrate the disclosure and does not pose a limitation on the scope of the claims. No language in the specification should be construed as indicating any non-claimed element essential to the practice of the disclosure.

Unless expressly specified otherwise, the term “comprising” is used in the context of the present document to indicate that further members may optionally be present in addition to the members of the list introduced by “comprising”. It is, however, contemplated as a specific embodiment of the present disclosure that the term “comprising” encompasses the possibility of no further members being present, i.e., for the purpose of this embodiment “comprising” is to be understood as having the meaning of “consisting of” or “consisting essentially of”.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the present disclosure was not entitled to antedate such disclosure.

In the following, definitions will be provided which apply to all aspects of the present disclosure. The following terms have the following meanings unless otherwise indicated. Any undefined terms have their art recognized meanings.
Agent: As used herein, the term “agent”, may refer to a physical entity or phenomenon. In some embodiments, an agent may be characterized by a particular feature and/or effect. In some embodiments, an agent may be a compound, molecule, or entity of any chemical class including, for example, a small molecule, polypeptide, nucleic acid, saccharide, lipid, metal, or a combination or complex thereof. In some embodiments, the term “agent” may refer to a compound, molecule, or entity that comprises a polymer. In some embodiments, the term may refer to a compound or entity that comprises one or more polymeric moieties. In some embodiments, the term “agent” may refer to a compound, molecule, or entity that is substantially free of a particular polymer or polymeric moiety. In some embodiments, the term may refer to a compound, molecule, or entity that lacks or is substantially free of any polymer or polymeric moiety.
Amino acid: in its broadest sense, as used herein, the term “amino acid” refers to a compound and/or substance that can be, is, or has been incorporated into a polypeptide chain, e.g., through formation of one or more peptide bonds. In some embodiments, an amino acid has the general structure H₂N—C(H)(R)—COOH. In some embodiments, an amino acid is a naturally-occurring amino acid. In some embodiments, an amino acid is a non-natural amino acid; in some embodiments, an amino acid is a D-amino acid; in some embodiments, an amino acid is an L-amino acid. “Standard amino acid” refers to any of the twenty standard L-amino acids commonly found in naturally occurring peptides. “Nonstandard amino acid” refers to any amino acid, other than the standard amino acids, regardless of whether it is prepared synthetically or obtained from a natural source. In some embodiments, an amino acid, including a carboxy- and/or amino-terminal amino acid in a polypeptide, can contain a structural modification as compared with the general structure above. For example, in some embodiments, an amino acid may be modified by methylation, amidation, acetylation, pegylation, glycosylation, phosphorylation, and/or substitution (e.g., of the amino group, the carboxylic acid group, one or more protons, and/or the hydroxyl group) as compared with the general structure. In some embodiments, such modification may, for example, alter the circulating half-life of a polypeptide containing the modified amino acid as compared with one containing an otherwise identical unmodified amino acid. In some embodiments, such modification does not significantly alter a relevant activity of a polypeptide containing the modified amino acid, as compared with one containing an otherwise identical unmodified amino acid. As will be clear from context, in some embodiments, the term “amino acid” may be used to refer to a free amino acid; in some embodiments it may be used to refer to an amino acid residue of a polypeptide.
Analog: As used herein, the term “analog” refers to a substance that shares one or more particular structural features, elements, components, or moieties with a reference substance. Typically, an “analog” shows significant structural similarity with the reference substance, for example sharing a core or consensus structure, but also differs in certain discrete ways. In some embodiments, an analog is a substance that can be generated from the reference substance, e.g., by chemical manipulation of the reference substance. In some embodiments, an analog is a substance that can be generated through performance of a synthetic process substantially similar to (e.g., sharing a plurality of steps with) one that generates the reference substance. In some embodiments, an analog is or can be generated through performance of a synthetic process different from that used to generate the reference substance.
Antibody agent: As used herein, the term “antibody agent” refers to an agent that specifically binds to a particular antigen. In some embodiments, the term encompasses a polypeptide or polypeptide complex that includes immunoglobulin structural elements sufficient to confer specific binding. For example, in some embodiments, an antibody agent is or comprises a polypeptide whose amino acid sequence includes one or more structural elements recognized by those skilled in the art as a complementarity determining region (CDR); in some embodiments an antibody agent is or comprises a polypeptide whose amino acid sequence includes at least one CDR (e.g., at least one heavy chain CDR and/or at least one light chain CDR) that is substantially identical to one found in a reference antibody. In some embodiments an included CDR is substantially identical to a reference CDR in that it is either identical in sequence or contains between 1-5 amino acid substitutions as compared with the reference CDR. In some embodiments an included CDR is substantially identical to a reference CDR in that it shows at least 85%, 86%, 87%, 88%, 89%, 90%, 91%, 92%, 93%, 94%, 95%, 96%, 97%, 98%, 99%, or 100% sequence identity with the reference CDR. In some embodiments an included CDR is substantially identical to a reference CDR in that it shows at least 96%, 96%, 97%, 98%, 99%, or 100% sequence identity with the reference CDR. In some embodiments an included CDR is substantially identical to a reference CDR in that at least one amino acid within the included CDR is deleted, added, or substituted as compared with the reference CDR but the included CDR has an amino acid sequence that is otherwise identical with that of the reference CDR. In some embodiments an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments an included CDR is substantially identical to a reference CDR in that at least one amino acid within the included CDR is substituted as compared with the reference CDR but the included CDR has an amino acid sequence that is otherwise identical with that of the reference CDR. In some embodiments an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments, an antibody agent is or comprises a polypeptide whose amino acid sequence includes structural elements recognized by those skilled in the art as an immunoglobulin variable domain. In some embodiments, an antibody agent in or comprises a polypeptide whose amino acid sequence includes structural elements recognized by those skilled in the art to correspond to CDRs 1, 2, and 3 of an antibody variable domain; in some such embodiments, an antibody agent in or comprises a polypeptide or set of polypeptides whose amino acid sequence(s) together include structural elements recognized by those skilled in the art to correspond to both heavy chain and light chain variable region CDRs, e.g., heavy chain CDRs 1, 2, and/or 3 and light chain CDRs 1, 2, and/or 3. In some embodiments, an antibody agent is a polypeptide protein having a binding domain which is homologous or largely homologous to an immunoglobulin-binding domain. In some embodiments, an antibody agent may be or comprise a polyclonal antibody preparation. In some embodiments, an antibody agent may be or comprise a monoclonal antibody preparation. In some embodiments, an antibody agent may include one or more constant region sequences that are characteristic of a particular organism, such as a camel, human, mouse, primate, rabbit, rat; in many embodiments, an antibody agent may include one or more constant region sequences that are characteristic of a human. In some embodiments, an antibody agent may include one or more sequence elements that would be recognized by one skilled in the art as a humanized sequence, a primatized sequence, a chimeric sequence, etc. In some embodiments, an antibody agent may be a canonical antibody (e.g., may comprise two heavy chains and two light chains). In some embodiments, an antibody agent may be in a format selected from, but not limited to, intact IgA, IgG, IgE or IgM antibodies; bi- or multi-specific antibodies (e.g., Zybodies®, etc); antibody fragments such as Fab fragments, Fab′ fragments, F(ab′)2 fragments, Fd′ fragments, Fd fragments, and isolated CDRs or sets thereof, single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPs™”); single chain or Tandem diabodies (TandAb®); VHHs; Anticalins®; Nanobodies® minibodies; BiTE®s; ankyrin repeat proteins or DARPINs®; Avimers®; DARTs; TCR-like antibodies, Adnectins®; Affilins®; Trans-bodies®; Affibodies®; TrimerX®; MicroProteins; Fynomers®, Centyrins®; and KALBITOR®s. In some embodiments, an antibody may lack a covalent modification (e.g., attachment of a glycan) that it would have if produced naturally. In some embodiments, an antibody may contain a covalent modification (e.g., attachment of a glycan, a payload [e.g., a detectable moiety, a therapeutic moiety, a catalytic moiety, etc], or other pendant group [e.g., poly-ethylene glycol, etc.].
Associated: Two events or entities are “associated” with one another, as that term is used herein, if the presence, level, degree, type and/or form of one is correlated with that of the other. For example, a particular entity (e.g., polypeptide, genetic signature, metabolite, microbe, etc) is considered to be associated with a particular disease, disorder, or condition, if its presence, level and/or form correlates with incidence of, susceptibility to, severity of, stage of, etc the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.
Binding: It will be understood that the term “binding”, as used herein, typically refers to a non-covalent association between or among two or more entities. “Direct” binding involves physical contact between entities or moieties; indirect binding involves physical interaction by way of physical contact with one or more intermediate entities. Binding between two or more entities can typically be assessed in any of a variety of contexts—including where interacting entities or moieties are studied in isolation or in the context of more complex systems (e.g., while covalently or otherwise associated with a carrier entity and/or in a biological system or cell). Binding between two entities may be considered “specific” if, under the conditions assessed, the relevant entities are more likely to associate with one another than with other available binding partners.
Biological Sample: As used herein, the term “biological sample” typically refers to a sample obtained or derived from a biological source (e.g., a tissue or organism or cell culture) of interest, as described herein. In some embodiments, a source of interest comprises an organism, such as an animal or human. In some embodiments, a biological sample is or comprises biological tissue or fluid. In some embodiments, a biological sample may be or comprise bone marrow; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as a ductal lavages or broncheoalveolar lavages; aspirates; scrapings; bone marrow specimens; tissue biopsy specimens; surgical specimens; feces, other body fluids, secretions, and/or excretions; and/or cells therefrom, etc. In some embodiments, a biological sample is or comprises cells obtained from an individual. In some embodiments, obtained cells are or include cells from an individual from whom the sample is obtained. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood, lymph, feces etc.), etc. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to techniques such as amplification or reverse transcription of mRNA, isolation and/or purification of certain components, etc.
Combination therapy: As used herein, the term “combination therapy” refers to those situations in which a subject is simultaneously exposed to two or more therapeutic regimens (e.g., two or more therapeutic agents). In some embodiments, the two or more regimens may be administered simultaneously; in some embodiments, such regimens may be administered sequentially (e.g., all “doses” of a first regimen are administered prior to administration of any doses of a second regimen); in some embodiments, such agents are administered in overlapping dosing regimens. In some embodiments, “administration” of combination therapy may involve administration of one or more agent(s) or modality(ies) to a subject receiving the other agent(s) or modality(ies) in the combination. For clarity, combination therapy does not require that individual agents be administered together in a single composition (or even necessarily at the same time), although in some embodiments, two or more agents, or active moieties thereof, may be administered together in a combination composition, or even in a combination compound (e.g., as part of a single chemical complex or covalent entity).
Complementary: As used herein, the term “complementary” is used in reference to oligonucleotide hybridization related by base-pairing rules. For example, the sequence “C-A-G-T” is complementary to the sequence “G-T-C-A.” Complementarity can be partial or total. Thus, any degree of partial complementarity is intended to be included within the scope of the term “complementary” provided that the partial complementarity permits oligonucleotide hybridization. Partial complementarity is where one or more nucleic acid bases is not matched according to the base pairing rules. Total or complete complementarity between nucleic acids is where each and every nucleic acid base is matched with another base under the base pairing rules.
Comparable: As used herein, the term “comparable” refers to two or more agents, entities, situations, sets of conditions, etc., that may not be identical to one another but that are sufficiently similar to permit comparison there between so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.
Corresponding to: As used herein, the term “corresponding to” refers to a relationship between two or more entities. For example, the term “corresponding to” may be used to designate the position/identity of a structural element in a compound or composition relative to another compound or composition (e.g., to an appropriate reference compound or composition). For example, in some embodiments, a monomeric residue in a polymer (e.g., an amino acid residue in a polypeptide or a nucleic acid residue in a polynucleotide) may be identified as “corresponding to” a residue in an appropriate reference polymer. For example, those of ordinary skill will appreciate that, for purposes of simplicity, residues in a polypeptide are often designated using a canonical numbering system based on a reference related polypeptide, so that an amino acid “corresponding to” a residue at position 190, for example, need not actually be the 190^{th} amino acid in a particular amino acid chain but rather corresponds to the residue found at 190 in the reference polypeptide; those of ordinary skill in the art readily appreciate how to identify “corresponding” amino acids. For example, those skilled in the art will be aware of various sequence alignment strategies, including software programs such as, for example, BLAST, CS-BLAST, CUSASW++, DIAMOND, FASTA, GGSEARCH/GLSEARCH, Genoogle, HMMER, HHpred/HHsearch, IDF, Infernal, KLAST, USEARCH, parasail, PSI-BLAST, PSI-Search, ScalaBLAST, Sequilab, SAM, SSEARCH, SWAPHI, SWAPHI-LS, SWIMM, or SWIPE that can be utilized, for example, to identify “corresponding” residues in polypeptides and/or nucleic acids in accordance with the present disclosure. Those of skill in the art will also appreciate that, in some instances, the term “corresponding to” may be used to describe an event or entity that shares a relevant similarity with another event or entity (e.g., an appropriate reference event or entity). To give but one example, a gene or protein in one organism may be described as “corresponding to” a gene or protein from another organism in order to indicate, in some embodiments, that it plays an analogous role or performs an analogous function and/or that it shows a particular degree of sequence identity or homology, or shares a particular characteristic sequence element.
Designed: As used herein, the term “designed” refers to an agent (i) whose structure is or was selected by the hand of man; (ii) that is produced by a process requiring the hand of man; and/or (iii) that is distinct from natural substances and other known agents.
Dosing regimen: Those skilled in the art will appreciate that the term “dosing regimen” may be used to refer to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which is separated in time from other doses. In some embodiments, individual doses are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).
Encode: As used herein, the term “encode” or “encoding” refers to sequence information of a first molecule that guides production of a second molecule having a defined sequence of nucleotides (e.g., mRNA) or a defined sequence of amino acids. For example, a DNA molecule can encode an RNA molecule (e.g., by a transcription process that includes a DNA-dependent RNA polymerase enzyme). An RNA molecule can encode a polypeptide (e.g., by a translation process). Thus, a gene, a cDNA, or a single-stranded RNA (e.g., an mRNA) encodes a polypeptide if transcription and translation of mRNA corresponding to that gene produces the polypeptide in a cell or other biological system. In some embodiments, a coding region of a single-stranded RNA encoding a target polypeptide agent refers to a coding strand, the nucleotide sequence of which is identical to the mRNA sequence of such a target polypeptide agent. In some embodiments, a coding region of a single-stranded RNA encoding a target polypeptide agent refers to a non-coding strand of such a target polypeptide agent, which may be used as a template for transcription of a gene or cDNA.
Engineered: In general, the term “engineered” refers to the aspect of having been manipulated by the hand of man. For example, a polynucleotide is considered to be “engineered” when two or more sequences that are not linked together in that order in nature are manipulated by the hand of man to be directly linked to one another in the engineered polynucleotide and/or when a particular residue in a polynucleotide is non-naturally occurring and/or is caused through action of the hand of man to be linked with an entity or moiety with which it is not linked in nature.
Epitope: as used herein, the term “epitope” refers to a moiety that is specifically recognized by an immunoglobulin (e.g., antibody or receptor) binding component. In some embodiments, an epitope is comprised of a plurality of chemical atoms or groups on an antigen. In some embodiments, such chemical atoms or groups are surface-exposed when the antigen adopts a relevant three-dimensional conformation. In some embodiments, such chemical atoms or groups are physically near to each other in space when the antigen adopts such a conformation. In some embodiments, at least some such chemical atoms are groups are physically separated from one another when the antigen adopts an alternative conformation (e.g., is linearized).
Expression: As used herein, the term “expression” of a nucleic acid sequence refers to the generation of any gene product from the nucleic acid sequence. In some embodiments, a gene product can be a transcript. In some embodiments, a gene product can be a polypeptide. In some embodiments, expression of a nucleic acid sequence involves one or more of the following: (1) production of an RNA template from a DNA sequence (e.g., by transcription); (2) processing of an RNA transcript (e.g., by splicing, editing, etc); (3) translation of an RNA into a polypeptide or protein; and/or (4) post-translational modification of a polypeptide or protein.
Improved, increased or reduced: As used herein, these terms, or grammatically comparable comparative terms, indicate values that are relative to a comparable reference measurement. For example, in some embodiments, an assessed value achieved with an agent of interest may be “improved” relative to that obtained with a comparable reference agent. Alternatively or additionally, in some embodiments, an assessed value achieved in a subject or system of interest may be “improved” relative to that obtained in the same subject or system under different conditions (e.g., prior to or after an event such as administration of an agent of interest), or in a different, comparable subject (e.g., in a comparable subject or system that differs from the subject or system of interest in presence of one or more indicators of a particular disease, disorder or condition of interest, or in prior exposure to a condition or agent, etc.). In some embodiments, comparative terms refer to statistically relevant differences (e.g., that are of a prevalence and/or magnitude sufficient to achieve statistical relevance). Those skilled in the art will be aware, or will readily be able to determine, in a given context, a degree and/or prevalence of difference that is required or sufficient to achieve such statistical significance.
In vitro: The term “in vitro” as used herein refers to events that occur in an artificial environment, e.g., in a test tube or reaction vessel (e.g., a bioreactor), in cell culture, etc., rather than within a multi-cellular organism.
In vitro transcription: As used herein, the term “in vitro transcription” or “IVT” refers to the process whereby transcription occurs in vitro in a non-cellular system to produce a synthetic RNA product for use in various applications, including, e.g., production of protein or polypeptides. Such synthetic RNA products can be translated in vitro or introduced directly into cells, where they can be translated. Such synthetic RNA products include, e.g., but not limited to mRNAs, antisense RNA molecules, shRNA molecules, long non-coding RNA molecules, ribozymes, aptamers, guide RNAs (e.g., for CRISPR), ribosomal RNAs, small nuclear RNAs, small nucleolar RNAs, and the like. An IVT reaction typically utilizes a DNA template (e.g., a linear DNA template) as described and/or utilized herein, ribonucleotides (e.g., non-modified ribonucleotide triphosphates or modified ribonucleotide triphosphates), and an appropriate RNA polymerase.
Pharmaceutical composition: As used herein, the term “pharmaceutical composition” refers to an active agent, formulated together with one or more pharmaceutically acceptable carriers. In some embodiments, active agent is present in unit dose amount appropriate for administration in a therapeutic regimen that shows a statistically significant probability of achieving a predetermined therapeutic effect when administered to a relevant population. In some embodiments, pharmaceutical compositions may be specially formulated for parenteral administration, for example, by subcutaneous, intramuscular, intravenous or epidural injection as, for example, a sterile solution or suspension, or sustained-release formulation.
Polypeptide: As used herein refers to a polymeric chain of amino acids. In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, a polypeptide may comprise or consist of natural amino acids, non-natural amino acids, or both. In some embodiments, a polypeptide may comprise or consist of only natural amino acids or only non-natural amino acids. In some embodiments, a polypeptide may comprise D-amino acids, L-amino acids, or both. In some embodiments, a polypeptide may comprise only D-amino acids. In some embodiments, a polypeptide may comprise only L-amino acids. In some embodiments, a polypeptide may include one or more pendant groups or other modifications, e.g., modifying or attached to one or more amino acid side chains, at the polypeptide's N-terminus, at the polypeptide's C-terminus, or any combination thereof. In some embodiments, such pendant groups or modifications may be selected from the group consisting of acetylation, amidation, lipidation, methylation, pegylation, etc., including combinations thereof. In some embodiments, a polypeptide may be cyclic, and/or may comprise a cyclic portion. In some embodiments, a polypeptide is not cyclic and/or does not comprise any cyclic portion. In some embodiments, a polypeptide is linear. In some embodiments, a polypeptide may be or comprise a stapled polypeptide. In some embodiments, the term “polypeptide” may be appended to a name of a reference polypeptide, activity, or structure; in such instances it is used herein to refer to polypeptides that share the relevant activity or structure and thus can be considered to be members of the same class or family of polypeptides. For each such class, the present specification provides and/or those skilled in the art will be aware of exemplary polypeptides within the class whose amino acid sequences and/or functions are known; in some embodiments, such exemplary polypeptides are reference polypeptides for the polypeptide class or family. In some embodiments, a member of a polypeptide class or family shows significant sequence homology or identity with, shares a common sequence motif (e.g., a characteristic sequence element) with, and/or shares a common activity (in some embodiments at a comparable level or within a designated range) with a reference polypeptide of the class; in some embodiments with all polypeptides within the class). For example, in some embodiments, a member polypeptide shows an overall degree of sequence homology or identity with a reference polypeptide that is at least about 30-40%, and is often greater than about 50%, 60%, 70%, 80%, 90%, 91%, 92%, 93%, 94%, 95%, 96%, 97%, 98%, 99% or more and/or includes at least one region (e.g., a conserved region that may in some embodiments be or comprise a characteristic sequence element) that shows very high sequence identity, often greater than 90% or even 95%, 96%, 97%, 98%, or 99%. Such a conserved region usually encompasses at least 3-4 and often up to 20 or more amino acids; in some embodiments, a conserved region encompasses at least one stretch of at least 2, 3, 4, 5, 6, 7, 8, 9, 10, 11, 12, 13, 14, 15 or more contiguous amino acids. In some embodiments, a relevant polypeptide may comprise or consist of a fragment of a parent polypeptide.
Prevent or prevention: as used herein when used in connection with the occurrence of a disease, disorder, and/or condition, refers to reducing the risk of developing the disease, disorder and/or condition and/or to delaying onset of one or more characteristics or symptoms of the disease, disorder or condition. Prevention may be considered complete when onset of a disease, disorder or condition has been delayed for a predefined period of time.
Pure or Purified: As used herein, an agent or entity is “pure” or “purified” if it is substantially free of other components. For example, a preparation that contains more than about 90% of a particular agent or entity is typically considered to be a pure preparation. In some embodiments, an agent or entity is at least 91%, at least 92%, at least 93%, at least 94%, at least 95%, at least 96%, at least 97%, at least 98%, or at least 99% pure in a preparation.
Reference: As used herein describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.
Ribonucleotide: As used herein, the term “ribonucleotide” encompasses unmodified ribonucleotides and modified ribonucleotides. For example, unmodified ribonucleotides include the purine bases adenine (A) and guanine (G), and the pyrimidine bases cytosine (C) and uracil (U). Modified ribonucleotides may include one or more modifications including, but not limited to, for example, (a) end modifications, e.g., 5′ end modifications (e.g., phosphorylation, dephosphorylation, conjugation, inverted linkages, etc.), 3′ end modifications (e.g., conjugation, inverted linkages, etc.), (b) base modifications, e.g., replacement with modified bases, stabilizing bases, destabilizing bases, or bases that base pair with an expanded repertoire of partners, or conjugated bases, (c) sugar modifications (e.g., at the 2′ position or 4′ position) or replacement of the sugar, and (d) internucleoside linkage modifications, including modification or replacement of the phosphodiester linkages. The term “ribonucleotide” also encompasses ribonucleotide triphosphates including modified and non-modified ribonucleotide triphosphates.
Risk: as will be understood from context, “risk” of a disease, disorder, and/or condition refers to a likelihood that a particular individual will develop the disease, disorder, and/or condition. In some embodiments, risk is expressed as a percentage. In some embodiments, risk is from 0, 1, 2, 3, 4, 5, 6, 7, 8, 9, 10, 20, 30, 40, 50, 60, 70, 80, 90 up to 100%. In some embodiments risk is expressed as a risk relative to a risk associated with a reference sample or group of reference samples. In some embodiments, a reference sample or group of reference samples have a known risk of a disease, disorder, condition and/or event. In some embodiments a reference sample or group of reference samples are from individuals comparable to a particular individual. In some embodiments, relative risk is 0, 1, 2, 3, 4, 5, 6, 7, 8, 9, 10, or more. In some embodiments, risk may reflect one or more genetic attributes, e.g., which may predispose an individual toward development (or not) of a particular disease, disorder and/or condition. In some embodiments, risk may reflect one or more epigenetic events or attributes and/or one or more lifestyle or environmental events or attributes.
Susceptible to: An individual who is “susceptible to” a disease, disorder, and/or condition is one who has a higher risk of developing the disease, disorder, and/or condition than does a member of the general public. In some embodiments, an individual who is susceptible to a disease, disorder and/or condition may not have been diagnosed with the disease, disorder, and/or condition. In some embodiments, an individual who is susceptible to a disease, disorder, and/or condition may exhibit symptoms of the disease, disorder, and/or condition. In some embodiments, an individual who is susceptible to a disease, disorder, and/or condition may not exhibit symptoms of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.
Vaccination: As used herein, the term “vaccination” refers to the administration of a composition intended to generate an immune response, for example to a disease-associated (e.g., disease-causing) agent. In some embodiments, vaccination can be administered before, during, and/or after exposure to a disease-associated agent, and in certain embodiments, before, during, and/or shortly after exposure to the agent. In some embodiments, vaccination includes multiple administrations, appropriately spaced in time, of a vaccine composition. In some embodiments, vaccination generates an immune response to an infectious agent. In some embodiments, vaccination generates an immune response to a tumor; in some such embodiments, vaccination is “personalized” in that it is partly or wholly directed to epitope(s) (e.g., which may be or include one or more neoepitopes) determined to be present in a particular individual's tumors.
Variant: As used herein in the context of molecules, e.g., nucleic acids, proteins, or small molecules, the term “variant” refers to a molecule that shows significant structural identity with a reference molecule but differs structurally from the reference molecule, e.g., in the presence or absence or in the level of one or more chemical moieties as compared to the reference entity. In some embodiments, a variant also differs functionally from its reference molecule. In general, whether a particular molecule is properly considered to be a “variant” of a reference molecule is based on its degree of structural identity with the reference molecule. As will be appreciated by those skilled in the art, any biological or chemical reference molecule has certain characteristic structural elements. A variant, by definition, is a distinct molecule that shares one or more such characteristic structural elements but differs in at least one aspect from the reference molecule. In some embodiments, a variant polypeptide or nucleic acid may differ from a reference polypeptide or nucleic acid as a result of one or more differences in amino acid or nucleotide sequence and/or one or more differences in chemical moieties (e.g., carbohydrates, lipids, phosphate groups) that are covalently components of the polypeptide or nucleic acid (e.g., that are attached to the polypeptide or nucleic acid backbone). In some embodiments, a variant polypeptide or nucleic acid shows an overall sequence identity with a reference polypeptide or nucleic acid that is at least 85%, 86%, 87%, 88%, 89%, 90%, 91%, 92%, 93%, 94%, 95%, 96%, 97%, or 99%. In some embodiments, a variant polypeptide or nucleic acid does not share at least one characteristic sequence element with a reference polypeptide or nucleic acid. In some embodiments, a reference polypeptide or nucleic acid has one or more biological activities. In some embodiments, a variant polypeptide or nucleic acid shares one or more of the biological activities of the reference polypeptide or nucleic acid. In some embodiments, a variant polypeptide or nucleic acid lacks one or more of the biological activities of the reference polypeptide or nucleic acid. In some embodiments, a variant polypeptide or nucleic acid shows a reduced level of one or more biological activities as compared to the reference polypeptide or nucleic acid. In some embodiments, a polypeptide or nucleic acid of interest is considered to be a “variant” of a reference polypeptide or nucleic acid if it has an amino acid or nucleotide sequence that is identical to that of the reference but for a small number of sequence alterations at particular positions. Typically, fewer than about 20%, about 15%, about 10%, about 9%, about 8%, about 7%, about 6%, about 5%, about 4%, about 3%, or about 2% of the residues in a variant are substituted, inserted, or deleted, as compared to the reference. In some embodiments, a variant polypeptide or nucleic acid comprises about 10, about 9, about 8, about 7, about 6, about 5, about 4, about 3, about 2, or about 1 substituted residues as compared to a reference. Often, a variant polypeptide or nucleic acid comprises a very small number (e.g., fewer than about 5, about 4, about 3, about 2, or about 1) number of substituted, inserted, or deleted, functional residues (i.e., residues that participate in a particular biological activity) relative to the reference. In some embodiments, a variant polypeptide or nucleic acid comprises not more than about 5, about 4, about 3, about 2, or about 1 addition or deletion, and, in some embodiments, comprises no additions or deletions, as compared to the reference. In some embodiments, a variant polypeptide or nucleic acid comprises fewer than about 25, about 20, about 19, about 18, about 17, about 16, about 15, about 14, about 13, about 10, about 9, about 8, about 7, about 6, and commonly fewer than about 5, about 4, about 3, or about 2 additions or deletions as compared to the reference. In some embodiments, a reference polypeptide or nucleic acid is one found in nature.

### DETAILED DESCRIPTION OF CERTAIN EMBODIMENTS

The present disclosure provides, among other things, an RNA polynucleotide comprising (i) a 5′ cap; (ii) a 5′ UTR sequence comprising a cap proximal sequence, e.g., as disclosed herein; and (iii) a sequence encoding a payload. Also provided herein are compositions and medical preparations comprising the same, as well as methods of making and using the same. In some embodiments, translation efficiency of an RNA encoding a payload, and/or expression of a payload encoded by an RNA, can be improved with an RNA polynucleotide comprising a 5′ cap comprising a structure disclosed herein; a 5′ UTR comprising a cap proximal sequence disclosed herein, and a sequence encoding a payload. In some embodiments, absence of a self-hybridizing sequence in an RNA polynucleotide encoding a payload can further improve translation efficiency of an RNA encoding a payload, and/or expression of a payload encoded by an RNA payload.

### RNA Polynucleotides

The term “polynucleotide” or “nucleic acid”, as used herein, refers to DNA and RNA such as genomic DNA, cDNA, mRNA, recombinantly produced and chemically synthesized molecules. A nucleic acid may be single-stranded or double-stranded. RNA includes in vitro transcribed RNA (IVT RNA) or synthetic RNA. According to the invention, a polynucleotide is preferably isolated.

In some embodiments, nucleic acids may be comprised in a vector. The term “vector” as used herein includes any vectors known to the skilled person including plasmid vectors, cosmid vectors, phage vectors such as lambda phage, viral vectors such as retroviral, adenoviral or baculoviral vectors, or artificial chromosome vectors such as bacterial artificial chromosomes (BAC), yeast artificial chromosomes (YAC), or P1 artificial chromosomes (PAC). In some embodiments, a vector may be an expression vector; alternatively or additionally, in some embodiments, a vector may be a cloning vector. Those skilled in the art will appreciate that, in some embodiments, an expression vector may be, for example, a plasmid; alternatively or additionally, in some embodiments, an expression vector may be a viral vector. Typically, an expression vector will contain a desired coding sequence and appropriate other sequences necessary for the expression of the operably linked coding sequence in a particular host organism (e.g., bacteria, yeast, plant, insect, or mammal) or in in vitro expression systems. Cloning vectors are generally used to engineer and amplify a certain desired fragment (typically a DNA fragment), and may lack functional sequences needed for expression of the desired fragment(s).

In some embodiments, a nucleic acid as described and/or utilized herein may be or comprise recombinant and/or isolated molecules.

Those skilled in the art, reading the present disclosure, will understand that the term “RNA” typically refers to a nucleic acid molecule which includes ribonucleotide residues. In some embodiments, an RNA contains all or a majority of ribonucleotide residues. As used herein, “ribonucleotide” refers to a nucleotide with a hydroxyl group at the 2′-position of a 3-D-ribofuranosyl group. In some embodiments, an RNA may be partly or fully double stranded RNA; in some embodiments, an RNA may comprise two or more distinct nucleic acid strands (e.g., separate molecules) that are partly or fully hybridized with one another. In many embodiments, an RNA is a single strand, which may in some embodiments, self-hybridize or otherwise fold into secondary and/or tertiary structures. In some embodiments, an RNA as described and/or utilized herein does not self-hybridize, at least with respect to certain sequences as described herein. In some embodiments, an RNA may be an isolated RNA such as partially purified RNA, essentially pure RNA, synthetic RNA, recombinantly produced RNA, and/or a modified RNA (where the term “modified” is understood to indicate that one or more residues or other structural elements of the RNA differs from naturally occurring RNA; for example, in some embodiments, a modified RNA differs by the addition, deletion, substitution and/or alteration of one or more nucleotides and/or by one or more moieties or characteristics of a nucleotide—e.g., of a nucleoside or of a backbone structure or linkage). In some embodiments, a modification may be or comprise addition of non-nucleotide material to internal RNA nucleotides or to the end(s) of RNA. It is also contemplated herein that nucleotides in RNA (e.g., in a modified RNA) may be non-standard nucleotides, such as chemically synthesized nucleotides or deoxynucleotides. For the present disclosure, these altered RNAs are considered analogs of naturally-occurring RNA.

As appreciated by a skilled artisan in the art, the RNA polynucleotides disclosed herein can comprise or consist of naturally occurring ribonucleotides and/or modified ribonucleotides. Therefore, a skilled artisan in the art will understand references to A, U, G, or C throughout the specification described herein can refer to a naturally occurring ribonucleotide and/or a modified ribonucleotide described herein. For example, in some embodiments, a U is uridine. In some embodiments, a U is modified uridine (e.g., pseudouridine, 1-methyl pseudouridine).

In some embodiments of the present disclosure, an RNA is or comprises messenger RNA (mRNA) that relates to an RNA transcript which encodes a polypeptide.

In some embodiments, an RNA disclosed herein comprises: a 5′ cap comprising a 5′ cap disclosed herein; a 5′ untranslated region comprising a cap proximal sequence (5′-UTR), a sequence encoding a payload (e.g., a polypeptide); a 3′ untranslated region (3′-UTR); and/or a polyadenylate (PolyA) sequence.

In some embodiments, an RNA disclosed herein comprises the following components in 5′ to 3′ orientation: a 5′ cap comprising a 5′ cap disclosed herein; a 5′ untranslated region comprising a cap proximal sequence (5′-UTR), a sequence encoding a payload (e.g., a polypeptide); a 3′ untranslated region (3′-UTR); and a PolyA sequence.

In some embodiments, an RNA is produced by in vitro transcription or chemical synthesis. In some embodiments, an mRNA is produced by in vitro transcription using a DNA template where DNA refers to a nucleic acid that contains deoxyribonucleotides.

In some embodiments, an RNA disclosed herein is in vitro transcribed RNA (IVT-RNA) and may be obtained by in vitro transcription of an appropriate DNA template. The promoter for controlling transcription can be any promoter for any RNA polymerase. A DNA template for in vitro transcription may be obtained by cloning of a nucleic acid, in particular cDNA, and introducing it into an appropriate vector for in vitro transcription. The cDNA may be obtained by reverse transcription of RNA.

In some embodiments, an RNA is “replicon RNA” or simply a “replicon”, in particular “self-replicating RNA” or “self-amplifying RNA”. In some embodiments, a replicon or self-replicating RNA is derived from or comprises elements derived from a ssRNA virus, in particular a positive-stranded ssRNA virus such as an alphavirus. Alphaviruses are typical representatives of positive-stranded RNA viruses. Alphaviruses replicate in the cytoplasm of infected cells (for review of the alphaviral life cycle see Jose et al., Future Microbiol., 2009, vol. 4, pp. 837-856). The total genome length of many alphaviruses typically ranges between 11,000 and 12,000 nucleotides, and the genomic RNA typically has a 5′-cap, and a 3′ poly(A) tail. The genome of alphaviruses encodes non-structural proteins (involved in transcription, modification and replication of viral RNA and in protein modification) and structural proteins (forming the virus particle). There are typically two open reading frames (ORFs) in the genome. The four non-structural proteins (nsP1-nsP4) are typically encoded together by a first ORF beginning near the 5′ terminus of the genome, while alphavirus structural proteins are encoded together by a second ORF which is found downstream of the first ORF and extends near the 3′ terminus of the genome. Typically, the first ORF is larger than the second ORF, the ratio being roughly 2:1. In cells infected by an alphavirus, only the nucleic acid sequence encoding non-structural proteins is translated from the genomic RNA, while the genetic information encoding structural proteins is translatable from a subgenomic transcript, which is an RNA polynucleotide that resembles eukaryotic messenger RNA (mRNA; Gould et al., 2010, Antiviral Res., vol. 87 pp. 111-124). Following infection, i.e. at early stages of the viral life cycle, the (+) stranded genomic RNA directly acts like a messenger RNA for the translation of the open reading frame encoding the non-structural poly-protein (nsP1234). Alphavirus-derived vectors have been proposed for delivery of foreign genetic information into target cells or target organisms. In simple approaches, the open reading frame encoding alphaviral structural proteins is replaced by an open reading frame encoding a protein of interest. Alphavirus-based trans-replication systems rely on alphavirus nucleotide sequence elements on two separate nucleic acid molecules: one nucleic acid molecule encodes a viral replicase, and the other nucleic acid molecule is capable of being replicated by said replicase in trans (hence the designation trans-replication system). Trans-replication requires the presence of both these nucleic acid molecules in a given host cell. The nucleic acid molecule capable of being replicated by the replicase in trans must comprise certain alphaviral sequence elements to allow recognition and RNA synthesis by the alphaviral replicase.

In some embodiments, an RNA described herein may have modified nucleosides. In some embodiments, an RNA comprises a modified nucleoside in place of at least one (e.g., every) uridine.

The term “uracil,” as used herein, describes one of the nucleobases that can occur in the nucleic acid of RNA. The structure of uracil is:

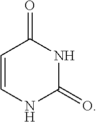

The term “uridine,” as used herein, describes one of the nucleosides that can occur in RNA. The structure of uridine is:

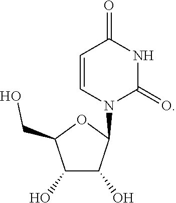 [SMILES: O=c1ccn([C@@H]2O[C@H](CO)[C@@H](O)[C@H]2O)c(=O)[nH]1]

UTP (uridine 5′-triphosphate) has the following structure:

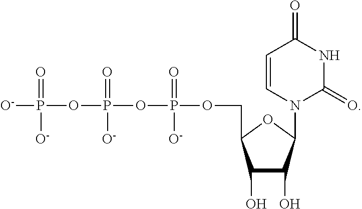

Pseudo-UTP (pseudouridine-5′-triphosphate) has the following structure:

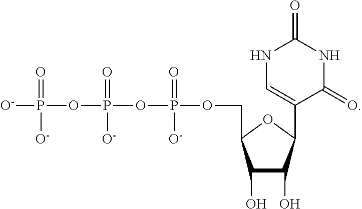

“Pseudouridine” is one example of a modified nucleoside that is an isomer of uridine, where the uracil is attached to the pentose ring via a carbon-carbon bond instead of a nitrogen-carbon glycosidic bond.

Another exemplary modified nucleoside is N1-methylpseudouridine (m1Ψ), which has the structure:

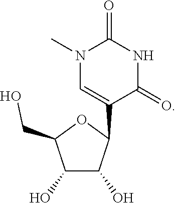 [SMILES: Cn1cc([C@@H]2O[C@H](CO)[C@@H](O)[C@H]2O)c(=O)[nH]c1=O]

N1-methylpseudouridine-5′-triphosphate (m1ΨTP) has the following structure:

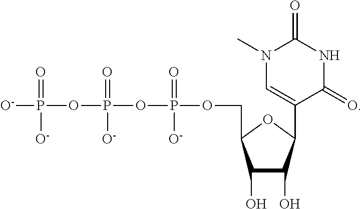

Another exemplary modified nucleoside is 5-methyluridine (m5U), which has the structure:

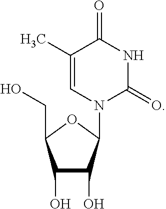

In some embodiments, one or more uridine in an RNA described herein is replaced by a modified nucleoside. In some embodiments, a modified nucleoside is a modified uridine. In some embodiments, an RNA comprises a modified nucleoside in place of at least one uridine. In some embodiments, an RNA comprises a modified nucleoside in place of each uridine.

In some embodiments, a modified nucleoside is independently selected from pseudouridine (Ψ), N1-methylpseudouridine (m1Ψ), and 5-methyluridine (m5U). In some embodiments, a modified nucleoside comprises pseudouridine (Ψ). In some embodiments, a modified nucleoside comprises N1-methyl-pseudouridine (m1Ψ). In some embodiments, a modified nucleoside comprises 5-methyluridine (m5U). In some embodiments, an RNA may comprise more than one type of modified nucleoside, and a modified nucleosides are independently selected from pseudouridine (Ψ), N1-methylpseudouridine (m1Ψ), and 5-methyluridine (m5U). In some embodiments, a modified nucleosides comprise pseudouridine (Ψ) and N1-methylpseudouridine (m1Ψ). In some embodiments, a modified nucleosides comprise pseudouridine (Ψ) and 5-methyluridine (m5U). In some embodiments, a modified nucleosides comprise N1-methylpseudouridine (m1Ψ) and 5-methyluridine (m5U). In some embodiments, a modified nucleosides comprise pseudouridine (Ψ), N1-methylpseudouridine (m1Ψ), and 5-methyluridine (m5U).

In some embodiments, a modified nucleoside replacing one or more, e.g., all, uridine in the RNA may be any one or more of 3-methyl-uridine (m³U), 5-methoxy-uridine (mo⁵U), 5-aza-uridine, 6-aza-uridine, 2-thio-5-aza-uridine, 2-thio-uridine (s²U), 4-thio-uridine (s⁴U), 4-thio-pseudouridine, 2-thio-pseudouridine, 5-hydroxy-uridine (ho⁵U), 5-aminoallyl-uridine, 5-halo-uridine (e.g., 5-iodo-uridine or 5-bromo-uridine), uridine 5-oxyacetic acid (cmo⁵U), uridine 5-oxyacetic acid methyl ester (mcmo⁵U), 5-carboxymethyl-uridine (cm⁵U), 1-carboxymethyl-pseudouridine, 5-carboxyhydroxymethyl-uridine (chm⁵U), 5-carboxyhydroxymethyl-uridine methyl ester (mchm⁵U), 5-methoxycarbonylmethyl-uridine (mcm⁵U), 5-methoxycarbonylmethyl-2-thio-uridine (mcm⁵s²U), 5-aminomethyl-2-thio-uridine (nm⁵s²U), 5-methylaminomethyl-uridine (mnm⁵U), 1-ethyl-pseudouridine, 5-methylaminomethyl-2-thio-uridine (mnm⁵s²U), 5-methylaminomethyl-2-seleno-uridine (mnm⁵se²U), 5-carbamoylmethyl-uridine (ncm⁵U), 5-carboxymethylaminomethyl-uridine (cmnm⁵U), 5-carboxymethylaminomethyl-2-thio-uridine (cmnm⁵s²U), 5-propynyl-uridine, 1-propynyl-pseudouridine, 5-taurinomethyl-uridine (τm⁵U), 1-taurinomethyl-pseudouridine, 5-taurinomethyl-2-thio-uridine(τm5s2U), 1-taurinomethyl-4-thio-pseudouridine), 5-methyl-2-thio-uridine (m⁵s²U), 1-methyl-4-thio-pseudouridine (m¹s⁴Ψ), 4-thio-1-methyl-pseudouridine, 3-methyl-pseudouridine (m³Ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m⁵D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl)uridine (acp³U), 1-methyl-3-(3-amino-3-carboxypropyl)pseudouridine (acp³Ψ), 5-(isopentenylaminomethyl)uridine (inm⁵U), 5-(isopentenylaminomethyl)-2-thio-uridine (inm⁵s²U), α-thio-uridine, 2′-O-methyl-uridine (Um), 5,2′-O-dimethyl-uridine (m⁵Um), 2′-O-methyl-pseudouridine (Ψm), 2-thio-2′-O-methyl-uridine (s²Um), 5-methoxycarbonylmethyl-2′-O-methyl-uridine (mcm⁵Um), 5-carbamoylmethyl-2′-O-methyl-uridine (ncm⁵Um), 5-carboxymethylaminomethyl-2′-O-methyl-uridine (cmnm⁵Um), 3,2′-O-dimethyl-uridine (m³Um), 5-(isopentenylaminomethyl)-2′-O-methyl-uridine (inm⁵Um), 1-thio-uridine, deoxythymidine, 2′-F-ara-uridine, 2′-F-uridine, 2′-OH-ara-uridine, 5-(2-carbomethoxyvinyl) uridine, 5-[3-(1-E-propenylamino)uridine, or any other modified uridine known in the art.

In some embodiments, an RNA comprises other modified nucleosides or comprises further modified nucleosides, e.g., modified cytidine. For example, in some embodiments, in an RNA 5-methylcytidine is substituted partially or completely, preferably completely, for cytidine. In some embodiments, an RNA comprises 5-methylcytidine and one or more selected from pseudouridine (Ψ), N1-methyl-pseudouridine (m1Ψ), and 5-methyl-uridine (m5U). In some embodiments, an RNA comprises 5-methylcytidine and N1-methyl-pseudouridine (m1Ψ). In some embodiments, the RNA comprises 5-methylcytidine in place of each cytidine and N1-methyl-pseudouridine (m1Ψ) in place of each uridine.

In some embodiments, an RNA encoding a payload, e.g., a vaccine antigen, is expressed in cells of a subject treated to provide a payload, e.g., vaccine antigen. In some embodiments, the RNA is transiently expressed in cells of the subject. In some embodiments, the RNA is in vitro transcribed RNA. In some embodiments, expression of a payload, e.g., a vaccine antigen is at the cell surface. In some embodiments, a payload, e.g., a vaccine antigen is expressed and presented in the context of MHC. In some embodiments, expression of a payload, e.g., a vaccine antigen is into the extracellular space, i.e., the vaccine antigen is secreted.

In the context of the present disclosure, the term “transcription” relates to a process, wherein the genetic code in a DNA sequence is transcribed into RNA. Subsequently, the RNA may be translated into peptide or protein.

According to the present invention, the term “transcription” comprises “in vitro transcription”, wherein the term “in vitro transcription” relates to a process wherein RNA, in particular mRNA, is in vitro synthesized in a cell-free system, preferably using appropriate cell extracts. Preferably, cloning vectors are applied for the generation of transcripts. These cloning vectors are generally designated as transcription vectors and are according to the present invention encompassed by the term “vector”. According to the present invention, the RNA used in the present invention preferably is in vitro transcribed RNA (IVT-RNA) and may be obtained by in vitro transcription of an appropriate DNA template. The promoter for controlling transcription can be any promoter for any RNA polymerase. Particular examples of RNA polymerases are the T7, T3, and SP6 RNA polymerases. Preferably, the in vitro transcription according to the invention is controlled by a T7 or SP6 promoter. A DNA template for in vitro transcription may be obtained by cloning of a nucleic acid, in particular cDNA, and introducing it into an appropriate vector for in vitro transcription. The cDNA may be obtained by reverse transcription of RNA.

With respect to RNA, the term “expression” or “translation” relates to the process in the ribosomes of a cell by which a strand of mRNA directs the assembly of a sequence of amino acids to make a peptide or protein.

In some embodiments, after administration of an RNA described herein, e.g., formulated as RNA lipid particles, at least a portion of the RNA is delivered to a target cell. In some embodiments, at least a portion of the RNA is delivered to the cytosol of the target cell. In some embodiments, the RNA is translated by the target cell to produce the peptide or protein it encodes. In some embodiments, the target cell is a spleen cell. In some embodiments, the target cell is an antigen presenting cell such as a professional antigen presenting cell in the spleen. In some embodiments, the target cell is a dendritic cell or macrophage. RNA particles such as RNA lipid particles described herein may be used for delivering RNA to such target cell. Accordingly, the present disclosure also relates to a method for delivering RNA to a target cell in a subject comprising the administration of the RNA particles described herein to the subject. In some embodiments, the RNA is delivered to the cytosol of the target cell. In some embodiments, the RNA is translated by the target cell to produce the peptide or protein encoded by the RNA. “Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

In some embodiments, nucleic acid compositions described herein, e.g., compositions comprising a lipid nanoparticle encapsulated mRNA are characterized by (e.g., when administered to a subject) sustained expression of an encoded polypeptide. For example, in some embodiments, such compositions are characterized in that, when administered to a human, they achieve detectable polypeptide expression in a biological sample (e.g., serum) from such human and, in some embodiments, such expression persists for a period of time that is at least at least 36 hours or longer, including, e.g., at least 48 hours, at least 60 hours, at least 72 hours, at least 96 hours, at least 120 hours, at least 148 hours, or longer.

In some embodiments, an RNA encoding a payload to be administered according to the present disclosure is non-immunogenic. RNA encoding immunostimulant may be administered according to the invention to provide an adjuvant effect. The RNA encoding immunostimulant may be standard RNA or non-immunogenic RNA.

The term “non-immunogenic RNA” as used herein refers to RNA that does not induce a response by the immune system upon administration, e.g., to a mammal, or induces a weaker response than would have been induced by the same RNA that differs only in that it has not been subjected to the modifications and treatments that render the non-immunogenic RNA non-immunogenic, i.e., than would have been induced by standard RNA (stdRNA). In one preferred embodiment, non-immunogenic RNA, which is also termed modified RNA (modRNA) herein, is rendered non-immunogenic by incorporating modified nucleosides suppressing RNA-mediated activation of innate immune receptors into the RNA and removing double-stranded RNA (dsRNA).

For rendering the non-immunogenic RNA non-immunogenic by the incorporation of modified nucleosides, any modified nucleoside may be used as long as it lowers or suppresses immunogenicity of the RNA. Particularly preferred are modified nucleosides that suppress RNA-mediated activation of innate immune receptors. In some embodiments, the modified nucleosides comprises a replacement of one or more uridines with a nucleoside comprising a modified nucleobase. In some embodiments, the modified nucleobase is a modified uracil. In some embodiments, the nucleoside comprising a modified nucleobase is selected from the group consisting of 3-methyl-uridine (m³U), 5-methoxy-uridine (mo⁵U), 5-aza-uridine, 6-aza-uridine, 2-thio-5-aza-uridine, 2-thio-uridine (s²U), 4-thio-uridine (s⁴U), 4-thio-pseudouridine, 2-thio-pseudouridine, 5-hydroxy-uridine (ho⁵U), 5-aminoallyl-uridine, 5-halo-uridine (e.g., 5-iodo-uridine or 5-bromo-uridine), uridine 5-oxyacetic acid (cmo⁵U), uridine 5-oxyacetic acid methyl ester (mcmo⁵U), 5-carboxymethyl-uridine (cm⁵U), 1-carboxymethyl-pseudouridine, 5-carboxyhydroxymethyl-uridine (chm⁵U), 5-carboxyhydroxymethyl-uridine methyl ester (mchm⁵U), 5-methoxycarbonylmethyl-uridine (mcm^{S}U), 5-methoxycarbonylmethyl-2-thio-uridine (mcm⁵s²U), 5-aminomethyl-2-thio-uridine (nm⁵s²U), 5-methylaminomethyl-uridine (mnm⁵U), 1-ethyl-pseudouridine, 5-methylaminomethyl-2-thio-uridine (mnm⁵s²U), 5-methylaminomethyl-2-seleno-uridine (mnm⁵se²U), 5-carbamoylmethyl-uridine (ncm⁵U), 5-carboxymethylaminomethyl-uridine (cmnm⁵U), 5-carboxymethylaminomethyl-2-thio-uridine (cmnm⁵s²U), 5-propynyl-uridine, 1-propynyl-pseudouridine, 5-taurinomethyl-uridine (τm⁵U), 1-taurinomethyl-pseudouridine, 5-taurinomethyl-2-thio-uridine(τm⁵s²U), 1-taurinomethyl-4-thio-pseudouridine), 5-methyl-2-thio-uridine (m⁵s²U), 1-methyl-4-thio-pseudouridine (m¹s⁴ψ), 4-thio-1-methyl-pseudouridine, 3-methyl-pseudouridine (m³ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m⁵D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl)uridine (acp³U), 1-methyl-3-(3-amino-3-carboxypropyl)pseudouridine (acp³ψ), 5-(isopentenylaminomethyl)uridine (inm⁵U), 5-(isopentenylaminomethyl)-2-thio-uridine (inm⁵s²U), α-thio-uridine, 2′-O-methyl-uridine (Um), 5,2′-O-dimethyl-uridine (m⁵Um), 2′-O-methyl-pseudouridine (ψm), 2-thio-2′-O-methyl-uridine (s²Um), 5-methoxycarbonylmethyl-2′-O-methyl-uridine (mcm⁵Um), 5-carbamoylmethyl-2′-O-methyl-uridine (ncm⁵Um), 5-carboxymethylaminomethyl-2′-O-methyl-uridine (cmnm⁵Um), 3,2′-O-dimethyl-uridine (m³Um), 5-(isopentenylaminomethyl)-2′-O-methyl-uridine (inm⁵Um), 1-thio-uridine, deoxythymidine, 2′-F-ara-uridine, 2′-F-uridine, 2′-OH-ara-uridine, 5-(2-carbomethoxyvinyl) uridine, and 5-[3-(1-E-propenylamino)uridine. In one particularly preferred embodiment, the nucleoside comprising a modified nucleobase is pseudouridine (ψ), N1-methyl-pseudouridine (m1ψ) or 5-methyl-uridine (m5U), in particular N1-methyl-pseudouridine.

In some embodiments, the replacement of one or more uridines with a nucleoside comprising a modified nucleobase comprises a replacement of at least 1%, at least 2%, at least 3%, at least 4%, at least 5%, at least 10%, at least 25%, at least 50%, at least 75%, at least 90%, at least 95%, at least 96%, at least 97%, at least 98%, at least 99% or 100% of the uridines. During synthesis of mRNA by in vitro transcription (IVT) using T7 RNA polymerase significant amounts of aberrant products, including double-stranded RNA (dsRNA) are produced due to unconventional activity of the enzyme. dsRNA induces inflammatory cytokines and activates effector enzymes leading to protein synthesis inhibition. dsRNA can be removed from RNA such as IVT RNA, for example, by ion-pair reversed phase HPLC using a non-porous or porous C-18 polystyrene-divinylbenzene (PS-DVB) matrix. Alternatively, an enzymatic based method using E. coli RNaseIII that specifically hydrolyzes dsRNA but not ssRNA, thereby eliminating dsRNA contaminants from IVT RNA preparations can be used. Furthermore, dsRNA can be separated from ssRNA by using a cellulose material. In some embodiments, an RNA preparation is contacted with a cellulose material and the ssRNA is separated from the cellulose material under conditions which allow binding of dsRNA to the cellulose material and do not allow binding of ssRNA to the cellulose material.

As the term is used herein, “remove” or “removal” refers to the characteristic of a population of first substances, such as non-immunogenic RNA, being separated from the proximity of a population of second substances, such as dsRNA, wherein the population of first substances is not necessarily devoid of the second substance, and the population of second substances is not necessarily devoid of the first substance. However, a population of first substances characterized by the removal of a population of second substances has a measurably lower content of second substances as compared to the non-separated mixture of first and second substances.

In some embodiments, the removal of dsRNA from non-immunogenic RNA comprises a removal of dsRNA such that less than 10%, less than 5%, less than 4%, less than 3%, less than 2%, less than 1%, less than 0.5%, less than 0.3%, or less than 0.1% of the RNA in the non-immunogenic RNA composition is dsRNA. In some embodiments, the non-immunogenic RNA is free or essentially free of dsRNA. In some embodiments, the non-immunogenic RNA composition comprises a purified preparation of single-stranded nucleoside modified RNA. For example, in some embodiments, the purified preparation of single-stranded nucleoside modified RNA is substantially free of double stranded RNA (dsRNA). In some embodiments, the purified preparation is at least 90%, at least 91%, at least 92%, at least 93%, at least 94%, at least 95%, at least 96%, at least 97%, at least 98%, at least 99%, at least 99.5%, or at least 99.9% single stranded nucleoside modified RNA, relative to all other nucleic acid molecules (DNA, dsRNA, etc.).

In some embodiments, the non-immunogenic RNA is translated in a cell more efficiently than standard RNA with the same sequence. In some embodiments, translation is enhanced by a factor of 2-fold relative to its unmodified counterpart. In some embodiments, translation is enhanced by a 3-fold factor. In some embodiments, translation is enhanced by a 4-fold factor. In some embodiments, translation is enhanced by a 5-fold factor. In some embodiments, translation is enhanced by a 6-fold factor. In some embodiments, translation is enhanced by a 7-fold factor. In some embodiments, translation is enhanced by an 8-fold factor. In some embodiments, translation is enhanced by a 9-fold factor. In some embodiments, translation is enhanced by a 10-fold factor. In some embodiments, translation is enhanced by a 15-fold factor. In some embodiments, translation is enhanced by a 20-fold factor. In some embodiments, translation is enhanced by a 50-fold factor. In some embodiments, translation is enhanced by a 100-fold factor. In some embodiments, translation is enhanced by a 200-fold factor. In some embodiments, translation is enhanced by a 500-fold factor. In some embodiments, translation is enhanced by a 1000-fold factor. In some embodiments, translation is enhanced by a 2000-fold factor. In some embodiments, the factor is 10-1000-fold. In some embodiments, the factor is 10-100-fold. In some embodiments, the factor is 10-200-fold. In some embodiments, the factor is 10-300-fold. In some embodiments, the factor is 10-500-fold. In some embodiments, the factor is 20-1000-fold. In some embodiments, the factor is 30-1000-fold. In some embodiments, the factor is 50-1000-fold. In some embodiments, the factor is 100-1000-fold. In some embodiments, the factor is 200-1000-fold. In some embodiments, translation is enhanced by any other significant amount or range of amounts.

In some embodiments, the non-immunogenic RNA exhibits significantly less innate immunogenicity than standard RNA with the same sequence. In some embodiments, the non-immunogenic RNA exhibits an innate immune response that is 2-fold less than its unmodified counterpart. In some embodiments, innate immunogenicity is reduced by a 3-fold factor. In some embodiments, innate immunogenicity is reduced by a 4-fold factor. In some embodiments, innate immunogenicity is reduced by a 5-fold factor. In some embodiments, innate immunogenicity is reduced by a 6-fold factor. In some embodiments, innate immunogenicity is reduced by a 7-fold factor. In some embodiments, innate immunogenicity is reduced by a 8-fold factor. In some embodiments, innate immunogenicity is reduced by a 9-fold factor. In some embodiments, innate immunogenicity is reduced by a 10-fold factor. In some embodiments, innate immunogenicity is reduced by a 15-fold factor. In some embodiments, innate immunogenicity is reduced by a 20-fold factor. In some embodiments, innate immunogenicity is reduced by a 50-fold factor. In some embodiments, innate immunogenicity is reduced by a 100-fold factor. In some embodiments, innate immunogenicity is reduced by a 200-fold factor. In some embodiments, innate immunogenicity is reduced by a 500-fold factor. In some embodiments, innate immunogenicity is reduced by a 1000-fold factor. In some embodiments, innate immunogenicity is reduced by a 2000-fold factor.

The term “exhibits significantly less innate immunogenicity” refers to a detectable decrease in innate immunogenicity. In some embodiments, the term refers to a decrease such that an effective amount of the non-immunogenic RNA can be administered without triggering a detectable innate immune response. In some embodiments, the term refers to a decrease such that the non-immunogenic RNA can be repeatedly administered without eliciting an innate immune response sufficient to detectably reduce production of the protein encoded by the non-immunogenic RNA. In some embodiments, the decrease is such that the non-immunogenic RNA can be repeatedly administered without eliciting an innate immune response sufficient to eliminate detectable production of the protein encoded by the non-immunogenic RNA. “Immunogenicity” is the ability of a foreign substance, such as RNA, to provoke an immune response in the body of a human or other animal. The innate immune system is the component of the immune system that is relatively unspecific and immediate. It is one of two main components of the vertebrate immune system, along with the adaptive immune system.

As used herein “endogenous” refers to any material from or produced inside an organism, cell, tissue or system.

As used herein, the term “exogenous” refers to any material introduced from or produced outside an organism, cell, tissue or system.

The term “expression” as used herein is defined as the transcription and/or translation of a particular nucleotide sequence.

As used herein, the terms “linked,” “fused”, or “fusion” are used interchangeably. These terms refer to the joining together of two or more elements or components or domains.

In some embodiments, the present disclosure provides an RNA polynucleotide comprising:
a 5′ cap; a cap proximal sequence comprising positions +1, +2, +3, +4, and +5 of the RNA polynucleotide; and a sequence encoding a payload, wherein:
   (i) the 5′ cap is a trinucleotide cap structure comprising N₁pN₂, wherein N₁ is position +1 of the RNA polynucleotide, and N₂ is position +2 of the RNA polynucleotide, and wherein N₁ and N₂ are selected from one of the following combinations: (a) N₁ is C and N₂ is G; (b) N₁ is U and N₂ is G; or (c) N₁ is A and N₂ is G; and
   (ii) the cap proximal sequence comprises:
N₁ and N₂ of the trinucleotide cap structure and a sequence comprising N₃N₄N₅ at positions +3, +4, and +5 respectively of the RNA polynucleotide, wherein N₃ and N₄ are G, and N₅ is selected from: A, C, G, and U.

In some embodiments, the present disclosure provides an RNA polynucleotide comprising:
a 5′ cap; a cap proximal sequence comprising positions +1, +2, +3, +4, and +5 of the RNA polynucleotide; and a sequence encoding a payload, wherein:
   (i) the 5′ cap is a trinucleotide cap structure comprising N₁pN₂, wherein N₁ is position +1 of the RNA polynucleotide, and N₂ is position +2 of the RNA polynucleotide, and wherein N₁ and N₂ are each G; and
   (ii) the cap proximal sequence comprises:
N₁ and N₂ of the trinucleotide cap structure and a sequence comprising N₃N₄N₅ at positions +3, +4, and +5 respectively of the RNA polynucleotide, wherein N₃ is G, and each N₄ and N₅ is selected from: A, C, G, and U.

In some embodiments, the present disclosure provides an RNA polynucleotide comprising:
a 5′ cap; a cap proximal sequence comprising positions +1, +2, +3, +4, and +5 of the RNA polynucleotide; and a sequence encoding a payload, wherein:
   (i) the 5′ cap is a dinucleotide cap structure comprising N₁, wherein N₁ is position +1 of the RNA polynucleotide, and wherein N₁ is G; and
   (ii) the cap proximal sequence comprises:
      N₁ of the dinucleotide cap structure and a sequence comprising N₂N₃N₄N₅ at positions +2, +3, +4, and +5 respectively of the RNA polynucleotide, wherein each N₂ and N₃ is G, and each N₄ and N₅ is selected from: A, C, G, and U.

In some embodiments, the present disclosure provides an RNA polynucleotide comprising:
a 5′ cap; a cap proximal sequence comprising positions +1, +2, +3, +4, and +5 of the RNA polynucleotide; and a sequence encoding a payload, wherein:
   (i) the 5′ cap is a tetranucleotide cap structure comprising N₁pN₂pN₃, wherein N₁ is position +1 of the RNA polynucleotide, N₂ is position +2 of the RNA polynucleotide, and N₃ is position +3 of the polynucleotide, and wherein N₁, N₂, and N₃ are selected from one of the following combinations: (a) N₁ is C, N₂ is G, and N₃ is G; (b) N₁ is U, N₂ is G, and N₃ is G; or (c) N₁ is A, N₂ is G, and N₃ is G; and
   (ii) the cap proximal sequence comprises:
      N₁, N₂, and N₃ of the tetranucleotide cap structure and a sequence comprising N₄N₅ at positions +4 and +5 respectively of the RNA polynucleotide, wherein N₄ is G, and N₅ is selected from: A, C, G, and U.

In some embodiments, the present disclosure provides an RNA polynucleotide comprising:
a 5′ cap; a cap proximal sequence comprising positions +1, +2, +3, +4, and +5 of the RNA polynucleotide; and a sequence encoding a payload, wherein:
   (i) the 5′ cap is a tetranucleotide cap structure comprising N₁pN₂pN₃, wherein N₁ is position +1 of the RNA polynucleotide, N₂ is position +2 of the RNA polynucleotide, and N₃ is position +3 of the polynucleotide, and wherein N₁ is G, N₂ is G, and N₃ is G; and
   (ii) the cap proximal sequence comprises:
      N₁, N₂, and N₃ of the tetranucleotide cap structure and a sequence comprising N₄N₅ at positions +4 and +5 respectively of the RNA polynucleotide, wherein each N₄ and N₅ is selected from: A, C, G, and U.

In some embodiments, the present disclosure provides an RNA polynucleotide comprising:
a 5′ cap; a cap proximal sequence comprising positions +1, +2, +3, +4, and +5 of the RNA polynucleotide; and a sequence encoding a payload, wherein:
   (i) the 5′ cap is a trinucleotide cap structure comprising N₁pN₂, wherein N₁ is position +1 of the RNA polynucleotide, and N₂ is position +2 of the RNA polynucleotide, and wherein N₁ and N₂ are selected from one of the following combinations: (a) N₁ is G and N₂ is G; (b) N₁ is U and N₂ is G; (c) N₁ is A and N₂ is G; or (d) N₁ is C and N₂ is G; and
   (ii) the cap proximal sequence comprises:
N₁ and N₂ of the trinucleotide cap structure and a sequence comprising N₃N₄N₅ at positions +3, +4, and +5 respectively of the RNA polynucleotide, wherein N₃ is C, N₄ is G, and N₅ is selected from: A, C, G, and U.

In some embodiments, the present disclosure provides an RNA polynucleotide comprising:
a 5′ cap; a cap proximal sequence comprising positions +1, +2, +3, +4, and +5 of the RNA polynucleotide; and a sequence encoding a payload, wherein:
   (i) the 5′ cap is a trinucleotide cap structure comprising N₁pN₂, wherein N₁ is position +1 of the RNA polynucleotide, and N₂ is position +2 of the RNA polynucleotide, and wherein N₁ is G and N₂ is C; and
   (ii) the cap proximal sequence comprises:
      N₁ and N₂ of the trinucleotide cap structure and a sequence comprising N₃N₄N₅ at positions +3, +4, and +5 respectively of the RNA polynucleotide, wherein N₃ is G, and each N₄ and N₅ is selected from: A, C, G, and U.

In some embodiments, the present disclosure provides an RNA polynucleotide comprising:
a 5′ cap; a cap proximal sequence comprising positions +1, +2, +3, +4, and +5 of the RNA polynucleotide; and a sequence encoding a payload, wherein:
(i) the 5′ cap is a dinucleotide cap structure comprising N₁, wherein N₁ is position +1 of the RNA polynucleotide, and wherein N₁ is G; and
(ii) the cap proximal sequence comprises: N₁ of the dinucleotide cap structure and a sequence comprising N₂N₃N₄N₅ at positions +2, +3, +4, and +5 respectively of the RNA polynucleotide, wherein N₂ is a pyrimidine (e.g., C or U), and each of N₃, N₄ and N₅ is selected from: A, C, G, and U. In some embodiments N₃ is G or A, and N₄ and N₅ are each separately and independently selected from: A, C, G, and U.

In some embodiments, the present disclosure provides an RNA polynucleotide comprising:
a 5′ cap; a cap proximal sequence comprising positions +1, +2, +3, +4, and +5 of the RNA polynucleotide; and a sequence encoding a payload, wherein:
   (i) the 5′ cap is a dinucleotide cap structure comprising N₁, wherein N₁ is position +1 of the RNA polynucleotide, and wherein N₁ is G; and
   (ii) the cap proximal sequence comprises:
      N₁ of the dinucleotide cap structure and a sequence comprising N₂N₃N₄N₅ at positions +2, +3, +4, and +5 respectively of the RNA polynucleotide, wherein N₂ is C, N₃ is G, and each N₄ and N₅ is selected from: A, C, G, and U.

In some embodiments, the present disclosure provides an RNA polynucleotide comprising:
a 5′ cap; a cap proximal sequence comprising positions +1, +2, +3, +4, and +5 of the RNA polynucleotide; and a sequence encoding a payload, wherein:
   (i) the 5′ cap is a tetranucleotide cap structure comprising N₁pN₂pN₃, wherein N₁ is position +1 of the RNA polynucleotide, N₂ is position +2 of the RNA polynucleotide, and N₃ is position +3 of the polynucleotide, and wherein N₁, N₂, and N₃ are selected from one of the following combinations: (a) N₁ is C, N₂ is G, and N₃ is C; (b) N₁ is U, N₂ is G, and N₃ is C; or (c) N₁ is A, N₂ is G, and N₃ is C; and
   (ii) the cap proximal sequence comprises:
      N₁, N₂, and N₃ of the tetranucleotide cap structure and a sequence comprising N₄N₅ at positions +4 and +5 respectively of the RNA polynucleotide, wherein N₄ is G, and N₅ is selected from: A, C, G, and U.

In some embodiments, the present disclosure provides an RNA polynucleotide comprising:
a 5′ cap; a cap proximal sequence comprising positions +1, +2, +3, +4, and +5 of the RNA polynucleotide; and a sequence encoding a payload, wherein:
   (i) the 5′ cap is a tetranucleotide cap structure comprising N₁pN₂pN₃, wherein N₁ is position +1 of the RNA polynucleotide, N₂ is position +2 of the RNA polynucleotide, and N₃ is position +3 of the polynucleotide, and wherein N₁ is G, N₂ is C, and N₃ is G; and
   (ii) the cap proximal sequence comprises:
      N₁, N₂, and N₃ of the tetranucleotide cap structure and a sequence comprising N₄N₅ at positions +4 and +5 respectively of the RNA polynucleotide, wherein each N₄ and N₅ is selected from: A, C, G, and U.

In some embodiments, the present disclosure provides an RNA polynucleotide comprising:
a 5′ cap; a cap proximal sequence comprising positions +1, +2, +3, +4, and +5 of the RNA polynucleotide; and a sequence encoding a payload, wherein:
   (i) the 5′ cap is a trinucleotide cap structure comprising N₁pN₂, wherein N₁ is position +1 of the RNA polynucleotide, and N₂ is position +2 of the RNA polynucleotide, and wherein N₁ and N₂ are selected from one of the following combinations: (a) N₁ is G and N₂ is C; (b) N₁ is U and N₂ is C; (c) N₁ is A and N₂ is C; or (d) N₁ is C and N₂ is C; and
   (ii) the cap proximal sequence comprises:
      N₁ and N₂ of the trinucleotide cap structure and a sequence comprising N₃N₄N₅ at positions +3, +4, and +5 respectively of the RNA polynucleotide, wherein N₃ is G, N₄ is C, and N₅ is selected from: A, C, G, and U.

In some embodiments, the present disclosure provides an RNA polynucleotide comprising:
a 5′ cap; a cap proximal sequence comprising positions +1, +2, +3, +4, and +5 of the RNA polynucleotide; and a sequence encoding a payload, wherein:
   (i) the 5′ cap is a trinucleotide cap structure comprising N₁pN₂, wherein N₁ is position +1 of the RNA polynucleotide, and N₂ is position +2 of the RNA polynucleotide, and wherein N₁ is C and N₂ is G; and
   (ii) the cap proximal sequence comprises:
      N₁ and N₂ of the trinucleotide cap structure and a sequence comprising N₃N₄N₅ at positions +3, +4, and +5 respectively of the RNA polynucleotide, wherein N₃ is C, and each N₄ and N₅ is selected from: A, C, G, and U.

In some embodiments, the present disclosure provides an RNA polynucleotide comprising:
a 5′ cap; a cap proximal sequence comprising positions +1, +2, +3, +4, and +5 of the RNA polynucleotide; and a sequence encoding a payload, wherein:
   (i) the 5′ cap is a tetranucleotide cap structure comprising N₁pN₂pN₃, wherein N₁ is position +1 of the RNA polynucleotide, N₂ is position +2 of the RNA polynucleotide, and N₃ is position +3 of the polynucleotide, and wherein N₁, N₂, and N₃ are selected from one of the following combinations: (a) N₁ is G, N₂ is C, and N₃ is G; (b) N₁ is U, N₂ is C, and N₃ is G; or (c) N₁ is A, N₂ is C, and N₃ is G; and
   (ii) the cap proximal sequence comprises:
      N₁, N₂, and N₃ of the tetranucleotide cap structure and a sequence comprising N₄N₅ at positions +4 and +5 respectively of the RNA polynucleotide, wherein N₄ is C, and N₅ is selected from: A, C, G, and U.

In some embodiments, the present disclosure provides an RNA polynucleotide comprising:
a 5′ cap; a cap proximal sequence comprising positions +1, +2, +3, +4, and +5 of the RNA polynucleotide; and a sequence encoding a payload, wherein:
   (i) the 5′ cap is a tetranucleotide cap structure comprising N₁pN₂pN₃, wherein N₁ is position +1 of the RNA polynucleotide, N₂ is position +2 of the RNA polynucleotide, and N₃ is position +3 of the polynucleotide, and wherein N₁ is C, N₂ is G, and N₃ is C; and
   (ii) the cap proximal sequence comprises:
      N₁, N₂, and N₃ of the tetranucleotide cap structure and a sequence comprising N₄N₅ at positions +4 and +5 respectively of the RNA polynucleotide, wherein each N₄ and N₅ is selected from: A, C, G, and U.

In some embodiments, the present disclosure provides an RNA polynucleotide comprising:
a 5′ cap; a cap proximal sequence comprising positions +1, +2, +3, +4, and +5 of the RNA polynucleotide; and a sequence encoding a payload, wherein:
   (i) the 5′ cap is a trinucleotide cap structure comprising N₁pN₂, wherein N₁ is position +1 of the RNA polynucleotide, and N₂ is position +2 of the RNA polynucleotide, and wherein N₁ is A and N₂ is U; and
   (ii) the cap proximal sequence comprises:
      N₁ and N₂ of the trinucleotide cap structure and a sequence comprising N₃N₄N₅ at positions +3, +4, and +5 respectively of the RNA polynucleotide, wherein N₃ is A, and each N₄ and N₅ is selected from: A, C, G, and U.

In some embodiments, the present disclosure provides an RNA polynucleotide comprising:
a 5′ cap; a cap proximal sequence comprising positions +1, +2, +3, +4, and +5 of the RNA polynucleotide; and a sequence encoding a payload, wherein:
   (i) the 5′ cap is a tetranucleotide cap structure comprising N₁pN₂pN₃, wherein N₁ is position +1 of the RNA polynucleotide, N₂ is position +2 of the RNA polynucleotide, and N₃ is position +3 of the polynucleotide, and wherein N₁ is A, N₂ is U, and N₃ is A; and
   (ii) the cap proximal sequence comprises:
      N₁, N₂, and N₃ of the tetranucleotide cap structure and a sequence comprising N₄N₅ at positions +4 and +5 respectively of the RNA polynucleotide, wherein each N₄ and N₅ is selected from: A, C, G, and U.

In some embodiments, the present disclosure provides an RNA polynucleotide comprising:
a 5′ cap; a cap proximal sequence comprising positions +1, +2, +3, +4, and +5 of the RNA polynucleotide; and a sequence encoding a payload, wherein:
   (i) the 5′ cap is m₂^{(7,3′O)}Gppp^{(m2′O)}A₁pG₂, wherein A₁ is position +1 of the RNA polynucleotide, and G₂ is position +2 of the RNA polynucleotide; and
   (ii) the cap proximal sequence comprises:
      A₁ and G₂ of the 5′ cap and a sequence comprising N₃N₄N₅ at positions +3, +4, and +5 respectively of the RNA polynucleotide, wherein N₃ is A, and N₄ and N₅ are selected from: A, C, G, and U.

In some embodiments, the present disclosure provides an RNA polynucleotide comprising:
a 5′ cap; a cap proximal sequence comprising positions +1, +2, +3, +4, and +5 of the RNA polynucleotide; and a sequence encoding a payload, wherein:
   (i) the 5′ cap is m₂^{(7,3′O)}Gppp^{(m2′O)}A₁pG₂, wherein A₁ is position +1 of the RNA polynucleotide, and G₂ is position +2 of the RNA polynucleotide; and
   (ii) the cap proximal sequence comprises:
      A₁ and G₂ of the 5′ cap and a sequence comprising N₃N₄N₅ at positions +3, +4, and +5 respectively of the RNA polynucleotide, wherein N₃ and N₄ are G, and N₅ is selected from: A, C, G, and U.

In some embodiments, the present disclosure provides an RNA polynucleotide comprising:
a 5′ cap; a cap proximal sequence comprising positions +1, +2, +3, +4, and +5 of the RNA polynucleotide; and a sequence encoding a payload, wherein:
   (i) the 5′ cap is m₂^{(7,3′O)}Gppp^{(m2′O)}A₁pG₂, wherein A₁ is position +1 of the RNA polynucleotide, and G₂ is position +2 of the RNA polynucleotide; and
   (ii) the cap proximal sequence comprises:
      A₁ and G₂ of the 5′ cap and a sequence comprising N₃N₄N₅ at positions +3, +4, and +5 respectively of the RNA polynucleotide, wherein N₃ is C, N₄ is G, and N₅ is selected from: A, C, G, and U.

### Codon Optimization

In some embodiments, a payload (e.g., a polypeptide) described herein is encoded by a coding sequence which is codon-optimized and/or the G/C content of which is increased compared to wild type coding sequence. In some embodiments, one or more sequence regions of the coding sequence are codon-optimized and/or increased in the G/C content compared to the corresponding sequence regions of the wild type coding sequence. In some embodiments, codon-optimization and/or increased the G/C content does not change the sequence of the encoded amino acid sequence.

The term “codon-optimized” is understood by those in the art to refer to alteration of codons in the coding region of a nucleic acid molecule to reflect the typical codon usage of a host organism without preferably altering the amino acid sequence encoded by the nucleic acid molecule. Within the context of the present disclosure, coding regions are preferably codon-optimized for optimal expression in a subject to be treated using an RNA polynucleotide described herein. Codon-optimization is based on the finding that the translation efficiency is also determined by a different frequency in the occurrence of tRNAs in cells. Thus, the sequence of RNA may be modified such that codons for which frequently occurring tRNAs are available are inserted in place of “rare codons”.

In some embodiments, guanosine/cytidine (G/C) content of a coding region (e.g., of a payload sequence) of an RNA is increased compared to the G/C content of the corresponding coding sequence of a wild type RNA encoding the payload, wherein the amino acid sequence encoded by the RNA is preferably not modified compared to the amino acid sequence encoded by the wild type RNA. This modification of the RNA sequence is based on the fact that the sequence of any RNA region to be translated is important for efficient translation of that mRNA. Sequences having an increased G (guanosine)/C (cytidine) content are more stable than sequences having an increased A (adenosine)/U (uridine) content. In respect to the fact that several codons code for one and the same amino acid (so-called degeneration of the genetic code), the most favourable codons for the stability can be determined (so-called alternative codon usage). Depending on the amino acid to be encoded by the RNA, there are various possibilities for modification of the RNA sequence, compared to its wild type sequence. In particular, codons which contain A and/or U nucleosides can be modified by substituting these codons by other codons, which code for the same amino acids but contain no A and/or U or contain a lower content of A and/or U nucleosides.

In some embodiments, G/C content of a coding region of an RNA described herein is increased by at least 10%, at least 20%, at least 30%, at least 40%, at least 50%, at least 55%, or even more compared to the G/C content of a coding region of a wild type RNA.

### 5′ Cap

A structural feature of mRNAs is cap structure at five-prime end (5′). Natural eukaryotic mRNA comprises a 7-methylguanosine cap linked to the mRNA via a 5′ to 5′-triphosphate bridge resulting in cap0 structure (m7GpppN). In most eukaryotic mRNA and some viral mRNA, further modifications can occur at the 2′-hydroxy-group (2′-OH) (e.g., the 2′-hydroxyl group may be methylated to form 2′-O-Me) of the first and subsequent nucleotides producing “cap1” and “cap2” five-prime ends, respectively. Diamond, et al., (2014) Cytokine & growth Factor Reviews, 25:543-550 reported that cap0-mRNA cannot be translated as efficiently as cap1-mRNA in which the role of 2′-O-Me in the penultimate position at the mRNA 5′ end is determinant. Lack of the 2′-O-met has been shown to trigger innate immunity and activate IFN response. Daffis, et al. (2010) Nature, 468:452-456; and Züst et al. (2011) Nature Immunology, 12:137-143.

RNA capping is well researched and is described, e.g., in Decroly E et al. (2012) Nature Reviews 10: 51-65; and in Ramanathan A. et al., (2016) Nucleic Acids Res; 44(16): 7511-7526, the entire contents of each of which is hereby incorporated by reference. In some embodiments, to imitate the 5′ cap structure of natural mRNA, in vitro-transcribed mRNA (IVT mRNA) can be capped either post-transcriptionally using recombinant Vaccinia virus-derived enzymes (see., e.g., Kyrieleis, et al. (1993) Structure 22:452-465; and Corbett, et al. (2020) The New England Journal of Medicine 383:1544-1555) or co-transcriptionally by adding caps immediately into the in vitro transcription reaction (see, e.g., Jemielity, et al. (2003) RNA 9:1108-1122; and Kocmik, et al. (2018) Cell Cycle 17:1624-1636). In some embodiments, enzymatic capping can yield cap1-mRNA, but can be time-consuming since it requires an extra purification step, additionally demands a heating step to improve the accessibility of structured 5′ends, thereby increasing further the risk of RNA degradation. Among other things, capping with this method is highly reproducible and less expensive than enzymatic capping. mRNA generated in the presence of these caps are more resistance to the human decapping enzymes (Kowalska et al. (2008) RNA 14:1119-1131) and/or interferon-induced proteins with tetratricopeptide repeats (IFITs) which inhibits cap0-dependent translation (Diamond et al. (2014) Cytokine & Growth Factor Reviews 25:543-550; and Miedziak, et al. (2019) RNA 26:58-68). However, using this approach, GTP is typically competing with caps during transcription that can lead to poor capping efficiency resulting in weak translational capacity. Certain cap1 structures can be incorporated into IVT mRNA in the right orientation for producing cap1-mRNA with high capping efficiency in a rapid co-transcriptional reaction. Henderson et al., (2021) Current Protocols 1:e39. For example, a trinucleotide cap1 structure requires AG initiator, avoiding the slippage of RNA polymerases on template DNA strand as opposed to those contain a G triplet as a transcriptional start site. Imburgio, et al. (2000) Biochemistry 39:10419-10430.

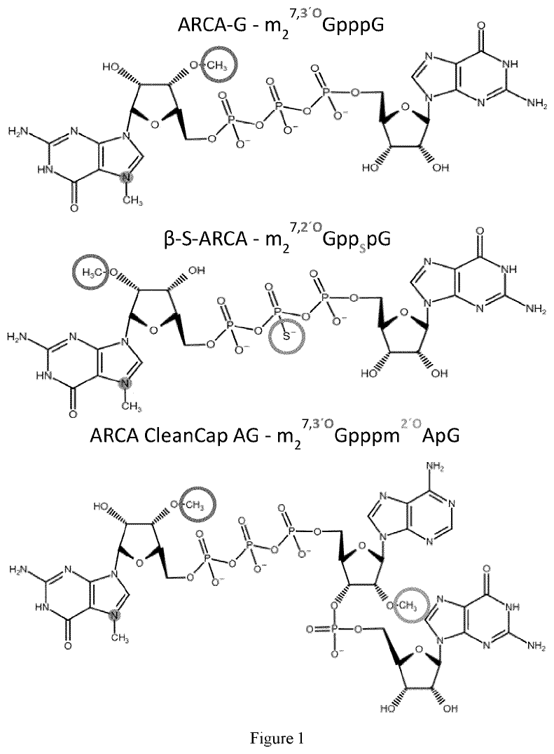

In some embodiments, a 5′ cap includes a Cap-0 (also referred herein as “Cap0”), a Cap-1 (also referred herein as “Cap1”), or Cap-2 (also referred herein as “Cap2”). See, e.g., FIG. 1 of Ramanathan A et al., and FIG. 1 of Decroly E et al.

The term “5′-cap” as used herein refers to a structure found on the 5′-end of an RNA, e.g., mRNA, and generally includes a guanosine nucleotide connected to an RNA, e.g., mRNA, via a 5′- to 5′-triphosphate linkage (also referred to as Gppp or G(5′)ppp(5′)). In some embodiments, a guanosine nucleoside included in a 5′ cap may be modified, for example, by methylation at one or more positions (e.g., at the 7-position) on a base (guanine), and/or by methylation at one or more positions of a ribose. In some embodiments, a guanosine nucleoside included in a 5′ cap comprises a 3′O methylation at a ribose (denoted as “(m^{3′-O})G” or “3′OMeG”). In some embodiments, a guanosine nucleoside included in a 5′ cap comprises methylation at the 7-position of guanine (denoted as “(m⁷)G” or “m7G”). In some embodiments, a guanosine nucleoside included in a 5′ cap comprises methylation at the 7-position of guanine and a 3′ O methylation at a ribose (denoted as “(m₂^{7,3′-O})G” or “m7(3′OMeG)”). In some embodiments, a guanosine nucleoside included in a 5′ cap comprises a 2′O methylation at a ribose (denoted as “(m^{2′-O})G” or “2′OMeG”). In some embodiments, a guanosine nucleoside included in a 5′ cap comprises methylation at the 7-position of guanine and a 2′ O methylation at a ribose (denoted as “(m₂^{7,2′-O})G” or “m7(2′OMeG)”). It will be understood that the notation used in the above paragraph, e.g., “(m₂^{7,3′-O})G” or “m7(3′OMeG)”, applies to other structures described herein.

In some embodiments, providing an RNA with a 5′-cap disclosed herein or a 5′-cap may be achieved by in vitro transcription, in which a 5′-cap is co-transcriptionally expressed into an RNA strand, or may be attached to an RNA post-transcriptionally using capping enzymes. In some embodiments, co-transcriptional capping with a cap disclosed herein, e.g., a cap0, cap1, or cap2 structure, improves the capping efficiency of an RNA compared to co-transcriptional capping with an appropriate reference comparator. In some embodiments, improving capping efficiency can increase a translation efficiency and/or translation rate of an RNA, and/or increase expression of an encoded polypeptide.

In some embodiments, T7 RNA polymerase prefers G as the initial site. Accordingly, in some such embodiments, the present disclosure provides caps (e.g., trinucleotide and tetranucleotide caps described herein) wherein the 3′end of the trinucleotide (e.g., N₂) or tetranucleotide cap (e.g., N₃) is G.

In some embodiments, it will be appreciated that all compounds or structures (e.g., 5′ caps) provided herein encompass the free base or salt form (e.g., an Na⁺ salt) comprising a suitable counterion (e.g., Na⁺). Compounds or structures (e.g., 5′ caps) depicted as a salt also encompass the free base and include suitable counterions (e.g., Na⁺).

In some embodiments, an RNA described herein comprises a 5′-cap or a 5′ cap, e.g., a Cap0, a Cap1 or a Cap2. In some embodiments, a provided RNA does not have uncapped 5′-triphosphates. In some embodiments, an RNA may be capped with a 5′-cap. In some embodiments, an RNA described herein comprises a Cap0. In some embodiments, an RNA described herein comprises a Cap1, e.g., as described herein. In some embodiments, an RNA described herein comprises a Cap2.

In some embodiments, a Cap0 structure comprises a guanosine nucleoside methylated at the 7-position of guanine (m7G). In some embodiments, a Cap0 structure is connected to an RNA via a 5′- to 5′-triphosphate linkage and is also referred to herein as m7Gppp or m7G(5′)ppp(5′).

In some embodiments, a Cap1 structure comprises a guanosine nucleoside methylated at the 7-position of guanine (m7G) and a 2′O methylated first nucleotide in an RNA (2′OMeN₁). In some embodiments, a Cap1 structure is connected to an RNA via a 5′- to 5′-triphosphate linkage and is also referred to herein as m7Gppp(2′OMeN₁) or m7G(5′)ppp(5′)(2′OMeN₁), wherein N₁ is as defined and described herein.

In some embodiments, a m7G(5′)ppp(5′)(2′OMeN₁) Cap1 structure comprises a second nucleotide, N₂ which is a cap proximal nucleotide at position 2 and is chosen from A, G, C, or U (m7G(5′)ppp(5′)(2′OMeN₁)N₂) wherein each of N₁ and N₂ is as defined and described herein.

In some embodiments, the 5′ cap is a dinucleotide cap structure. In some embodiments, the 5′ cap is a dinucleotide cap structure comprising N₁, wherein N₁ is as defined and described herein. In some embodiments, the 5′ cap is a dinucleotide cap G*N₁, wherein N₁ is as defined above and herein, and:

G* comprises a structure of formula (I):

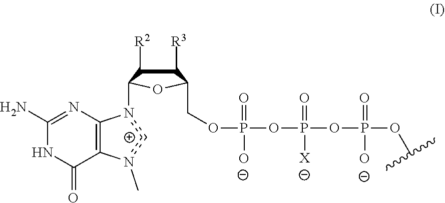

or a salt thereof,
wherein
   each R² and R³ is —OH or —OCH₃; and
   X is O or S.

In some embodiments, R² is —OH. In some embodiments, R² is —OCH₃. In some embodiments, R³ is —OH. In some embodiments, R³ is —OCH₃. In some embodiments, R² is —OH and R³ is —OH. In some embodiments, R² is —OH and R³ is —CH₃. In some embodiments, R² is —CH₃ and R³ is —OH. In some embodiments, R² is —CH₃ and R³ is —CH₃.

In some embodiments, X is O. In some embodiments, X is S.

In some embodiments, the 5′ cap is a dinucleotide Cap0 structure (e.g., (m⁷)GpppN₁, (m₂^{7,2′-O})GpppN₁, (m₂^{7,3′-O})GpppN₁, (m⁷)GppSpN₁, (m₂^{7,2′-O})GppSpN₁, or (m₂^{7,3′-O})GppSpN₁, wherein N₁ is as defined and described herein. In some embodiments, the 5′ cap is a dinucleotide Cap0 structure (e.g., (m⁷)GpppN₁, (m₂^{7,2′-O})GpppN₁, (m₂^{7,3′-O})GpppN₁, (m⁷)GppSpN₁, (m₂^{7,2′}—O)GppSpN₁, or (m₂^{7,3′-O})GppSpN₁, wherein N₁ is G. In some embodiments, the 5′ cap is a dinucleotide Cap0 structure (e.g., (m⁷)GpppN₁, (m₂^{7,2′-O})GpppN₁, (m₂^{7,3′-O})GpppN₁, (m⁷)GppSpN₁, (m₂^{7,2′-O})GppSpN₁, or (m₂^{7,3′-O})GppSpN₁, wherein N₁ is A, U, or C. In some embodiments, the 5′ cap is a dinucleotide Cap1 structure (e.g., (m⁷)Gppp(m^{2′-O})N₁, (m₂^{7,2′-O})Gppp(m^{2′-O})N₁, (m₂^{7,3′-O})Gppp(m^{2′-O})N₁, (m⁷)GppSp(m^{2′-O})N₁, (m₂^{7,2′-O})GppSp(m^{2′-O})N₁, or (m₂^{7,3′-O})GppSp(m^{2′-O})N₁, wherein N₁ is as defined and described herein. In some embodiments, the 5′ cap is selected from the group consisting of (m⁷)GpppG (“Ecap0”), (m⁷)Gppp(m^{2′-O})G (“Ecap1”), (m₂^{7,3′-O})GpppG (“ARCA” or “D1”), and (m₂^{7,2′-O})GppSpG (“beta-S-ARCA”). In some embodiments, the 5′ cap is (m⁷)GpppG (“Ecap0”), having a structure:

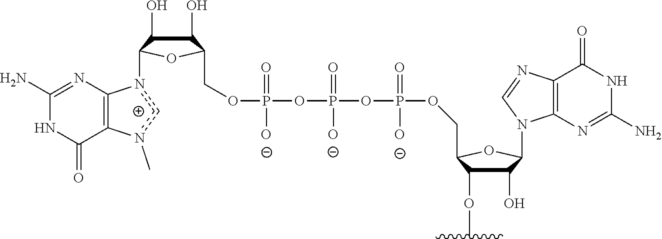

or a salt thereof,

In some embodiments, the 5′ cap is (m⁷)Gppp(m^{2′-O})G (“Ecap1”), having a structure:

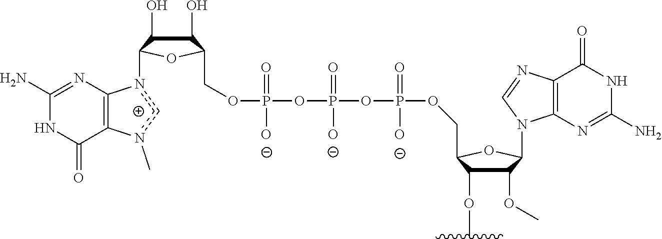

or a salt thereof.

In some embodiments, the 5′ cap is (m₂^{7,3′-O})GpppG (“ARCA” or “D1”), having a structure:

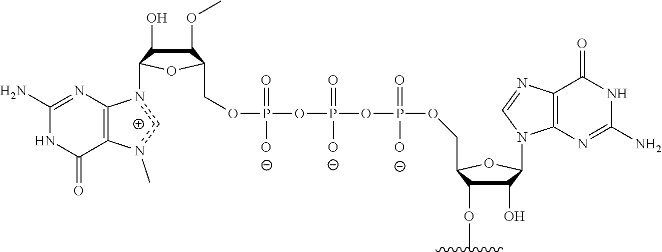

or a salt thereof.

In some embodiments, the 5′ cap is (m₂^{7,2′-O})GppSpG (“beta-S-ARCA”), having a structure:

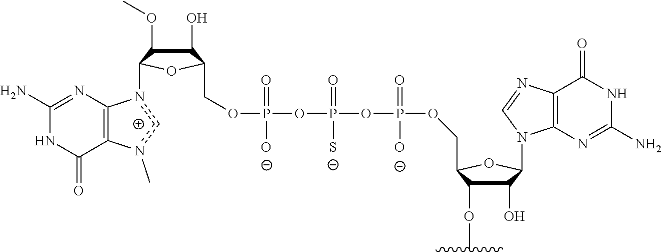

or a salt thereof.

In some embodiments, the 5′ cap is a trinucleotide cap structure. In some embodiments, the 5′ cap is a trinucleotide cap structure comprising N₁pN₂, wherein N₁ and N₂ are as defined and described herein. In some embodiments, the 5′ cap is a trinucleotide cap G*N¹pN₂, wherein N₁ and N₂ are as defined above and herein, and:

G* comprises a structure of formula (I):

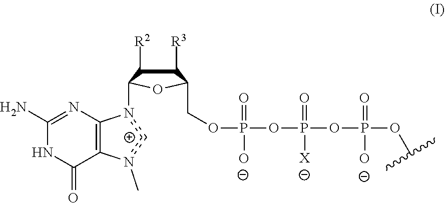

or a salt thereof, wherein R², R³, and X are as defined and described herein.

In some embodiments, the 5′ cap is a trinucleotide Cap0 structure (e.g. (m⁷)GpppN₁pN₂, (m₂^{7,2′-O})GpppN₁pN₂, or (m₂^{7,3′-O})GpppN₁pN₂, wherein N₁ and N₂ are as defined and described herein). In some embodiments, the 5′ cap is a trinucleotide Cap1 structure (e.g., (m⁷)Gppp(m^{2′-O})N₁pN₂, (m₂^{7,2′-O})Gppp(m^{2′-O})N₁pN₂, (m₂^{7,3′-O})Gppp(m^{2′-O})N₁pN₂, wherein N₁ and N₂ are as defined and described herein. In some embodiments, the 5′ cap is a trinucleotide Cap2 structure (e.g., (m⁷)Gppp(m^{2′-O})N₁p(m^{2′-O})N₂, (m₂^{7,2′-O})Gppp(m^{2′-O})N₁p(m^{2′-O})N₂, (m₂^{7,3′-O})Gppp(m^{2′-O})Np(m^{2′-O})N₂, wherein N₁ and N₂ are as defined and described herein.

In some embodiments, the 5′ cap is selected from the group consisting of (m₂^{7,3′-O})Gppp(m^{2′-O})ApG (“CleanCap AG”, “CC413”), (m₂^{7,3′-O})Gppp(m^{2′-O})GpG (“CleanCap GG”), (m⁷)Gppp(m^{2′-O})ApG, and (m₂^{7,3′-O})Gppp(m₂^{6,2′-O})ApG, and (m⁷)Gppp(m^{2′-O})ApU. In some embodiments, the 5′ cap is selected from the group consisting of (m₂^{7,3′-O})Gppp(m^{2′-O})ApG (“CleanCap AG”, “CC413”), (m₂^{7,3′-O})Gppp(m^{2′-O})GpG (“CleanCap GG”), (m⁷)Gppp(m^{2′-O})ApG, and (m₂^{7,3′-O})Gppp(m₂^{6,2′-O})ApG, (m⁷)Gppp(m^{2′-O})ApU, and (m₂^{7,3′-O})GPPp(m^{2′-O})CpG.

In some embodiments, the 5′ cap is (m₂^{7,3′-O})Gppp(m^{2′-O})ApG (“CleanCap AG 3′ OMe”, “CC413”), having a structure:

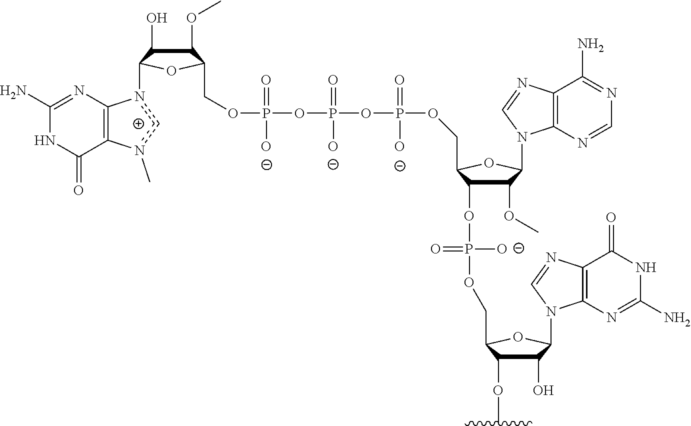

or a salt thereof.

In some embodiments, the 5′ cap is (m₂^{7,3′-O})Gppp(m^{2′-O})GpG (“CleanCap GG”), having a structure:

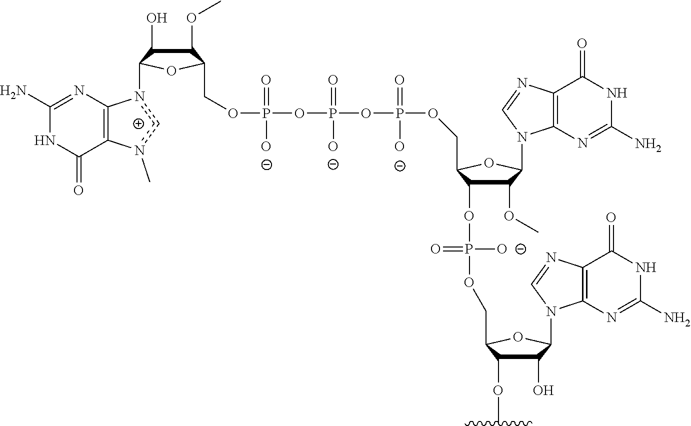

or a salt thereof.

In some embodiments, the 5′ cap is (m⁷)Gppp(m^{2′-O})ApG, having a structure:

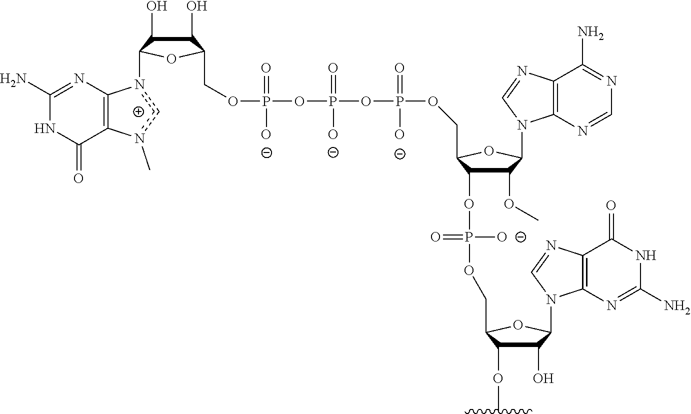

or a salt thereof.

In some embodiments, the 5′ cap is (m₂^{7,3′-O})Gppp(m₂^{6,2′-O})ApG, having a structure:

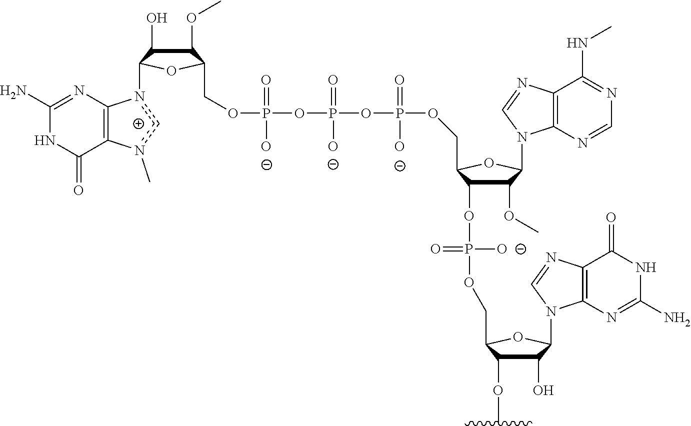

or a salt thereof.

In some embodiments, the 5′ cap is (m⁷)Gppp(m^{2′-O})ApU, having a structure:

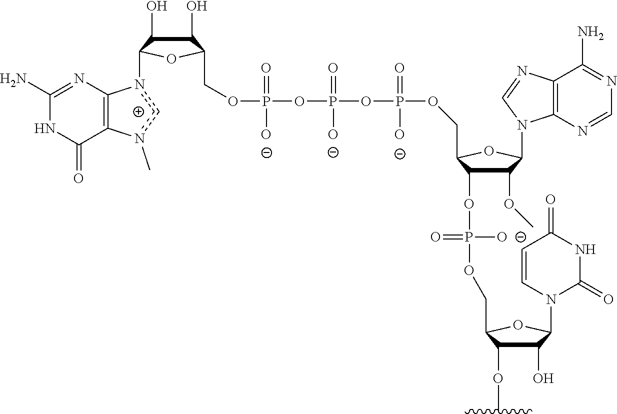

or a salt thereof.

In some embodiments, the 5′ cap is (m₂^{7,3′-O})Gppp(m^{2′-O})CpG, having a structure:

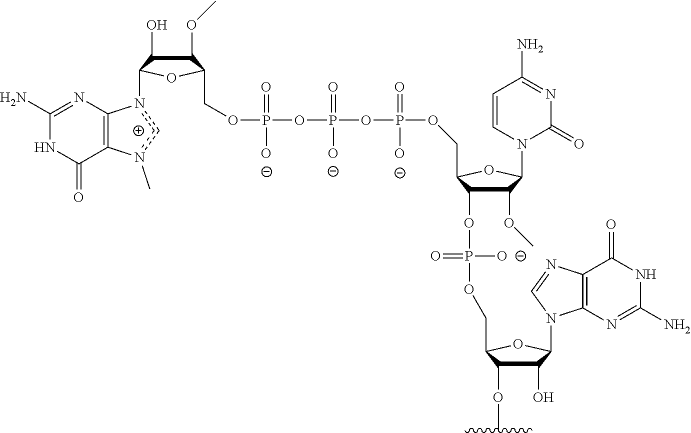

or a salt thereof.

In some embodiments, the 5′ cap is a tetranucleotide cap structure. In some embodiments, the 5′ cap is a tetranucleotide cap structure comprising N₁pN₂pN₃, wherein N₁, N₂, and N₃ are as defined and described herein. In some embodiments, the 5′ cap is a tetranucleotide cap G*N¹pN₂pN₃, wherein N₁, N₂, and N₃ are as defined above and herein, and: G* comprises a structure of formula (I):

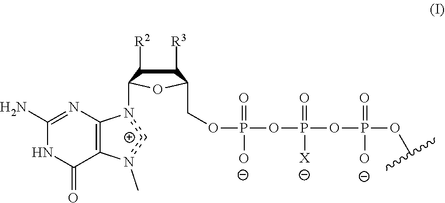

or a salt thereof, wherein R², R³, and X are as defined and described herein.

In some embodiments, the 5′ cap is a tetranucleotide Cap0 structure (e.g. (m⁷)GpppN₁pN₂pN₃, (m₂^{7,2′-O})GpppN₁pN₂pN₃, or (m₂^{7,3′-O})GpppN₁N₂pN₃, wherein N₁, N₂, and N₃ are as defined and described herein). In some embodiments, the 5′ cap is a tetranucleotide Cap1 structure (e.g., (m⁷)Gppp(m^{2′-O})N₁pN₂pN₃, (m₂^{7,2′-O})Gppp(m^{2′-O})N¹pN₂pN₃, or (m₂^{7,3′-O})Gppp(m^{2′-O})N₁pN₂N₃, wherein N₁, N₂, and N₃ are as defined and described herein). In some embodiments, the 5′ cap is a tetranucleotide Cap2 structure (e.g., (m⁷)Gppp(m^{2′-O})N₁p(m^{2′-O})N₂pN₃, (m₂^{7,2′-O})Gppp(m^{2′-O})N₁p(m^{2′-O})N₂pN₃, (m₂^{7,3′-O})Gppp(m^{2′-O})N₁p(m^{2′-O})N₂pN₃, wherein N₁, N₂, and N₃ are as defined and described herein). In some embodiments, the 5′ cap is selected from the group consisting of (m₂^{7,3′-O})Gppp(m^{2′-O})Ap(m^{2′-O})GpG, (m₂^{7,3′-O})Gppp(m^{2′-O})Gp(m^{2′-O})GpC, (m⁷)Gppp(m^{2-O})Ap(m^{2′-O})UpA, and (m⁷)Gppp(m^{2-O})Ap(m^{2′-O})GpG.

In some embodiments, the 5′ cap is (m₂^{7,3′-O})Gppp(m^{2′-O})Ap(m^{2′-O})GpG, having a structure:

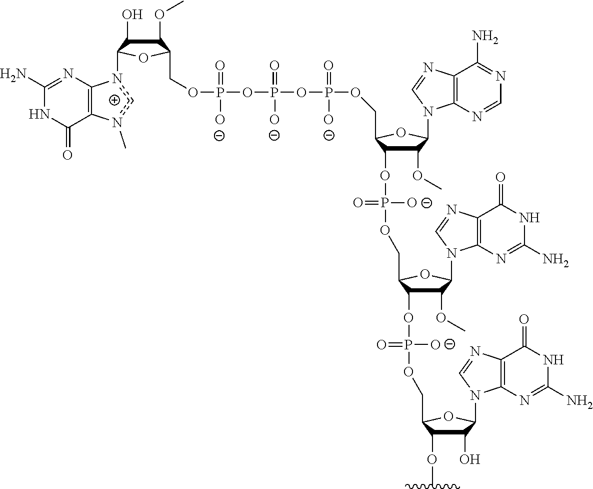

or a salt thereof.

In some embodiments, the 5′ cap is (m₂^{7,3′-O})Gppp(m^{2′-O})Gp(m^{2′-O})GpC, having a structure:

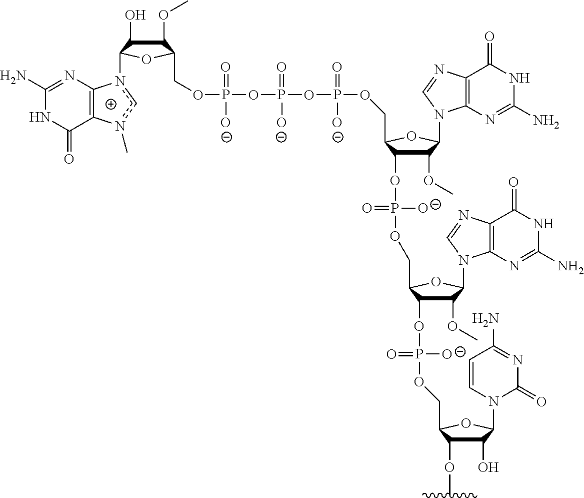

or a salt thereof.

In some embodiments, the 5′ cap is (m⁷)Gppp(m^{2′-O})Ap(m^{2′-O})UpA, having a structure:

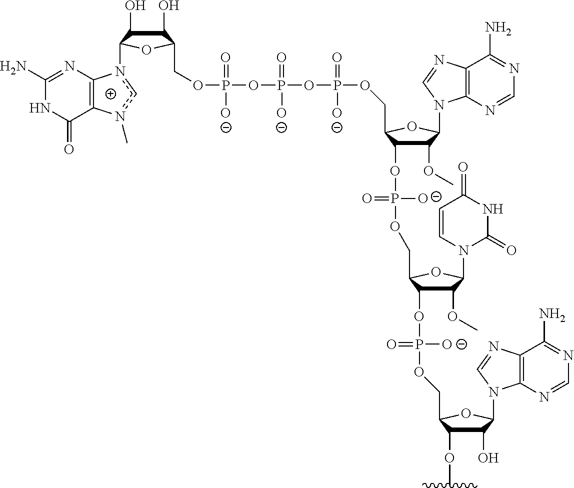

or a salt thereof.

In some embodiments, the 5′ cap is (m⁷)Gppp(m^{2′-O})Ap(m^{2′-O})GpG, having a structure:

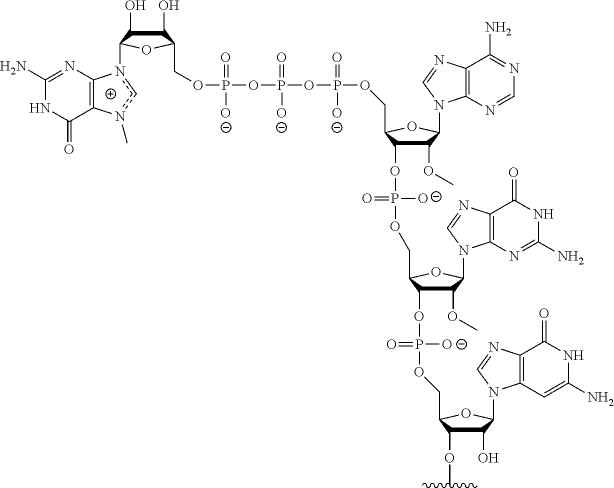

or a salt thereof.

In some embodiments, a cap1 structure is or comprises m7G(5′)ppp(5′)(2′OMeA₁)pG₂ wherein A is a cap proximal nucleotide at position +1 and G is a cap proximal nucleotide at position +2, and has the following structure:

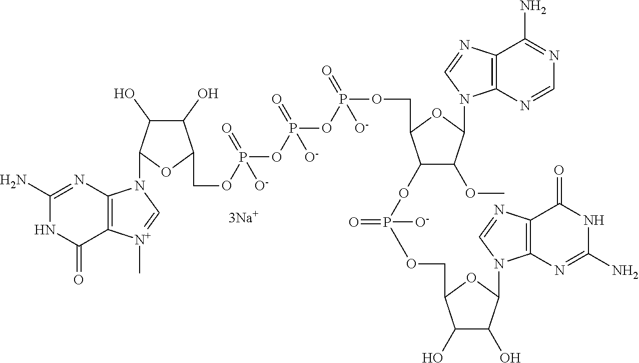 [SMILES: COC1C(OP(=O)([O-])OCC2OC(n3cnc4c(=O)[nH]c(N)nc43)C(O)C2O)C(COP(=O)([O-])OP(=O)([O-])OP(=O)([O-])OCC2OC(n3c[n+](C)c4c(=O)[nH]c(N)nc43)C(O)C2O)OC1n1cnc2c(N)ncnc21]

In some embodiments, a cap1 structure is or comprises m7G(5′)ppp(5′)(2′OMeA₁)pU₂ wherein A is a cap proximal nucleotide at position 1 and U is a cap proximal nucleotide at position 2, and has the following structure:

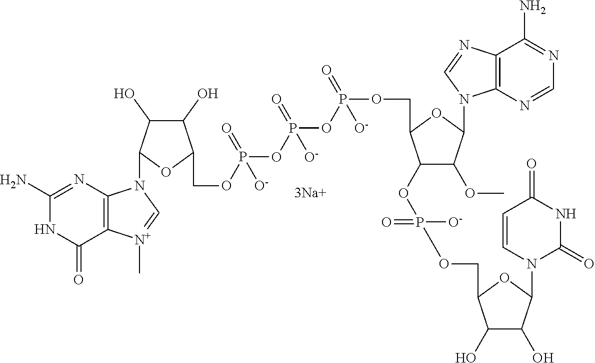 [SMILES: COC1C(OP(=O)([O-])OCC2OC(n3ccc(=O)[nH]c3=O)C(O)C2O)C(COP(=O)([O-])OP(=O)([O-])OP(=O)([O-])OCC2OC(n3c[n+](C)c4c(=O)[nH]c(N)nc43)C(O)C2O)OC1n1cnc2c(N)ncnc21]

In some embodiments, a cap1 structure is or comprises m7G(5′)ppp(5′)(2′OMeG₁)pG₂ wherein G is a cap proximal nucleotide at position 1 and G is a cap proximal nucleotide at position 2, and has the following structure:

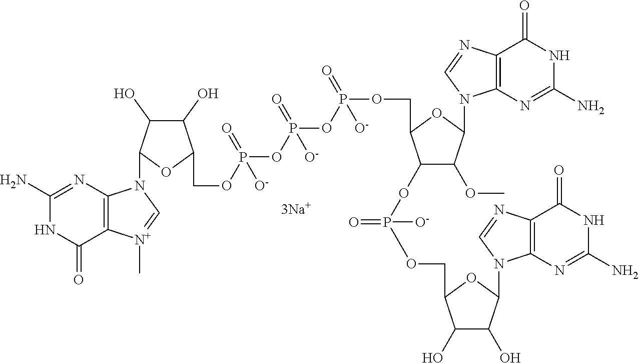 [SMILES: COC1C(OP(=O)([O-])OCC2OC(n3cnc4c(=O)[nH]c(N)nc43)C(O)C2O)C(COP(=O)([O-])OP(=O)([O-])OP(=O)([O-])OCC2OC(n3c[n+](C)c4c(=O)[nH]c(N)nc43)C(O)C2O)OC1n1cnc2c(=O)[nH]c(N)nc21]

In some embodiments, a 5′ cap is or comprises m7(3′OMeG)(5′)ppp(5′)(2′OMeG₁)pG₂ wherein G is a cap proximal nucleotide at position 1 and G is a cap proximal nucleotide at position 2, and has the following structure:

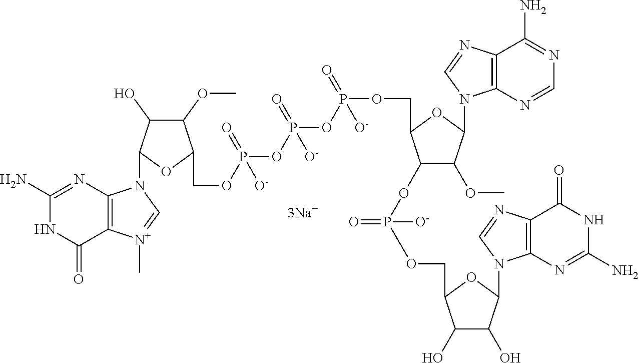 [SMILES: COC1C(COP(=O)([O-])OP(=O)([O-])OP(=O)([O-])OCC2OC(n3cnc4c(N)ncnc43)C(OC)C2OP(=O)([O-])OCC2OC(n3cnc4c(=O)[nH]c(N)nc43)C(O)C2O)OC(n2c[n+](C)c3c(=O)[nH]c(N)nc32)C1O]

In some embodiments, a 5′ cap is or comprises m7(3′OMeG)(5′)ppp(5′)(2′OMeG₁)pG₂ wherein G is a cap proximal nucleotide at position 1 and G is a cap proximal nucleotide at position 2, and has the following structure:

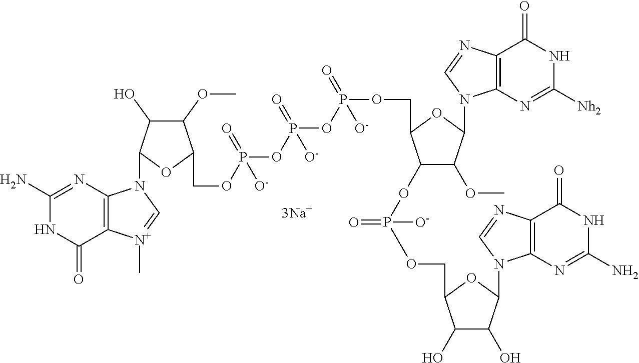

In some embodiments, a second nucleotide in a Cap1 structure can comprise one or more modifications, e.g., methylation. In some embodiments, a Cap1 structure comprising a second nucleotide comprising a 2′O methylation is a Cap2 structure.

In some embodiments, an RNA polynucleotide comprising a Cap1 structure has increased translation efficiency, increased translation rate and/or increased expression of an encoded payload relative to an appropriate reference comparator. In some embodiments, an RNA polynucleotide comprising a cap1 structure having m7(3′OMeG)(5′)ppp(5′)(2′OMeA₁)pG₂ wherein A is a cap proximal nucleotide at position 1 and G is a cap proximal nucleotide at position 2, has increased translation efficiency relative to an RNA polynucleotide comprising a cap1 structure having m7(3′OMeG)(5′)ppp(5′)(2′OMeG₁)pG₂ wherein G₁ is a cap proximal nucleotide at position 1 and G₂ is a cap proximal nucleotide at position 2. In some embodiments, increased translation efficiency is assessed upon administration of an RNA polynucleotide to a cell or an organism.

In some embodiments, a cap used in an RNA polynucleotide is m₂^{7,3′—O} Gppp(m₁^{2′-O})ApG (also sometimes referred to as m₂^{7,3′O} G(5′)ppp(5′)m^{2′-O}ApG or m7(3′OMeG)(5′)ppp(5′)(2′OMeA)pG), which has the following structure:

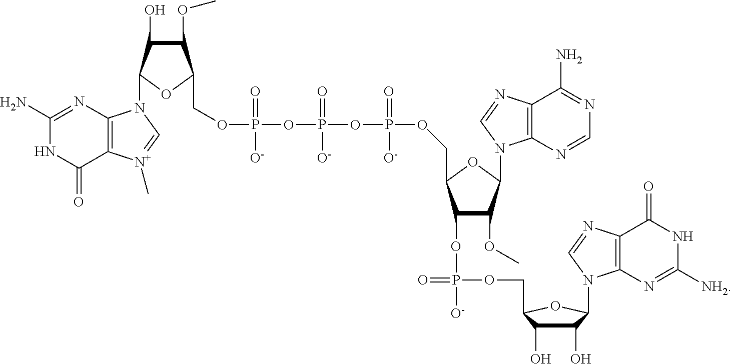

Below is an exemplary Cap1 RNA, which comprises RNA and m₂^{7,3′O}G(5′)ppp(5′)m^{2′-O}ApG:

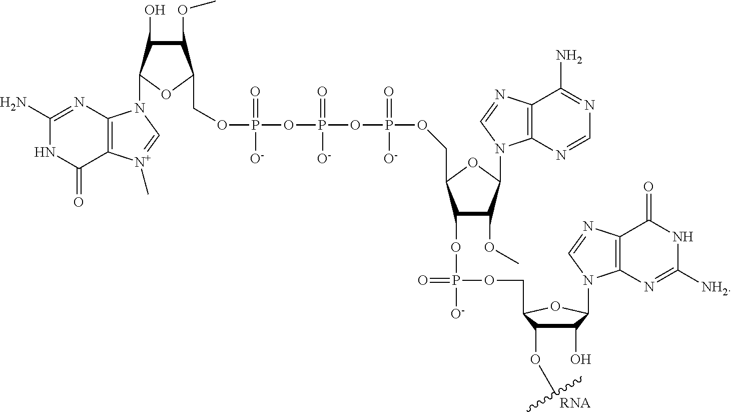

Below is another exemplary Cap1 RNA:

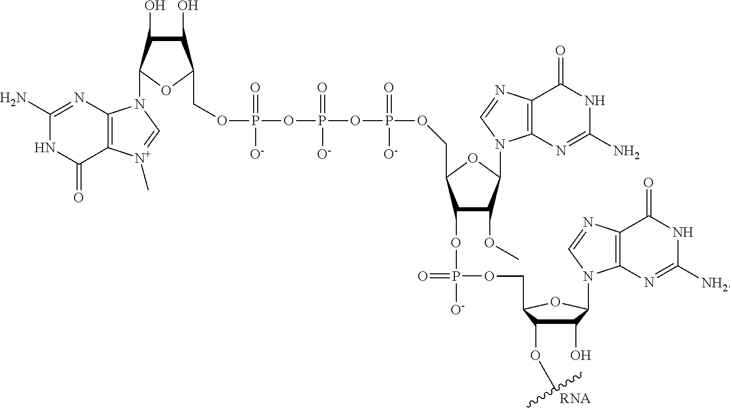

### 5′ UTR and Cap Proximal Sequences

In some embodiments, an RNA disclosed herein comprises a 5′-UTR. The term “untranslated region” or “UTR” relates to a region in a DNA molecule which is transcribed but is not translated into an amino acid sequence, or to the corresponding region in an RNA polynucleotide, such as an mRNA molecule. An untranslated region (UTR) can be present 5′ (upstream) of an open reading frame (5′-UTR) and/or 3′ (downstream) of an open reading frame (3′-UTR). A 5′-UTR, if present, is located at the 5′ end, upstream of the start codon of a protein-encoding region. A 5′-UTR is downstream of the 5′-cap (if present), e.g. directly adjacent to the 5′-cap.

In some embodiments, a 5′ UTR disclosed herein comprises a cap proximal sequence, e.g., as disclosed herein. In some embodiments, a cap proximal sequence comprises a sequence adjacent to a 5′ cap. In some embodiments, a cap proximal sequence comprises nucleotides in positions +1, +2, +3, +4, and/or +5 of an RNA polynucleotide.

In some embodiments, a Cap structure comprises one or more polynucleotides of a cap proximal sequence. In some embodiments, a Cap structure comprises an m7 Guanosine cap and nucleotide +1 (N₁) of an RNA polynucleotide. In some embodiments, a Cap structure comprises an m7 Guanosine cap and nucleotide +2 (N₂) of an RNA polynucleotide. In some embodiments, a Cap structure comprises an m7 Guanosine cap and nucleotides +1 and +2 (N₁ and N₂) of an RNA polynucleotide. In some embodiments, a Cap structure comprises an m7 Guanosine cap and nucleotides +1, +2, and +3 (N₁, N₂, and N₃) of an RNA polynucleotide.

Those skilled in the art, reading the present disclosure, will appreciate that, in some embodiments, one or more residues of a cap proximal sequence (e.g., one or more of residues +1, +2, +3, +4, and/or +5) may be included in an RNA by virtue of having been included in a cap entity (e.g., a Cap1 or Cap2 structure, etc); alternatively, in some embodiments, at least some of the residues in a cap proximal sequence may be enzymatically added (e.g., by a polymerase such as a T7 polymerase). For example, in certain exemplified embodiments where a m₂^{7,3′-O}Gppp(m₁^{2′-O})ApG cap is utilized, +1 (i.e., N₁) and +2 (i.e. N₂) are the (m₁^{2′-O})A and G residues of the cap, and +3, +4, and +5 are added by polymerase (e.g., T7 polymerase).

### A. Cap Proximal Sequence Comprising Nucleotide of Dinucleotide 5′ Cap

In some embodiments, the 5′ cap is a dinucleotide cap structure, wherein the cap proximal sequence comprises N₁ of the 5′ cap, where N₁ is any nucleotide, e.g., A, C, G or U. In some embodiments, the 5′ cap is a dinucleotide cap structure, wherein the cap proximal sequence comprises N₁ of the 5′ cap, where N₁ is G.

### B. Cap Proximal Sequence Comprising Nucleotide(s) of Trinucleotide 5′ Cap

In some embodiments, the 5′ cap is a trinucleotide cap structure (e.g., the trinucleotide cap structures described above and herein), wherein the cap proximal sequence comprises N₁ and N₂ of the 5′ cap, wherein N₁ and N₂ are independently any nucleotide, e.g., A, C, G or U. In some embodiments, the 5′ cap is a trinucleotide cap structure (e.g., the trinucleotide cap structures described above and herein), wherein the cap proximal sequence comprises N₁ and N₂ of the 5′ cap, wherein N₁ and N₂ are A. In some embodiments, the 5′ cap is a trinucleotide cap structure (e.g., the trinucleotide cap structures described above and herein), wherein the cap proximal sequence comprises N₁ and N₂ of the 5′ cap, wherein N₁ and N₂ are C. In some embodiments, the 5′ cap is a trinucleotide cap structure (e.g., the trinucleotide cap structures described above and herein), wherein the cap proximal sequence comprises N₁ and N₂ of the 5′ cap, wherein N₁ and N₂ are G. In some embodiments, the 5′ cap is a trinucleotide cap structure (e.g., the trinucleotide cap structures described above and herein), wherein the cap proximal sequence comprises N₁ and N₂ of the 5′ cap, wherein N₁ and N₂ are U. In some embodiments, the 5′ cap is a trinucleotide cap structure (e.g., the trinucleotide cap structures described above and herein), wherein the cap proximal sequence comprises N₁ and N₂ of the 5′ cap, wherein N₁ is A and N₂ is C. In some embodiments, the 5′ cap is a trinucleotide cap structure (e.g., the trinucleotide cap structures described above and herein), wherein the cap proximal sequence comprises N₁ and N₂ of the 5′ cap, wherein N₁ is A and N₂ is G. In some embodiments, the 5′ cap is a trinucleotide cap structure (e.g., the trinucleotide cap structures described above and herein), wherein the cap proximal sequence comprises N₁ and N₂ of the 5′ cap, wherein N₁ is A and N₂ is U. In some embodiments, the 5′ cap is a trinucleotide cap structure (e.g., the trinucleotide cap structures described above and herein), wherein the cap proximal sequence comprises N₁ and N₂ of the 5′ cap, wherein N₁ is C and N₂ is A. In some embodiments, the 5′ cap is a trinucleotide cap structure (e.g., the trinucleotide cap structures described above and herein), wherein the cap proximal sequence comprises N₁ and N₂ of the 5′ cap, wherein N₁ is C and N₂ is G. In some embodiments, the 5′ cap is a trinucleotide cap structure (e.g., the trinucleotide cap structures described above and herein), wherein the cap proximal sequence comprises N₁ and N₂ of the 5′ cap, wherein N₁ is C and N₂ is U. In some embodiments, the 5′ cap is a trinucleotide cap structure (e.g., the trinucleotide cap structures described above and herein), wherein the cap proximal sequence comprises N₁ and N₂ of the 5′ cap, wherein N₁ is G and N₂ is A. In some embodiments, the 5′ cap is a trinucleotide cap structure (e.g., the trinucleotide cap structures described above and herein), wherein the cap proximal sequence comprises N₁ and N₂ of the 5′ cap, wherein N₁ is G and N₂ is C. In some embodiments, the 5′ cap is a trinucleotide cap structure (e.g., the trinucleotide cap structures described above and herein), wherein the cap proximal sequence comprises N₁ and N₂ of the 5′ cap, wherein N₁ is G and N₂ is U. In some embodiments, the 5′ cap is a trinucleotide cap structure (e.g., the trinucleotide cap structures described above and herein), wherein the cap proximal sequence comprises N₁ and N₂ of the 5′ cap, wherein N₁ is U and N₂ is A. In some embodiments, the 5′ cap is a trinucleotide cap structure (e.g., the trinucleotide cap structures described above and herein), wherein the cap proximal sequence comprises N₁ and N₂ of the 5′ cap, wherein N₁ is U and N₂ is C. In some embodiments, the 5′ cap is a trinucleotide cap structure (e.g., the trinucleotide cap structures described above and herein), wherein the cap proximal sequence comprises N₁ and N₂ of the 5′ cap, wherein N₁ is U and N₂ is G.

In some embodiments, e.g., where the 5′ cap is a trinucleotide cap structure and cap proximal sequence are as described in the previous paragraph, N₃ is G. In some embodiments, e.g., where the 5′ cap is a trinucleotide cap structure and cap proximal sequence are as described in the previous paragraph, N₄ is G.

### C. Cap Proximal Sequence Comprising Nucleotide(s) of Tetranucleotide 5′ Cap

In some embodiments, the 5′ cap is a tetranucleotide cap structure (e.g., the trinucleotide cap structures described above and herein), wherein the cap proximal sequence comprises N₁, N₂, and N₃ of the 5′ cap, wherein N₁, N₂, and N₃ are any nucleotide, e.g., A, C, G or U.

i. Exemplary Embodiments where N₁ is A

In some embodiments, the 5′ cap is a tetranucleotide cap structure (e.g., the trinucleotide cap structures described above and herein), wherein the cap proximal sequence comprises N₁, N₂, and N₃ of the 5′ cap, wherein N₁, N₂, and N₃ are A. In some embodiments, the 5′ cap is a tetranucleotide cap structure (e.g., the trinucleotide cap structures described above and herein), wherein the cap proximal sequence comprises N₁, N₂, and N₃ of the 5′ cap, wherein N₁ is A, N₂ is A, and N₃ is C. In some embodiments, the 5′ cap is a tetranucleotide cap structure (e.g., the trinucleotide cap structures described above and herein), wherein the cap proximal sequence comprises N₁, N₂, and N₃ of the 5′ cap, wherein N₁ is A, N₂ is A, and N₃ is G. In some embodiments, the 5′ cap is a tetranucleotide cap structure (e.g., the trinucleotide cap structures described above and herein), wherein the cap proximal sequence comprises N₁, N₂, and N₃ of the 5′ cap, wherein N₁ is A, N₂ is A, and N₃ is U. In some embodiments, the 5′ cap is a tetranucleotide cap structure (e.g., the trinucleotide cap structures described above and herein), wherein the cap proximal sequence comprises N₁, N₂, and N₃ of the 5′ cap, wherein N₁ is A, N₂ is C, and N₃ is A. In some embodiments, the 5′ cap is a tetranucleotide cap structure (e.g., the trinucleotide cap structures described above and herein), wherein the cap proximal sequence comprises N₁, N₂, and N₃ of the 5′ cap, wherein N₁ is A, N₂ is C, and N₃ is C. In some embodiments, the 5′ cap is a tetranucleotide cap structure (e.g., the trinucleotide cap structures described above and herein), wherein the cap proximal sequence comprises N₁, N₂, and N₃ of the 5′ cap, wherein N₁ is A, N₂ is C, and N₃ is G. In some embodiments, the 5′ cap is a tetranucleotide cap structure (e.g., the trinucleotide cap structures described above and herein), wherein the cap proximal sequence comprises N₁, N₂, and N₃ of the 5′ cap, wherein N₁ is A, N₂ is C, and N₃ is U. In some embodiments, N₁ is A, N₂ is G, and N₃ is A. In some embodiments, the 5′ cap is a tetranucleotide cap structure (e.g., the trinucleotide cap structures described above and herein), wherein the cap proximal sequence comprises N₁, N₂, and N₃ of the 5′ cap, wherein N₁ is A, N₂ is G, and N₃ is C. In some embodiments, the 5′ cap is a tetranucleotide cap structure (e.g., the trinucleotide cap structures described above and herein), wherein the cap proximal sequence comprises N₁, N₂, and N₃ of the 5′ cap, wherein N₁ is A, N₂ is G, and N₃ is G. In some embodiments, the 5′ cap is a tetranucleotide cap structure (e.g., the trinucleotide cap structures described above and herein), wherein the cap proximal sequence comprises N₁, N₂, and N₃ of the 5′ cap, wherein N₁ is A, N₂ is G, and N₃ is U. In some embodiments, the 5′ cap is a tetranucleotide cap structure (e.g., the trinucleotide cap structures described above and herein), wherein the cap proximal sequence comprises N₁, N₂, and N₃ of the 5′ cap, wherein N₁ is A, N₂ is U, and N₃ is A. In some embodiments, the 5′ cap is a tetranucleotide cap structure (e.g., the trinucleotide cap structures described above and herein), wherein the cap proximal sequence comprises N₁, N₂, and N₃ of the 5′ cap, wherein N₁ is A, N₂ is U, and N₃ is C. In some embodiments, the 5′ cap is a tetranucleotide cap structure (e.g., the trinucleotide cap structures described above and herein), wherein the cap proximal sequence comprises N₁, N₂, and N₃ of the 5′ cap, wherein N₁ is A, N₂ is U, and N₃ is G. In some embodiments, the 5′ cap is a tetranucleotide cap structure (e.g., the trinucleotide cap structures described above and herein), wherein the cap proximal sequence comprises N₁, N₂, and N₃ of the 5′ cap, wherein N₁ is A, N₂ is U, and N₃ is U.

ii. Exemplary Embodiments where N₁ is C

In some embodiments, the 5′ cap is a tetranucleotide cap structure (e.g., the trinucleotide cap structures described above and herein), wherein the cap proximal sequence comprises N₁, N₂, and N₃ of the 5′ cap, wherein N₁, N₂, and N₃ are C. In some embodiments, the 5′ cap is a tetranucleotide cap structure (e.g., the trinucleotide cap structures described above and herein), wherein the cap proximal sequence comprises N₁, N₂, and N₃ of the 5′ cap, wherein N₁ is C, N₂ is A, and N₃ is A. In some embodiments, the 5′ cap is a tetranucleotide cap structure (e.g., the trinucleotide cap structures described above and herein), wherein the cap proximal sequence comprises N₁, N₂, and N₃ of the 5′ cap, wherein N₁ is C, N₂ is A, and N₃ is C. In some embodiments, the 5′ cap is a tetranucleotide cap structure (e.g., the trinucleotide cap structures described above and herein), wherein the cap proximal sequence comprises N₁, N₂, and N₃ of the 5′ cap, wherein N₁ is C, N₂ is A, and N₃ is G. In some embodiments, the 5′ cap is a tetranucleotide cap structure (e.g., the trinucleotide cap structures described above and herein), wherein the cap proximal sequence comprises N₁, N₂, and N₃ of the 5′ cap, wherein N₁ is C, N₂ is A, and N₃ is U. In some embodiments, the 5′ cap is a tetranucleotide cap structure (e.g., the trinucleotide cap structures described above and herein), wherein the cap proximal sequence comprises N₁, N₂, and N₃ of the 5′ cap, wherein N₁ is C, N₂ is C, and N₃ is A. In some embodiments, N₁ is C, N₂ is C, and N₃ is C. In some embodiments, the 5′ cap is a tetranucleotide cap structure (e.g., the trinucleotide cap structures described above and herein), wherein the cap proximal sequence comprises N₁, N₂, and N₃ of the 5′ cap, wherein N₁ is C, N₂ is C, and N₃ is G. In some embodiments, the 5′ cap is a tetranucleotide cap structure (e.g., the trinucleotide cap structures described above and herein), wherein the cap proximal sequence comprises N₁, N₂, and N₃ of the 5′ cap, wherein N₁ is C, N₂ is C, and N₃ is U. In some embodiments, the 5′ cap is a tetranucleotide cap structure (e.g., the trinucleotide cap structures described above and herein), wherein the cap proximal sequence comprises N₁, N₂, and N₃ of the 5′ cap, wherein N₁ is C, N₂ is G, and N₃ is A. In some embodiments, the 5′ cap is a tetranucleotide cap structure (e.g., the trinucleotide cap structures described above and herein), wherein the cap proximal sequence comprises N₁, N₂, and N₃ of the 5′ cap, wherein N₁ is C, N₂ is G, and N₃ is C. In some embodiments, the 5′ cap is a tetranucleotide cap structure (e.g., the trinucleotide cap structures described above and herein), wherein the cap proximal sequence comprises N₁, N₂, and N₃ of the 5′ cap, wherein N₁ is C, N₂ is G, and N₃ is G. In some embodiments, the 5′ cap is a tetranucleotide cap structure (e.g., the trinucleotide cap structures described above and herein), wherein the cap proximal sequence comprises N₁, N₂, and N₃ of the 5′ cap, wherein N₁ is C, N₂ is G, and N₃ is U. In some embodiments, the 5′ cap is a tetranucleotide cap structure (e.g., the trinucleotide cap structures described above and herein), wherein the cap proximal sequence comprises N₁, N₂, and N₃ of the 5′ cap, wherein N₁ is C, N₂ is U, and N₃ is A. In some embodiments, the 5′ cap is a tetranucleotide cap structure (e.g., the trinucleotide cap structures described above and herein), wherein the cap proximal sequence comprises N₁, N₂, and N₃ of the 5′ cap, wherein N₁ is C, N₂ is U, and N₃ is C. In some embodiments, the 5′ cap is a tetranucleotide cap structure (e.g., the trinucleotide cap structures described above and herein), wherein the cap proximal sequence comprises N₁, N₂, and N₃ of the 5′ cap, wherein N₁ is C, N₂ is U, and N₃ is G. In some embodiments, the 5′ cap is a tetranucleotide cap structure (e.g., the trinucleotide cap structures described above and herein), wherein the cap proximal sequence comprises N₁, N₂, and N₃ of the 5′ cap, wherein N₁ is C, N₂ is U, and N₃ is U.

iii. Exemplary Embodiments where N₁ is G

In some embodiments, the 5′ cap is a tetranucleotide cap structure (e.g., the trinucleotide cap structures described above and herein), wherein the cap proximal sequence comprises N₁, N₂, and N₃ of the 5′ cap, wherein N₁, N₂, and N₃ are G. In some embodiments, the 5′ cap is a tetranucleotide cap structure (e.g., the trinucleotide cap structures described above and herein), wherein the cap proximal sequence comprises N₁, N₂, and N₃ of the 5′ cap, wherein N₁ is G, N₂ is A, and N₃ is A. In some embodiments, the 5′ cap is a tetranucleotide cap structure (e.g., the trinucleotide cap structures described above and herein), wherein the cap proximal sequence comprises N₁, N₂, and N₃ of the 5′ cap, wherein N₁ is G, N₂ is A, and N₃ is C. In some embodiments, the 5′ cap is a tetranucleotide cap structure (e.g., the trinucleotide cap structures described above and herein), wherein the cap proximal sequence comprises N₁, N₂, and N₃ of the 5′ cap, wherein N₁ is G, N₂ is A, and N₃ is G. In some embodiments, the 5′ cap is a tetranucleotide cap structure (e.g., the trinucleotide cap structures described above and herein), wherein the cap proximal sequence comprises N₁, N₂, and N₃ of the 5′ cap, wherein N₁ is G, N₂ is A, and N₃ is U. In some embodiments, the 5′ cap is a tetranucleotide cap structure (e.g., the trinucleotide cap structures described above and herein), wherein the cap proximal sequence comprises N₁, N₂, and N₃ of the 5′ cap, wherein N₁ is G, N₂ is C, and N₃ is A. In some embodiments, the 5′ cap is a tetranucleotide cap structure (e.g., the trinucleotide cap structures described above and herein), wherein the cap proximal sequence comprises N₁, N₂, and N₃ of the 5′ cap, wherein N₁ is G, N₂ is C, and N₃ is C. In some embodiments, the 5′ cap is a tetranucleotide cap structure (e.g., the trinucleotide cap structures described above and herein), wherein the cap proximal sequence comprises N₁, N₂, and N₃ of the 5′ cap, wherein N₁ is G, N₂ is C, and N₃ is G. In some embodiments, the 5′ cap is a tetranucleotide cap structure (e.g., the trinucleotide cap structures described above and herein), wherein the cap proximal sequence comprises N₁, N₂, and N₃ of the 5′ cap, wherein N₁ is G, N₂ is C, and N₃ is U. In some embodiments, the 5′ cap is a tetranucleotide cap structure (e.g., the trinucleotide cap structures described above and herein), wherein the cap proximal sequence comprises N₁, N₂, and N₃ of the 5′ cap, wherein N₁ is G, N₂ is G, and N₃ is A In some embodiments, the 5′ cap is a tetranucleotide cap structure (e.g., the trinucleotide cap structures described above and herein), wherein the cap proximal sequence comprises N₁, N₂, and N₃ of the 5′ cap, wherein N₁ is G, N₂ is G, and N₃ is C. In some embodiments, the 5′ cap is a tetranucleotide cap structure (e.g., the trinucleotide cap structures described above and herein), wherein the cap proximal sequence comprises N₁, N₂, and N₃ of the 5′ cap, wherein N₁ is G, N₂ is G, and N₃ is G. In some embodiments, the 5′ cap is a tetranucleotide cap structure (e.g., the trinucleotide cap structures described above and herein), wherein the cap proximal sequence comprises N₁, N₂, and N₃ of the 5′ cap, wherein N₁ is G, N₂ is G, and N₃ is U. In some embodiments, the 5′ cap is a tetranucleotide cap structure (e.g., the trinucleotide cap structures described above and herein), wherein the cap proximal sequence comprises N₁, N₂, and N₃ of the 5′ cap, wherein N₁ is G, N₂ is U, and N₃ is A. In some embodiments, the 5′ cap is a tetranucleotide cap structure (e.g., the trinucleotide cap structures described above and herein), wherein the cap proximal sequence comprises N₁, N₂, and N₃ of the 5′ cap, wherein N₁ is G, N₂ is U, and N₃ is C. In some embodiments, the 5′ cap is a tetranucleotide cap structure (e.g., the trinucleotide cap structures described above and herein), wherein the cap proximal sequence comprises N₁, N₂, and N₃ of the 5′ cap, wherein N₁ is G, N₂ is U, and N₃ is G. In some embodiments, the 5′ cap is a tetranucleotide cap structure (e.g., the trinucleotide cap structures described above and herein), wherein the cap proximal sequence comprises N₁, N₂, and N₃ of the 5′ cap, wherein N₁ is G, N₂ is U, and N₃ is U.

iv. Exemplary Embodiments where N₁ is U

In some embodiments, the 5′ cap is a tetranucleotide cap structure (e.g., the trinucleotide cap structures described above and herein), wherein the cap proximal sequence comprises N₁, N₂, and N₃ of the 5′ cap, wherein N₁, N₂, and N₃ are U. In some embodiments, the 5′ cap is a tetranucleotide cap structure (e.g., the trinucleotide cap structures described above and herein), wherein the cap proximal sequence comprises N₁, N₂, and N₃ of the 5′ cap, wherein N₁ is U, N₂ is A, and N₃ is A. In some embodiments, the 5′ cap is a tetranucleotide cap structure (e.g., the trinucleotide cap structures described above and herein), wherein the cap proximal sequence comprises N₁, N₂, and N₃ of the 5′ cap, wherein N₁ is U, N₂ is A, and N₃ is C. In some embodiments, the 5′ cap is a tetranucleotide cap structure (e.g., the trinucleotide cap structures described above and herein), wherein the cap proximal sequence comprises N₁, N₂, and N₃ of the 5′ cap, wherein N₁ is U, N₂ is A, and N₃ is G. In some embodiments, the 5′ cap is a tetranucleotide cap structure (e.g., the trinucleotide cap structures described above and herein), wherein the cap proximal sequence comprises N₁, N₂, and N₃ of the 5′ cap, wherein N₁ is U, N₂ is A, and N₃ is U. In some embodiments, the 5′ cap is a tetranucleotide cap structure (e.g., the trinucleotide cap structures described above and herein), wherein the cap proximal sequence comprises N₁, N₂, and N₃ of the 5′ cap, wherein N₁ is U, N₂ is C, and N₃ is A. In some embodiments, the 5′ cap is a tetranucleotide cap structure (e.g., the trinucleotide cap structures described above and herein), wherein the cap proximal sequence comprises N₁, N₂, and N₃ of the 5′ cap, wherein N₁ is U, N₂ is C, and N₃ is C. In some embodiments, the 5′ cap is a tetranucleotide cap structure (e.g., the trinucleotide cap structures described above and herein), wherein the cap proximal sequence comprises N₁, N₂, and N₃ of the 5′ cap, wherein N₁ is U, N₂ is C, and N₃ is G. In some embodiments, the 5′ cap is a tetranucleotide cap structure (e.g., the trinucleotide cap structures described above and herein), wherein the cap proximal sequence comprises N₁, N₂, and N₃ of the 5′ cap, wherein N₁ is U, N₂ is C, and N₃ is U. In some embodiments, the 5′ cap is a tetranucleotide cap structure (e.g., the trinucleotide cap structures described above and herein), wherein the cap proximal sequence comprises N₁, N₂, and N₃ of the 5′ cap, wherein N₁ is U, N₂ is G, and N₃ is A. In some embodiments, the 5′ cap is a tetranucleotide cap structure (e.g., the trinucleotide cap structures described above and herein), wherein the cap proximal sequence comprises N₁, N₂, and N₃ of the 5′ cap, wherein N₁ is U, N₂ is G, and N₃ is C. In some embodiments, the 5′ cap is a tetranucleotide cap structure (e.g., the trinucleotide cap structures described above and herein), wherein the cap proximal sequence comprises N₁, N₂, and N₃ of the 5′ cap, wherein N₁ is U, N₂ is G, and N₃ is G. In some embodiments, the 5′ cap is a tetranucleotide cap structure (e.g., the trinucleotide cap structures described above and herein), wherein the cap proximal sequence comprises N₁, N₂, and N₃ of the 5′ cap, wherein N₁ is U, N₂ is G, and N₃ is U. In some embodiments, the 5′ cap is a tetranucleotide cap structure (e.g., the trinucleotide cap structures described above and herein), wherein the cap proximal sequence comprises N₁, N₂, and N₃ of the 5′ cap, wherein N₁ is U, N₂ is U, and N₃ is A. In some embodiments, the 5′ cap is a tetranucleotide cap structure (e.g., the trinucleotide cap structures described above and herein), wherein the cap proximal sequence comprises N₁, N₂, and N₃ of the 5′ cap, wherein N₁ is U, N₂ is U, and N₃ is C. In some embodiments, the 5′ cap is a tetranucleotide cap structure (e.g., the trinucleotide cap structures described above and herein), wherein the cap proximal sequence comprises N₁, N₂, and N₃ of the 5′ cap, wherein N₁ is U, N₂ is U, and N₃ is G. In some embodiments, the 5′ cap is a tetranucleotide cap structure (e.g., the trinucleotide cap structures described above and herein), wherein the cap proximal sequence comprises N₁, N₂, and N₃ of the 5′ cap, wherein N₁ is U, N₂ is U, and N₃ is U.

### D. Exemplary Cap Proximal Sequences

In some embodiments, e.g., where the 5′ cap is a dinucleotide cap structure, a cap proximal sequence comprises N₁ of a the 5′ cap, and N₂, N₃, N₄ and N₅, wherein N₁ to N₅ correspond to positions +1, +2, +3, +4, and/or +5 of an RNA polynucleotide. In some embodiments, e.g., where the 5′ cap is a trinucleotide cap structure, a cap proximal sequence comprises N₁ and N₂ of a the 5′ cap, and N₃, N₄ and N₅, wherein N₁ to N₅ correspond to positions +1, +2, +3, +4, and/or +5 of an RNA polynucleotide. In some embodiments, e.g., where the 5′ cap is a tetranucleotide cap structure, a cap proximal sequence comprises N₁, N₂, and N₃ of a the 5′ cap, and N₄ and N₅, wherein N₁ to N₅ correspond to positions +1, +2, +3, +4, and/or +5 of an RNA polynucleotide.

i. Exemplary Cap Proximal Sequences where N₁ is A, and N₂ is A.

In some embodiments, N₁ is A, N₂ is A, N₃ is A, N₄ is A, and N₅ is A. In some embodiments, N₁ is A, N₂ is A, N₃ is A, N₄ is A, and N₅ is C. In some embodiments, N₁ is A, N₂ is A, N₃ is A, N₄ is A, and N₅ is G. In some embodiments, N₁ is A, N₂ is A, N₃ is A, N₄ is A, and N₅ is U. In some embodiments, N₁ is A, N₂ is A, N₃ is A, N₄ is C, and N₅ is A. In some embodiments, N₁ is A, N₂ is A, N₃ is A, N₄ is C, and N₅ is C. In some embodiments, N₁ is A, N₂ is A, N₃ is A, N₄ is C, and N₅ is G. In some embodiments, N₁ is A, N₂ is A, N₃ is A, N₄ is C, and N₅ is U. In some embodiments, N₁ is A, N₂ is A, N₃ is A, N₄ is G, and N₅ is A. In some embodiments, N₁ is A, N₂ is A, N₃ is A, N₄ is G, and N₅ is C. In some embodiments, N₁ is A, N₂ is A, N₃ is A, N₄ is G, and N₅ is G. In some embodiments, N₁ is A, N₂ is A, N₃ is A, N₄ is G, and N₅ is U. In some embodiments, N₁ is A, N₂ is A, N₃ is A, N₄ is U, and N₅ is A. In some embodiments, N₁ is A, N₂ is A, N₃ is A, N₄ is U, and N₅ is C. In some embodiments, N₁ is A, N₂ is A, N₃ is A, N₄ is U, and N₅ is G. In some embodiments, N₁ is A, N₂ is A, N₃ is A, N₄ is U, and N₅ is U.

In some embodiments, N₁ is A, N₂ is A, N₃ is C, N₄ is A, and N₅ is A. In some embodiments, N₁ is A, N₂ is A, N₃ is C, N₄ is A, and N₅ is C. In some embodiments, N₁ is A, N₂ is A, N₃ is C, N₄ is A, and N₅ is G. In some embodiments, N₁ is A, N₂ is A, N₃ is C, N₄ is A, and N₅ is U. In some embodiments, N₁ is A, N₂ is A, N₃ is C, N₄ is C, and N₅ is A. In some embodiments, N₁ is A, N₂ is A, N₃ is C, N₄ is C, and N₅ is C. In some embodiments, N₁ is A, N₂ is A, N₃ is C, N₄ is C, and N₅ is G. In some embodiments, N₁ is A, N₂ is A, N₃ is C, N₄ is C, and N₅ is U. In some embodiments, N₁ is A, N₂ is A, N₃ is C, N₄ is G, and N₅ is A. In some embodiments, N₁ is A, N₂ is A, N₃ is C, N₄ is G, and N₅ is C. In some embodiments, N₁ is A, N₂ is A, N₃ is C, N₄ is G, and N₅ is G. In some embodiments, N₁ is A, N₂ is A, N₃ is C, N₄ is G, and N₅ is U. In some embodiments, N₁ is A, N₂ is A, N₃ is C, N₄ is U, and N₅ is A. In some embodiments, N₁ is A, N₂ is A, N₃ is C, N₄ is U, and N₅ is C. In some embodiments, N₁ is A, N₂ is A, N₃ is C, N₄ is U, and N₅ is G. In some embodiments, N₁ is A, N₂ is A, N₃ is C, N₄ is U, and N₅ is U.

In some embodiments, N₁ is A, N₂ is A, N₃ is G, N₄ is A, and N₅ is A. In some embodiments, N₁ is A, N₂ is A, N₃ is G, N₄ is A, and N₅ is C. In some embodiments, N₁ is A, N₂ is A, N₃ is G, N₄ is A, and N₅ is G. In some embodiments, N₁ is A, N₂ is A, N₃ is G, N₄ is A, and N₅ is U. In some embodiments, N₁ is A, N₂ is A, N₃ is G, N₄ is C, and N₅ is A. In some embodiments, N₁ is A, N₂ is A, N₃ is G, N₄ is C, and N₅ is C. In some embodiments, N₁ is A, N₂ is A, N₃ is G, N₄ is C, and N₅ is G. In some embodiments, N₁ is A, N₂ is A, N₃ is G, N₄ is C, and N₅ is U. In some embodiments, N₁ is A, N₂ is A, N₃ is G, N₄ is G, and N₅ is A. In some embodiments, N₁ is A, N₂ is A, N₃ is G, N₄ is G, and N₅ is C. In some embodiments, N₁ is A, N₂ is A, N₃ is G, N₄ is G, and N₅ is G. In some embodiments, N₁ is A, N₂ is A, N₃ is G, N₄ is G, and N₅ is U. In some embodiments, N₁ is A, N₂ is A, N₃ is G, N₄ is U, and N₅ is A. In some embodiments, N₁ is A, N₂ is A, N₃ is G, N₄ is U, and N₅ is C. In some embodiments, N₁ is A, N₂ is A, N₃ is G, N₄ is U, and N₅ is G. In some embodiments, N₁ is A, N₂ is A, N₃ is G, N₄ is U, and N₅ is U.

In some embodiments, N₁ is A, N₂ is A, N₃ is U, N₄ is A, and N₅ is A. In some embodiments, N₁ is A, N₂ is A, N₃ is U, N₄ is A, and N₅ is C. In some embodiments, N₁ is A, N₂ is A, N₃ is U, N₄ is A, and N₅ is G. In some embodiments, N₁ is A, N₂ is A, N₃ is U, N₄ is A, and N₅ is U. In some embodiments, N₁ is A, N₂ is A, N₃ is U, N₄ is C, and N₅ is A. In some embodiments, N₁ is A, N₂ is A, N₃ is U, N₄ is C, and N₅ is C. In some embodiments, N₁ is A, N₂ is A, N₃ is U, N₄ is C, and N₅ is G. In some embodiments, N₁ is A, N₂ is A, N₃ is U, N₄ is C, and N₅ is U. In some embodiments, N₁ is A, N₂ is A, N₃ is U, N₄ is G, and N₅ is A. In some embodiments, N₁ is A, N₂ is A, N₃ is U, N₄ is G, and N₅ is C. In some embodiments, N₁ is A, N₂ is A, N₃ is U, N₄ is G, and N₅ is G. In some embodiments, N₁ is A, N₂ is A, N₃ is U, N₄ is G, and N₅ is U. In some embodiments, N₁ is A, N₂ is A, N₃ is U, N₄ is U, and N₅ is A. In some embodiments, N₁ is A, N₂ is A, N₃ is U, N₄ is U, and N₅ is C. In some embodiments, N₁ is A, N₂ is A, N₃ is U, N₄ is U, and N₅ is G. In some embodiments, N₁ is A, N₂ is A, N₃ is U, N₄ is U, and N₅ is U.

ii. Exemplary Cap Proximal Sequences where N₁ is A, and N₂ is C.

In some embodiments, N₁ is A, N₂ is C, N₃ is A, N₄ is A, and N₅ is A. In some embodiments, N₁ is A, N₂ is C, N₃ is A, N₄ is A, and N₅ is C. In some embodiments, N₁ is A, N₂ is C, N₃ is A, N₄ is A, and N₅ is G. In some embodiments, N₁ is A, N₂ is C, N₃ is A, N₄ is A, and N₅ is U. In some embodiments, N₁ is A, N₂ is C, N₃ is A, N₄ is C, and N₅ is A. In some embodiments, N₁ is A, N₂ is C, N₃ is A, N₄ is C, and N₅ is C. In some embodiments, N₁ is A, N₂ is C, N₃ is A, N₄ is C, and N₅ is G. In some embodiments, N₁ is A, N₂ is C, N₃ is A, N₄ is C, and N₅ is U. In some embodiments, N₁ is A, N₂ is C, N₃ is A, N₄ is G, and N₅ is A. In some embodiments, N₁ is A, N₂ is C, N₃ is A, N₄ is G, and N₅ is C. In some embodiments, N₁ is A, N₂ is C, N₃ is A, N₄ is G, and N₅ is G. In some embodiments, N₁ is A, N₂ is C, N₃ is A, N₄ is G, and N₅ is U. In some embodiments, N₁ is A, N₂ is C, N₃ is A, N₄ is U, and N₅ is A. In some embodiments, N₁ is A, N₂ is C, N₃ is A, N₄ is U, and N₅ is C. In some embodiments, N₁ is A, N₂ is C, N₃ is A, N₄ is U, and N₅ is G. In some embodiments, N₁ is A, N₂ is C, N₃ is A, N₄ is U, and N₅ is U.

In some embodiments, N₁ is A, N₂ is C, N₃ is C, N₄ is A, and N₅ is A. In some embodiments, N₁ is A, N₂ is C, N₃ is C, N₄ is A, and N₅ is C. In some embodiments, N₁ is A, N₂ is C, N₃ is C, N₄ is A, and N₅ is G. In some embodiments, N₁ is A, N₂ is C, N₃ is C, N₄ is A, and N₅ is U. In some embodiments, N₁ is A, N₂ is C, N₃ is C, N₄ is C, and N₅ is A. In some embodiments, N₁ is A, N₂ is C, N₃ is C, N₄ is C, and N₅ is C. In some embodiments, N₁ is A, N₂ is C, N₃ is C, N₄ is C, and N₅ is G. In some embodiments, N₁ is A, N₂ is C, N₃ is C, N₄ is C, and N₅ is U. In some embodiments, N₁ is A, N₂ is C, N₃ is C, N₄ is G, and N₅ is A. In some embodiments, N₁ is A, N₂ is C, N₃ is C, N₄ is G, and N₅ is C. In some embodiments, N₁ is A, N₂ is C, N₃ is C, N₄ is G, and N₅ is G. In some embodiments, N₁ is A, N₂ is C, N₃ is C, N₄ is G, and N₅ is U. In some embodiments, N₁ is A, N₂ is C, N₃ is C, N₄ is U, and N₅ is A. In some embodiments, N₁ is A, N₂ is C, N₃ is C, N₄ is U, and N₅ is C. In some embodiments, N₁ is A, N₂ is C, N₃ is C, N₄ is U, and N₅ is G. In some embodiments, N₁ is A, N₂ is C, N₃ is C, N₄ is U, and N₅ is U.

In some embodiments, N₁ is A, N₂ is C, N₃ is G, N₄ is A, and N₅ is A. In some embodiments, N₁ is A, N₂ is C, N₃ is G, N₄ is A, and N₅ is C. In some embodiments, N₁ is A, N₂ is C, N₃ is G, N₄ is A, and N₅ is G. In some embodiments, N₁ is A, N₂ is C, N₃ is G, N₄ is A, and N₅ is U. In some embodiments, N₁ is A, N₂ is C, N₃ is G, N₄ is C, and N₅ is A. In some embodiments, N₁ is A, N₂ is C, N₃ is G, N₄ is C, and N₅ is C. In some embodiments, N₁ is A, N₂ is C, N₃ is G, N₄ is C, and N₅ is G. In some embodiments, N₁ is A, N₂ is C, N₃ is G, N₄ is C, and N₅ is U. In some embodiments, N₁ is A, N₂ is C, N₃ is G, N₄ is G, and N₅ is A. In some embodiments, N₁ is A, N₂ is C, N₃ is G, N₄ is G, and N₅ is C. In some embodiments, N₁ is A, N₂ is C, N₃ is G, N₄ is G, and N₅ is G. In some embodiments, N₁ is A, N₂ is C, N₃ is G, N₄ is G, and N₅ is U. In some embodiments, N₁ is A, N₂ is C, N₃ is G, N₄ is U, and N₅ is A. In some embodiments, N₁ is A, N₂ is C, N₃ is G, N₄ is U, and N₅ is C. In some embodiments, N₁ is A, N₂ is C, N₃ is G, N₄ is U, and N₅ is G. In some embodiments, N₁ is A, N₂ is C, N₃ is G, N₄ is U, and N₅ is U.

In some embodiments, N₁ is A, N₂ is C, N₃ is U, N₄ is A, and N₅ is A. In some embodiments, N₁ is A, N₂ is C, N₃ is U, N₄ is A, and N₅ is C. In some embodiments, N₁ is A, N₂ is C, N₃ is U, N₄ is A, and N₅ is G. In some embodiments, N₁ is A, N₂ is C, N₃ is U, N₄ is A, and N₅ is U. In some embodiments, N₁ is A, N₂ is C, N₃ is U, N₄ is C, and N₅ is A. In some embodiments, N₁ is A, N₂ is C, N₃ is U, N₄ is C, and N₅ is C. In some embodiments, N₁ is A, N₂ is C, N₃ is U, N₄ is C, and N₅ is G. In some embodiments, N₁ is A, N₂ is C, N₃ is U, N₄ is C, and N₅ is U. In some embodiments, N₁ is A, N₂ is C, N₃ is U, N₄ is G, and N₅ is A. In some embodiments, N₁ is A, N₂ is C, N₃ is U, N₄ is G, and N₅ is C. In some embodiments, N₁ is A, N₂ is C, N₃ is U, N₄ is G, and N₅ is G. In some embodiments, N₁ is A, N₂ is C, N₃ is U, N₄ is G, and N₅ is U. In some embodiments, N₁ is A, N₂ is C, N₃ is U, N₄ is U, and N₅ is A. In some embodiments, N₁ is A, N₂ is C, N₃ is U, N₄ is U, and N₅ is C. In some embodiments, N₁ is A, N₂ is C, N₃ is U, N₄ is U, and N₅ is G. In some embodiments, N₁ is A, N₂ is A, N₃ is U, N₄ is U, and N₅ is U.

iii. Exemplary Cap Proximal Sequences where N₁ is A, and N₂ is G.

In some embodiments, N₁ is A, N₂ is G, N₃ is A, N₄ is A, and N₅ is A. In some embodiments, N₁ is A, N₂ is G, N₃ is A, N₄ is A, and N₅ is C. In some embodiments, N₁ is A, N₂ is G, N₃ is A, N₄ is A, and N₅ is G. In some embodiments, N₁ is A, N₂ is G, N₃ is A, N₄ is A, and N₅ is U. In some embodiments, N₁ is A, N₂ is G, N₃ is A, N₄ is C, and N₅ is A. In some embodiments, N₁ is A, N₂ is G, N₃ is A, N₄ is C, and N₅ is C. In some embodiments, N₁ is A, N₂ is G, N₃ is A, N₄ is C, and N₅ is G. In some embodiments, N₁ is A, N₂ is G, N₃ is A, N₄ is C, and N₅ is U. In some embodiments, N₁ is A, N₂ is G, N₃ is A, N₄ is G, and N₅ is A. In some embodiments, N₁ is A, N₂ is G, N₃ is A, N₄ is G, and N₅ is C. In some embodiments, N₁ is A, N₂ is G, N₃ is A, N₄ is G, and N₅ is G. In some embodiments, N₁ is A, N₂ is G, N₃ is A, N₄ is G, and N₅ is U. In some embodiments, N₁ is A, N₂ is G, N₃ is A, N₄ is U, and N₅ is A. In some embodiments, N₁ is A, N₂ is G, N₃ is A, N₄ is U, and N₅ is C. In some embodiments, N₁ is A, N₂ is G, N₃ is A, N₄ is U, and N₅ is G. In some embodiments, N₁ is A, N₂ is G, N₃ is A, N₄ is U, and N₅ is U.

In some embodiments, N₁ is A, N₂ is G, N₃ is C, N₄ is A, and N₅ is A. In some embodiments, N₁ is A, N₂ is G, N₃ is C, N₄ is A, and N₅ is C. In some embodiments, N₁ is A, N₂ is G, N₃ is C, N₄ is A, and N₅ is G. In some embodiments, N₁ is A, N₂ is G, N₃ is C, N₄ is A, and N₅ is U. In some embodiments, N₁ is A, N₂ is G, N₃ is C, N₄ is C, and N₅ is A. In some embodiments, N₁ is A, N₂ is G, N₃ is C, N₄ is C, and N₅ is C. In some embodiments, N₁ is A, N₂ is G, N₃ is C, N₄ is C, and N₅ is G. In some embodiments, N₁ is A, N₂ is G, N₃ is C, N₄ is C, and N₅ is U. In some embodiments, N₁ is A, N₂ is G, N₃ is C, N₄ is G, and N₅ is A. In some embodiments, N₁ is A, N₂ is G, N₃ is C, N₄ is G, and N₅ is C. In some embodiments, N₁ is A, N₂ is G, N₃ is C, N₄ is G, and N₅ is G. In some embodiments, N₁ is A, N₂ is G, N₃ is C, N₄ is G, and N₅ is U. In some embodiments, N₁ is A, N₂ is G, N₃ is C, N₄ is U, and N₅ is A. In some embodiments, N₁ is A, N₂ is G, N₃ is C, N₄ is U, and N₅ is C. In some embodiments, N₁ is A, N₂ is G, N₃ is C, N₄ is U, and N₅ is G. In some embodiments, N₁ is A, N₂ is G, N₃ is C, N₄ is U, and N₅ is U.

In some embodiments, N₁ is A, N₂ is G, N₃ is G, N₄ is A, and N₅ is A. In some embodiments, N₁ is A, N₂ is G, N₃ is G, N₄ is A, and N₅ is C. In some embodiments, N₁ is A, N₂ is G, N₃ is G, N₄ is A, and N₅ is G. In some embodiments, N₁ is A, N₂ is G, N₃ is G, N₄ is A, and N₅ is U. In some embodiments, N₁ is A, N₂ is G, N₃ is G, N₄ is C, and N₅ is A. In some embodiments, N₁ is A, N₂ is G, N₃ is G, N₄ is C, and N₅ is C. In some embodiments, N₁ is A, N₂ is G, N₃ is G, N₄ is C, and N₅ is G. In some embodiments, N₁ is A, N₂ is G, N₃ is G, N₄ is C, and N₅ is U. In some embodiments, N₁ is A, N₂ is G, N₃ is G, N₄ is G, and N₅ is A. In some embodiments, N₁ is A, N₂ is G, N₃ is G, N₄ is G, and N₅ is C. In some embodiments, N₁ is A, N₂ is G, N₃ is G, N₄ is G, and N₅ is G. In some embodiments, N₁ is A, N₂ is G, N₃ is G, N₄ is G, and N₅ is U. In some embodiments, N₁ is A, N₂ is G, N₃ is G, N₄ is U, and N₅ is A. In some embodiments, N₁ is A, N₂ is G, N₃ is G, N₄ is U, and N₅ is C. In some embodiments, N₁ is A, N₂ is G, N₃ is G, N₄ is U, and N₅ is G. In some embodiments, N₁ is A, N₂ is G, N₃ is G, N₄ is U, and N₅ is U.

In some embodiments, N₁ is A, N₂ is G, N₃ is U, N₄ is A, and N₅ is A. In some embodiments, N₁ is A, N₂ is G, N₃ is U, N₄ is A, and N₅ is C. In some embodiments, N₁ is A, N₂ is G, N₃ is U, N₄ is A, and N₅ is G. In some embodiments, N₁ is A, N₂ is G, N₃ is U, N₄ is A, and N₅ is U. In some embodiments, N₁ is A, N₂ is G, N₃ is U, N₄ is C, and N₅ is A. In some embodiments, N₁ is A, N₂ is G, N₃ is U, N₄ is C, and N₅ is C. In some embodiments, N₁ is A, N₂ is G, N₃ is U, N₄ is C, and N₅ is G. In some embodiments, N₁ is A, N₂ is G, N₃ is U, N₄ is C, and N₅ is U. In some embodiments, N₁ is A, N₂ is G, N₃ is U, N₄ is G, and N₅ is A. In some embodiments, N₁ is A, N₂ is G, N₃ is U, N₄ is G, and N₅ is C. In some embodiments, N₁ is A, N₂ is G, N₃ is U, N₄ is G, and N₅ is G. In some embodiments, N₁ is A, N₂ is G, N₃ is U, N₄ is G, and N₅ is U. In some embodiments, N₁ is A, N₂ is G, N₃ is U, N₄ is U, and N₅ is A. In some embodiments, N₁ is A, N₂ is G, N₃ is U, N₄ is U, and N₅ is C. In some embodiments, N₁ is A, N₂ is G, N₃ is U, N₄ is U, and N₅ is G. In some embodiments, N₁ is A, N₂ is G, N₃ is U, N₄ is U, and N₅ is U.

iv. Exemplary Cap Proximal Sequences where N₁ is A, and N₂ is U.

In some embodiments, N₁ is A, N₂ is U, N₃ is A, N₄ is A, and N₅ is A. In some embodiments, N₁ is A, N₂ is U, N₃ is A, N₄ is A, and N₅ is C. In some embodiments, N₁ is A, N₂ is U, N₃ is A, N₄ is A, and N₅ is G. In some embodiments, N₁ is A, N₂ is U, N₃ is A, N₄ is A, and N₅ is U. In some embodiments, N₁ is A, N₂ is U, N₃ is A, N₄ is C, and N₅ is A. In some embodiments, N₁ is A, N₂ is U, N₃ is A, N₄ is C, and N₅ is C. In some embodiments, N₁ is A, N₂ is U, N₃ is A, N₄ is C, and N₅ is G. In some embodiments, N₁ is A, N₂ is U, N₃ is A, N₄ is C, and N₅ is U. In some embodiments, N₁ is A, N₂ is U, N₃ is A, N₄ is G, and N₅ is A. In some embodiments, N₁ is A, N₂ is U, N₃ is A, N₄ is G, and N₅ is C. In some embodiments, N₁ is A, N₂ is U, N₃ is A, N₄ is G, and N₅ is G. In some embodiments, N₁ is A, N₂ is U, N₃ is A, N₄ is G, and N₅ is U. In some embodiments, N₁ is A, N₂ is U, N₃ is A, N₄ is U, and N₅ is A. In some embodiments, N₁ is A, N₂ is U, N₃ is A, N₄ is U, and N₅ is C. In some embodiments, N₁ is A, N₂ is U, N₃ is A, N₄ is U, and N₅ is G. In some embodiments, N₁ is A, N₂ is U, N₃ is A, N₄ is U, and N₅ is U.

In some embodiments, N₁ is A, N₂ is U, N₃ is C, N₄ is A, and N₅ is A. In some embodiments, N₁ is A, N₂ is U, N₃ is C, N₄ is A, and N₅ is C. In some embodiments, N₁ is A, N₂ is U, N₃ is C, N₄ is A, and N₅ is G. In some embodiments, N₁ is A, N₂ is U, N₃ is C, N₄ is A, and N₅ is U. In some embodiments, N₁ is A, N₂ is U, N₃ is C, N₄ is C, and N₅ is A. In some embodiments, N₁ is A, N₂ is U, N₃ is C, N₄ is C, and N₅ is C. In some embodiments, N₁ is A, N₂ is U, N₃ is C, N₄ is C, and N₅ is G. In some embodiments, N₁ is A, N₂ is U, N₃ is C, N₄ is C, and N₅ is U. In some embodiments, N₁ is A, N₂ is U, N₃ is C, N₄ is G, and N₅ is A. In some embodiments, N₁ is A, N₂ is U, N₃ is C, N₄ is G, and N₅ is C. In some embodiments, N₁ is A, N₂ is U, N₃ is C, N₄ is G, and N₅ is G. In some embodiments, N₁ is A, N₂ is U, N₃ is C, N₄ is G, and N₅ is U. In some embodiments, N₁ is A, N₂ is U, N₃ is C, N₄ is U, and N₅ is A. In some embodiments, N₁ is A, N₂ is U, N₃ is C, N₄ is U, and N₅ is C. In some embodiments, N₁ is A, N₂ is U, N₃ is C, N₄ is U, and N₅ is G. In some embodiments, N₁ is A, N₂ is U, N₃ is C, N₄ is U, and N₅ is U.

In some embodiments, N₁ is A, N₂ is U, N₃ is G, N₄ is A, and N₅ is A. In some embodiments, N₁ is A, N₂ is U, N₃ is G, N₄ is A, and N₅ is C. In some embodiments, N₁ is A, N₂ is U, N₃ is G, N₄ is A, and N₅ is G. In some embodiments, N₁ is A, N₂ is U, N₃ is G, N₄ is A, and N₅ is U. In some embodiments, N₁ is A, N₂ is U, N₃ is G, N₄ is C, and N₅ is A. In some embodiments, N₁ is A, N₂ is U, N₃ is G, N₄ is C, and N₅ is C. In some embodiments, N₁ is A, N₂ is U, N₃ is G, N₄ is C, and N₅ is G. In some embodiments, N₁ is A, N₂ is U, N₃ is G, N₄ is C, and N₅ is U. In some embodiments, N₁ is A, N₂ is U, N₃ is G, N₄ is G, and N₅ is A. In some embodiments, N₁ is A, N₂ is U, N₃ is G, N₄ is G, and N₅ is C. In some embodiments, N₁ is A, N₂ is U, N₃ is G, N₄ is G, and N₅ is G. In some embodiments, N₁ is A, N₂ is U, N₃ is G, N₄ is G, and N₅ is U. In some embodiments, N₁ is A, N₂ is U, N₃ is G, N₄ is U, and N₅ is A. In some embodiments, N₁ is A, N₂ is U, N₃ is G, N₄ is U, and N₅ is C. In some embodiments, N₁ is A, N₂ is U, N₃ is G, N₄ is U, and N₅ is G. In some embodiments, N₁ is A, N₂ is U, N₃ is G, N₄ is U, and N₅ is U.

In some embodiments, N₁ is A, N₂ is U, N₃ is U, N₄ is A, and N₅ is A. In some embodiments, N₁ is A, N₂ is U, N₃ is U, N₄ is A, and N₅ is C. In some embodiments, N₁ is A, N₂ is U, N₃ is U, N₄ is A, and N₅ is G. In some embodiments, N₁ is A, N₂ is U, N₃ is U, N₄ is A, and N₅ is U. In some embodiments, N₁ is A, N₂ is U, N₃ is U, N₄ is C, and N₅ is A. In some embodiments, N₁ is A, N₂ is U, N₃ is U, N₄ is C, and N₅ is C. In some embodiments, N₁ is A, N₂ is U, N₃ is U, N₄ is C, and N₅ is G. In some embodiments, N₁ is A, N₂ is U, N₃ is U, N₄ is C, and N₅ is U. In some embodiments, N₁ is A, N₂ is U, N₃ is U, N₄ is G, and N₅ is A. In some embodiments, N₁ is A, N₂ is U, N₃ is U, N₄ is G, and N₅ is C. In some embodiments, N₁ is A, N₂ is U, N₃ is U, N₄ is G, and N₅ is G. In some embodiments, N₁ is A, N₂ is U, N₃ is U, N₄ is G, and N₅ is U. In some embodiments, N₁ is A, N₂ is U, N₃ is U, N₄ is U, and N₅ is A. In some embodiments, N₁ is A, N₂ is U, N₃ is U, N₄ is U, and N₅ is C. In some embodiments, N₁ is A, N₂ is U, N₃ is U, N₄ is U, and N₅ is G. In some embodiments, N₁ is A, N₂ is U, N₃ is U, N₄ is U, and N₅ is U.

v. Exemplary Cap Proximal Sequences where N₁ is C, and N₂ is A.

In some embodiments, N₁ is C, N₂ is A, N₃ is A, N₄ is A, and N₅ is A. In some embodiments, N₁ is C, N₂ is A, N₃ is A, N₄ is A, and N₅ is C. In some embodiments, N₁ is C, N₂ is A, N₃ is A, N₄ is A, and N₅ is G. In some embodiments, N₁ is C, N₂ is A, N₃ is A, N₄ is A, and N₅ is U. In some embodiments, N₁ is C, N₂ is A, N₃ is A, N₄ is C, and N₅ is A. In some embodiments, N₁ is C, N₂ is A, N₃ is A, N₄ is C, and N₅ is C. In some embodiments, N₁ is C, N₂ is A, N₃ is A, N₄ is C, and N₅ is G. In some embodiments, N₁ is C, N₂ is A, N₃ is A, N₄ is C, and N₅ is U. In some embodiments, N₁ is C, N₂ is A, N₃ is A, N₄ is G, and N₅ is A. In some embodiments, N₁ is C, N₂ is A, N₃ is A, N₄ is G, and N₅ is C. In some embodiments, N₁ is C, N₂ is A, N₃ is A, N₄ is G, and N₅ is G. In some embodiments, N₁ is C, N₂ is A, N₃ is A, N₄ is G, and N₅ is U. In some embodiments, N₁ is C, N₂ is A, N₃ is A, N₄ is U, and N₅ is A. In some embodiments, N₁ is C, N₂ is A, N₃ is A, N₄ is U, and N₅ is C. In some embodiments, N₁ is C, N₂ is A, N₃ is A, N₄ is U, and N₅ is G. In some embodiments, N₁ is C, N₂ is A, N₃ is A, N₄ is U, and N₅ is U.

In some embodiments, N₁ is C, N₂ is A, N₃ is C, N₄ is A, and N₅ is A. In some embodiments, N₁ is C, N₂ is A, N₃ is C, N₄ is A, and N₅ is C. In some embodiments, N₁ is C, N₂ is A, N₃ is C, N₄ is A, and N₅ is G. In some embodiments, N₁ is C, N₂ is A, N₃ is C, N₄ is A, and N₅ is U. In some embodiments, N₁ is C, N₂ is A, N₃ is C, N₄ is C, and N₅ is A. In some embodiments, N₁ is C, N₂ is A, N₃ is C, N₄ is C, and N₅ is C. In some embodiments, N₁ is C, N₂ is A, N₃ is C, N₄ is C, and N₅ is G. In some embodiments, N₁ is C, N₂ is A, N₃ is C, N₄ is C, and N₅ is U. In some embodiments, N₁ is C, N₂ is A, N₃ is C, N₄ is G, and N₅ is A. In some embodiments, N₁ is C, N₂ is A, N₃ is C, N₄ is G, and N₅ is C. In some embodiments, N₁ is C, N₂ is A, N₃ is C, N₄ is G, and N₅ is G. In some embodiments, N₁ is C, N₂ is A, N₃ is C, N₄ is G, and N₅ is U. In some embodiments, N₁ is C, N₂ is A, N₃ is C, N₄ is U, and N₅ is A. In some embodiments, N₁ is C, N₂ is A, N₃ is C, N₄ is U, and N₅ is C. In some embodiments, N₁ is C, N₂ is A, N₃ is C, N₄ is U, and N₅ is G. In some embodiments, N₁ is C, N₂ is A, N₃ is C, N₄ is U, and N₅ is U.

In some embodiments, N₁ is C, N₂ is A, N₃ is G, N₄ is A, and N₅ is A. In some embodiments, N₁ is C, N₂ is A, N₃ is G, N₄ is A, and N₅ is C. In some embodiments, N₁ is C, N₂ is A, N₃ is G, N₄ is A, and N₅ is G. In some embodiments, N₁ is C, N₂ is A, N₃ is G, N₄ is A, and N₅ is U. In some embodiments, N₁ is C, N₂ is A, N₃ is G, N₄ is C, and N₅ is A. In some embodiments, N₁ is C, N₂ is A, N₃ is G, N₄ is C, and N₅ is C. In some embodiments, N₁ is C, N₂ is A, N₃ is G, N₄ is C, and N₅ is G. In some embodiments, N₁ is C, N₂ is A, N₃ is G, N₄ is C, and N₅ is U. In some embodiments, N₁ is C, N₂ is A, N₃ is G, N₄ is G, and N₅ is A. In some embodiments, N₁ is C, N₂ is A, N₃ is G, N₄ is G, and N₅ is C. In some embodiments, N₁ is C, N₂ is A, N₃ is G, N₄ is G, and N₅ is G. In some embodiments, N₁ is C, N₂ is A, N₃ is G, N₄ is G, and N₅ is U. In some embodiments, N₁ is C, N₂ is A, N₃ is G, N₄ is U, and N₅ is A. In some embodiments, N₁ is C, N₂ is A, N₃ is G, N₄ is U, and N₅ is C. In some embodiments, N₁ is C, N₂ is A, N₃ is G, N₄ is U, and N₅ is G. In some embodiments, N₁ is C, N₂ is A, N₃ is G, N₄ is U, and N₅ is U.

In some embodiments, N₁ is C, N₂ is A, N₃ is U, N₄ is A, and N₅ is A. In some embodiments, N₁ is C, N₂ is A, N₃ is U, N₄ is A, and N₅ is C. In some embodiments, N₁ is C, N₂ is A, N₃ is U, N₄ is A, and N₅ is G. In some embodiments, N₁ is C, N₂ is A, N₃ is U, N₄ is A, and N₅ is U. In some embodiments, N₁ is C, N₂ is A, N₃ is U, N₄ is C, and N₅ is A. In some embodiments, N₁ is C, N₂ is A, N₃ is U, N₄ is C, and N₅ is C. In some embodiments, N₁ is C, N₂ is A, N₃ is U, N₄ is C, and N₅ is G. In some embodiments, N₁ is C, N₂ is A, N₃ is U, N₄ is C, and N₅ is U. In some embodiments, N₁ is C, N₂ is A, N₃ is U, N₄ is G, and N₅ is A. In some embodiments, N₁ is C, N₂ is A, N₃ is U, N₄ is G, and N₅ is C. In some embodiments, N₁ is C, N₂ is A, N₃ is U, N₄ is G, and N₅ is G. In some embodiments, N₁ is C, N₂ is A, N₃ is U, N₄ is G, and N₅ is U. In some embodiments, N₁ is C, N₂ is A, N₃ is U, N₄ is U, and N₅ is A. In some embodiments, N₁ is C, N₂ is A, N₃ is U, N₄ is U, and N₅ is C. In some embodiments, N₁ is C, N₂ is A, N₃ is U, N₄ is U, and N₅ is G. In some embodiments, N₁ is C, N₂ is A, N₃ is U, N₄ is U, and N₅ is U.

vi. Exemplary Cap Proximal Sequences where N₁ is C, and N₂ is C.

In some embodiments, N₁ is C, N₂ is C, N₃ is A, N₄ is A, and N₅ is A. In some embodiments, N₁ is C, N₂ is C, N₃ is A, N₄ is A, and N₅ is C. In some embodiments, N₁ is C, N₂ is C, N₃ is A, N₄ is A, and N₅ is G. In some embodiments, N₁ is C, N₂ is C, N₃ is A, N₄ is A, and N₅ is U. In some embodiments, N₁ is C, N₂ is C, N₃ is A, N₄ is C, and N₅ is A. In some embodiments, N₁ is C, N₂ is C, N₃ is A, N₄ is C, and N₅ is C. In some embodiments, N₁ is C, N₂ is C, N₃ is A, N₄ is C, and N₅ is G. In some embodiments, N₁ is C, N₂ is C, N₃ is A, N₄ is C, and N₅ is U. In some embodiments, N₁ is C, N₂ is C, N₃ is A, N₄ is G, and N₅ is A. In some embodiments, N₁ is C, N₂ is C, N₃ is A, N₄ is G, and N₅ is C. In some embodiments, N₁ is C, N₂ is C, N₃ is A, N₄ is G, and N₅ is G. In some embodiments, N₁ is C, N₂ is C, N₃ is A, N₄ is G, and N₅ is U. In some embodiments, N₁ is C, N₂ is C, N₃ is A, N₄ is U, and N₅ is A. In some embodiments, N₁ is C, N₂ is C, N₃ is A, N₄ is U, and N₅ is C. In some embodiments, N₁ is C, N₂ is C, N₃ is A, N₄ is U, and N₅ is G. In some embodiments, N₁ is C, N₂ is C, N₃ is A, N₄ is U, and N₅ is U.

In some embodiments, N₁ is C, N₂ is C, N₃ is C, N₄ is A, and N₅ is A. In some embodiments, N₁ is C, N₂ is C, N₃ is C, N₄ is A, and N₅ is C. In some embodiments, N₁ is C, N₂ is C, N₃ is C, N₄ is A, and N₅ is G. In some embodiments, N₁ is C, N₂ is C, N₃ is C, N₄ is A, and N₅ is U. In some embodiments, N₁ is C, N₂ is C, N₃ is C, N₄ is C, and N₅ is A. In some embodiments, N₁ is C, N₂ is C, N₃ is C, N₄ is C, and N₅ is C. In some embodiments, N₁ is C, N₂ is C, N₃ is C, N₄ is C, and N₅ is G. In some embodiments, N₁ is C, N₂ is C, N₃ is C, N₄ is C, and N₅ is U. In some embodiments, N₁ is C, N₂ is C, N₃ is C, N₄ is G, and N₅ is A. In some embodiments, N₁ is C, N₂ is C, N₃ is C, N₄ is G, and N₅ is C. In some embodiments, N₁ is C, N₂ is C, N₃ is C, N₄ is G, and N₅ is G. In some embodiments, N₁ is C, N₂ is C, N₃ is C, N₄ is G, and N₅ is U. In some embodiments, N₁ is C, N₂ is C, N₃ is C, N₄ is U, and N₅ is A. In some embodiments, N₁ is C, N₂ is C, N₃ is C, N₄ is U, and N₅ is C. In some embodiments, N₁ is C, N₂ is C, N₃ is C, N₄ is U, and N₅ is G. In some embodiments, N₁ is C, N₂ is C, N₃ is C, N₄ is U, and N₅ is U.

In some embodiments, N₁ is C, N₂ is C, N₃ is G, N₄ is A, and N₅ is A. In some embodiments, N₁ is C, N₂ is C, N₃ is G, N₄ is A, and N₅ is C. In some embodiments, N₁ is C, N₂ is C, N₃ is G, N₄ is A, and N₅ is G. In some embodiments, N₁ is C, N₂ is C, N₃ is G, N₄ is A, and N₅ is U. In some embodiments, N₁ is C, N₂ is C, N₃ is G, N₄ is C, and N₅ is A. In some embodiments, N₁ is C, N₂ is C, N₃ is G, N₄ is C, and N₅ is C. In some embodiments, N₁ is C, N₂ is C, N₃ is G, N₄ is C, and N₅ is G. In some embodiments, N₁ is C, N₂ is C, N₃ is G, N₄ is C, and N₅ is U. In some embodiments, N₁ is C, N₂ is C, N₃ is G, N₄ is G, and N₅ is A. In some embodiments, N₁ is C, N₂ is C, N₃ is G, N₄ is G, and N₅ is C. In some embodiments, N₁ is C, N₂ is C, N₃ is G, N₄ is G, and N₅ is G. In some embodiments, N₁ is C, N₂ is C, N₃ is G, N₄ is G, and N₅ is U. In some embodiments, N₁ is C, N₂ is C, N₃ is G, N₄ is U, and N₅ is A. In some embodiments, N₁ is C, N₂ is C, N₃ is G, N₄ is U, and N₅ is C. In some embodiments, N₁ is C, N₂ is C, N₃ is G, N₄ is U, and N₅ is G. In some embodiments, N₁ is C, N₂ is C, N₃ is G, N₄ is U, and N₅ is U.

In some embodiments, N₁ is C, N₂ is C, N₃ is U, N₄ is A, and N₅ is A. In some embodiments, N₁ is C, N₂ is C, N₃ is U, N₄ is A, and N₅ is C. In some embodiments, N₁ is C, N₂ is C, N₃ is U, N₄ is A, and N₅ is G. In some embodiments, N₁ is C, N₂ is C, N₃ is U, N₄ is A, and N₅ is U. In some embodiments, N₁ is C, N₂ is C, N₃ is U, N₄ is C, and N₅ is A. In some embodiments, N₁ is C, N₂ is C, N₃ is U, N₄ is C, and N₅ is C. In some embodiments, N₁ is C, N₂ is C, N₃ is U, N₄ is C, and N₅ is G. In some embodiments, N₁ is C, N₂ is C, N₃ is U, N₄ is C, and N₅ is U. In some embodiments, N₁ is C, N₂ is C, N₃ is U, N₄ is G, and N₅ is A. In some embodiments, N₁ is C, N₂ is C, N₃ is U, N₄ is G, and N₅ is C. In some embodiments, N₁ is C, N₂ is C, N₃ is U, N₄ is G, and N₅ is G. In some embodiments, N₁ is C, N₂ is C, N₃ is U, N₄ is G, and N₅ is U. In some embodiments, N₁ is C, N₂ is C, N₃ is U, N₄ is U, and N₅ is A. In some embodiments, N₁ is C, N₂ is C, N₃ is U, N₄ is U, and N₅ is C. In some embodiments, N₁ is C, N₂ is C, N₃ is U, N₄ is U, and N₅ is G. In some embodiments, N₁ is C, N₂ is A, N₃ is U, N₄ is U, and N₅ is U.

vii. Exemplary Cap Proximal Sequences where N₁ is C, and N₂ is G.

In some embodiments, N₁ is C, N₂ is G, N₃ is A, N₄ is A, and N₅ is A. In some embodiments, N₁ is C, N₂ is G, N₃ is A, N₄ is A, and N₅ is C. In some embodiments, N₁ is C, N₂ is G, N₃ is A, N₄ is A, and N₅ is G. In some embodiments, N₁ is C, N₂ is G, N₃ is A, N₄ is A, and N₅ is U. In some embodiments, N₁ is C, N₂ is G, N₃ is A, N₄ is C, and N₅ is A. In some embodiments, N₁ is C, N₂ is G, N₃ is A, N₄ is C, and N₅ is C. In some embodiments, N₁ is C, N₂ is G, N₃ is A, N₄ is C, and N₅ is G. In some embodiments, N₁ is C, N₂ is G, N₃ is A, N₄ is C, and N₅ is U. In some embodiments, N₁ is C, N₂ is G, N₃ is A, N₄ is G, and N₅ is A. In some embodiments, N₁ is C, N₂ is G, N₃ is A, N₄ is G, and N₅ is C. In some embodiments, N₁ is C, N₂ is G, N₃ is A, N₄ is G, and N₅ is G. In some embodiments, N₁ is C, N₂ is G, N₃ is A, N₄ is G, and N₅ is U. In some embodiments, N₁ is C, N₂ is G, N₃ is A, N₄ is U, and N₅ is A. In some embodiments, N₁ is C, N₂ is G, N₃ is A, N₄ is U, and N₅ is C. In some embodiments, N₁ is C, N₂ is G, N₃ is A, N₄ is U, and N₅ is G. In some embodiments, N₁ is C, N₂ is G, N₃ is A, N₄ is U, and N₅ is U.

In some embodiments, N₁ is C, N₂ is G, N₃ is C, N₄ is A, and N₅ is A. In some embodiments, N₁ is C, N₂ is G, N₃ is C, N₄ is A, and N₅ is C. In some embodiments, N₁ is C, N₂ is G, N₃ is C, N₄ is A, and N₅ is G. In some embodiments, N₁ is C, N₂ is G, N₃ is C, N₄ is A, and N₅ is U. In some embodiments, N₁ is C, N₂ is G, N₃ is C, N₄ is C, and N₅ is A. In some embodiments, N₁ is C, N₂ is G, N₃ is C, N₄ is C, and N₅ is C. In some embodiments, N₁ is C, N₂ is G, N₃ is C, N₄ is C, and N₅ is G. In some embodiments, N₁ is C, N₂ is G, N₃ is C, N₄ is C, and N₅ is U. In some embodiments, N₁ is C, N₂ is G, N₃ is C, N₄ is G, and N₅ is A. In some embodiments, N₁ is C, N₂ is G, N₃ is C, N₄ is G, and N₅ is C. In some embodiments, N₁ is C, N₂ is G, N₃ is C, N₄ is G, and N₅ is G. In some embodiments, N₁ is C, N₂ is G, N₃ is C, N₄ is G, and N₅ is U. In some embodiments, N₁ is C, N₂ is G, N₃ is C, N₄ is U, and N₅ is A. In some embodiments, N₁ is C, N₂ is G, N₃ is C, N₄ is U, and N₅ is C. In some embodiments, N₁ is C, N₂ is G, N₃ is C, N₄ is U, and N₅ is G. In some embodiments, N₁ is C, N₂ is G, N₃ is C, N₄ is U, and N₅ is U.

In some embodiments, N₁ is C, N₂ is G, N₃ is G, N₄ is A, and N₅ is A. In some embodiments, N₁ is C, N₂ is G, N₃ is G, N₄ is A, and N₅ is C. In some embodiments, N₁ is C, N₂ is G, N₃ is G, N₄ is A, and N₅ is G. In some embodiments, N₁ is C, N₂ is G, N₃ is G, N₄ is A, and N₅ is U. In some embodiments, N₁ is C, N₂ is G, N₃ is G, N₄ is C, and N₅ is A. In some embodiments, N₁ is C, N₂ is G, N₃ is G, N₄ is C, and N₅ is C. In some embodiments, N₁ is C, N₂ is G, N₃ is G, N₄ is C, and N₅ is G. In some embodiments, N₁ is C, N₂ is G, N₃ is G, N₄ is C, and N₅ is U. In some embodiments, N₁ is C, N₂ is G, N₃ is G, N₄ is G, and N₅ is A. In some embodiments, N₁ is C, N₂ is G, N₃ is G, N₄ is G, and N₅ is C. In some embodiments, N₁ is C, N₂ is G, N₃ is G, N₄ is G, and N₅ is G. In some embodiments, N₁ is C, N₂ is G, N₃ is G, N₄ is G, and N₅ is U. In some embodiments, N₁ is C, N₂ is G, N₃ is G, N₄ is U, and N₅ is A. In some embodiments, N₁ is C, N₂ is G, N₃ is G, N₄ is U, and N₅ is C. In some embodiments, N₁ is C, N₂ is G, N₃ is G, N₄ is U, and N₅ is G. In some embodiments, N₁ is C, N₂ is G, N₃ is G, N₄ is U, and N₅ is U.

In some embodiments, N₁ is C, N₂ is G, N₃ is U, N₄ is A, and N₅ is A. In some embodiments, N₁ is C, N₂ is G, N₃ is U, N₄ is A, and N₅ is C. In some embodiments, N₁ is C, N₂ is G, N₃ is U, N₄ is A, and N₅ is G. In some embodiments, N₁ is C, N₂ is G, N₃ is U, N₄ is A, and N₅ is U. In some embodiments, N₁ is C, N₂ is G, N₃ is U, N₄ is C, and N₅ is A. In some embodiments, N₁ is C, N₂ is G, N₃ is U, N₄ is C, and N₅ is C. In some embodiments, N₁ is C, N₂ is G, N₃ is U, N₄ is C, and N₅ is G. In some embodiments, N₁ is C, N₂ is G, N₃ is U, N₄ is C, and N₅ is U. In some embodiments, N₁ is C, N₂ is G, N₃ is U, N₄ is G, and N₅ is A. In some embodiments, N₁ is C, N₂ is G, N₃ is U, N₄ is G, and N₅ is C. In some embodiments, N₁ is C, N₂ is G, N₃ is U, N₄ is G, and N₅ is G. In some embodiments, N₁ is C, N₂ is G, N₃ is U, N₄ is G, and N₅ is U. In some embodiments, N₁ is C, N₂ is G, N₃ is U, N₄ is U, and N₅ is A. In some embodiments, N₁ is C, N₂ is G, N₃ is U, N₄ is U, and N₅ is C. In some embodiments, N₁ is C, N₂ is G, N₃ is U, N₄ is U, and N₅ is G. In some embodiments, N₁ is C, N₂ is G, N₃ is U, N₄ is U, and N₅ is U.

viii. Exemplary Cap Proximal Sequences where N₁ is C, and N₂ is U.

In some embodiments, N₁ is C, N₂ is U, N₃ is A, N₄ is A, and N₅ is A. In some embodiments, N₁ is C, N₂ is U, N₃ is A, N₄ is A, and N₅ is C. In some embodiments, N₁ is C, N₂ is U, N₃ is A, N₄ is A, and N₅ is G. In some embodiments, N₁ is C, N₂ is U, N₃ is A, N₄ is A, and N₅ is U. In some embodiments, N₁ is C, N₂ is U, N₃ is A, N₄ is C, and N₅ is A. In some embodiments, N₁ is C, N₂ is U, N₃ is A, N₄ is C, and N₅ is C. In some embodiments, N₁ is C, N₂ is U, N₃ is A, N₄ is C, and N₅ is G. In some embodiments, N₁ is C, N₂ is U, N₃ is A, N₄ is C, and N₅ is U. In some embodiments, N₁ is C, N₂ is U, N₃ is A, N₄ is G, and N₅ is A. In some embodiments, N₁ is C, N₂ is U, N₃ is A, N₄ is G, and N₅ is C. In some embodiments, N₁ is C, N₂ is U, N₃ is A, N₄ is G, and N₅ is G. In some embodiments, N₁ is C, N₂ is U, N₃ is A, N₄ is G, and N₅ is U. In some embodiments, N₁ is C, N₂ is U, N₃ is A, N₄ is U, and N₅ is A. In some embodiments, N₁ is C, N₂ is U, N₃ is A, N₄ is U, and N₅ is C. In some embodiments, N₁ is C, N₂ is U, N₃ is A, N₄ is U, and N₅ is G. In some embodiments, N₁ is C, N₂ is U, N₃ is A, N₄ is U, and N₅ is U.

In some embodiments, N₁ is C, N₂ is U, N₃ is C, N₄ is A, and N₅ is A. In some embodiments, N₁ is C, N₂ is U, N₃ is C, N₄ is A, and N₅ is C. In some embodiments, N₁ is C, N₂ is U, N₃ is C, N₄ is A, and N₅ is G. In some embodiments, N₁ is C, N₂ is U, N₃ is C, N₄ is A, and N₅ is U. In some embodiments, N₁ is C, N₂ is U, N₃ is C, N₄ is C, and N₅ is A. In some embodiments, N₁ is C, N₂ is U, N₃ is C, N₄ is C, and N₅ is C. In some embodiments, N₁ is C, N₂ is U, N₃ is C, N₄ is C, and N₅ is G. In some embodiments, N₁ is C, N₂ is U, N₃ is C, N₄ is C, and N₅ is U. In some embodiments, N₁ is C, N₂ is U, N₃ is C, N₄ is G, and N₅ is A. In some embodiments, N₁ is C, N₂ is U, N₃ is C, N₄ is G, and N₅ is C. In some embodiments, N₁ is C, N₂ is U, N₃ is C, N₄ is G, and N₅ is G. In some embodiments, N₁ is C, N₂ is U, N₃ is C, N₄ is G, and N₅ is U. In some embodiments, N₁ is C, N₂ is U, N₃ is C, N₄ is U, and N₅ is A. In some embodiments, N₁ is C, N₂ is U, N₃ is C, N₄ is U, and N₅ is C. In some embodiments, N₁ is C, N₂ is U, N₃ is C, N₄ is U, and N₅ is G. In some embodiments, N₁ is C, N₂ is U, N₃ is C, N₄ is U, and N₅ is U.

In some embodiments, N₁ is C, N₂ is U, N₃ is G, N₄ is A, and N₅ is A. In some embodiments, N₁ is C, N₂ is U, N₃ is G, N₄ is A, and N₅ is C. In some embodiments, N₁ is C, N₂ is U, N₃ is G, N₄ is A, and N₅ is G. In some embodiments, N₁ is C, N₂ is U, N₃ is G, N₄ is A, and N₅ is U. In some embodiments, N₁ is C, N₂ is U, N₃ is G, N₄ is C, and N₅ is A. In some embodiments, N₁ is C, N₂ is U, N₃ is G, N₄ is C, and N₅ is C. In some embodiments, N₁ is C, N₂ is U, N₃ is G, N₄ is C, and N₅ is G. In some embodiments, N₁ is C, N₂ is U, N₃ is G, N₄ is C, and N₅ is U. In some embodiments, N₁ is C, N₂ is U, N₃ is G, N₄ is G, and N₅ is A. In some embodiments, N₁ is C, N₂ is U, N₃ is G, N₄ is G, and N₅ is C. In some embodiments, N₁ is C, N₂ is U, N₃ is G, N₄ is G, and N₅ is G. In some embodiments, N₁ is C, N₂ is U, N₃ is G, N₄ is G, and N₅ is U. In some embodiments, N₁ is C, N₂ is U, N₃ is G, N₄ is U, and N₅ is A. In some embodiments, N₁ is C, N₂ is U, N₃ is G, N₄ is U, and N₅ is C. In some embodiments, N₁ is C, N₂ is U, N₃ is G, N₄ is U, and N₅ is G. In some embodiments, N₁ is C, N₂ is U, N₃ is G, N₄ is U, and N₅ is U.

In some embodiments, N₁ is C, N₂ is U, N₃ is U, N₄ is A, and N₅ is A. In some embodiments, N₁ is C, N₂ is U, N₃ is U, N₄ is A, and N₅ is C. In some embodiments, N₁ is C, N₂ is U, N₃ is U, N₄ is A, and N₅ is G. In some embodiments, N₁ is C, N₂ is U, N₃ is U, N₄ is A, and N₅ is U. In some embodiments, N₁ is C, N₂ is U, N₃ is U, N₄ is C, and N₅ is A. In some embodiments, N₁ is C, N₂ is U, N₃ is U, N₄ is C, and N₅ is C. In some embodiments, N₁ is C, N₂ is U, N₃ is U, N₄ is C, and N₅ is G. In some embodiments, N₁ is C, N₂ is U, N₃ is U, N₄ is C, and N₅ is U. In some embodiments, N₁ is C, N₂ is U, N₃ is U, N₄ is G, and N₅ is A. In some embodiments, N₁ is C, N₂ is U, N₃ is U, N₄ is G, and N₅ is C. In some embodiments, N₁ is C, N₂ is U, N₃ is U, N₄ is G, and N₅ is G. In some embodiments, N₁ is C, N₂ is U, N₃ is U, N₄ is G, and N₅ is U. In some embodiments, N₁ is C, N₂ is U, N₃ is U, N₄ is U, and N₅ is A. In some embodiments, N₁ is C, N₂ is U, N₃ is U, N₄ is U, and N₅ is C. In some embodiments, N₁ is C, N₂ is U, N₃ is U, N₄ is U, and N₅ is G. In some embodiments, N₁ is C, N₂ is U, N₃ is U, N₄ is U, and N₅ is U.

ix. Exemplary Cap Proximal Sequences where N₁ is G, and N₂ is A.

In some embodiments, N₁ is G, N₂ is A, N₃ is A, N₄ is A, and N₅ is A. In some embodiments, N₁ is G, N₂ is A, N₃ is A, N₄ is A, and N₅ is C. In some embodiments, N₁ is G, N₂ is A, N₃ is A, N₄ is A, and N₅ is G. In some embodiments, N₁ is G, N₂ is A, N₃ is A, N₄ is A, and N₅ is U. In some embodiments, N₁ is G, N₂ is A, N₃ is A, N₄ is C, and N₅ is A. In some embodiments, N₁ is G, N₂ is A, N₃ is A, N₄ is C, and N₅ is C. In some embodiments, N₁ is G, N₂ is A, N₃ is A, N₄ is C, and N₅ is G. In some embodiments, N₁ is G, N₂ is A, N₃ is A, N₄ is C, and N₅ is U. In some embodiments, N₁ is G, N₂ is A, N₃ is A, N₄ is G, and N₅ is A. In some embodiments, N₁ is G, N₂ is A, N₃ is A, N₄ is G, and N₅ is C. In some embodiments, N₁ is G, N₂ is A, N₃ is A, N₄ is G, and N₅ is G. In some embodiments, N₁ is G, N₂ is A, N₃ is A, N₄ is G, and N₅ is U. In some embodiments, N₁ is G, N₂ is A, N₃ is A, N₄ is U, and N₅ is A. In some embodiments, N₁ is G, N₂ is A, N₃ is A, N₄ is U, and N₅ is C. In some embodiments, N₁ is G, N₂ is A, N₃ is A, N₄ is U, and N₅ is G. In some embodiments, N₁ is G, N₂ is A, N₃ is A, N₄ is U, and N₅ is U.

In some embodiments, N₁ is G, N₂ is A, N₃ is C, N₄ is A, and N₅ is A. In some embodiments, N₁ is G, N₂ is A, N₃ is C, N₄ is A, and N₅ is C. In some embodiments, N₁ is G, N₂ is A, N₃ is C, N₄ is A, and N₅ is G. In some embodiments, N₁ is G, N₂ is A, N₃ is C, N₄ is A, and N₅ is U. In some embodiments, N₁ is G, N₂ is A, N₃ is C, N₄ is C, and N₅ is A. In some embodiments, N₁ is G, N₂ is A, N₃ is C, N₄ is C, and N₅ is C. In some embodiments, N₁ is G, N₂ is A, N₃ is C, N₄ is C, and N₅ is G. In some embodiments, N₁ is G, N₂ is A, N₃ is C, N₄ is C, and N₅ is U. In some embodiments, N₁ is G, N₂ is A, N₃ is C, N₄ is G, and N₅ is A. In some embodiments, N₁ is G, N₂ is A, N₃ is C, N₄ is G, and N₅ is C. In some embodiments, N₁ is G, N₂ is A, N₃ is C, N₄ is G, and N₅ is G. In some embodiments, N₁ is G, N₂ is A, N₃ is C, N₄ is G, and N₅ is U. In some embodiments, N₁ is G, N₂ is A, N₃ is C, N₄ is U, and N₅ is A. In some embodiments, N₁ is G, N₂ is A, N₃ is C, N₄ is U, and N₅ is C. In some embodiments, N₁ is G, N₂ is A, N₃ is C, N₄ is U, and N₅ is G. In some embodiments, N₁ is G, N₂ is A, N₃ is C, N₄ is U, and N₅ is U.

In some embodiments, N₁ is G, N₂ is A, N₃ is G, N₄ is A, and N₅ is A. In some embodiments, N₁ is G, N₂ is A, N₃ is G, N₄ is A, and N₅ is C. In some embodiments, N₁ is G, N₂ is A, N₃ is G, N₄ is A, and N₅ is G. In some embodiments, N₁ is G, N₂ is A, N₃ is G, N₄ is A, and N₅ is U. In some embodiments, N₁ is G, N₂ is A, N₃ is G, N₄ is C, and N₅ is A. In some embodiments, N₁ is G, N₂ is A, N₃ is G, N₄ is C, and N₅ is C. In some embodiments, N₁ is G, N₂ is A, N₃ is G, N₄ is C, and N₅ is G. In some embodiments, N₁ is G, N₂ is A, N₃ is G, N₄ is C, and N₅ is U. In some embodiments, N₁ is G, N₂ is A, N₃ is G, N₄ is G, and N₅ is A. In some embodiments, N₁ is G, N₂ is A, N₃ is G, N₄ is G, and N₅ is C. In some embodiments, N₁ is G, N₂ is A, N₃ is G, N₄ is G, and N₅ is G. In some embodiments, N₁ is G, N₂ is A, N₃ is G, N₄ is G, and N₅ is U. In some embodiments, N₁ is G, N₂ is A, N₃ is G, N₄ is U, and N₅ is A. In some embodiments, N₁ is G, N₂ is A, N₃ is G, N₄ is U, and N₅ is C. In some embodiments, N₁ is G, N₂ is A, N₃ is G, N₄ is U, and N₅ is G. In some embodiments, N₁ is G, N₂ is A, N₃ is G, N₄ is U, and N₅ is U.

In some embodiments, N₁ is G, N₂ is A, N₃ is U, N₄ is A, and N₅ is A. In some embodiments, N₁ is G, N₂ is A, N₃ is U, N₄ is A, and N₅ is C. In some embodiments, N₁ is G, N₂ is A, N₃ is U, N₄ is A, and N₅ is G. In some embodiments, N₁ is G, N₂ is A, N₃ is U, N₄ is A, and N₅ is U. In some embodiments, N₁ is G, N₂ is A, N₃ is U, N₄ is C, and N₅ is A. In some embodiments, N₁ is G, N₂ is A, N₃ is U, N₄ is C, and N₅ is C. In some embodiments, N₁ is G, N₂ is A, N₃ is U, N₄ is C, and N₅ is G. In some embodiments, N₁ is G, N₂ is A, N₃ is U, N₄ is C, and N₅ is U. In some embodiments, N₁ is G, N₂ is A, N₃ is U, N₄ is G, and N₅ is A. In some embodiments, N₁ is G, N₂ is A, N₃ is U, N₄ is G, and N₅ is C. In some embodiments, N₁ is G, N₂ is A, N₃ is U, N₄ is G, and N₅ is G. In some embodiments, N₁ is G, N₂ is A, N₃ is U, N₄ is G, and N₅ is U. In some embodiments, N₁ is G, N₂ is A, N₃ is U, N₄ is U, and N₅ is A. In some embodiments, N₁ is G, N₂ is A, N₃ is U, N₄ is U, and N₅ is C. In some embodiments, N₁ is G, N₂ is A, N₃ is U, N₄ is U, and N₅ is G. In some embodiments, N₁ is G, N₂ is A, N₃ is U, N₄ is U, and N₅ is U.

x. Exemplary Cap Proximal Sequences where N₁ is G, and N₂ is C.

In some embodiments, N₁ is G, N₂ is C, N₃ is A, N₄ is A, and N₅ is A. In some embodiments, N₁ is G, N₂ is C, N₃ is A, N₄ is A, and N₅ is C. In some embodiments, N₁ is G, N₂ is C, N₃ is A, N₄ is A, and N₅ is G. In some embodiments, N₁ is G, N₂ is C, N₃ is A, N₄ is A, and N₅ is U. In some embodiments, N₁ is G, N₂ is C, N₃ is A, N₄ is C, and N₅ is A. In some embodiments, N₁ is G, N₂ is C, N₃ is A, N₄ is C, and N₅ is C. In some embodiments, N₁ is G, N₂ is C, N₃ is A, N₄ is C, and N₅ is G. In some embodiments, N₁ is G, N₂ is C, N₃ is A, N₄ is C, and N₅ is U. In some embodiments, N₁ is G, N₂ is C, N₃ is A, N₄ is G, and N₅ is A. In some embodiments, N₁ is G, N₂ is C, N₃ is A, N₄ is G, and N₅ is C. In some embodiments, N₁ is G, N₂ is C, N₃ is A, N₄ is G, and N₅ is G. In some embodiments, N₁ is G, N₂ is C, N₃ is A, N₄ is G, and N₅ is U. In some embodiments, N₁ is G, N₂ is C, N₃ is A, N₄ is U, and N₅ is A. In some embodiments, N₁ is G, N₂ is C, N₃ is A, N₄ is U, and N₅ is C. In some embodiments, N₁ is G, N₂ is C, N₃ is A, N₄ is U, and N₅ is G. In some embodiments, N₁ is G, N₂ is C, N₃ is A, N₄ is U, and N₅ is U.

In some embodiments, N₁ is G, N₂ is C, N₃ is C, N₄ is A, and N₅ is A. In some embodiments, N₁ is G, N₂ is C, N₃ is C, N₄ is A, and N₅ is C. In some embodiments, N₁ is G, N₂ is C, N₃ is C, N₄ is A, and N₅ is G. In some embodiments, N₁ is G, N₂ is C, N₃ is C, N₄ is A, and N₅ is U. In some embodiments, N₁ is G, N₂ is C, N₃ is C, N₄ is C, and N₅ is A. In some embodiments, N₁ is G, N₂ is C, N₃ is C, N₄ is C, and N₅ is C. In some embodiments, N₁ is G, N₂ is C, N₃ is C, N₄ is C, and N₅ is G. In some embodiments, N₁ is G, N₂ is C, N₃ is C, N₄ is C, and N₅ is U. In some embodiments, N₁ is G, N₂ is C, N₃ is C, N₄ is G, and N₅ is A. In some embodiments, N₁ is G, N₂ is C, N₃ is C, N₄ is G, and N₅ is C. In some embodiments, N₁ is G, N₂ is C, N₃ is C, N₄ is G, and N₅ is G. In some embodiments, N₁ is G, N₂ is C, N₃ is C, N₄ is G, and N₅ is U. In some embodiments, N₁ is G, N₂ is C, N₃ is C, N₄ is U, and N₅ is A. In some embodiments, N₁ is G, N₂ is C, N₃ is C, N₄ is U, and N₅ is C. In some embodiments, N₁ is G, N₂ is C, N₃ is C, N₄ is U, and N₅ is G. In some embodiments, N₁ is G, N₂ is C, N₃ is C, N₄ is U, and N₅ is U.

In some embodiments, N₁ is G, N₂ is C, N₃ is G, N₄ is A, and N₅ is A. In some embodiments, N₁ is G, N₂ is C, N₃ is G, N₄ is A, and N₅ is C. In some embodiments, N₁ is G, N₂ is C, N₃ is G, N₄ is A, and N₅ is G. In some embodiments, N₁ is G, N₂ is C, N₃ is G, N₄ is A, and N₅ is U. In some embodiments, N₁ is G, N₂ is C, N₃ is G, N₄ is C, and N₅ is A. In some embodiments, N₁ is G, N₂ is C, N₃ is G, N₄ is C, and N₅ is C. In some embodiments, N₁ is G, N₂ is C, N₃ is G, N₄ is C, and N₅ is G. In some embodiments, N₁ is G, N₂ is C, N₃ is G, N₄ is C, and N₅ is U. In some embodiments, N₁ is G, N₂ is C, N₃ is G, N₄ is G, and N₅ is A. In some embodiments, N₁ is G, N₂ is C, N₃ is G, N₄ is G, and N₅ is C. In some embodiments, N₁ is G, N₂ is C, N₃ is G, N₄ is G, and N₅ is G. In some embodiments, N₁ is G, N₂ is C, N₃ is G, N₄ is G, and N₅ is U. In some embodiments, N₁ is G, N₂ is C, N₃ is G, N₄ is U, and N₅ is A. In some embodiments, N₁ is G, N₂ is C, N₃ is G, N₄ is U, and N₅ is C. In some embodiments, N₁ is G, N₂ is C, N₃ is G, N₄ is U, and N₅ is G. In some embodiments, N₁ is G, N₂ is C, N₃ is G, N₄ is U, and N₅ is U.

In some embodiments, N₁ is G, N₂ is C, N₃ is U, N₄ is A, and N₅ is A. In some embodiments, N₁ is G, N₂ is C, N₃ is U, N₄ is A, and N₅ is C. In some embodiments, N₁ is G, N₂ is C, N₃ is U, N₄ is A, and N₅ is G. In some embodiments, N₁ is G, N₂ is C, N₃ is U, N₄ is A, and N₅ is U. In some embodiments, N₁ is G, N₂ is C, N₃ is U, N₄ is C, and N₅ is A. In some embodiments, N₁ is G, N₂ is C, N₃ is U, N₄ is C, and N₅ is C. In some embodiments, N₁ is G, N₂ is C, N₃ is U, N₄ is C, and N₅ is G. In some embodiments, N₁ is G, N₂ is C, N₃ is U, N₄ is C, and N₅ is U. In some embodiments, N₁ is G, N₂ is C, N₃ is U, N₄ is G, and N₅ is A. In some embodiments, N₁ is G, N₂ is C, N₃ is U, N₄ is G, and N₅ is C. In some embodiments, N₁ is G, N₂ is C, N₃ is U, N₄ is G, and N₅ is G. In some embodiments, N₁ is G, N₂ is C, N₃ is U, N₄ is G, and N₅ is U. In some embodiments, N₁ is G, N₂ is C, N₃ is U, N₄ is U, and N₅ is A. In some embodiments, N₁ is G, N₂ is C, N₃ is U, N₄ is U, and N₅ is C. In some embodiments, N₁ is G, N₂ is C, N₃ is U, N₄ is U, and N₅ is G. In some embodiments, N₁ is G, N₂ is A, N₃ is U, N₄ is U, and N₅ is U.

xi. Exemplary Cap Proximal Sequences where N₁ is G, and N₂ is G.

In some embodiments, N₁ is G, N₂ is G, N₃ is A, N₄ is A, and N₅ is A. In some embodiments, N₁ is G, N₂ is G, N₃ is A, N₄ is A, and N₅ is C. In some embodiments, N₁ is G, N₂ is G, N₃ is A, N₄ is A, and N₅ is G. In some embodiments, N₁ is G, N₂ is G, N₃ is A, N₄ is A, and N₅ is U. In some embodiments, N₁ is G, N₂ is G, N₃ is A, N₄ is C, and N₅ is A. In some embodiments, N₁ is G, N₂ is G, N₃ is A, N₄ is C, and N₅ is C. In some embodiments, N₁ is G, N₂ is G, N₃ is A, N₄ is C, and N₅ is G. In some embodiments, N₁ is G, N₂ is G, N₃ is A, N₄ is C, and N₅ is U. In some embodiments, N₁ is G, N₂ is G, N₃ is A, N₄ is G, and N₅ is A. In some embodiments, N₁ is G, N₂ is G, N₃ is A, N₄ is G, and N₅ is C. In some embodiments, N₁ is G, N₂ is G, N₃ is A, N₄ is G, and N₅ is G. In some embodiments, N₁ is G, N₂ is G, N₃ is A, N₄ is G, and N₅ is U. In some embodiments, N₁ is G, N₂ is G, N₃ is A, N₄ is U, and N₅ is A. In some embodiments, N₁ is G, N₂ is G, N₃ is A, N₄ is U, and N₅ is C. In some embodiments, N₁ is G, N₂ is G, N₃ is A, N₄ is U, and N₅ is G. In some embodiments, N₁ is G, N₂ is G, N₃ is A, N₄ is U, and N₅ is U.

In some embodiments, N₁ is G, N₂ is G, N₃ is C, N₄ is A, and N₅ is A. In some embodiments, N₁ is G, N₂ is G, N₃ is C, N₄ is A, and N₅ is C. In some embodiments, N₁ is G, N₂ is G, N₃ is C, N₄ is A, and N₅ is G. In some embodiments, N₁ is G, N₂ is G, N₃ is C, N₄ is A, and N₅ is U. In some embodiments, N₁ is G, N₂ is G, N₃ is C, N₄ is C, and N₅ is A. In some embodiments, N₁ is G, N₂ is G, N₃ is C, N₄ is C, and N₅ is C. In some embodiments, N₁ is G, N₂ is G, N₃ is C, N₄ is C, and N₅ is G. In some embodiments, N₁ is G, N₂ is G, N₃ is C, N₄ is C, and N₅ is U. In some embodiments, N₁ is G, N₂ is G, N₃ is C, N₄ is G, and N₅ is A. In some embodiments, N₁ is G, N₂ is G, N₃ is C, N₄ is G, and N₅ is C. In some embodiments, N₁ is G, N₂ is G, N₃ is C, N₄ is G, and N₅ is G. In some embodiments, N₁ is G, N₂ is G, N₃ is C, N₄ is G, and N₅ is U. In some embodiments, N₁ is G, N₂ is G, N₃ is C, N₄ is U, and N₅ is A. In some embodiments, N₁ is G, N₂ is G, N₃ is C, N₄ is U, and N₅ is C. In some embodiments, N₁ is G, N₂ is G, N₃ is C, N₄ is U, and N₅ is G. In some embodiments, N₁ is G, N₂ is G, N₃ is C, N₄ is U, and N₅ is U.

In some embodiments, N₁ is G, N₂ is G, N₃ is G, N₄ is A, and N₅ is A. In some embodiments, N₁ is G, N₂ is G, N₃ is G, N₄ is A, and N₅ is C. In some embodiments, N₁ is G, N₂ is G, N₃ is G, N₄ is A, and N₅ is G. In some embodiments, N₁ is G, N₂ is G, N₃ is G, N₄ is A, and N₅ is U. In some embodiments, N₁ is G, N₂ is G, N₃ is G, N₄ is C, and N₅ is A. In some embodiments, N₁ is G, N₂ is G, N₃ is G, N₄ is C, and N₅ is C. In some embodiments, N₁ is G, N₂ is G, N₃ is G, N₄ is C, and N₅ is G. In some embodiments, N₁ is G, N₂ is G, N₃ is G, N₄ is C, and N₅ is U. In some embodiments, N₁ is G, N₂ is G, N₃ is G, N₄ is G, and N₅ is A. In some embodiments, N₁ is G, N₂ is G, N₃ is G, N₄ is G, and N₅ is C. In some embodiments, N₁ is G, N₂ is G, N₃ is G, N₄ is G, and N₅ is G. In some embodiments, N₁ is G, N₂ is G, N₃ is G, N₄ is G, and N₅ is U. In some embodiments, N₁ is G, N₂ is G, N₃ is G, N₄ is U, and N₅ is A. In some embodiments, N₁ is G, N₂ is G, N₃ is G, N₄ is U, and N₅ is C. In some embodiments, N₁ is G, N₂ is G, N₃ is G, N₄ is U, and N₅ is G. In some embodiments, N₁ is G, N₂ is G, N₃ is G, N₄ is U, and N₅ is U.

In some embodiments, N₁ is G, N₂ is G, N₃ is U, N₄ is A, and N₅ is A. In some embodiments, N₁ is G, N₂ is G, N₃ is U, N₄ is A, and N₅ is C. In some embodiments, N₁ is G, N₂ is G, N₃ is U, N₄ is A, and N₅ is G. In some embodiments, N₁ is G, N₂ is G, N₃ is U, N₄ is A, and N₅ is U. In some embodiments, N₁ is G, N₂ is G, N₃ is U, N₄ is C, and N₅ is A. In some embodiments, N₁ is G, N₂ is G, N₃ is U, N₄ is C, and N₅ is C. In some embodiments, N₁ is G, N₂ is G, N₃ is U, N₄ is C, and N₅ is G. In some embodiments, N₁ is G, N₂ is G, N₃ is U, N₄ is C, and N₅ is U. In some embodiments, N₁ is G, N₂ is G, N₃ is U, N₄ is G, and N₅ is A. In some embodiments, N₁ is G, N₂ is G, N₃ is U, N₄ is G, and N₅ is C. In some embodiments, N₁ is G, N₂ is G, N₃ is U, N₄ is G, and N₅ is G. In some embodiments, N₁ is G, N₂ is G, N₃ is U, N₄ is G, and N₅ is U. In some embodiments, N₁ is G, N₂ is G, N₃ is U, N₄ is U, and N₅ is A. In some embodiments, N₁ is G, N₂ is G, N₃ is U, N₄ is U, and N₅ is C. In some embodiments, N₁ is G, N₂ is G, N₃ is U, N₄ is U, and N₅ is G. In some embodiments, N₁ is G, N₂ is G, N₃ is U, N₄ is U, and N₅ is U.

xii. Exemplary Cap Proximal Sequences where N₁ is G, and N₂ is U.

In some embodiments, N₁ is G, N₂ is U, N₃ is A, N₄ is A, and N₅ is A. In some embodiments, N₁ is G, N₂ is U, N₃ is A, N₄ is A, and N₅ is C. In some embodiments, N₁ is G, N₂ is U, N₃ is A, N₄ is A, and N₅ is G. In some embodiments, N₁ is G, N₂ is U, N₃ is A, N₄ is A, and N₅ is U. In some embodiments, N₁ is G, N₂ is U, N₃ is A, N₄ is C, and N₅ is A. In some embodiments, N₁ is G, N₂ is U, N₃ is A, N₄ is C, and N₅ is C. In some embodiments, N₁ is G, N₂ is U, N₃ is A, N₄ is C, and N₅ is G. In some embodiments, N₁ is G, N₂ is U, N₃ is A, N₄ is C, and N₅ is U. In some embodiments, N₁ is G, N₂ is U, N₃ is A, N₄ is G, and N₅ is A. In some embodiments, N₁ is G, N₂ is U, N₃ is A, N₄ is G, and N₅ is C. In some embodiments, N₁ is G, N₂ is U, N₃ is A, N₄ is G, and N₅ is G. In some embodiments, N₁ is G, N₂ is U, N₃ is A, N₄ is G, and N₅ is U. In some embodiments, N₁ is G, N₂ is U, N₃ is A, N₄ is U, and N₅ is A. In some embodiments, N₁ is G, N₂ is U, N₃ is A, N₄ is U, and N₅ is C. In some embodiments, N₁ is G, N₂ is U, N₃ is A, N₄ is U, and N₅ is G. In some embodiments, N₁ is G, N₂ is U, N₃ is A, N₄ is U, and N₅ is U.

In some embodiments, N₁ is G, N₂ is U, N₃ is C, N₄ is A, and N₅ is A. In some embodiments, N₁ is G, N₂ is U, N₃ is C, N₄ is A, and N₅ is C. In some embodiments, N₁ is G, N₂ is U, N₃ is C, N₄ is A, and N₅ is G. In some embodiments, N₁ is G, N₂ is U, N₃ is C, N₄ is A, and N₅ is U. In some embodiments, N₁ is G, N₂ is U, N₃ is C, N₄ is C, and N₅ is A. In some embodiments, N₁ is G, N₂ is U, N₃ is C, N₄ is C, and N₅ is C. In some embodiments, N₁ is G, N₂ is U, N₃ is C, N₄ is C, and N₅ is G. In some embodiments, N₁ is G, N₂ is U, N₃ is C, N₄ is C, and N₅ is U. In some embodiments, N₁ is G, N₂ is U, N₃ is C, N₄ is G, and N₅ is A. In some embodiments, N₁ is G, N₂ is U, N₃ is C, N₄ is G, and N₅ is C. In some embodiments, N₁ is G, N₂ is U, N₃ is C, N₄ is G, and N₅ is G. In some embodiments, N₁ is G, N₂ is U, N₃ is C, N₄ is G, and N₅ is U. In some embodiments, N₁ is G, N₂ is U, N₃ is C, N₄ is U, and N₅ is A. In some embodiments, N₁ is G, N₂ is U, N₃ is C, N₄ is U, and N₅ is C. In some embodiments, N₁ is G, N₂ is U, N₃ is C, N₄ is U, and N₅ is G. In some embodiments, N₁ is G, N₂ is U, N₃ is C, N₄ is U, and N₅ is U.

In some embodiments, N₁ is G, N₂ is U, N₃ is G, N₄ is A, and N₅ is A. In some embodiments, N₁ is G, N₂ is U, N₃ is G, N₄ is A, and N₅ is C. In some embodiments, N₁ is G, N₂ is U, N₃ is G, N₄ is A, and N₅ is G. In some embodiments, N₁ is G, N₂ is U, N₃ is G, N₄ is A, and N₅ is U. In some embodiments, N₁ is G, N₂ is U, N₃ is G, N₄ is C, and N₅ is A. In some embodiments, N₁ is G, N₂ is U, N₃ is G, N₄ is C, and N₅ is C. In some embodiments, N₁ is G, N₂ is U, N₃ is G, N₄ is C, and N₅ is G. In some embodiments, N₁ is G, N₂ is U, N₃ is G, N₄ is C, and N₅ is U. In some embodiments, N₁ is G, N₂ is U, N₃ is G, N₄ is G, and N₅ is A. In some embodiments, N₁ is G, N₂ is U, N₃ is G, N₄ is G, and N₅ is C. In some embodiments, N₁ is G, N₂ is U, N₃ is G, N₄ is G, and N₅ is G. In some embodiments, N₁ is G, N₂ is U, N₃ is G, N₄ is G, and N₅ is U. In some embodiments, N₁ is G, N₂ is U, N₃ is G, N₄ is U, and N₅ is A. In some embodiments, N₁ is G, N₂ is U, N₃ is G, N₄ is U, and N₅ is C. In some embodiments, N₁ is G, N₂ is U, N₃ is G, N₄ is U, and N₅ is G. In some embodiments, N₁ is G, N₂ is U, N₃ is G, N₄ is U, and N₅ is U.

In some embodiments, N₁ is G, N₂ is U, N₃ is U, N₄ is A, and N₅ is A. In some embodiments, N₁ is G, N₂ is U, N₃ is U, N₄ is A, and N₅ is C. In some embodiments, N₁ is G, N₂ is U, N₃ is U, N₄ is A, and N₅ is G. In some embodiments, N₁ is G, N₂ is U, N₃ is U, N₄ is A, and N₅ is U. In some embodiments, N₁ is G, N₂ is U, N₃ is U, N₄ is C, and N₅ is A. In some embodiments, N₁ is G, N₂ is U, N₃ is U, N₄ is C, and N₅ is C. In some embodiments, N₁ is G, N₂ is U, N₃ is U, N₄ is C, and N₅ is G. In some embodiments, N₁ is G, N₂ is U, N₃ is U, N₄ is C, and N₅ is U. In some embodiments, N₁ is G, N₂ is U, N₃ is U, N₄ is G, and N₅ is A. In some embodiments, N₁ is G, N₂ is U, N₃ is U, N₄ is G, and N₅ is C. In some embodiments, N₁ is G, N₂ is U, N₃ is U, N₄ is G, and N₅ is G. In some embodiments, N₁ is G, N₂ is U, N₃ is U, N₄ is G, and N₅ is U. In some embodiments, N₁ is G, N₂ is U, N₃ is U, N₄ is U, and N₅ is A. In some embodiments, N₁ is G, N₂ is U, N₃ is U, N₄ is U, and N₅ is C. In some embodiments, N₁ is G, N₂ is U, N₃ is U, N₄ is U, and N₅ is G. In some embodiments, N₁ is G, N₂ is U, N₃ is U, N₄ is U, and N₅ is U.

xiii. Exemplary Cap Proximal Sequences where N₁ is U, and N₂ is A.

In some embodiments, N₁ is U, N₂ is A, N₃ is A, N₄ is A, and N₅ is A. In some embodiments, N₁ is U, N₂ is A, N₃ is A, N₄ is A, and N₅ is C. In some embodiments, N₁ is U, N₂ is A, N₃ is A, N₄ is A, and N₅ is G. In some embodiments, N₁ is U, N₂ is A, N₃ is A, N₄ is A, and N₅ is U. In some embodiments, N₁ is U, N₂ is A, N₃ is A, N₄ is C, and N₅ is A. In some embodiments, N₁ is U, N₂ is A, N₃ is A, N₄ is C, and N₅ is C. In some embodiments, N₁ is U, N₂ is A, N₃ is A, N₄ is C, and N₅ is G. In some embodiments, N₁ is U, N₂ is A, N₃ is A, N₄ is C, and N₅ is U. In some embodiments, N₁ is U, N₂ is A, N₃ is A, N₄ is G, and N₅ is A. In some embodiments, N₁ is U, N₂ is A, N₃ is A, N₄ is G, and N₅ is C. In some embodiments, N₁ is U, N₂ is A, N₃ is A, N₄ is G, and N₅ is G. In some embodiments, N₁ is U, N₂ is A, N₃ is A, N₄ is G, and N₅ is U. In some embodiments, N₁ is U, N₂ is A, N₃ is A, N₄ is U, and N₅ is A. In some embodiments, N₁ is U, N₂ is A, N₃ is A, N₄ is U, and N₅ is C. In some embodiments, N₁ is U, N₂ is A, N₃ is A, N₄ is U, and N₅ is G. In some embodiments, N₁ is U, N₂ is A, N₃ is A, N₄ is U, and N₅ is U.

In some embodiments, N₁ is U, N₂ is A, N₃ is C, N₄ is A, and N₅ is A. In some embodiments, N₁ is U, N₂ is A, N₃ is C, N₄ is A, and N₅ is C. In some embodiments, N₁ is U, N₂ is A, N₃ is C, N₄ is A, and N₅ is G. In some embodiments, N₁ is U, N₂ is A, N₃ is C, N₄ is A, and N₅ is U. In some embodiments, N₁ is U, N₂ is A, N₃ is C, N₄ is C, and N₅ is A. In some embodiments, N₁ is U, N₂ is A, N₃ is C, N₄ is C, and N₅ is C. In some embodiments, N₁ is U, N₂ is A, N₃ is C, N₄ is C, and N₅ is G. In some embodiments, N₁ is U, N₂ is A, N₃ is C, N₄ is C, and N₅ is U. In some embodiments, N₁ is U, N₂ is A, N₃ is C, N₄ is G, and N₅ is A. In some embodiments, N₁ is U, N₂ is A, N₃ is C, N₄ is G, and N₅ is C. In some embodiments, N₁ is U, N₂ is A, N₃ is C, N₄ is G, and N₅ is G. In some embodiments, N₁ is U, N₂ is A, N₃ is C, N₄ is G, and N₅ is U. In some embodiments, N₁ is U, N₂ is A, N₃ is C, N₄ is U, and N₅ is A. In some embodiments, N₁ is U, N₂ is A, N₃ is C, N₄ is U, and N₅ is C. In some embodiments, N₁ is U, N₂ is A, N₃ is C, N₄ is U, and N₅ is G. In some embodiments, N₁ is U, N₂ is A, N₃ is C, N₄ is U, and N₅ is U.

In some embodiments, N₁ is U, N₂ is A, N₃ is G, N₄ is A, and N₅ is A. In some embodiments, N₁ is U, N₂ is A, N₃ is G, N₄ is A, and N₅ is C. In some embodiments, N₁ is U, N₂ is A, N₃ is G, N₄ is A, and N₅ is G. In some embodiments, N₁ is U, N₂ is A, N₃ is G, N₄ is A, and N₅ is U. In some embodiments, N₁ is U, N₂ is A, N₃ is G, N₄ is C, and N₅ is A. In some embodiments, N₁ is U, N₂ is A, N₃ is G, N₄ is C, and N₅ is C. In some embodiments, N₁ is U, N₂ is A, N₃ is G, N₄ is C, and N₅ is G. In some embodiments, N₁ is U, N₂ is A, N₃ is G, N₄ is C, and N₅ is U. In some embodiments, N₁ is U, N₂ is A, N₃ is G, N₄ is G, and N₅ is A. In some embodiments, N₁ is U, N₂ is A, N₃ is G, N₄ is G, and N₅ is C. In some embodiments, N₁ is U, N₂ is A, N₃ is G, N₄ is G, and N₅ is G. In some embodiments, N₁ is U, N₂ is A, N₃ is G, N₄ is G, and N₅ is U. In some embodiments, N₁ is U, N₂ is A, N₃ is G, N₄ is U, and N₅ is A. In some embodiments, N₁ is U, N₂ is A, N₃ is G, N₄ is U, and N₅ is C. In some embodiments, N₁ is U, N₂ is A, N₃ is G, N₄ is U, and N₅ is G. In some embodiments, N₁ is U, N₂ is A, N₃ is G, N₄ is U, and N₅ is U.

In some embodiments, N₁ is U, N₂ is A, N₃ is U, N₄ is A, and N₅ is A. In some embodiments, N₁ is U, N₂ is A, N₃ is U, N₄ is A, and N₅ is C. In some embodiments, N₁ is U, N₂ is A, N₃ is U, N₄ is A, and N₅ is G. In some embodiments, N₁ is U, N₂ is A, N₃ is U, N₄ is A, and N₅ is U. In some embodiments, N₁ is U, N₂ is A, N₃ is U, N₄ is C, and N₅ is A. In some embodiments, N₁ is U, N₂ is A, N₃ is U, N₄ is C, and N₅ is C. In some embodiments, N₁ is U, N₂ is A, N₃ is U, N₄ is C, and N₅ is G. In some embodiments, N₁ is U, N₂ is A, N₃ is U, N₄ is C, and N₅ is U. In some embodiments, N₁ is U, N₂ is A, N₃ is U, N₄ is G, and N₅ is A. In some embodiments, N₁ is U, N₂ is A, N₃ is U, N₄ is G, and N₅ is C. In some embodiments, N₁ is U, N₂ is A, N₃ is U, N₄ is G, and N₅ is G. In some embodiments, N₁ is U, N₂ is A, N₃ is U, N₄ is G, and N₅ is U. In some embodiments, N₁ is U, N₂ is A, N₃ is U, N₄ is U, and N₅ is A. In some embodiments, N₁ is U, N₂ is A, N₃ is U, N₄ is U, and N₅ is C. In some embodiments, N₁ is U, N₂ is A, N₃ is U, N₄ is U, and N₅ is G. In some embodiments, N₁ is U, N₂ is A, N₃ is U, N₄ is U, and N₅ is U.

xiv. Exemplary Cap Proximal Sequences where N₁ is U, and N₂ is C.

In some embodiments, N₁ is U, N₂ is C, N₃ is A, N₄ is A, and N₅ is A. In some embodiments, N₁ is U, N₂ is C, N₃ is A, N₄ is A, and N₅ is C. In some embodiments, N₁ is U, N₂ is C, N₃ is A, N₄ is A, and N₅ is G. In some embodiments, N₁ is U, N₂ is C, N₃ is A, N₄ is A, and N₅ is U. In some embodiments, N₁ is U, N₂ is C, N₃ is A, N₄ is C, and N₅ is A. In some embodiments, N₁ is U, N₂ is C, N₃ is A, N₄ is C, and N₅ is C. In some embodiments, N₁ is U, N₂ is C, N₃ is A, N₄ is C, and N₅ is G. In some embodiments, N₁ is U, N₂ is C, N₃ is A, N₄ is C, and N₅ is U. In some embodiments, N₁ is U, N₂ is C, N₃ is A, N₄ is G, and N₅ is A. In some embodiments, N₁ is U, N₂ is C, N₃ is A, N₄ is G, and N₅ is C. In some embodiments, N₁ is U, N₂ is C, N₃ is A, N₄ is G, and N₅ is G. In some embodiments, N₁ is U, N₂ is C, N₃ is A, N₄ is G, and N₅ is U. In some embodiments, N₁ is U, N₂ is C, N₃ is A, N₄ is U, and N₅ is A. In some embodiments, N₁ is U, N₂ is C, N₃ is A, N₄ is U, and N₅ is C. In some embodiments, N₁ is U, N₂ is C, N₃ is A, N₄ is U, and N₅ is G. In some embodiments, N₁ is U, N₂ is C, N₃ is A, N₄ is U, and N₅ is U.

In some embodiments, N₁ is U, N₂ is C, N₃ is C, N₄ is A, and N₅ is A. In some embodiments, N₁ is U, N₂ is C, N₃ is C, N₄ is A, and N₅ is C. In some embodiments, N₁ is U, N₂ is C, N₃ is C, N₄ is A, and N₅ is G. In some embodiments, N₁ is U, N₂ is C, N₃ is C, N₄ is A, and N₅ is U. In some embodiments, N₁ is U, N₂ is C, N₃ is C, N₄ is C, and N₅ is A. In some embodiments, N₁ is U, N₂ is C, N₃ is C, N₄ is C, and N₅ is C. In some embodiments, N₁ is U, N₂ is C, N₃ is C, N₄ is C, and N₅ is G. In some embodiments, N₁ is U, N₂ is C, N₃ is C, N₄ is C, and N₅ is U. In some embodiments, N₁ is U, N₂ is C, N₃ is C, N₄ is G, and N₅ is A. In some embodiments, N₁ is U, N₂ is C, N₃ is C, N₄ is G, and N₅ is C. In some embodiments, N₁ is U, N₂ is C, N₃ is C, N₄ is G, and N₅ is G. In some embodiments, N₁ is U, N₂ is C, N₃ is C, N₄ is G, and N₅ is U. In some embodiments, N₁ is U, N₂ is C, N₃ is C, N₄ is U, and N₅ is A. In some embodiments, N₁ is U, N₂ is C, N₃ is C, N₄ is U, and N₅ is C. In some embodiments, N₁ is U, N₂ is C, N₃ is C, N₄ is U, and N₅ is G. In some embodiments, N₁ is U, N₂ is C, N₃ is C, N₄ is U, and N₅ is U.

In some embodiments, N₁ is U, N₂ is C, N₃ is G, N₄ is A, and N₅ is A. In some embodiments, N₁ is U, N₂ is C, N₃ is G, N₄ is A, and N₅ is C. In some embodiments, N₁ is U, N₂ is C, N₃ is G, N₄ is A, and N₅ is G. In some embodiments, N₁ is U, N₂ is C, N₃ is G, N₄ is A, and N₅ is U. In some embodiments, N₁ is U, N₂ is C, N₃ is G, N₄ is C, and N₅ is A. In some embodiments, N₁ is U, N₂ is C, N₃ is G, N₄ is C, and N₅ is C. In some embodiments, N₁ is U, N₂ is C, N₃ is G, N₄ is C, and N₅ is G. In some embodiments, N₁ is U, N₂ is C, N₃ is G, N₄ is C, and N₅ is U. In some embodiments, N₁ is U, N₂ is C, N₃ is G, N₄ is G, and N₅ is A. In some embodiments, N₁ is U, N₂ is C, N₃ is G, N₄ is G, and N₅ is C. In some embodiments, N₁ is U, N₂ is C, N₃ is G, N₄ is G, and N₅ is G. In some embodiments, N₁ is U, N₂ is C, N₃ is G, N₄ is G, and N₅ is U. In some embodiments, N₁ is U, N₂ is C, N₃ is G, N₄ is U, and N₅ is A. In some embodiments, N₁ is U, N₂ is C, N₃ is G, N₄ is U, and N₅ is C. In some embodiments, N₁ is U, N₂ is C, N₃ is G, N₄ is U, and N₅ is G. In some embodiments, N₁ is U, N₂ is C, N₃ is G, N₄ is U, and N₅ is U.

In some embodiments, N₁ is U, N₂ is C, N₃ is U, N₄ is A, and N₅ is A. In some embodiments, N₁ is U, N₂ is C, N₃ is U, N₄ is A, and N₅ is C. In some embodiments, N₁ is U, N₂ is C, N₃ is U, N₄ is A, and N₅ is G. In some embodiments, N₁ is U, N₂ is C, N₃ is U, N₄ is A, and N₅ is U. In some embodiments, N₁ is U, N₂ is C, N₃ is U, N₄ is C, and N₅ is A. In some embodiments, N₁ is U, N₂ is C, N₃ is U, N₄ is C, and N₅ is C. In some embodiments, N₁ is U, N₂ is C, N₃ is U, N₄ is C, and N₅ is G. In some embodiments, N₁ is U, N₂ is C, N₃ is U, N₄ is C, and N₅ is U. In some embodiments, N₁ is U, N₂ is C, N₃ is U, N₄ is G, and N₅ is A. In some embodiments, N₁ is U, N₂ is C, N₃ is U, N₄ is G, and N₅ is C. In some embodiments, N₁ is U, N₂ is C, N₃ is U, N₄ is G, and N₅ is G. In some embodiments, N₁ is U, N₂ is C, N₃ is U, N₄ is G, and N₅ is U. In some embodiments, N₁ is U, N₂ is C, N₃ is U, N₄ is U, and N₅ is A. In some embodiments, N₁ is U, N₂ is C, N₃ is U, N₄ is U, and N₅ is C. In some embodiments, N₁ is U, N₂ is C, N₃ is U, N₄ is U, and N₅ is G. In some embodiments, N₁ is U, N₂ is A, N₃ is U, N₄ is U, and N₅ is U.

xv. Exemplary Cap Proximal Sequences where N₁ is U, and N₂ is G.

In some embodiments, N₁ is U, N₂ is G, N₃ is A, N₄ is A, and N₅ is A. In some embodiments, N₁ is U, N₂ is G, N₃ is A, N₄ is A, and N₅ is C. In some embodiments, N₁ is U, N₂ is G, N₃ is A, N₄ is A, and N₅ is G. In some embodiments, N₁ is U, N₂ is G, N₃ is A, N₄ is A, and N₅ is U. In some embodiments, N₁ is U, N₂ is G, N₃ is A, N₄ is C, and N₅ is A. In some embodiments, N₁ is U, N₂ is G, N₃ is A, N₄ is C, and N₅ is C. In some embodiments, N₁ is U, N₂ is G, N₃ is A, N₄ is C, and N₅ is G. In some embodiments, N₁ is U, N₂ is G, N₃ is A, N₄ is C, and N₅ is U. In some embodiments, N₁ is U, N₂ is G, N₃ is A, N₄ is G, and N₅ is A. In some embodiments, N₁ is U, N₂ is G, N₃ is A, N₄ is G, and N₅ is C. In some embodiments, N₁ is U, N₂ is G, N₃ is A, N₄ is G, and N₅ is G. In some embodiments, N₁ is U, N₂ is G, N₃ is A, N₄ is G, and N₅ is U. In some embodiments, N₁ is U, N₂ is G, N₃ is A, N₄ is U, and N₅ is A. In some embodiments, N₁ is U, N₂ is G, N₃ is A, N₄ is U, and N₅ is C. In some embodiments, N₁ is U, N₂ is G, N₃ is A, N₄ is U, and N₅ is G. In some embodiments, N₁ is U, N₂ is G, N₃ is A, N₄ is U, and N₅ is U.

In some embodiments, N₁ is U, N₂ is G, N₃ is C, N₄ is A, and N₅ is A. In some embodiments, N₁ is U, N₂ is G, N₃ is C, N₄ is A, and N₅ is C. In some embodiments, N₁ is U, N₂ is G, N₃ is C, N₄ is A, and N₅ is G. In some embodiments, N₁ is U, N₂ is G, N₃ is C, N₄ is A, and N₅ is U. In some embodiments, N₁ is U, N₂ is G, N₃ is C, N₄ is C, and N₅ is A. In some embodiments, N₁ is U, N₂ is G, N₃ is C, N₄ is C, and N₅ is C. In some embodiments, N₁ is U, N₂ is G, N₃ is C, N₄ is C, and N₅ is G. In some embodiments, N₁ is U, N₂ is G, N₃ is C, N₄ is C, and N₅ is U. In some embodiments, N₁ is U, N₂ is G, N₃ is C, N₄ is G, and N₅ is A. In some embodiments, N₁ is U, N₂ is G, N₃ is C, N₄ is G, and N₅ is C. In some embodiments, N₁ is U, N₂ is G, N₃ is C, N₄ is G, and N₅ is G. In some embodiments, N₁ is U, N₂ is G, N₃ is C, N₄ is G, and N₅ is U. In some embodiments, N₁ is U, N₂ is G, N₃ is C, N₄ is U, and N₅ is A. In some embodiments, N₁ is U, N₂ is G, N₃ is C, N₄ is U, and N₅ is C. In some embodiments, N₁ is U, N₂ is G, N₃ is C, N₄ is U, and N₅ is G. In some embodiments, N₁ is U, N₂ is G, N₃ is C, N₄ is U, and N₅ is U.

In some embodiments, N₁ is U, N₂ is G, N₃ is G, N₄ is A, and N₅ is A. In some embodiments, N₁ is U, N₂ is G, N₃ is G, N₄ is A, and N₅ is C. In some embodiments, N₁ is U, N₂ is G, N₃ is G, N₄ is A, and N₅ is G. In some embodiments, N₁ is U, N₂ is G, N₃ is G, N₄ is A, and N₅ is U. In some embodiments, N₁ is U, N₂ is G, N₃ is G, N₄ is C, and N₅ is A. In some embodiments, N₁ is U, N₂ is G, N₃ is G, N₄ is C, and N₅ is C. In some embodiments, N₁ is U, N₂ is G, N₃ is G, N₄ is C, and N₅ is G. In some embodiments, N₁ is U, N₂ is G, N₃ is G, N₄ is C, and N₅ is U. In some embodiments, N₁ is U, N₂ is G, N₃ is G, N₄ is G, and N₅ is A. In some embodiments, N₁ is U, N₂ is G, N₃ is G, N₄ is G, and N₅ is C. In some embodiments, N₁ is U, N₂ is G, N₃ is G, N₄ is G, and N₅ is G. In some embodiments, N₁ is U, N₂ is G, N₃ is G, N₄ is G, and N₅ is U. In some embodiments, N₁ is U, N₂ is G, N₃ is G, N₄ is U, and N₅ is A. In some embodiments, N₁ is U, N₂ is G, N₃ is G, N₄ is U, and N₅ is C. In some embodiments, N₁ is U, N₂ is G, N₃ is G, N₄ is U, and N₅ is G. In some embodiments, N₁ is U, N₂ is G, N₃ is G, N₄ is U, and N₅ is U.

In some embodiments, N₁ is U, N₂ is G, N₃ is U, N₄ is A, and N₅ is A. In some embodiments, N₁ is U, N₂ is G, N₃ is U, N₄ is A, and N₅ is C. In some embodiments, N₁ is U, N₂ is G, N₃ is U, N₄ is A, and N₅ is G. In some embodiments, N₁ is U, N₂ is G, N₃ is U, N₄ is A, and N₅ is U. In some embodiments, N₁ is U, N₂ is G, N₃ is U, N₄ is C, and N₅ is A. In some embodiments, N₁ is U, N₂ is G, N₃ is U, N₄ is C, and N₅ is C. In some embodiments, N₁ is U, N₂ is G, N₃ is U, N₄ is C, and N₅ is G. In some embodiments, N₁ is U, N₂ is G, N₃ is U, N₄ is C, and N₅ is U. In some embodiments, N₁ is U, N₂ is G, N₃ is U, N₄ is G, and N₅ is A. In some embodiments, N₁ is U, N₂ is G, N₃ is U, N₄ is G, and N₅ is C. In some embodiments, N₁ is U, N₂ is G, N₃ is U, N₄ is G, and N₅ is G. In some embodiments, N₁ is U, N₂ is G, N₃ is U, N₄ is G, and N₅ is U. In some embodiments, N₁ is U, N₂ is G, N₃ is U, N₄ is U, and N₅ is A. In some embodiments, N₁ is U, N₂ is G, N₃ is U, N₄ is U, and N₅ is C. In some embodiments, N₁ is U, N₂ is G, N₃ is U, N₄ is U, and N₅ is G. In some embodiments, N₁ is U, N₂ is G, N₃ is U, N₄ is U, and N₅ is U.

xvi. Exemplary Cap Proximal Sequences where N₁ is U, and N₂ is U.

In some embodiments, N₁ is U, N₂ is U, N₃ is A, N₄ is A, and N₅ is A. In some embodiments, N₁ is U, N₂ is U, N₃ is A, N₄ is A, and N₅ is C. In some embodiments, N₁ is U, N₂ is U, N₃ is A, N₄ is A, and N₅ is G. In some embodiments, N₁ is U, N₂ is U, N₃ is A, N₄ is A, and N₅ is U. In some embodiments, N₁ is U, N₂ is U, N₃ is A, N₄ is C, and N₅ is A. In some embodiments, N₁ is U, N₂ is U, N₃ is A, N₄ is C, and N₅ is C. In some embodiments, N₁ is U, N₂ is U, N₃ is A, N₄ is C, and N₅ is G. In some embodiments, N₁ is U, N₂ is U, N₃ is A, N₄ is C, and N₅ is U. In some embodiments, N₁ is U, N₂ is U, N₃ is A, N₄ is G, and N₅ is A. In some embodiments, N₁ is U, N₂ is U, N₃ is A, N₄ is G, and N₅ is C. In some embodiments, N₁ is U, N₂ is U, N₃ is A, N₄ is G, and N₅ is G. In some embodiments, N₁ is U, N₂ is U, N₃ is A, N₄ is G, and N₅ is U. In some embodiments, N₁ is U, N₂ is U, N₃ is A, N₄ is U, and N₅ is A. In some embodiments, N₁ is U, N₂ is U, N₃ is A, N₄ is U, and N₅ is C. In some embodiments, N₁ is U, N₂ is U, N₃ is A, N₄ is U, and N₅ is G. In some embodiments, N₁ is U, N₂ is U, N₃ is A, N₄ is U, and N₅ is U.

In some embodiments, N₁ is U, N₂ is U, N₃ is C, N₄ is A, and N₅ is A. In some embodiments, N₁ is U, N₂ is U, N₃ is C, N₄ is A, and N₅ is C. In some embodiments, N₁ is U, N₂ is U, N₃ is C, N₄ is A, and N₅ is G. In some embodiments, N₁ is U, N₂ is U, N₃ is C, N₄ is A, and N₅ is U. In some embodiments, N₁ is U, N₂ is U, N₃ is C, N₄ is C, and N₅ is A. In some embodiments, N₁ is U, N₂ is U, N₃ is C, N₄ is C, and N₅ is C. In some embodiments, N₁ is U, N₂ is U, N₃ is C, N₄ is C, and N₅ is G. In some embodiments, N₁ is U, N₂ is U, N₃ is C, N₄ is C, and N₅ is U. In some embodiments, N₁ is U, N₂ is U, N₃ is C, N₄ is G, and N₅ is A. In some embodiments, N₁ is U, N₂ is U, N₃ is C, N₄ is G, and N₅ is C. In some embodiments, N₁ is U, N₂ is U, N₃ is C, N₄ is G, and N₅ is G. In some embodiments, N₁ is U, N₂ is U, N₃ is C, N₄ is G, and N₅ is U. In some embodiments, N₁ is U, N₂ is U, N₃ is C, N₄ is U, and N₅ is A. In some embodiments, N₁ is U, N₂ is U, N₃ is C, N₄ is U, and N₅ is C. In some embodiments, N₁ is U, N₂ is U, N₃ is C, N₄ is U, and N₅ is G. In some embodiments, N₁ is U, N₂ is U, N₃ is C, N₄ is U, and N₅ is U.

In some embodiments, N₁ is U, N₂ is U, N₃ is G, N₄ is A, and N₅ is A. In some embodiments, N₁ is U, N₂ is U, N₃ is G, N₄ is A, and N₅ is C. In some embodiments, N₁ is U, N₂ is U, N₃ is G, N₄ is A, and N₅ is G. In some embodiments, N₁ is U, N₂ is U, N₃ is G, N₄ is A, and N₅ is U. In some embodiments, N₁ is U, N₂ is U, N₃ is G, N₄ is C, and N₅ is A. In some embodiments, N₁ is U, N₂ is U, N₃ is G, N₄ is C, and N₅ is C. In some embodiments, N₁ is U, N₂ is U, N₃ is G, N₄ is C, and N₅ is G. In some embodiments, N₁ is U, N₂ is U, N₃ is G, N₄ is C, and N₅ is U. In some embodiments, N₁ is U, N₂ is U, N₃ is G, N₄ is G, and N₅ is A. In some embodiments, N₁ is U, N₂ is U, N₃ is G, N₄ is G, and N₅ is C. In some embodiments, N₁ is U, N₂ is U, N₃ is G, N₄ is G, and N₅ is G. In some embodiments, N₁ is U, N₂ is U, N₃ is G, N₄ is G, and N₅ is U. In some embodiments, N₁ is U, N₂ is U, N₃ is G, N₄ is U, and N₅ is A. In some embodiments, N₁ is U, N₂ is U, N₃ is G, N₄ is U, and N₅ is C. In some embodiments, N₁ is U, N₂ is U, N₃ is G, N₄ is U, and N₅ is G. In some embodiments, N₁ is U, N₂ is U, N₃ is G, N₄ is U, and N₅ is U.

In some embodiments, N₁ is U, N₂ is U, N₃ is U, N₄ is A, and N₅ is A. In some embodiments, N₁ is U, N₂ is U, N₃ is U, N₄ is A, and N₅ is C. In some embodiments, N₁ is U, N₂ is U, N₃ is U, N₄ is A, and N₅ is G. In some embodiments, N₁ is U, N₂ is U, N₃ is U, N₄ is A, and N₅ is U. In some embodiments, N₁ is U, N₂ is U, N₃ is U, N₄ is C, and N₅ is A. In some embodiments, N₁ is U, N₂ is U, N₃ is U, N₄ is C, and N₅ is C. In some embodiments, N₁ is U, N₂ is U, N₃ is U, N₄ is C, and N₅ is G. In some embodiments, N₁ is U, N₂ is U, N₃ is U, N₄ is C, and N₅ is U. In some embodiments, N₁ is U, N₂ is U, N₃ is U, N₄ is G, and N₅ is A. In some embodiments, N₁ is U, N₂ is U, N₃ is U, N₄ is G, and N₅ is C. In some embodiments, N₁ is U, N₂ is U, N₃ is U, N₄ is G, and N₅ is G. In some embodiments, N₁ is U, N₂ is U, N₃ is U, N₄ is G, and N₅ is U. In some embodiments, N₁ is U, N₂ is U, N₃ is U, N₄ is U, and N₅ is A. In some embodiments, N₁ is U, N₂ is U, N₃ is U, N₄ is U, and N₅ is C. In some embodiments, N₁ is U, N₂ is U, N₃ is U, N₄ is U, and N₅ is G. In some embodiments, N₁ is U, N₂ is U, N₃ is U, N₄ is U, and N₅ is U.

It will be understand, that embodiments to variables described above and herein (e.g., in sections D-i through D-xvi) may be combined with other embodiments to other variables described above and herein (e.g., 5′ caps).

Exemplary 5′ UTRs include a human alpha globin (hAg) 5′UTR or a fragment thereof, a TEV 5′ UTR or a fragment thereof, a HSP70 5′ UTR or a fragment thereof, or a c-Jun 5′ UTR or a fragment thereof.

In some embodiments, an RNA disclosed herein comprises a hAg 5′ UTR or a fragment thereof. In some embodiments, an RNA disclosed herein comprises a hAg 5′ UTR having 99%, 98%, 97%, 96%, 95%, 90%, 85%, or 80% identity to a human alpha globin 5′ UTR provided in SEQ ID NO: 11. In some embodiments, an RNA disclosed herein comprises a hAg 5′ UTR provided in SEQ ID NO: 11. In some embodiments, an RNA disclosed herein comprises a hAg 5′ UTR having 99%, 98%, 97%, 96%, 95%, 90%, 85%, or 80% identity to a human alpha globin 5′ UTR provided in SEQ ID NO: 12. In some embodiments, an RNA disclosed herein comprises a hAg 5′ UTR provided in SEQ ID NO: 12.

### 3′ UTR

In some embodiments, an RNA disclosed herein comprises a 3′-UTR. A 3′-UTR, if present, is located at the 3′ end, downstream of the termination codon of a protein-encoding region, but the term “3′-UTR” does preferably not include the poly(A) sequence. Thus, the 3′-UTR is upstream of the poly(A) sequence (if present), e.g. directly adjacent to the poly(A) sequence.

In some embodiments, an RNA disclosed herein comprises a 3′ UTR comprising a sequence element derived from the “amino terminal enhancer of split” (AES) mRNA and/or a sequence element from the mitochondrial encoded 12S ribosomal RNA (MT-RNR1). In some embodiments, an RNA disclosed herein comprises a 3′ UTR comprising a 3′ UTR of AES or a fragment or variant thereof. In some embodiments, an RNA disclosed herein comprises a 3′ UTR comprising a non-coding RNA of MT-RNR1 or a fragment or variant thereof. In some embodiments, an RNA disclosed herein comprises a 3′ UTR comprising a combination of (i) 3′ UTR of AES or a fragment or variant thereof and (ii) a non-coding RNA of MT-RNR1 or a fragment or variant thereof. Such and additional 3′ UTR sequences were identified by an ex vivo selection process of sequences that confer RNA stability and augment total protein expression (see, for example, WO 2017/060314, the entire content of which is incorporated herein by reference for the purposes described herein). In some embodiments, an RNA disclosed herein comprises a 3′ UTR having 99%, 98%, 97%, 96%, 95%, 90%, 85%, or 80% identity to a 3′ UTR provided in SEQ ID NO: 13. In some embodiments, an RNA disclosed herein comprises a 3′ UTR provided in SEQ ID NO: 13.

In some embodiments, an RNA disclosed herein comprises a 3′ UTR comprising two copies of 3′ UTR of a heterologous gene. For example, in some embodiments, an RNA disclosed herein comprises a 3′ UTR comprising two copies of 3′ UTR of human globin mRNA, for example, in some embodiments 3′ UTR of human beta-globin mRNA. See, for example, WO 2007/036366, the entire content of which is incorporated herein by reference for the purposes described herein.

In some embodiments, a 3′ UTR or a proximal sequence thereto comprises a restriction site. In some embodiments, a restriction site is a BamHI site. In some embodiments, a restriction site is a XhoI site.

### PolyA

In some embodiments, an RNA disclosed herein comprises a polyadenylate (PolyA) sequence, e.g., as described herein. In some embodiments, a PolyA sequence is situated downstream of a 3′-UTR, e.g., adjacent to a 3′-UTR.

As used herein, the term “poly(A) sequence” or “poly-A tail” refers to an uninterrupted or interrupted sequence of adenylate residues which is typically located at the 3′-end of an RNA polynucleotide. Poly(A) sequences are known to those of skill in the art and may follow the 3′-UTR in the RNAs described herein. An uninterrupted poly(A) sequence is characterized by consecutive adenylate residues. In nature, an uninterrupted poly(A) sequence is typical. RNAs disclosed herein can have a poly(A) sequence attached to the free 3′-end of the RNA by a template-independent RNA polymerase after transcription or a poly(A) sequence encoded by DNA and transcribed by a template-dependent RNA polymerase.

It has been demonstrated that in some embodiments, a poly(A) sequence of about 120 A nucleotides has a beneficial influence on the levels of RNA in transfected eukaryotic cells, as well as on the levels of protein that is translated from an open reading frame that is present upstream (5′) of the poly(A) sequence (Holtkamp et al., 2006, Blood, vol. 108, pp. 4009-4017).

The poly(A) sequence may be of any length. In some embodiments, a poly(A) sequence comprises, essentially consists of, or consists of at least 20, at least 30, at least 40, at least 80, or at least 100 and up to 500, up to 400, up to 300, up to 200, or up to 150 A nucleotides, and, in particular, about 120 A nucleotides. In this context, “essentially consists of” means that most nucleotides in the poly(A) sequence, typically at least 75%, at least 80%, at least 85%, at least 90%, at least 95%, at least 96%, at least 97%, at least 98%, or at least 99% by number of nucleotides in the poly(A) sequence are A nucleotides, but permits that remaining nucleotides are nucleotides other than A nucleotides, such as U nucleotides (uridylate), G nucleotides (guanylate), or C nucleotides (cytidylate). In this context, “consists of” means that all nucleotides in the poly(A) sequence, i.e., 100% by number of nucleotides in the poly(A) sequence, are A nucleotides. The term “A nucleotide” or “A” refers to adenylate.

In some embodiments, a poly(A) sequence is attached during RNA transcription, e.g., during preparation of in vitro transcribed RNA, based on a DNA template comprising repeated dT nucleotides (deoxythymidylate) in the strand complementary to the coding strand. The DNA sequence encoding a poly(A) sequence (coding strand) is referred to as poly(A) cassette.

In some embodiments, the poly(A) cassette present in the coding strand of DNA essentially consists of dA nucleotides, but is interrupted by a random sequence of the four nucleotides (dA, dC, dG, and dT). Such random sequence may be 5 to 50, 10 to 30, or 10 to 20 nucleotides in length. Such a cassette is disclosed in WO 2016/005324 A1, hereby incorporated by reference. Any poly(A) cassette disclosed in WO 2016/005324 A1 may be used in the present invention. A poly(A) cassette that essentially consists of dA nucleotides, but is interrupted by a random sequence having an equal distribution of the four nucleotides (dA, dC, dG, dT) and having a length of e.g., 5 to 50 nucleotides shows, on DNA level, constant propagation of plasmid DNA in E. coli and is still associated, on RNA level, with the beneficial properties with respect to supporting RNA stability and translational efficiency is encompassed. In some embodiments, the poly(A) sequence contained in an RNA polynucleotide described herein essentially consists of A nucleotides, but is interrupted by a random sequence of the four nucleotides (A, C, G, U). Such random sequence may be 5 to 50, 10 to 30, or 10 to 20 nucleotides in length. In some embodiments, an interrupted polyA sequence in accordance with the present disclosure is described in WO 2016/005324, the entire content of which is incorporated herein by reference for purposes described herein.

In some embodiments, no nucleotides other than A nucleotides flank a poly(A) sequence at its 3′-end, i.e., the poly(A) sequence is not masked or followed at its 3′-end by a nucleotide other than A.

In some embodiments, the poly(A) sequence may comprise at least 20, at least 30, at least 40, at least 80, or at least 100 and up to 500, up to 400, up to 300, up to 200, or up to 150 nucleotides. In some embodiments, the poly(A) sequence may essentially consist of at least 20, at least 30, at least 40, at least 80, or at least 100 and up to 500, up to 400, up to 300, up to 200, or up to 150 nucleotides. In some embodiments, the poly(A) sequence may consist of at least 20, at least 30, at least 40, at least 80, or at least 100 and up to 500, up to 400, up to 300, up to 200, or up to 150 nucleotides. In some embodiments, the poly(A) sequence comprises at least 100 nucleotides. In some embodiments, the poly(A) sequence comprises about 150 nucleotides. In some embodiments, the poly(A) sequence comprises about 120 nucleotides.

In some embodiments, an RNA disclosed herein comprises a poly(A) sequence comprising the nucleotide sequence of SEQ ID NO: 14, or a nucleotide sequence having at least 99%, 98%, 97%, 96%, 95%, 90%, 85%, or 80% identity to the nucleotide sequence of SEQ ID NO: 14. In some embodiments, an RNA disclosed herein comprises a poly(A) sequence of SEQ ID NO: 14.

### Payloads

In some embodiments, an RNA polynucleotide disclosed herein comprises a sequence encoding a payload, e.g., as described herein. In some embodiments, a sequence encoding a payload comprises a promoter sequence. In some embodiments, a sequence encoding a payload comprises a sequence encoding a secretory signal peptide.

In some embodiments, a payload is chosen from: a protein replacement polypeptide; an antibody agent; a cytokine; an antigenic polypeptide; a gene editing component; a regenerative medicine component or combinations thereof.

In some embodiments, a payload is or comprises a protein replacement polypeptide. In some embodiments, a protein replacement polypeptide comprises a polypeptide with aberrant expression in a disease or disorder. In some embodiments, a protein replacement polypeptide comprises an intracellular protein, an extracellular protein, or a transmembrane protein. In some embodiments, a protein replacement polypeptide comprises an enzyme.

In some embodiments, a disease or disorder with aberrant expression of a polypeptide includes but is not limited to: a rare disease, a metabolic disorder, a muscular dystrophy, a cardiovascular disease, or a monogenic disease.

In some embodiments, a payload is or comprises an antibody agent. In some embodiments, an antibody agent binds to a polypeptide expressed on a cell. In some embodiments, an antibody agent comprises a CD3 antibody, a Claudin 6 antibody, or a combination thereof.

In some embodiments, a payload is or comprises a cytokine or a fragment or a variant thereof. In some embodiments, a cytokine comprises: IL-12 or a fragment or variant or a fusion thereof, IL-15 or a fragment or a variant or a fusion thereof, GM-CSF or a fragment or a variant thereof; or IFN-alpha or a fragment or a variant thereof.

In some embodiments, a payload is or comprises an antigenic polypeptide or an immunogenic variant or an immunogenic fragment thereof. In some embodiments, an antigenic polypeptide comprises one epitope from an antigen. In some embodiments, an antigenic polypeptide comprises a plurality of distinct epitopes from an antigen. In some embodiments, an antigenic polypeptide comprises a plurality of distinct epitopes from at least two or more antigens. In some embodiments, an antigenic polypeptide comprising a plurality of distinct epitopes from one or more antigens is polyepitopic.

In some embodiments, an antigenic polypeptide comprises: an antigenic polypeptide from an allergen, a viral antigenic polypeptide, a bacterial antigenic polypeptide, a fungal antigenic polypeptide, a parasitic antigenic polypeptide, an antigenic polypeptide from an infectious agent, an antigenic polypeptide from a pathogen, a tumor antigenic polypeptide, or a self-antigenic polypeptide.

In some embodiments, an antigenic polypeptide comprises one or more antigenic polypeptides from Influenza virus, Pneumoviridae (e.g., parainfluenza (PIV3), henipavirus), Paramyxoviridae (e.g., Respiratory syncytial virus (RSV)), Metapneumovirus (e.g., hMPV), coronavirus, herpes simplex virus (HSV) Type 1 and/or Type 2, staph aureus, tuberculosis, ebola/alphaviruses, malaria, varicella-zoster virus, cytomegalovirus (CMV), norovirus, Zika virus, shingles, monkeypox virus, Hepatitis C virus, or human immunodeficiency virus (HIV), or combinations thereof.

In some embodiments, a parasitic antigenic polypeptide comprises a malarial antigenic polypeptide.

In some embodiments, a viral antigenic polypeptide comprises an HIV antigenic polypeptide, an influenza antigenic polypeptide, a Coronavirus antigenic polypeptide, a Rabies antigenic polypeptide, a varicella-zoster virus antigenic polypeptide, a cytomegalovirus (CMV) antigenic polypeptide, a norovirus antigenic polypeptide, or a Zika virus antigenic polypeptide.

In some embodiments, a viral antigenic polypeptide comprises an antigen from a virus that is associated with a zoonotic disease. In some such embodiments, a viral antigenic polypeptide comprises a monkeypox virus antigenic polypeptide.

In some embodiments, a viral antigenic polypeptide is or comprises a Coronavirus antigenic polypeptide. In some embodiments, a viral antigenic polypeptide is or comprises a alpha-coronavirus antigenic polypeptide. In some embodiments, a viral antigenic polypeptide is or comprises a beta-coronavirus antigenic polypeptide. In some embodiments, a Coronavirus antigen is or comprises a SARS-CoV-2 protein. In some embodiments, a SARS-CoV-2 protein comprises a SARS-CoV-2 Spike (S) protein, or an immunogenic variant or an immunogenic fragment thereof. In some embodiments, a SARS-CoV-2 protein comprises at least two proline substitutions (including, e.g., at least three, at least four, at least five, at least six proline substitutions). In some embodiments, a SARS-CoV-2 protein, or immunogenic variant or immunogenic fragment thereof, comprises proline residues at positions corresponding to positions 986 and 987 of SARS-CoV-2 S protein from Wuhan strain. Additionally or alternatively, in some embodiments, a SARS-CoV-2 protein, or immunogenic variant or immunogenic fragment thereof, comprises proline residues at positions corresponding to positions 817, 892, 899, and 942 of SARS-CoV-2 S protein from Wuhan strain. See, for example, WO 2021/243122, the entire contents of which are incorporated herein by reference for purposes described herein.

In some embodiments, a SARS-CoV-2 S polypeptide has at least 99%, 98%, 97%, 96%, 95%, 90%, 85%, or 80% identity to a SARS-CoV-2 S polypeptide disclosed herein. In some embodiments, a SARS-CoV-2 S polypeptide has at least 99%, 98%, 97%, 96%, 95%, 90%, 85%, or 80% identity to SEQ ID NO: 9.

In some embodiments, a SARS-CoV-2 S polypeptide is encoded by an RNA having at least 99%, 98%, 97%, 96%, 95%, 90%, 85%, or 80% identity to a SARS-CoV-2 S polynucleotide disclosed herein. In some embodiments, a SARS-CoV-2 S polypeptide is encoded by an RNA having at least 99%, 98%, 97%, 96%, 95%, 90%, 85%, or 80% identity to SEQ ID NO: 10.

In some embodiments, a SARS-CoV-2 S polypeptide comprises one or more mutations characteristic of a SARS-CoV-2 variant, for example, a SARS-CoV-2 variant that is or is predicted to be prevalent and/or rapidly spreading in a relevant jurisdictions. In some embodiments, such variants may be identified based on publicly available data (e.g., data provided in the GISAID Initiative database: https://www.gisaid.org, and/or data provided by the World Health Organization WHO (e.g., as provided at https://www.who.int/activities/tracking-SARS-CoV-2-variants). Mutations that characteristics of SARS-CoV-2 variants are known in the art. For example, the following strains, their SARS-CoV-2 S protein amino acid sequences and, in particular, modifications thereof compared to wildtype SARS-CoV-2 S protein amino acid sequence, e.g., as compared to SEQ ID NO: 9, can be useful in accordance with the present disclosure.

### B.1.1.7 (“Variant of Concern 202012/01” (VOC-202012/01)

B.1.1.7 is a variant of SARS-CoV-2 which was first detected in October 2020 during the COVID-19 pandemic in the United Kingdom from a sample taken the previous month, and quickly began to spread by mid-December. It was correlated with a significant increase in the rate of COVID-19 infection in United Kingdom; this increase is thought to be at least partly because of change N501Y inside the spike glycoprotein's receptor-binding domain, which is needed for binding to ACE2 in human cells. The B.1.1.7 variant is defined by 23 mutations: 13 non-synonymous mutations, 4 deletions, and 6 synonymous mutations (i.e., there are 17 mutations that change proteins and six that do not). The spike protein changes in B.1.1.7 include deletion 69-70, deletion 144, N501Y, A570D, D614G, P681H, T716I, S982A, and D1118H.

### B.1.351 (501.V2)

B.1.351 lineage and colloquially known as South African COVID-19 variant, is a variant of SARS-CoV-2. Preliminary results indicated that this variant may have an increased transmissibility. The B.1.351 variant is defined by multiple spike protein changes including: L18F, D80A, D215G, deletion 242-244, R246I, K417N, E484K, N501Y, D614G and A701V. There are three mutations of particular interest in the spike region of the B.1.351 genome: K417N, E484K, N501Y.

### B.1.1.298 (Cluster 5)

B.1.1.298 was discovered in North Jutland, Denmark, and is believed to have been spread from minks to humans via mink farms. Several different mutations in the spike protein of the virus have been confirmed. The specific mutations include deletion 69-70, Y453F, D614G, I692V, M1229I, and optionally S1147L.

### P.1 (B.1.1.248)

Lineage B.1.1.248, known as the Brazil(ian) variant, is one of the variants of SARS-CoV-2 which has been named P.1 lineage. P.1 has a number of S-protein modifications [L18F, T20N, P26S, D138Y, R190S, K417T, E484K, N501Y, D614G, H655Y, T1027I, V1176F] and is similar in certain key RBD positions (K417, E484, N501) to variant B.1.351 from South Africa.

### B.1.427/B.1.429 (CAL.20C)

Lineage B.1.427/B.1.429, also known as CAL.20C, is defined by the following modifications in the S-protein: S131, W152C, L452R, and D614G of which the L452R modification is of particular concern. CDC has listed B.1.427/B.1.429 as “variant of concern”.

### B.1.525

B.1.525 carries the same E484K modification as found in the P.1, and B.1.351 variants, and also carries the same ΔH69/ΔV70 deletion as found in B.1.1.7, and B.1.1.298. It also carries the modifications D614G, Q677H and F888L.

### B.1.526

B.1.526 was detected as an emerging lineage of viral isolates in the New York region that shares mutations with previously reported variants. The most common sets of spike mutations in this lineage are L5F, T95I, D253G, E484K, D614G, and A701V.

### B.1.1.529

B.1.529 was first detected in South Africa in November 2021. Omicron multiplies around 70 times faster than Delta variants, and quickly became the dominant strain of SARS-CoV-2 worldwide. Since its initial detection, a number of Omicron sublineages have arisen. Listed below are the current Omicron variants of concern, along with certain characteristic mutations associated with the S protein of each. The S protein of BA.4 and BA.5 have the same set of characteristic mutations, which is why the below table has a single row for “BA.4 or BA.5”, and why the present disclosure refers to a “BA.4/5” S protein in some embodiments.

TABLE 2
Omicron Variants of Concern and Characteristic Mutations

| Subvariant | Characteristic mutations |
|---|---|
| BA.1 | A67V, Δ69-70, T95I, G142D, Δ143-145, Δ211, |
| | L212I, ins214EPE, G339D, S371L, S373P, S375F, |
| | K417N, N440K, G446S, S477N, T478K, E484A, |
| | Q493R, G496S, Q498R, N501Y, Y505H, T547K, |
| | D614G, H655Y, N679K, P681H, N764K, D796Y, |
| | N856K, Q954H, N969K, and L981F |
| BA.2 | T19I, Δ24-26, A27S, G142D, V213G, G339D, |
| | S371F, S373P, S375F, T376A, D405N, R408S, |
| | K417N, N440K, S477N, T478K, E484A, Q493R, |
| | Q498R, N501Y, Y505H, D614G, H655Y, N679K, |
| | P681H, N764K, D796Y, Q954H, and N969K |
| BA.2.12.1 | T19I, Δ24-26, A27S, G142D, V213G, G339D, |
| | S371F, S373P, S375F, T376A, D405N, R408S, |
| | K417N, N440K, L452Q, S477N, T478K, E484A, |
| | Q493R, Q498R, N501Y, Y505H, D614G, H655Y, |
| | N679K, P681H, S704L, N764K, D796Y, Q954H, |
| | and N969K |
| BA.4 or | T19I, Δ24-26, A27S, Δ69/70, G142D, V213G, |
| BA.5 | G339D, S371F, S373P, S375F, T376A, D405N, |
| | R408S, K417N, N440K, L452R, S477N, T478K, |
| | E484A, F486V, Q498R, N501Y, Y505H, D614G, |
| | H655Y, N679K, P681H, N764K, D796Y, Q954H, |
| | and N969K |
| BA.2.75 | T19I, Δ24-26, A27S, G142D, K147E, W152R, |
| | F157L, I210V, V213G, G257S, G339H, N354D, |
| | S371F, S373P, S375F, T376A, D405N, R408S, |
| | K417N, N440K, G446S, N460K, S477N, T478K, |
| | E484A, Q498R, N501Y, Y505H D614G, H655Y, |
| | N679K, P681H, N764K, D796Y, Q954H, and N969K |
| BA.2.75.2 | T19I, Δ24-26, A27S, G142D, K147E, W152R, |
| | F157L, I210V, V213G, G257S, G339H, N354D, |
| | S371F, S373P, S375F, T376A, D405N, R408S, |
| | K417N, R346T, N440K, G446S, N460K, S477N, |
| | T478K, E484A, F486S, Q498R, N501Y, Y505H |
| | D614G, H655Y, N679K, P681H, N764K, D796Y, |
| | Q954H, N969K, and D1199N |
| BJ.1 | T19I, Δ24-26, A27S, V83A, G142D, Δ144, H146Q, |
| | Q183E, V213E, G339H, R346T, L368I, S371F, |
| | S373P, S375F, T376A, D405N, R408S, K417N, |
| | N440K, V445P, G446S, S477N, T478K, V483A, |
| | E484A, F490V, Q493R, Q498R, N501Y, Y505H, |
| | D614G, H655Y, N679K, P681H, N764K, D796Y, |
| | G798D, Q954H, N969K, and S1003I |
| BA.4.6 | T19I, Δ24-26, A27S, Δ69/70, G142D, V213G, |
| | G339D, R346T, S371F, S373P, S375F, T376A, |
| | D405N, R408S, K417N, N440K, L452R, S477N, |
| | T478K, E484A, F486V, Q498R, N501Y, Y505H, |
| | D614G, H655Y, N658S, N679K, P681H, N764K, |
| | D796Y, Q954H, and N969K |
| XBB | T191, Δ24-26, A27S, V83A, G142D, Δ 144, H146Q, |
| | Q183E, V213E, G252V, G339H, R346T, L368I, |
| | S371F, S373P, S375F, T376A, D405N, R408S, |
| | K417N, N440K, V445P, G446S, F486S, N460K, |
| | F490S, S477N, T478K, E484A, Q493R, Q498R, |
| | N501Y, Y505H, D614G, H655Y, N679K, P681H, |
| | N764K, D796Y, Q954H, and N969K |
| BA.2.3.20 | T19I, Δ24-26, A27S, G142D, M153T, N164K, |
| | V213G, H245N, G257D, G339D, S371F, S373P, |
| | S375F, T376A, D405N, R408S, K417N, N440K, |
| | K444R, E484R N450D, L452M, N460K, S477N, |
| | T478K, E484A, Q493R, Q498R, N501Y, Y505H, |
| | D614G, H655Y, N679K, P681H, N764K, D796Y, |
| | Q954H, and N969K |

In addition to the above Omicron variants, further variants of BA.5 have been observed (such variants including, e.g., BF.7, BF. 14, and BQ.1) comprising one of more of the following mutations in the S protein (positions shown relative to SEQ ID NO: 9): R346X, K444X, V445X, N450D, and S:N460X.

In one embodiment, a vaccine antigen described herein comprises, consists essentially of or consists of a spike protein (S) of SARS-CoV-2, a variant thereof, or a fragment thereof. In some embodiments, RNA described herein comprises a nucleotide sequence encoding a SARS-CoV-2 S protein comprising one or more mutations (including, e.g., 2, 3, 4, 5, 6, 7, 8, 9, 10, 11, 12, 13, 14, 15, 16, 17, 18, 19, 20, or more) that are characteristic of an Omicron variant. In some embodiments, an RNA comprises a nucleotide sequence encoding a SARS-CoV-2 S protein comprising one or more mutations (including, e.g., 2, 3, 4, 5, 6, 7, 8, 9, 10, 11, 12, 13, 14, 15, 16, 17, 18, 19, 20, or more) listed in Table 2. In some such embodiments, one or more mutations may come from two or more variants as listed in Table 2. In some embodiments, an RNA comprises a nucleotide sequence encoding a SARS-CoV-2 S protein comprising each of the mutations identified in Table 2 as being characteristic of a certain Omicron variant (e.g., in some embodiments, an RNA comprises a nucleotide sequence encoding a SARS-CoV-2 S protein comprising each of the mutations listed in Table 2 as being characteristic of an Omicron BA. 1, BA.2, BA.2.12.1, BA.4/5, BA.2.75, BA.2.75.1, BA.4.6 or XBB variant).

In some embodiments, an RNA encodes a SARS-CoV-2 S protein comprising a subset of the mutations listed in Table 2. In some embodiments, an RNA encodes a SARS-CoV-2 S protein comprising the mutations listed in Table 2 that are most prevalent in a certain variant (e.g., mutations that have been detected in at least 30%, at least 40%, at least 50%, at least 60%, at least 70%, at least 80%, at least 90%, at least 95%, at least 96%, at least 97%, at least 98%, at least 99%, or 100% of sequences collected to date for a given variant sequenced). Mutation prevalence can be determined, e.g., based on published sequences (e.g., sequences that are collected and made available to the public by GISAID).

In some embodiments, an RNA described herein encodes a SARS-CoV-2 S protein comprising one or more mutations (including, e.g., 2, 3, 4, 5, 6, 7, 8, 9, 10, 11, 12, 13, 14, 15, 16, 17, 18, 19, 20, or more) that are characteristic of a BA.4/5 variant.

In some embodiments, RNA described herein encodes a SARS-CoV-2 S protein comprising one or more (including, e.g., 2, 3, 4, 5, 6, 7, 8, 9, 10, 11, 12, 13, 14, 15, 16, 17, 18, 19, 20, or more) mutations characteristic of a BA.2.75 variant.

In some embodiments, RNA described herein encodes a SARS-CoV-2 S protein comprising one or more mutations (including, e.g., 2, 3, 4, 5, 6, 7, 8, 9, 10, 11, 12, 13, 14, 15, 16, 17, 18, 19, 20, or more) characteristic of a BA.2.75.2 variant.

In some embodiments, RNA described herein encodes a SARS-CoV-2 S protein comprising one or more mutations (including, e.g., 2, 3, 4, 5, 6, 7, 8, 9, 10, 11, 12, 13, 14, 15, 16, 17, 18, 19, 20, or more) characteristic of a BA.4.6 variant.

In some embodiments, RNA described herein encodes a SARS-CoV-2 S protein comprising one or more mutations (including, e.g., 2, 3, 4, 5, 6, 7, 8, 9, 10, 11, 12, 13, 14, 15, 16, 17, 18, 19, 20, or more) characteristic of an Omicron XBB variant.

In some embodiments, a payload is or comprises a tumor antigenic polypeptide or an immunogenic variant or an immunogenic fragment thereof. In some embodiments, a tumor antigenic polypeptide comprises a tumor specific antigen, a tumor associated antigen, a tumor neoantigen, or a combination thereof. In some embodiments, a tumor antigenic polypeptide comprises p53, ART-4, BAGE, ss-catenin/m, Bcr-abL CAMEL, CAP-1, CASP-8, CDC27/m, CDK4/m, CEA, CLAUDIN-12, c-MYC, CT, Cyp-B, DAM, ELF2M, ETV6-AML1, G250, GAGE, GnT-V, Gap100, HAGE, HER-2/neu, HPV-E7, HPV-E6, HAST-2, hTERT (or hTRT), LAGE, LDLR/FUT, MAGE-A, preferably MAGE-A1, MAGE-A2, MAGE-A3, MAGE-A4, MAGE-A5, MAGE-A6, MAGE-A7, MAGE-A8, MAGE-A9, MAGE-A10, MAGE-A11, or MAGE-A12, MAGE-B, MAGE-C, MART-1/Melan-A, MC1R, Myosin/m, MUC1, MUM-1, MUM-2, MUM-3, NA88-A, NF1, NY-ESO-1, NY—BR-1, p190 minor BCR-abL, Plac-1, Pm1/RARa, PRAME, proteinase 3, PSA, PSM, RAGE, RU1 or RU2, SAGE, SART-1 or SART-3, SCGB3A2, SCP1, SCP2, SCP3, SSX, SURVIVIN, TEL/AML1, TPI/m, TRP-1, TRP-2, TRP-2/INT2, TPTE, WT, WT-1, or a combination thereof.

In some embodiments, a tumor antigenic polypeptide comprises a tumor antigen from a carcinoma, a sarcoma, a melanoma, a lymphoma, a leukemia, or a combination thereof. In some embodiments, a tumor antigenic polypeptide comprises a melanoma tumor antigen. In some embodiments, a tumor antigenic polypeptide comprises a prostate cancer antigen. In some embodiments, a tumor antigenic polypeptide comprises a HPV16 positive head and neck cancer antigen. In some embodiments, a tumor antigenic polypeptide comprises a breast cancer antigen. In some embodiments, a tumor antigenic polypeptide comprises an ovarian cancer antigen. In some embodiments, a tumor antigenic polypeptide comprises a lung cancer antigen. In some embodiments, a tumor antigenic polypeptide comprises an NSCLC antigen.

In some embodiments, a payload is or comprises a self-antigenic polypeptide or an immunogenic variant or an immunogenic fragment thereof. In some embodiments, a self-antigenic polypeptide comprises an antigen that is typically expressed on cells and is recognized as a self-antigen by an immune system. In some embodiments, a self-antigenic polypeptide comprises: a multiple sclerosis antigenic polypeptide, a Rheumatoid arthritis antigenic polypeptide, a lupus antigenic polypeptide, a celiac disease antigenic polypeptide, a Sjogren's syndrome antigenic polypeptide, or an ankylosing spondylitis antigenic polypeptide, or a combination thereof.

### In Vitro Synthesis of RNA Polynucleotides

Commonly, in vitro transcription reactions include a double stranded DNA template comprised of a template strand (also known as a non-coding strand) and a coding strand. As RNA synthesis proceeds in the 5′ to 3′ direction, an RNA polymerase reads a template strand in the 3′ to 5′ direction. Accordingly, those skilled in the art appreciate that when a template strand is described in the present disclosure to comprise a sequence comprising positions +1, +2, +3, . . . +N, these positions are read in the 3′ to 5′ direction. Similarly, those skilled in the art appreciate that when a RNA transcript is described in the present disclosure to comprise a sequence comprising positions +1, +2, +3, . . . +N, such positions are read in the 5′ to 3′ direction.

Those skilled in the art appreciate that a “Transcription Start Site” sequence, when presented as single stranded (SS) sequence, typically relates to the coding strand sequence and reflects the canonical position at which the relevant RNA polymerase begins transcription. Those skilled in the art, reading the present disclosure will appreciate that, in some embodiments, a cap (e.g., a co-transcriptional cap) may include one or more residues corresponding to a position of such a “transcriptional start site sequence”, such that the first residue added by the RNA polymerase may in fact represent the second (or later) residue of the canonical Transcription Start Site.

In some embodiments, a DNA template is a linear DNA molecule. In some embodiments, a DNA template is a circular DNA molecule. DNA can be obtained or generated using methods known in the art, including, e.g., gene synthesis, recombinant DNA technology, or a combination thereof. In some embodiments, a DNA template comprises a nucleotide sequence coding for a transcribed region of interest (e.g., coding for a RNA described herein) and a promoter sequence that is recognized by an RNA polymerase selected for use in in vitro transcription. Various RNA polymerases are known in the art, including, e.g., DNA dependent RNA polymerases (e.g., a T7 RNA polymerase, a T3 RNA polymerase, a SP6 RNA polymerase, a N₄ virion RNA polymerase, or a variant or functional domain thereof). A skilled artisan will readily understand that an RNA polymerase utilized herein may be a recombinant RNA polymerase, and/or a purified RNA polymerase, i.e., not as part of a cell extract, which contains other components in addition to the RNA polymerases. One skilled in the art will recognize an appropriate promoter sequence for the selected RNA polymerase. In some embodiments, a DNA template can comprise a promoter sequence for a T7 RNA polymerase.

In some embodiments, the present disclosure provides an insight that a double stranded DNA template containing a pyrimidine base (e.g., C or U) at +2 position of a Transcription Start Site downstream from a RNA polymerase promoter (e.g., T7 promoter) can be useful for improving capping efficiency (e.g., percentage of capped transcripts in an in vitro transcription reaction), quality of an RNA preparation (e.g., of an in vitro transcribed RNA, e.g., the amount of short polynucleotide byproducts produced), translation efficiency of an RNA encoding a payload, and/or expression of a polypeptide payload encoded by an RNA. In some embodiments, such improvements can be observed independent of the identity of a 5′ UTR, capping method (e.g., enzymatic capping vs. co-transcriptional capping), cap structures (e.g., Cap0, Cap1, or Cap2), coding sequences, types of ribonucleotides (e.g., modified nucleotides vs. non-modified nucleotides), formulation (e.g., lipoplex vs. lipid nanoparticles) or combinations thereof. In some particular embodiments, a double stranded DNA template comprises a pyrimidine base (e.g., C or U) at +2 position of a Transcription Start Site and a G at +1 position of the Transcription Start Site. While a pyrimidine base (e.g., C or U) or a purine base (e.g., G or A) can be present at +3 position of a Transcription Start Site of a double stranded DNA template, in some particular embodiments, such a double stranded DNA template comprises a G at +3 position of the Transcription Start Site.

As appreciated by a skilled artisan in the art, 3′ end of a cap structure can be extended by an RNA polymerase using naturally occurring ribonucleotides and/or modified ribonucleotides. Therefore, a skilled artisan in the art will understand references to A, U, G, or C throughout the specification described herein can mean a naturally occurring ribonucleotide and/or a modified ribonucleotide described herein. For example, in some embodiments, a U is uridine. In some embodiments, a U is modified uridine (e.g., pseudouridine, 1-methyl pseudouridine).

In some embodiments, provided RNA polynucleotides are produced by in vitro transcription reaction described herein, e.g., using different combinations of cap structures (e.g., as described herein) and transcription start sites.

### AGA Transcription Start Site

In some embodiments, a Transcription Start Site that may be useful in accordance with the present disclosure is AGA. In some embodiments, an in vitro transcription reaction comprises: (i) a template DNA strand comprising a polynucleotide sequence complementary to an RNA polynucleotide sequence described herein, wherein the template DNA strand comprises a sequence that is complementary to an AGA transcription start site; (ii) a polymerase (e.g., an RNA polymerase such as, e.g., T7 polymerase); (iii) ribonucleotides; and (iv) a trinucleotide cap comprising N₁pN₂; wherein N₁ is A and N₂ is G; or wherein N₁ is G, C, or U, and N₂ is A and wherein the sequence in the template DNA strand that is complementary to AGA is the start site of transcription by an RNA polymerase. In some embodiments, N₁ is G and N₂ is A. In some embodiments, N₁ is C and N₂ is A. In some embodiments, N₁ is U and N₂ is A. In some embodiments, N₁ is A and N₂ is A. A skilled person in the art reading the present disclosure will appreciate that when an AGA Transcription Start Site is referenced with respect to a double-stranded DNA template, a coding strand of the double-stranded DNA template comprises an AGA start sequence, while a template DNA strand of the double stranded DNA template comprises a TCT which is the start site of transcription by an RNA polymerase.

In some embodiments, such in vitro transcription reactions can produce an RNA polynucleotide comprising a 5′ cap, a cap proximal sequence comprising positions +1, +2, +3, +4, and +5 of the RNA polynucleotide; and a sequence encoding a payload, wherein: (i) N₁ is position +1 of the RNA polynucleotide, (ii) N₂ is position +2 of the RNA polynucleotide, wherein N₁ and N₂ are selected from one of the following combinations: (a) N₁ is G and N₂ is A; (b) N₁ is U and N₂ is A; (c) N₁ is C and N₂ is A; and (d) N₁ is A and N₂ is A; and (iii) the cap proximal sequence comprises: N₁ and N₂ of the cap structure and a sequence comprising N₃N₄N₅ at positions +3, +4, and +5 respectively of the RNA polynucleotide, wherein N₃ is G, N₄ is A, and N₅ is selected from: A, C, G, and U. By way of example only, in some embodiments, an RNA polynucleotide resulting from such an in vitro transcription reaction comprises a 5′ cap and a cap proximal sequence comprising G₁A₂G₃A₄N₅ or U₁A₂G₃A₄N₅ or C₁A₂G₃A₄N₅ or A₁A₂G₃A₄N₅, wherein N₅ is independently selected from A, U, G, or C. In some embodiments, an RNA polynucleotide resulting from such an in vitro transcription reaction can be an RNA polynucleotide described herein.

In some embodiments, such in vitro transcription reactions can produce an RNA polynucleotide comprising a 5′ cap, a cap proximal sequence comprising positions +1, +2, +3, +4, and +5 of the RNA polynucleotide; and a sequence encoding a payload, wherein: (i) N₁ is position +1 of the RNA polynucleotide, (ii) N₂ is position +2 of the RNA polynucleotide, wherein N₁ is A and N₂ is G; and (iii) the cap proximal sequence comprises: N₁ and N₂ of the cap structure and a sequence comprising N₃N₄N₅ at positions +3, +4, and +5 respectively of the RNA polynucleotide, wherein N₃ is A, and N₄ and N₅ are each selected from: A, C, G, and U. By way of examples only, in some such embodiments, an RNA polynucleotide resulting from such an in vitro transcription reaction comprises a 5′ cap and a cap proximal sequence comprising A₁G₂A₃A₄N₅. In some embodiments, an RNA polynucleotide resulting from such an in vitro transcription reaction comprises a 5′ cap and a cap proximal sequence comprising A₁G₂A₃U₄N₅. In some embodiments, an RNA polynucleotide resulting from such an in vitro transcription reaction comprises a 5′ cap and a cap proximal sequence comprising A₁G₂A₃G₄N₅. In some embodiments, an RNA polynucleotide resulting from such an in vitro transcription reaction comprises a 5′ cap and a cap proximal sequence comprising A₁G₂A₃C₄N₅. A skilled artisan reading the present disclosure will realize that other RNA polynucleotides having different A₁G₂A₃N₄N₅ sequences (e.g., as described herein) can be produced by such in vitro transcription reaction described herein.

In some embodiments, an in vitro transcription reaction comprises: (i) a template DNA strand comprising a polynucleotide sequence complementary to an RNA polynucleotide sequence described herein, wherein the template DNA strand comprises a sequence that is complementary to an AGA transcription start site; (ii) a polymerase (e.g., an RNA polymerase such as, e.g., T7 polymerase); (iii) ribonucleotides; and (iv) a dinucleotide cap (e.g., as described herein) comprising N₁; wherein N₁ is G, wherein the sequence in the template strand that is complementary to AGA is the start site of transcription by an RNA polymerase. In some embodiments, G₁ nucleotide of a dinucleotide cap can interact with the second nucleotide of a sequence that is complementary to a AGA transcription start site. Without wishing to be bound by a particular theory, if G₁ nucleotide of a dinucleotide cap interacts with the second nucleotide of a sequence that is complementary to a AGA transcription start site, the first A of the AGA transcription start site will be absent from a resulting RNA polynucleotide.

In some embodiments, such in vitro transcription reactions can produce an RNA polynucleotide comprising a 5′ cap, a cap proximal sequence comprising positions +1, +2, +3, +4, and +5 of the RNA polynucleotide; and a sequence encoding a payload, wherein: (i) N₁ is position +1 of the RNA polynucleotide, and (ii) the cap proximal sequence comprises: N₁ of the cap structure and a sequence comprising N₂N₃N₄N₅ at positions +2, +3, +4, and +5 respectively of the RNA polynucleotide, wherein N₂, N₃, N₄ and N₅ are each independently selected from: A, C, G, and U. In some embodiments, an RNA polynucleotide resulting from such an in vitro transcription reaction comprises a 5′ cap and a cap proximal sequence comprising G₁A₂N₃N₄N₅, wherein N₃, N₄, and N₅ are each independently selected from A, U, G, or C. In some embodiments, an RNA polynucleotide resulting from such an in vitro transcription reaction comprises a 5′ cap and a cap proximal sequence comprising G₁A₂U₃N₄N₅, wherein N₄ and N₅ are each independently selected from A, U, G, or C. In some embodiments, an RNA polynucleotide resulting from such an in vitro transcription reaction comprises a 5′ cap and a cap proximal sequence comprising G₁A₂A₃N₄N₅, wherein N₄ and N₅ are each independently selected from A, U, G, or C. In some embodiments, an RNA polynucleotide resulting from such an in vitro transcription reaction comprises a 5′ cap and a cap proximal sequence comprising G₁A₂C₃N₄N₅, wherein N₄ and N₅ are each independently selected from A, U, G, or C. In some embodiments, an RNA polynucleotide resulting from such an in vitro transcription reaction comprises a 5′ cap and a cap proximal sequence comprising G₁A₂G₃N₄N₅, wherein N₄ and N₅ are each independently selected from A, U, G, or C. A skilled artisan reading the present disclosure will realize that other RNA polynucleotides having different G₁A₂N₃N₄N₅ sequences (e.g., as described herein) can be produced by such in vitro transcription reaction described herein.

In some embodiments, an in vitro transcription reaction comprises: (i) a template DNA strand comprising a polynucleotide sequence complementary to an RNA polynucleotide sequence described herein, wherein the template DNA strand comprises a sequence that is complementary to an AGA transcription start site; (ii) a polymerase (e.g., an RNA polymerase such as, e.g., T7 polymerase); (iii) ribonucleotides; and (iv) a tetranucleotide cap (e.g., as described herein) comprising N₁pN₂pN₃; wherein N₁ is C, A, G or U, and N₂ is A and N₃ are G; and wherein the sequence in the template strand complementary AGA is the start site of transcription by an RNA polymerase. In some embodiments, a skilled artisan reading the present disclosure will realize that RNA polynucleotides (e.g., as described herein) can be produced by such in vitro transcription reaction described herein.

In some embodiments, such in vitro transcription reactions can produce an RNA polynucleotide comprising a 5′ cap, a cap proximal sequence comprising positions +1, +2, +3, +4, and +5 of the RNA polynucleotide; and a sequence encoding a payload, wherein: (i) N₁ is position +1 of the RNA polynucleotide, (ii) N₂ is position +2 of the RNA polynucleotide; (iii) N₃ is position +3 of the RNA polynucleotide, wherein N₁ is C, A, G or U, N₂ is A and N₃ is G; and (iv) the cap proximal sequence comprises: N₁, N₂, and N₃ of the cap structure and a sequence comprising N₄N₅ at positions +4 and +5 respectively of the RNA polynucleotide, wherein N₄ is A, and N₅ is selected from: A, C, G, and U. In some embodiments, an RNA polynucleotide resulting from such an in vitro transcription reaction can be an RNA polynucleotide described herein.

In some embodiments, an in vitro transcription reaction comprises: (i) a template DNA strand comprising a polynucleotide sequence complementary to an RNA polynucleotide sequence described herein, wherein the template DNA strand comprises a sequence that is complementary to an AGA transcription start site; (ii) a polymerase (e.g., an RNA polymerase such as, e.g., T7 polymerase); (iii) ribonucleotides; and (iv) a tetranucleotide cap (e.g., as described herein) comprising N₁pN₂pN₃; wherein N₁ is A, N₂ is G, and N₃ is A; and wherein the sequence in the template strand complementary to AGA is the start site of transcription by an RNA polymerase.

In some embodiments, such in vitro transcription reactions can produce an RNA polynucleotide comprising a 5′ cap, a cap proximal sequence comprising positions +1, +2, +3, +4, and +5 of the RNA polynucleotide; and a sequence encoding a payload, wherein: (i) N₁ is position +1 of the RNA polynucleotide, (ii) N₂ is position +2 of the RNA polynucleotide; (iii) N₃ is position +3 of the RNA polynucleotide, wherein N₁ is A, N₂ is G and N₃ is A; and (iv) the cap proximal sequence comprises: N₁, N₂, and N₃ of the cap structure and a sequence comprising N₄N₅ at positions +4 and +5 respectively of the RNA polynucleotide, wherein N₄ and N₅ are each independently selected from: A, C, G, and U. In some embodiments, an RNA polynucleotide resulting from such an in vitro transcription reaction can be an RNA polynucleotide described herein.

### AGC Transcription Start Site

In some embodiments, a Transcription Start Site that may be useful in accordance with the present disclosure is AGC. In some embodiments, an in vitro transcription reaction comprises: (i) a template DNA strand comprising a polynucleotide sequence complementary to an RNA polynucleotide sequence described herein, wherein the template DNA strand comprises a sequence that is complementary to an AGC transcription start site; (ii) a polymerase (e.g., an RNA polymerase such as, e.g., T7 polymerase); (iii) ribonucleotides; and (iv) a trinucleotide cap comprising N₁pN₂; wherein N₁ is A and N₂ is G; or wherein N₁ is G, C, A, or U, and N₂ is A and wherein the sequence in the template DNA strand that is complementary to AGC is the start site of transcription by an RNA polymerase. In some embodiments, N₁ is G and N₂ is A. In some embodiments, N₁ is C and N₂ is A. In some embodiments, N₁ is U and N₂ is A. In some embodiments, N₁ is A and N₂ is A. A skilled person in the art reading the present disclosure will appreciate that when an AGC Transcription Start Site is referenced with respect to a double-stranded DNA template, a coding strand of the double-stranded DNA template comprises an AGC start sequence, while a template DNA strand of the double stranded DNA template comprises a TCG which is the start site of transcription by an RNA polymerase.

In some embodiments, such in vitro transcription reactions can produce an RNA polynucleotide comprising a 5′ cap, a cap proximal sequence comprising positions +1, +2, +3, +4, and +5 of the RNA polynucleotide; and a sequence encoding a payload, wherein: (i) N₁ is position +1 of the RNA polynucleotide, (ii) N₂ is position +2 of the RNA polynucleotide, wherein N₁ and N₂ are selected from one of the following combinations: (a) N₁ is G and N₂ is A; (b) N₁ is U and N₂ is A; (c) N₁ is C and N₂ is A; and (d) N₁ is A and N₂ is A; and (iii) the cap proximal sequence comprises: N₁ and N₂ of the cap structure and a sequence comprising N₃N₄N₅ at positions +3, +4, and +5 respectively of the RNA polynucleotide, wherein N₃ is G, N₄ is C, and N₅ is selected from: A, C, G, and U. By way of example only, in some embodiments, an RNA polynucleotide resulting from such an in vitro transcription reaction comprises a 5′ cap and a cap proximal sequence comprising G₁A₂G₃C₄N₅ or U₁A₂G₃C₄N₅ or C₁A₂G₃C₄N₅ or A₁A₂G₃C₄N₅, wherein N₅ is independently selected from A, U, G, or C. In some embodiments, an RNA polynucleotide resulting from such an in vitro transcription reaction can be an RNA polynucleotide described herein.

In some embodiments, such in vitro transcription reactions can produce an RNA polynucleotide comprising a 5′ cap, a cap proximal sequence comprising positions +1, +2, +3, +4, and +5 of the RNA polynucleotide; and a sequence encoding a payload, wherein: (i) N₁ is position +1 of the RNA polynucleotide, (ii) N₂ is position +2 of the RNA polynucleotide, wherein N₁ is A and N₂ is G; and (iii) the cap proximal sequence comprises: N₁ and N₂ of the cap structure and a sequence comprising N₃N₄N₅ at positions +3, +4, and +5 respectively of the RNA polynucleotide, wherein N₃ is C, and N₄ and N₅ are each selected from: A, C, G, and U. By way of examples only, in some such embodiments, an RNA polynucleotide resulting from such an in vitro transcription reaction comprises a 5′ cap and a cap proximal sequence comprising A₁G₂C₃A₄N₅. In some embodiments, an RNA polynucleotide resulting from such an in vitro transcription reaction comprises a 5′ cap and a cap proximal sequence comprising A₁G₂C₃U₄N₅.

In some embodiments, an RNA polynucleotide resulting from such an in vitro transcription reaction comprises a 5′ cap and a cap proximal sequence comprising A₁G₂C₃G₄N₅. In some embodiments, an RNA polynucleotide resulting from such an in vitro transcription reaction comprises a 5′ cap and a cap proximal sequence comprising A₁G₂C₃C₄N₅. A skilled artisan reading the present disclosure will realize that other RNA polynucleotides having different A₁G₂C₃N₄N₅ sequences (e.g., as described herein) can be produced by such in vitro transcription reaction described herein.

In some embodiments, an in vitro transcription reaction comprises: (i) a template DNA strand comprising a polynucleotide sequence complementary to an RNA polynucleotide sequence described herein, wherein the template DNA strand comprises a sequence that is complementary to an AGC transcription start site; (ii) a polymerase (e.g., an RNA polymerase such as, e.g., T7 polymerase); (iii) ribonucleotides; and (iv) a dinucleotide cap (e.g., as described herein) comprising N₁; wherein N₁ is G, wherein the sequence in the template strand that is complementary to AGC is the start site of transcription by an RNA polymerase. In some embodiments, G₁ nucleotide of a dinucleotide cap can interact with the second nucleotide of a sequence that is complementary to an AGC transcription start site. Without wishing to be bound by a particular theory, if G₁ nucleotide of a dinucleotide cap interacts with the second nucleotide of a sequence that is complementary to an AGC transcription start site, the first A of the AGC transcription start site will be absent from a resulting RNA polynucleotide.

In some embodiments, such in vitro transcription reactions can produce an RNA polynucleotide comprising a 5′ cap, a cap proximal sequence comprising positions +1, +2, +3, +4, and +5 of the RNA polynucleotide; and a sequence encoding a payload, wherein: (i) N₁ is position +1 of the RNA polynucleotide, and (ii) the cap proximal sequence comprises: N₁ of the cap structure and a sequence comprising N₂N₃N₄N₅ at positions +2, +3, +4, and +5 respectively of the RNA polynucleotide, wherein N₂, N₃, N₄ and N₅ are each independently selected from: A, C, G, and U. In some embodiments, an RNA polynucleotide resulting from such an in vitro transcription reaction comprises a 5′ cap and a cap proximal sequence comprising G₁C₂N₃N₄N₅, wherein N₃, N₄, and N₅ are each independently selected from A, U, G, or C. In some embodiments, an RNA polynucleotide resulting from such an in vitro transcription reaction comprises a 5′ cap and a cap proximal sequence comprising G₁C₂U₃N₄N₅, wherein N₄ and N₅ are each independently selected from A, U, G, or C. In some embodiments, an RNA polynucleotide resulting from such an in vitro transcription reaction comprises a 5′ cap and a cap proximal sequence comprising G₁C₂A₃N₄N₅, wherein N₄ and N₅ are each independently selected from A, U, G, or C. In some embodiments, an RNA polynucleotide resulting from such an in vitro transcription reaction comprises a 5′ cap and a cap proximal sequence comprising G₁C₂C₃N₄N₅, wherein N₄ and N₅ are each independently selected from A, U, G, or C. In some embodiments, an RNA polynucleotide resulting from such an in vitro transcription reaction comprises a 5′ cap and a cap proximal sequence comprising G₁C₂G₃N₄N₅, wherein N₄ and N₅ are each independently selected from A, U, G, or C. A skilled artisan reading the present disclosure will realize that other RNA polynucleotides having different G₁C₂N₃N₄N₅ sequences (e.g., as described herein) can be produced by such in vitro transcription reaction described herein.

In some embodiments, an in vitro transcription reaction comprises: (i) a template DNA strand comprising a polynucleotide sequence complementary to an RNA polynucleotide sequence described herein, wherein the template DNA strand comprises a sequence that is complementary to an AGA transcription start site; (ii) a polymerase (e.g., an RNA polymerase such as, e.g., T7 polymerase); (iii) ribonucleotides; and (iv) a tetranucleotide cap (e.g., as described herein) comprising N₁pN₂pN₃; wherein N₁ is C, A, G or U, and N₂ is A and N₃ are G; and wherein the sequence in the template strand complementary AGC is the start site of transcription by an RNA polymerase. In some embodiments, a skilled artisan reading the present disclosure will realize that RNA polynucleotides (e.g., as described herein) can be produced by such in vitro transcription reaction described herein.

In some embodiments, such in vitro transcription reactions can produce an RNA polynucleotide comprising a 5′ cap, a cap proximal sequence comprising positions +1, +2, +3, +4, and +5 of the RNA polynucleotide; and a sequence encoding a payload, wherein: (i) N₁ is position +1 of the RNA polynucleotide, (ii) N₂ is position +2 of the RNA polynucleotide; (iii) N₃ is position +3 of the RNA polynucleotide, wherein N₁ is C, A, G or U, N₂ is A and N₃ is G; and (iv) the cap proximal sequence comprises: N₁, N₂, and N₃ of the cap structure and a sequence comprising N₄N₅ at positions +4 and +5 respectively of the RNA polynucleotide, wherein N₄ is C, and N₅ is selected from: A, C, G, and U. In some embodiments, an RNA polynucleotide resulting from such an in vitro transcription reaction can be an RNA polynucleotide described herein.

In some embodiments, an in vitro transcription reaction comprises: (i) a template DNA strand comprising a polynucleotide sequence complementary to an RNA polynucleotide sequence described herein, wherein the template DNA strand comprises a sequence that is complementary to an AGC transcription start site; (ii) a polymerase (e.g., an RNA polymerase such as, e.g., T7 polymerase); (iii) ribonucleotides; and (iv) a tetranucleotide cap (e.g., as described herein) comprising N₁pN₂pN₃; wherein N₁ is A, N₂ is G, and N₃ is C; and wherein the sequence in the template strand complementary to AGC is the start site of transcription by an RNA polymerase.

In some embodiments, such in vitro transcription reactions can produce an RNA polynucleotide comprising a 5′ cap, a cap proximal sequence comprising positions +1, +2, +3, +4, and +5 of the RNA polynucleotide; and a sequence encoding a payload, wherein: (i) N₁ is position +1 of the RNA polynucleotide, (ii) N₂ is position +2 of the RNA polynucleotide; (iii) N₃ is position +3 of the RNA polynucleotide, wherein N₁ is A, N₂ is G and N₃ is C; and (iv) the cap proximal sequence comprises: N₁, N₂, and N₃ of the cap structure and a sequence comprising N₄N₅ at positions +4 and +5 respectively of the RNA polynucleotide, wherein N₄ and N₅ are each independently selected from: A, C, G, and U. In some embodiments, an RNA polynucleotide resulting from such an in vitro transcription reaction can be an RNA polynucleotide described herein.

### AUA Transcription Start Site

In some embodiments, a Transcription Start Site that may be useful in accordance with the present disclosure is AUA. In some embodiments, an in vitro transcription reaction comprises: (i) a template DNA strand comprising a polynucleotide sequence complementary to an RNA polynucleotide sequence described herein, wherein the template DNA strand comprises a sequence that is complementary to an AUA transcription start site; (ii) a polymerase (e.g., an RNA polymerase such as, e.g., T7 polymerase); (iii) ribonucleotides; and (iv) a trinucleotide cap comprising N₁pN₂; wherein N₁ is A and N₂ is U; or wherein N₁ is G, C, A, or U, and N₂ is A and wherein the sequence in the template DNA strand that is complementary to AUA is the start site of transcription by an RNA polymerase. In some embodiments, N₁ is G and N₂ is A. In some embodiments, N₁ is C and N₂ is A. In some embodiments, N₁ is U and N₂ is A. In some embodiments, N₁ is A and N₂ is A. A skilled person in the art reading the present disclosure will appreciate that when an AUA Transcription Start Site is referenced with respect to a double-stranded DNA template, a coding strand of the double-stranded DNA template comprises an AUA start sequence, while a template DNA strand of the double stranded DNA template comprises a TAT which is the start site of transcription by an RNA polymerase.

In some embodiments, such in vitro transcription reactions can produce an RNA polynucleotide comprising a 5′ cap, a cap proximal sequence comprising positions +1, +2, +3, +4, and +5 of the RNA polynucleotide; and a sequence encoding a payload, wherein: (i) N₁ is position +1 of the RNA polynucleotide, (ii) N₂ is position +2 of the RNA polynucleotide, wherein N₁ and N₂ are selected from one of the following combinations: (a) N₁ is G and N₂ is A; (b) N₁ is U and N₂ is A; (c) N₁ is C and N₂ is A; and (d) N₁ is A and N₂ is A; and (iii) the cap proximal sequence comprises: N₁ and N₂ of the cap structure and a sequence comprising N₃N₄N₅ at positions +3, +4, and +5 respectively of the RNA polynucleotide, wherein N₃ is U, N₄ is A, and N₅ is selected from: A, C, G, and U. By way of example only, in some embodiments, an RNA polynucleotide resulting from such an in vitro transcription reaction comprises a 5′ cap and a cap proximal sequence comprising G₁A₂U₃A₄N₅ or U₁A₂U₃A₄N₅ or C₁A₂U₃A₄N₅ or A₁A₂U₃A₄N₅, wherein N₅ is independently selected from A, U, G, or C. In some embodiments, an RNA polynucleotide resulting from such an in vitro transcription reaction can be an RNA polynucleotide described herein.

In some embodiments, such in vitro transcription reactions can produce an RNA polynucleotide comprising a 5′ cap, a cap proximal sequence comprising positions +1, +2, +3, +4, and +5 of the RNA polynucleotide; and a sequence encoding a payload, wherein: (i) N₁ is position +1 of the RNA polynucleotide, (ii) N₂ is position +2 of the RNA polynucleotide, wherein N₁ is A and N₂ is U; and (iii) the cap proximal sequence comprises: N₁ and N₂ of the cap structure and a sequence comprising N₃N₄N₅ at positions +3, +4, and +5 respectively of the RNA polynucleotide, wherein N₃ is A, and N₄ and N₅ are each selected from: A, C, G, and U. By way of examples only, in some such embodiments, an RNA polynucleotide resulting from such an in vitro transcription reaction comprises a 5′ cap and a cap proximal sequence comprising A₁U₂A₃A₄N₅. In some embodiments, an RNA polynucleotide resulting from such an in vitro transcription reaction comprises a 5′ cap and a cap proximal sequence comprising A₁U₂A₃U₄N₅.

In some embodiments, an RNA polynucleotide resulting from such an in vitro transcription reaction comprises a 5′ cap and a cap proximal sequence comprising A₁U₂A₃G₄N₅. In some embodiments, an RNA polynucleotide resulting from such an in vitro transcription reaction comprises a 5′ cap and a cap proximal sequence comprising A₁U₂A₃C₄N₅. A skilled artisan reading the present disclosure will realize that other RNA polynucleotides having different A₁U₂A₃N₄N₅ sequences (e.g., as described herein) can be produced by such in vitro transcription reaction described herein.

In some embodiments, an in vitro transcription reaction comprises: (i) a template DNA strand comprising a polynucleotide sequence complementary to an RNA polynucleotide sequence described herein, wherein the template DNA strand comprises a sequence that is complementary to an AUA transcription start site; (ii) a polymerase (e.g., an RNA polymerase such as, e.g., T7 polymerase); (iii) ribonucleotides; and (iv) a tetranucleotide cap (e.g., as described herein) comprising N₁pN₂pN₃; wherein N₁ is C, A, G or U, and N₂ is A and N₃ are U; and wherein the sequence in the template strand complementary AUA is the start site of transcription by an RNA polymerase. In some embodiments, a skilled artisan reading the present disclosure will realize that RNA polynucleotides (e.g., as described herein) can be produced by such in vitro transcription reaction described herein.

In some embodiments, such in vitro transcription reactions can produce an RNA polynucleotide comprising a 5′ cap, a cap proximal sequence comprising positions +1, +2, +3, +4, and +5 of the RNA polynucleotide; and a sequence encoding a payload, wherein: (i) N₁ is position +1 of the RNA polynucleotide, (ii) N₂ is position +2 of the RNA polynucleotide; (iii) N₃ is position +3 of the RNA polynucleotide, wherein N₁ is C, A, G or U, N₂ is A and N₃ is U; and (iv) the cap proximal sequence comprises: N₁, N₂, and N₃ of the cap structure and a sequence comprising N₄N₅ at positions +4 and +5 respectively of the RNA polynucleotide, wherein N₄ is A, and N₅ is selected from: A, C, G, and U. In some embodiments, an RNA polynucleotide resulting from such an in vitro transcription reaction can be an RNA polynucleotide described herein.

In some embodiments, an in vitro transcription reaction comprises: (i) a template DNA strand comprising a polynucleotide sequence complementary to an RNA polynucleotide sequence described herein, wherein the template DNA strand comprises a sequence that is complementary to an AUA transcription start site; (ii) a polymerase (e.g., an RNA polymerase such as, e.g., T7 polymerase); (iii) ribonucleotides; and (iv) a tetranucleotide cap (e.g., as described herein) comprising N₁pN₂pN₃; wherein N₁ is A, N₂ is U, and N₃ is A; and wherein the sequence in the template strand complementary to AUA is the start site of transcription by an RNA polymerase.

In some embodiments, such in vitro transcription reactions can produce an RNA polynucleotide comprising a 5′ cap, a cap proximal sequence comprising positions +1, +2, +3, +4, and +5 of the RNA polynucleotide; and a sequence encoding a payload, wherein: (i) N₁ is position +1 of the RNA polynucleotide, (ii) N₂ is position +2 of the RNA polynucleotide; (iii) N₃ is position +3 of the RNA polynucleotide, wherein N₁ is A, N₂ is U and N₃ is A; and (iv) the cap proximal sequence comprises: N₁, N₂, and N₃ of the cap structure and a sequence comprising N₄N₅ at positions +4 and +5 respectively of the RNA polynucleotide, wherein N₄ and N₅ are each independently selected from: A, C, G, and U. In some embodiments, an RNA polynucleotide resulting from such an in vitro transcription reaction can be an RNA polynucleotide described herein.

### CGC Transcription Start Site

In some embodiments, a Transcription Start Site that may be useful in accordance with the present disclosure is CGC. In some embodiments, an in vitro transcription reaction comprises: (i) a template DNA strand comprising a polynucleotide sequence complementary to an RNA polynucleotide sequence described herein, wherein the template DNA strand comprises a sequence that is complementary to a CGC transcription start site; (ii) a polymerase (e.g., an RNA polymerase such as, e.g., T7 polymerase); (iii) ribonucleotides; and (iv) a trinucleotide cap comprising N₁pN₂; wherein N₁ is C and N₂ is G; or wherein N₁ is A, C, G, or U, and N₂ is C and wherein the sequence in the template DNA strand that is complementary to CGC is the start site of transcription by an RNA polymerase. In some embodiments, N₁ is G and N₂ is C. In some embodiments, N₁ is U and N₂ is C. In some embodiments, N₁ is A and N₂ is C. In some embodiments, N₁ is C and N₂ is C. A skilled person in the art reading the present disclosure will appreciate that when a CGC Transcription Start Site is referenced with respect to a double-stranded DNA template, a coding strand of the double-stranded DNA template comprises a CGC start sequence, while a template DNA strand of the double stranded DNA template comprises a GCG which is the start site of transcription by an RNA polymerase.

In some embodiments, such in vitro transcription reactions can produce an RNA polynucleotide comprising a 5′ cap, a cap proximal sequence comprising positions +1, +2, +3, +4, and +5 of the RNA polynucleotide; and a sequence encoding a payload, wherein: (i) N₁ is position +1 of the RNA polynucleotide, (ii) N₂ is position +2 of the RNA polynucleotide, wherein N₁ and N₂ are selected from one of the following combinations: (a) N₁ is G and N₂ is C; (b) N₁ is U and N₂ is C; and (c) N₁ is A and N₂ is C; and (iii) the cap proximal sequence comprises: N₁ and N₂ of the cap structure and a sequence comprising N₃N₄N₅ at positions +3, +4, and +5 respectively of the RNA polynucleotide, wherein N₃ is G, N₄ is C, and N₅ is selected from: A, C, G, and U. By way of example only, in some embodiments, an RNA polynucleotide resulting from such an in vitro transcription reaction comprises a 5′ cap and a cap proximal sequence comprising G₁C₂G₃C₄N₅ or U₁C₂G₃C₄N₅ or A₁C₂G₃C₄N₅, wherein N₅ is independently selected from A, U, G, or C. In some embodiments, an RNA polynucleotide resulting from such an in vitro transcription reaction can be an RNA polynucleotide described herein.

In some embodiments, such in vitro transcription reactions can produce an RNA polynucleotide comprising a 5′ cap, a cap proximal sequence comprising positions +1, +2, +3, +4, and +5 of the RNA polynucleotide; and a sequence encoding a payload, wherein: (i) N₁ is position +1 of the RNA polynucleotide, (ii) N₂ is position +2 of the RNA polynucleotide, wherein N₁ is C and N₂ is G; and (iii) the cap proximal sequence comprises: N₁ and N₂ of the cap structure and a sequence comprising N₃N₄N₅ at positions +3, +4, and +5 respectively of the RNA polynucleotide, wherein N₃ is C, and N₄ and N₅ are each selected from: A, C, G, and U. By way of examples only, in some such embodiments, an RNA polynucleotide resulting from such an in vitro transcription reaction comprises a 5′ cap and a cap proximal sequence comprising C₁G₂C₃A₄N₅. In some embodiments, an RNA polynucleotide resulting from such an in vitro transcription reaction comprises a 5′ cap and a cap proximal sequence comprising C₁G₂C₃U₄N₅.

In some embodiments, an RNA polynucleotide resulting from such an in vitro transcription reaction comprises a 5′ cap and a cap proximal sequence comprising C₁G₂C₃G₄N₅. In some embodiments, an RNA polynucleotide resulting from such an in vitro transcription reaction comprises a 5′ cap and a cap proximal sequence comprising C₁G₂C₃C₄N₅. A skilled artisan reading the present disclosure will realize that other RNA polynucleotides having different C₁G₂C₃N₄N₅ sequences (e.g., as described herein) can be produced by such in vitro transcription reaction described herein.

In some embodiments, an in vitro transcription reaction comprises: (i) a template DNA strand comprising a polynucleotide sequence complementary to an RNA polynucleotide sequence described herein, wherein the template DNA strand comprises a sequence that is complementary to a CGC transcription start site; (ii) a polymerase (e.g., an RNA polymerase such as, e.g., T7 polymerase); (iii) ribonucleotides; and (iv) a dinucleotide cap (e.g., as described herein) comprising N₁; wherein N₁ is G, wherein the sequence in the template strand that is complementary to CGC is the start site of transcription by an RNA polymerase. In some embodiments, G₁ nucleotide of a dinucleotide cap can interact with the second nucleotide of a sequence that is complementary to a CGC transcription start site. Without wishing to be bound by a particular theory, if G₁ nucleotide of a dinucleotide cap interacts with the second nucleotide of a sequence that is complementary to a CGC transcription start site, the first C of the CGC transcription start site will be absent from a resulting RNA polynucleotide.

In some embodiments, such in vitro transcription reactions can produce an RNA polynucleotide comprising a 5′ cap, a cap proximal sequence comprising positions +1, +2, +3, +4, and +5 of the RNA polynucleotide; and a sequence encoding a payload, wherein: (i) N₁ is position +1 of the RNA polynucleotide, and (ii) the cap proximal sequence comprises: N₁ of the cap structure and a sequence comprising N₂N₃N₄N₅ at positions +2, +3, +4, and +5 respectively of the RNA polynucleotide, wherein N₂, N₃, N₄ and N₅ are each independently selected from: A, C, G, and U. In some embodiments, an RNA polynucleotide resulting from such an in vitro transcription reaction comprises a 5′ cap and a cap proximal sequence comprising G₁C₂N₃N₄N₅, wherein N₃, N₄, and N₅ are each independently selected from A, U, G, or C. In some embodiments, an RNA polynucleotide resulting from such an in vitro transcription reaction comprises a 5′ cap and a cap proximal sequence comprising G₁C₂U₃N₄N₅, wherein N₄ and N₅ are each independently selected from A, U, G, or C. In some embodiments, an RNA polynucleotide resulting from such an in vitro transcription reaction comprises a 5′ cap and a cap proximal sequence comprising G₁C₂A₃N₄N₅, wherein N₄ and N₅ are each independently selected from A, U, G, or C. In some embodiments, an RNA polynucleotide resulting from such an in vitro transcription reaction comprises a 5′ cap and a cap proximal sequence comprising G₁C₂C₃N₄N₅, wherein N₄ and N₅ are each independently selected from A, U, G, or C. In some embodiments, an RNA polynucleotide resulting from such an in vitro transcription reaction comprises a 5′ cap and a cap proximal sequence comprising G₁C₂G₃N₄N₅, wherein N₄ and N₅ are each independently selected from A, U, G, or C. A skilled artisan reading the present disclosure will realize that other RNA polynucleotides having different G₁C₂N₃N₄N₅ sequences (e.g., as described herein) can be produced by such in vitro transcription reaction described herein.

In some embodiments, an in vitro transcription reaction comprises: (i) a template DNA strand comprising a polynucleotide sequence complementary to an RNA polynucleotide sequence described herein, wherein the template DNA strand comprises a sequence that is complementary to a CGC transcription start site; (ii) a polymerase (e.g., an RNA polymerase such as, e.g., T7 polymerase); (iii) ribonucleotides; and (iv) a tetranucleotide cap (e.g., as described herein) comprising N₁pN₂pN₃; wherein N₁ is C, A, G or U, and N₂ is C and N₃ are G; and wherein the sequence in the template strand complementary CGC is the start site of transcription by an RNA polymerase. In some embodiments, a skilled artisan reading the present disclosure will realize that RNA polynucleotides (e.g., as described herein) can be produced by such in vitro transcription reaction described herein.

In some embodiments, an in vitro transcription reaction comprises: (i) a template DNA strand comprising a polynucleotide sequence complementary to an RNA polynucleotide sequence described herein, wherein the template DNA strand comprises a sequence that is complementary to a CGC transcription start site; (ii) a polymerase (e.g., an RNA polymerase such as, e.g., T7 polymerase); (iii) ribonucleotides; and (iv) a tetranucleotide cap (e.g., as described herein) comprising N₁pN₂pN₃; wherein N₁ is G, and N₂ is C and N₃ are G; and wherein the sequence in the template strand complementary to CGC is the start site of transcription by an RNA polymerase.

In some embodiments, such in vitro transcription reactions can produce an RNA polynucleotide comprising a 5′ cap, a cap proximal sequence comprising positions +1, +2, +3, +4, and +5 of the RNA polynucleotide; and a sequence encoding a payload, wherein: (i) N₁ is position +1 of the RNA polynucleotide, (ii) N₂ is position +2 of the RNA polynucleotide; (iii) N₃ is position +3 of the RNA polynucleotide, wherein N₁ is G, N₂, is C, and N₃ is G; and (iv) the cap proximal sequence comprises: N₁, N₂, and N₃ of the cap structure and a sequence comprising N₄N₅ at positions +4 and +5 respectively of the RNA polynucleotide, wherein N₄ is C, and N₅ is selected from: A, C, G, and U. In some embodiments, an RNA polynucleotide resulting from such an in vitro transcription reaction can be an RNA polynucleotide described herein.

In some embodiments, an in vitro transcription reaction comprises: (i) a template DNA strand comprising a polynucleotide sequence complementary to an RNA polynucleotide sequence described herein, wherein the template DNA strand comprises a sequence that is complementary to a CGC transcription start site; (ii) a polymerase (e.g., an RNA polymerase such as, e.g., T7 polymerase); (iii) ribonucleotides; and (iv) a tetranucleotide cap (e.g., as described herein) comprising N₁pN₂pN₃; wherein N₁ is C, N₂ is G, and N₃ is C; and wherein the sequence in the template strand complementary to CGC is the start site of transcription by an RNA polymerase. A skilled artisan reading the present disclosure will realize that other RNA polynucleotides having different G₁G₂G₃N₄N₅ sequences (e.g., as described herein) can produced by such in vitro transcription reaction described herein.

### GCG Transcription Start Site

In some embodiments, a Transcription Start Site that may be useful in accordance with the present disclosure is GCG. In some embodiments, an in vitro transcription reaction comprises: (i) a template DNA strand comprising a polynucleotide sequence complementary to an RNA polynucleotide sequence described herein, wherein the template DNA strand comprises a sequence that is complementary to a GCG transcription start site; (ii) a polymerase (e.g., an RNA polymerase such as, e.g., T7 polymerase); (iii) ribonucleotides; and (iv) a trinucleotide cap (e.g., as described herein) comprising N₁pN₂; wherein N₁ is A, C, G, or U, and N₂ is G; or wherein N₁ is G and N₂ is C; and wherein the sequence in the template strand that is complementary to GCG is the start site of transcription by an RNA polymerase. In some embodiments, N₁ is G and N₂ is G. In some embodiments, N₁ is U and N₂ is G. In some embodiments, N₁ is A and N₂ is G. In some embodiments, N₁ is C and N₂ is G. In some embodiments, N₁ is G and N₂ is C. A skilled person in the art reading the present disclosure will appreciate that when a GCG Transcription Start Site is referenced with respect to a double-stranded DNA template, a coding strand of the double-stranded DNA template comprises a GCG start sequence, while a template DNA strand of the double stranded DNA template comprises a CGC which is the start site of transcription by an RNA polymerase.

In some embodiments, such in vitro transcription reactions can produce an RNA polynucleotide comprising a 5′ cap, a cap proximal sequence comprising positions +1, +2, +3, +4, and +5 of the RNA polynucleotide; and a sequence encoding a payload, wherein: (i) N₁ is position +1 of the RNA polynucleotide, (ii) N₂ is position +2 of the RNA polynucleotide, wherein N₁ and N₂ are selected from one of the following combinations: (a) N₁ is G and N₂ is G; (b) N₁ is U and N₂ is G; (c) N₁ is A and N₂ is G; and (d) N₁ is C and N₂ is G and (iii) the cap proximal sequence comprises: N₁ and N₂ of the cap structure and a sequence comprising N₃N₄N₅ at positions +3, +4, and +5 respectively of the RNA polynucleotide, wherein N₃ is C, N₄ is G, and N₅ is selected from: A, C, G, and U. By way of example only, in some embodiments, an RNA polynucleotide resulting from such an in vitro transcription reaction comprises a 5′ cap and a cap proximal sequence comprising G₁G₂C₃G₄N₅ or U₁G₂C₃G₄N₅ or A₁G₂C₃G₄N₅ or C₁G₂C₃G₄N₅, wherein N₅ is independently selected from A, U, G, or C. In some embodiments, an RNA polynucleotide resulting from such an in vitro transcription reaction can be an RNA polynucleotide described herein.

In some embodiments, such in vitro transcription reactions can produce an RNA polynucleotide comprising a 5′ cap, a cap proximal sequence comprising positions +1, +2, +3, +4, and +5 of the RNA polynucleotide; and a sequence encoding a payload, wherein: (i) N₁ is position +1 of the RNA polynucleotide, (ii) N₂ is position +2 of the RNA polynucleotide, wherein N₁ is G and N₂ is C; and (iii) the cap proximal sequence comprises: N₁ and N₂ of the cap structure and a sequence comprising N₃N₄N₅ at positions +3, +4, and +5 respectively of the RNA polynucleotide, wherein N₃ is G, and N₄ and N₅ are each selected from: A, C, G, and U. By way of examples only, in some such embodiments, an RNA polynucleotide resulting from such an in vitro transcription reaction comprises a 5′ cap and a cap proximal sequence comprising G₁C₂G₃A₄N₅. In some embodiments, an RNA polynucleotide resulting from such an in vitro transcription reaction comprises a 5′ cap and a cap proximal sequence comprising G₁C₂G₃U₄N₅.

In some embodiments, an RNA polynucleotide resulting from such an in vitro transcription reaction comprises a 5′ cap and a cap proximal sequence comprising G₁C₂G₃G₄N₅. In some embodiments, an RNA polynucleotide resulting from such an in vitro transcription reaction comprises a 5′ cap and a cap proximal sequence comprising G₁C₂G₃C₄N₅. A skilled artisan reading the present disclosure will realize that other RNA polynucleotides having different G₁C₂G₃N₄N₅ sequences (e.g., as described herein) can be produced by such in vitro transcription reaction described herein.

In some embodiments, an in vitro transcription reaction comprises: (i) a template DNA strand comprising a polynucleotide sequence complementary to an RNA polynucleotide sequence described herein, wherein the template DNA strand comprises a sequence that is complementary to a GCG transcription start site; (ii) a polymerase (e.g., an RNA polymerase such as, e.g., T7 polymerase); (iii) ribonucleotides; and (iv) a dinucleotide cap (e.g., as described herein) comprising N₁; wherein N₁ is G, wherein the sequence in the template strand that is complementary to GCG is the start site of transcription by an RNA polymerase. In some embodiments, G₁ nucleotide of a dinucleotide cap can interact with the first nucleotide or the third nucleotide of a sequence that is complementary to a GCG transcription start site. Without wishing to be bound by a particular theory, if G₁ nucleotide of a dinucleotide cap interacts with the third nucleotide of a sequence that is complementary to a GCG transcription start site, the first two GCs of the GCG transcription start site will be absent from a resulting RNA polynucleotide.

In some embodiments, such in vitro transcription reactions can produce an RNA polynucleotide comprising a 5′ cap, a cap proximal sequence comprising positions +1, +2, +3, +4, and +5 of the RNA polynucleotide; and a sequence encoding a payload, wherein: (i) N₁ is position +1 of the RNA polynucleotide, and (ii) the cap proximal sequence comprises: N₁ of the cap structure and a sequence comprising N₂N₃N₄N₅ at positions +2, +3, +4, and +5 respectively of the RNA polynucleotide, wherein N₂, N₃, N₄ and N₅ are each independently selected from: A, C, G, and U. In some embodiments, an RNA polynucleotide resulting from such an in vitro transcription reaction comprises a 5′ cap and a cap proximal sequence comprising G₁C₂G₃N₄N₅, wherein N₄ and N₅ are each independently selected from A, U, G, or C. In some embodiments, an RNA polynucleotide resulting from such an in vitro transcription reaction comprises a 5′ cap and a cap proximal sequence comprising G₁N₂N₃N₄N₅, wherein N₃, N₄ and N₅ are each independently selected from A, U, G, or C. By way of example only, in some such embodiments, an RNA polynucleotide resulting from such an in vitro transcription reaction comprises a 5′ cap and a cap proximal sequence comprising G₁C₂G₃A₄U₅. In some embodiments, an RNA polynucleotide resulting from such an in vitro transcription reaction comprises a 5′ cap and a cap proximal sequence comprising G₁C₂G₃A₄A₅. In some embodiments, an RNA polynucleotide resulting from such an in vitro transcription reaction comprises a 5′ cap and a cap proximal sequence comprising G₁A₂A₃A₄G₅. In some embodiments, an RNA polynucleotide resulting from such an in vitro transcription reaction comprises a 5′ cap and a cap proximal sequence comprising G₁U₂A₃A₄A₅. A skilled artisan reading the present disclosure will realize that other RNA polynucleotides having different G₁C₂G₃N₄N₅ or G₁N₂N₃N₄N₅ sequences (e.g., as described herein) can be produced by such in vitro transcription reaction described herein.

In some embodiments, an in vitro transcription reaction comprises: (i) a template DNA strand comprising a polynucleotide sequence complementary to an RNA polynucleotide sequence described herein, wherein the template DNA strand comprises a sequence that is complementary to a GCG transcription start site; (ii) a polymerase (e.g., an RNA polymerase such as, e.g., T7 polymerase); (iii) ribonucleotides; and (iv) a tetranucleotide cap (e.g., as described herein) comprising N₁pN₂pN₃; wherein N₁ is C, A, G or U, and N₂ is G and N₃ are C; and wherein the sequence in the template strand complementary to GCG is the start site of transcription by an RNA polymerase. In some embodiments, a skilled artisan reading the present disclosure will realize that RNA polynucleotides (e.g., as described herein) can be produced by such in vitro transcription reaction described herein.

In some embodiments, an in vitro transcription reaction comprises: (i) a template DNA strand comprising a polynucleotide sequence complementary to an RNA polynucleotide sequence described herein, wherein the template DNA strand comprises a sequence that is complementary to a GCG transcription start site; (ii) a polymerase (e.g., an RNA polymerase such as, e.g., T7 polymerase); (iii) ribonucleotides; and (iv) a tetranucleotide cap (e.g., as described herein) comprising N₁pN₂pN₃; wherein N₁ is G, N₂ is C, and N₃ is G; and wherein the sequence in the template strand complementary to GCG is the start site of transcription by an RNA polymerase.

In some embodiments, such in vitro transcription reactions can produce an RNA polynucleotide comprising a 5′ cap, a cap proximal sequence comprising positions +1, +2, +3, +4, and +5 of the RNA polynucleotide; and a sequence encoding a payload, wherein: (i) N₁ is position +1 of the RNA polynucleotide, (ii) N₂ is position +2 of the RNA polynucleotide, and N₃ is position +3 of the RNA polynucleotide, wherein N₁ is G, N₂ is C, and N₂ is G; and (iii) the cap proximal sequence comprises: N₁, N₂, and N₃ of a tetranucleotide cap structure and a sequence comprising N₄N₅ at positions +4 and +5 respectively of the RNA polynucleotide, wherein N₄ and N₅ are each independently is selected from: A, C, G, and U. In some embodiments, an RNA polynucleotide resulting from such an in vitro transcription reaction comprises a 5′ cap and a cap proximal sequence comprising G₁C₂G₃N₄N₅, wherein N₄ and N₅ are each independently selected from A, U, G, or C. By way of example only, in some embodiments, an RNA polynucleotide resulting from such an in vitro transcription reaction comprises a 5′ cap and a cap proximal sequence comprising G₁C₂G₃A₄U₅. In some embodiments, an RNA polynucleotide resulting from such an in vitro transcription reaction comprises a 5′ cap and a cap proximal sequence comprising G₁C₂G₃A₄A₅. In some embodiments, an RNA polynucleotide resulting from such an in vitro transcription reaction comprises a 5′ cap and a cap proximal sequence comprising G₁C₂G₃U₄A₅. A skilled artisan reading the present disclosure will realize that other RNA polynucleotides having different G₁C₂G₃N₄N₅ sequences (e.g., as described herein) can be produced by such in vitro transcription reaction described herein.

### GGG Transcription Start Site

In some embodiments, a Transcription Start Site that may be useful in accordance with the present disclosure is GGG. In some embodiments, an in vitro transcription reaction comprises: (i) a template DNA strand comprising a polynucleotide sequence complementary to an RNA polynucleotide sequence described herein, wherein the template DNA strand comprises a sequence that is complementary to a GGG transcription start site; (ii) a polymerase (e.g., an RNA polymerase such as, e.g., T7 polymerase); (iii) ribonucleotides; and (iv) a trinucleotide cap (e.g., as described herein) comprising N₁pN₂; wherein N₁ is selected from A, C, U, and G and N₂ is G, wherein the sequence in the template strand that is complementary to GGG is the start site of transcription by an RNA polymerase. In some embodiments, N₁ is C and N₂ is G. In some embodiments, N₁ is U and N₂ is G. In some embodiments, N₁ is A and N₂ is G. A skilled person in the art reading the present disclosure will appreciate that when a GGG Transcription Start Site is referenced with respect to a double-stranded DNA template, a coding strand of the double-stranded DNA template comprises a GGG start sequence, while a template DNA strand of the double stranded DNA template comprises a CCC which is the start site of transcription by an RNA polymerase.

In some embodiments, such in vitro transcription reactions can produce an RNA polynucleotide comprising a 5′ cap, a cap proximal sequence comprising positions +1, +2, +3, +4, and +5 of the RNA polynucleotide; and a sequence encoding a payload, wherein: (i) N₁ is position +1 of the RNA polynucleotide, (ii) N₂ is position +2 of the RNA polynucleotide, wherein N₁ and N₂ are selected from one of the following combinations: (a) N₁ is C and N₂ is G; (b) N₁ is U and N₂ is G; and (c) N₁ is A and N₂ is G; and (iii) the cap proximal sequence comprises: N₁ and N₂ of the cap structure and a sequence comprising N₃N₄N₅ at positions +3, +4, and +5 respectively of the RNA polynucleotide, wherein N₃ and N₄ are G, and N₅ is selected from: A, C, G, and U. By way of example only, in some embodiments, an RNA polynucleotide resulting from such an in vitro transcription reaction comprises a 5′ cap and a cap proximal sequence comprising C₁G₂G₃G₄N₅ or U₁G₂G₃G₄N₅ or A₁G₂G₃G₄N₅, wherein N₅ is independently selected from A, U, G, or C. In some embodiments, an RNA polynucleotide resulting from such an in vitro transcription reaction can be an RNA polynucleotide described herein.

In some embodiments, an in vitro transcription reaction comprises: (i) a template DNA strand comprising a polynucleotide sequence complementary to an RNA polynucleotide sequence described herein, wherein the template DNA strand comprises a sequence that is complementary to a GGG transcription start site; (ii) a polymerase (e.g., an RNA polymerase such as, e.g., T7 polymerase); (iii) ribonucleotides; and (iv) a dinucleotide cap (e.g., as described herein) comprising N₁; wherein N₁ is G, wherein the sequence in the template strand that is complementary to GGG is the start site of transcription by an RNA polymerase. In some embodiments, G₁ nucleotide of a dinucleotide cap can interact with the first nucleotide, the second nucleotide, or the third nucleotide of a sequence that is complementary to a GGG transcription start site. Without wishing to be bound by a particular theory, if G₁ nucleotide of a dinucleotide cap interacts with the second or third nucleotide of a sequence that is complementary to a GGG transcription start site, the first G or the first two Gs of the GGG transcription start site will be absent from a resulting RNA polynucleotide.

In some embodiments, such in vitro transcription reactions can produce an RNA polynucleotide comprising a 5′ cap, a cap proximal sequence comprising positions +1, +2, +3, +4, and +5 of the RNA polynucleotide; and a sequence encoding a payload, wherein: (i) N₁ is position +1 of the RNA polynucleotide, and (ii) the cap proximal sequence comprises: N₁ of the cap structure and a sequence comprising N₂N₃N₄N₅ at positions +2, +3, +4, and +5 respectively of the RNA polynucleotide, wherein N₂, N₃, N₄ and N₅ are each independently selected from: A, C, G, and U. In some embodiments, an RNA polynucleotide resulting from such an in vitro transcription reaction comprises a 5′ cap and a cap proximal sequence comprising G₁G₂G₃N₄N₅, wherein N₄ and N₅ are each independently selected from A, U, G, or C. In some embodiments, an RNA polynucleotide resulting from such an in vitro transcription reaction comprises a 5′ cap and a cap proximal sequence comprising G₁G₂N₃N₄N₅, wherein N₃, N₄ and N₅ are each independently selected from A, U, G, or C. In some embodiments, an RNA polynucleotide resulting from such an in vitro transcription reaction comprises a 5′ cap and a cap proximal sequence comprising G₁N₂N₃N₄N₅, wherein N₃, N₄ and N₅ are each independently selected from A, U, G, or C. By way of example only, in some such embodiments, an RNA polynucleotide resulting from such an in vitro transcription reaction comprises a 5′ cap and a cap proximal sequence comprising G₁G₂G₃A₄U₅. In some embodiments, an RNA polynucleotide resulting from such an in vitro transcription reaction comprises a 5′ cap and a cap proximal sequence comprising G₁G₂A₃A₄A₅. In some embodiments, an RNA polynucleotide resulting from such an in vitro transcription reaction comprises a 5′ cap and a cap proximal sequence comprising G₁G₂G₃A₄G₅. In some embodiments, an RNA polynucleotide resulting from such an in vitro transcription reaction comprises a 5′ cap and a cap proximal sequence comprising G₁A₂U₃A₄C₅. A skilled artisan reading the present disclosure will realize that other RNA polynucleotides having different G₁N₂N₃N₄N₅ or G₁G₂N₃N₄N₅ or G₁G₂G₃N₄N₅ sequences (e.g., as described herein) can be produced by such in vitro transcription reaction described herein.

In some embodiments, an in vitro transcription reaction comprises: (i) a template DNA strand comprising a polynucleotide sequence complementary to an RNA polynucleotide sequence described herein, wherein the template DNA strand comprises a sequence that is complementary to a GGG transcription start site; (ii) a polymerase (e.g., an RNA polymerase such as, e.g., T7 polymerase); (iii) ribonucleotides; and (iv) a tetranucleotide cap comprising N₁pN₂pN₃; wherein N₁ is G, N₂ is C, and N₃ are G; and wherein the sequence in the template strand complementary to GGG is the start site of transcription by an RNA polymerase. In some embodiments, a skilled artisan reading the present disclosure will realize that RNA polynucleotides (e.g., as described herein) can be produced by such in vitro transcription reaction described herein.

In some embodiments, an in vitro transcription reaction comprises: (i) a template DNA strand comprising a polynucleotide sequence complementary to an RNA polynucleotide sequence described herein, wherein the template DNA strand comprises a sequence that is complementary to a GGG transcription start site; (ii) a polymerase (e.g., an RNA polymerase such as, e.g., T7 polymerase); (iii) ribonucleotides; and (iv) a tetranucleotide cap comprising N₁pN₂pN₃; wherein N₁ is C, A, or U, and N₂ and N₃ are G; and wherein the sequence in the template strand complementary to GGG is the start site of transcription by an RNA polymerase. In some embodiments, a skilled artisan reading the present disclosure will realize that RNA polynucleotides (e.g., as described herein) can be produced by such in vitro transcription reaction described herein.

In some embodiments, an in vitro transcription reaction comprises: (i) a template DNA strand comprising a polynucleotide sequence complementary to an RNA polynucleotide sequence described herein, wherein the template DNA strand comprises a sequence that is complementary to a GGG transcription start site; (ii) a polymerase (e.g., an RNA polymerase such as, e.g., T7 polymerase); (iii) ribonucleotides; and (iv) a tetranucleotide cap (e.g., as described herein) comprising N₁pN₂pN₃; wherein N₁, N₂ and N₃ are each G; and wherein the sequence in the template strand complementary to GGG is the start site of transcription by an RNA polymerase. In some embodiments, a skilled artisan reading the present disclosure will realize that RNA polynucleotides (e.g., as described herein) can be produced by such in vitro transcription reaction described herein.

### GUG Transcription Start Site

In some embodiments, a Transcription Start Site that may be useful in accordance with the present disclosure is GUG. In some embodiments, an in vitro transcription reaction comprises: (i) a template DNA strand comprising a polynucleotide sequence complementary to an RNA polynucleotide sequence described herein, wherein the template DNA strand comprises a sequence that is complementary to a GUG transcription start site; (ii) a polymerase (e.g., an RNA polymerase such as, e.g., T7 polymerase); (iii) ribonucleotides; and (iv) a trinucleotide cap (e.g., as described herein) comprising N₁pN₂; wherein N₁ is A, C, G, or U, and N₂ is G; or wherein N₁ is G and N₂ is U; and wherein the sequence in the template strand that is complementary to GUG is the start site of transcription by an RNA polymerase. In some embodiments, N₁ is A and N₂ is G. In some embodiments, N₁ is C and N₂ is G. In some embodiments, N₁ is G and N₂ is G. In some embodiments, N₁ is U and N₂ is G. A skilled person in the art reading the present disclosure will appreciate that when a GUG Transcription Start Site is referenced with respect to a double-stranded DNA template, a coding strand of the double-stranded DNA template comprises a GUG start sequence, while a template DNA strand of the double stranded DNA template comprises a CAC which is the start site of transcription by an RNA polymerase.

In some embodiments, such in vitro transcription reactions can produce an RNA polynucleotide comprising a 5′ cap, a cap proximal sequence comprising positions +1, +2, +3, +4, and +5 of the RNA polynucleotide; and a sequence encoding a payload, wherein: (i) N₁ is position +1 of the RNA polynucleotide, (ii) N₂ is position +2 of the RNA polynucleotide, wherein N₁ and N₂ are selected from one of the following combinations: (a) N₁ is A and N₂ is G; (b) N₁ is U and N₂ is G; (c) N₁ is G and N₂ is G; and (d) N₁ is C and N₂ is G; and (iii) the cap proximal sequence comprises: N₁ and N₂ of the cap structure and a sequence comprising N₃N₄N₅ at positions +3, +4, and +5 respectively of the RNA polynucleotide, wherein N₃ is U, N₄ is G, and N₅ is selected from: A, C, G, and U. By way of example only, in some embodiments, an RNA polynucleotide resulting from such an in vitro transcription reaction comprises a 5′ cap and a cap proximal sequence comprising C₁G₂U₃G₄N₅, G₁G₂U₃G₄N₅ or U₁G₂U₃G₄N₅ or A₁G₂U₃G₄N₅, wherein N₅ is independently selected from A, U, G, or C. In some embodiments, an RNA polynucleotide resulting from such an in vitro transcription reaction can be an RNA polynucleotide described herein.

In some embodiments, such in vitro transcription reactions can produce an RNA polynucleotide comprising a 5′ cap, a cap proximal sequence comprising positions +1, +2, +3, +4, and +5 of the RNA polynucleotide; and a sequence encoding a payload, wherein: (i) N₁ is position +1 of the RNA polynucleotide, (ii) N₂ is position +2 of the RNA polynucleotide, wherein N₁ is G and N₂ is U; and (iii) the cap proximal sequence comprises: N₁ and N₂ of the cap structure and a sequence comprising N₃N₄N₅ at positions +3, +4, and +5 respectively of the RNA polynucleotide, wherein N₃ is G, and N₄ and N₅ are each selected from: A, C, G, and U. By way of examples only, in some such embodiments, an RNA polynucleotide resulting from such an in vitro transcription reaction comprises a 5′ cap and a cap proximal sequence comprising G₁U₂G₃A₄N₅. In some embodiments, an RNA polynucleotide resulting from such an in vitro transcription reaction comprises a 5′ cap and a cap proximal sequence comprising G₁U₂G₃U₄N₅.

In some embodiments, an RNA polynucleotide resulting from such an in vitro transcription reaction comprises a 5′ cap and a cap proximal sequence comprising G₁U₂G₃G₄N₅. In some embodiments, an RNA polynucleotide resulting from such an in vitro transcription reaction comprises a 5′ cap and a cap proximal sequence comprising G₁U₂G₃C₄N₅. A skilled artisan reading the present disclosure will realize that other RNA polynucleotides having different G₁U₂G₃N₄N₅ sequences (e.g., as described herein) can be produced by such in vitro transcription reaction described herein.

In some embodiments, an in vitro transcription reaction comprises: (i) a template DNA strand comprising a polynucleotide sequence complementary to an RNA polynucleotide sequence described herein, wherein the template DNA strand comprises a sequence that is complementary to a GUG transcription start site; (ii) a polymerase (e.g., an RNA polymerase such as, e.g., T7 polymerase); (iii) ribonucleotides; and (iv) a dinucleotide cap (e.g., as described herein) comprising N₁; wherein N₁ is G, wherein the sequence in the template strand that is complementary to GUG is the start site of transcription by an RNA polymerase. In some embodiments, G₁ nucleotide of a dinucleotide cap can interact with the first nucleotide or the third nucleotide of a sequence that is complementary to a GUG transcription start site. Without wishing to be bound by a particular theory, if G₁ nucleotide of a dinucleotide cap interacts with the third nucleotide of a sequence that is complementary to a GUG transcription start site, the first two GUs of the GUG transcription start site will be absent from a resulting RNA polynucleotide.

In some embodiments, such in vitro transcription reactions can produce an RNA polynucleotide comprising a 5′ cap, a cap proximal sequence comprising positions +1, +2, +3, +4, and +5 of the RNA polynucleotide; and a sequence encoding a payload, wherein: (i) N₁ is position +1 of the RNA polynucleotide, and (ii) the cap proximal sequence comprises: N₁ of the cap structure and a sequence comprising N₂N₃N₄N₅ at positions +2, +3, +4, and +5 respectively of the RNA polynucleotide, wherein N₂, N₃, N₄ and N₅ are each independently selected from: A, C, G, and U. In some embodiments, an RNA polynucleotide resulting from such an in vitro transcription reaction comprises a 5′ cap and a cap proximal sequence comprising G₁U₂G₃N₄N₅, wherein N₄ and N₅ are each independently selected from A, U, G, or C. In some embodiments, an RNA polynucleotide resulting from such an in vitro transcription reaction comprises a 5′ cap and a cap proximal sequence comprising G₁N₂N₃N₄N₅, wherein N₃, N₄ and N₅ are each independently selected from A, U, G, or C. By way of example only, in some such embodiments, an RNA polynucleotide resulting from such an in vitro transcription reaction comprises a 5′ cap and a cap proximal sequence comprising G₁U₂G₃A₄N₅, wherein N₅ is selected from A, U, G, or C. In some embodiments, an RNA polynucleotide resulting from such an in vitro transcription reaction comprises a 5′ cap and a cap proximal sequence comprising G₁U₂G₃U₄N₅, wherein N₅ is selected from A, U, G, or C. In some embodiments, an RNA polynucleotide resulting from such an in vitro transcription reaction comprises a 5′ cap and a cap proximal sequence comprising G₁U₂G₃G₄N₅, wherein N₅ is selected from A, U, G, or C. In some embodiments, an RNA polynucleotide resulting from such an in vitro transcription reaction comprises a 5′ cap and a cap proximal sequence comprising G₁U₂G₃C₄N₅, wherein N₅ is selected from A, U, G, or C. In some embodiments, an RNA polynucleotide resulting from such an in vitro transcription reaction comprises a 5′ cap and a cap proximal sequence comprising G₁A₂A₃A₄G₅. In some embodiments, an RNA polynucleotide resulting from such an in vitro transcription reaction comprises a 5′ cap and a cap proximal sequence comprising G₁U₂A₃A₄A₅. A skilled artisan reading the present disclosure will realize that other RNA polynucleotides having different G₁C₂G₃N₄N₅ or G₁N₂N₃N₄N₅ sequences (e.g., as described herein) can be produced by such in vitro transcription reaction described herein.

In some embodiments, an in vitro transcription reaction comprises: (i) a template DNA strand comprising a polynucleotide sequence complementary to an RNA polynucleotide sequence described herein, wherein the template DNA strand comprises a sequence that is complementary to a GCG transcription start site; (ii) a polymerase (e.g., an RNA polymerase such as, e.g., T7 polymerase); (iii) ribonucleotides; and (iv) a tetranucleotide cap (e.g., as described herein) comprising N₁pN₂pN₃; wherein N₁ is C, A, G or U, and N₂ is G and N₃ are C; and wherein the sequence in the template strand complementary to GCG is the start site of transcription by an RNA polymerase. In some embodiments, a skilled artisan reading the present disclosure will realize that RNA polynucleotides (e.g., as described herein) can be produced by such in vitro transcription reaction described herein.

In some embodiments, an in vitro transcription reaction comprises: (i) a template DNA strand comprising a polynucleotide sequence complementary to an RNA polynucleotide sequence described herein, wherein the template DNA strand comprises a sequence that is complementary to a GCG transcription start site; (ii) a polymerase (e.g., an RNA polymerase such as, e.g., T7 polymerase); (iii) ribonucleotides; and (iv) a tetranucleotide cap (e.g., as described herein) comprising N₁pN₂pN₃; wherein N₁ is G, N₂ is C, and N₃ is G; and wherein the sequence in the template strand complementary to GCG is the start site of transcription by an RNA polymerase.

In some embodiments, such in vitro transcription reactions can produce an RNA polynucleotide comprising a 5′ cap, a cap proximal sequence comprising positions +1, +2, +3, +4, and +5 of the RNA polynucleotide; and a sequence encoding a payload, wherein: (i) N₁ is position +1 of the RNA polynucleotide, (ii) N₂ is position +2 of the RNA polynucleotide, and N₃ is position +3 of the RNA polynucleotide, wherein N₁ is G, N₂ is C, and N₂ is G; and (iii) the cap proximal sequence comprises: N₁, N₂, and N₃ of a tetranucleotide cap structure and a sequence comprising N₄N₅ at positions +4 and +5 respectively of the RNA polynucleotide, wherein N₄ and N₅ are each independently is selected from: A, C, G, and U. In some embodiments, an RNA polynucleotide resulting from such an in vitro transcription reaction comprises a 5′ cap and a cap proximal sequence comprising G₁C₂G₃N₄N₅, wherein N₄ and N₅ are each independently selected from A, U, G, or C. By way of example only, in some embodiments, an RNA polynucleotide resulting from such an in vitro transcription reaction comprises a 5′ cap and a cap proximal sequence comprising G₁C₂G₃A₄U₅. In some embodiments, an RNA polynucleotide resulting from such an in vitro transcription reaction comprises a 5′ cap and a cap proximal sequence comprising G₁C₂G₃A₄A₅. In some embodiments, an RNA polynucleotide resulting from such an in vitro transcription reaction comprises a 5′ cap and a cap proximal sequence comprising G₁C₂G₃U₄A₅. A skilled artisan reading the present disclosure will realize that other RNA polynucleotides having different G₁C₂G₃N₄N₅ sequences (e.g., as described herein) can be produced by such in vitro transcription reaction described herein.

After RNA synthesis (e.g., in some embodiments RNA synthesis by in vitro transcription), one or more components (e.g., added reagents, reaction by products, and/or impurities) can be removed by one or more purification and/or separation processes known in the art. For example, without limitation, RNA preparation can be purified using phenol-chloroform extraction, enzymatic digestions of undesired components (e.g., protein components), precipitation, chromatography, spin column purification, membrane filtration, and/or affinity-based purification (e.g., in the form of a solid substrate, e.g., but not limited to magnetic beads or particles). In some embodiments, an RNA preparation can be subject to DNA and/or protein removal and/or digestion. In some embodiments, an RNA preparation can be purified by an affinity-based purification method, chromatography-based purification methods (e.g., size exclusion chromatography (SEC), high-performance liquid chromatography (HPLC), ion exchange chromatography (IEC)), and/or filtration methods (e.g., centrifugal ultrafiltration, membrane filtration, etc.).

### Complexes

In certain aspects, provided herein are complexes formed during in vitro transcription reactions described herein, e.g., using different combinations of caps (e.g., as described herein) and transcription start sites (e.g., as described herein).

In some embodiments, a complex comprises a template DNA strand and a 5′ cap comprising a structure of N₁pN₂, wherein the template DNA strand comprises an RNA polymerase promoter sequence and a sequence that is complementary to a transcription start site (e.g., ones described herein); wherein N₁ and N₂ are each independently chosen from: A, C, G, and U; wherein N₂ interacts with the +1 position of the template DNA strand (corresponding to the first nucleotide of the transcription start site) and N₁ does not interact with the +1 position of the template DNA strand; and wherein the sequence in the template strand that is complementary to the transcription start site is the start site of transcription by an RNA polymerase.

In some embodiments, N₁ is A and N₂ is G, and the +1 position of the sequence that is complementary to the transcription start site is C. In some embodiments, N₁ is U and N₂ is G, and the +1 position of the sequence that is complementary to the transcription start site is C. In some embodiments, N₁ is C and N₂ is G, and the +1 position of the sequence that is complementary to the transcription start site is C.

In some embodiments, the present disclosure provides a complex comprising a template DNA strand and a 5′ cap, wherein the template DNA strand comprises an RNA polymerase promoter sequence and a sequence that is complementary to a transcription start site; wherein the 5′ cap comprises a structure of N₁pN₂, and wherein N₁ and N₂ are each independently chosen from: A, C, G, and U; wherein N₁ interacts with the +1 position of the template DNA strand (corresponding to the first nucleotide of the transcription start site) and N₂ interacts with the +2 position of the template DNA strand (corresponding to the second nucleotide of the transcription start site); and wherein the sequence in the template strand that is complementary to the transcription start site is the start site of transcription by an RNA polymerase. In some embodiments, N₂ is U or C, and the +2 position of the template DNA strand is A or G. In some embodiments, N₃ is A or G, and the +3 position of the template DNA strand is T or C. In some embodiments, N₁ is A and N₂ is G, and position +1 is T and position +2 is C. In some embodiments, N₁ is G and N₂ is C, and position +1 and position +2 of the template DNA strand are C and G, respectively. In still further embodiments, N₁ is A and N₂ is U, and position +1 and position +2 of the template DNA strand are T and A, respectively.

In some embodiments, the present disclosure provides a complex comprising a template DNA strand and a 5′ cap, wherein the template DNA strand comprises an RNA polymerase promoter sequence and a sequence that is complementary to a transcription start site; wherein the 5′ cap is a tetranucleotide cap comprising the structure N₁pN₂pN₃, wherein N₁, N₂, and N₃ are each independently chosen from: A, C, G, and U; and wherein N₁, N₂, and N₃ interact with the +1, +2, and +3 positions of the template DNA strand (corresponding to the first, second, and third nucleotides, respectively of the transcription start site); and wherein the sequence in the template strand that is complementary to the transcription start site is the start site of transcription by an RNA polymerase. In some embodiments, N₂ is C or U and the +2 position of the template DNA strand is G or A. In some embodiments, N₂ is C or U, and N₃ is G or A and the +2 position of the template DNA strand is G or A and the +3 position of the template DNA strand is C or T. In some embodiments, N₁ is G, N₂ is C and N₃ is G and the +1, +2, and +3 positions of the template DNA strand are C, G, and C, respectively. In some embodiments, N₁ is A, N₂ is G, and N₃ is C and the +1, +2, and +3 positions of the template DNA strand are T, C, and G, respectively. In some embodiments, N₁ is A, N₂ is G, and N₃ is A and the +1, +2, and +3 positions of the template DNA strand are T, C, and T, respectively. In some embodiments, N₁ is A, N₂ is U, and N₃ is A and the +1, +2, and +3 positions of the template DNA strand are T, A, and T, respectively.

In some embodiments, the present disclosure provides a complex comprising a template DNA strand and a 5′ dinucleotide cap comprising a structure of N₁, wherein the template DNA strand comprises an RNA polymerase promoter sequence and a sequence that is complementary to a transcription start site; wherein N₁ is G; and wherein N₁ interacts with the +1 position of the template DNA strand (corresponding to the first nucleotide of the transcription start site); wherein the +2 position of the template DNA strand (corresponding to the second nucleotide of the transcription start site) is G, C, or A; and wherein the sequence in the template strand that is complementary to the transcription start site is the start site of transcription by an RNA polymerase. In some embodiments, the +3 position of the template DNA strand is T or C. In some embodiments, the +2 position of the template DNA strand is G or A. In some embodiments, the +1 position of the template DNA strand is C, the +2 position of the template DNA strand is G, and the +3 position of the template DNA strand is C. In some embodiments, the +1 position of the template DNA strand is C, the +2 position of the template DNA strand is A, and the +3 position of the template DNA strand is C.

In various aspects described herein, one or more nucleotides of a cap (e.g., ones described herein) interact with one or more nucleotides in the RNA polymerase start site the template DNA strand via canonical Watson-Crick base pairing. In some embodiments, a provided complex comprises a template DNA strand comprises an RNA polymerase promoter sequence, which in some embodiments may be or comprise a T7 RNA polymerase promoter sequence. In some embodiments, the complexes disclosed herein further comprise an RNA polymerase (e.g., a T7 RNA polymerase).

In some embodiments, complexes disclosed herein comprise a dinucleotide cap. In some embodiments, the complexes disclosed herein comprise a dinucleotide cap structure of G*N₁, wherein

G* comprises a structure of formula (I):

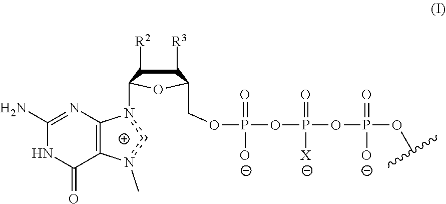

or a salt thereof,
wherein
   each R² and R³ is —OH or —OCH₃; and
   X is O or S.

In some embodiments, R² is —OH. In some embodiments, R² is —OCH₃. In some embodiments, R³ is —OH. In some embodiments, R³ is —OCH₃. In some embodiments, X is O. In some embodiments, X is S. In some embodiments, the dinucleotide cap structure comprises a Cap0 or Cap1 structure. In some embodiments, the dinucleotide cap structure comprises a Cap0 structure. In some embodiments, the dinucleotide cap structure comprises a Cap1 structure. In some embodiments, the dinucleotide cap structure comprises (m^{2′-O})N₁. In some embodiments, the dinucleotide cap structure is selected from the group consisting of (m⁷)GpppG (“Ecap0”), (m⁷)Gppp(^{2′-O})G (“Ecap1”), (m₂^{7,3′-O})GpppG (“ARCA” or “D1”), and (m₂^{7,2′-O})GppspG (“beta-S-ARCA”).

In some embodiments, complexes disclosed herein comprise a trinucleotide cap. In some embodiments, the trinucleotide cap structure has a structure: G*N¹pN₂, wherein

G* comprises a structure of formula (I):

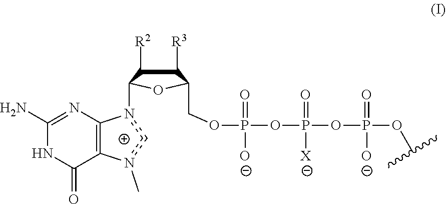

or a salt thereof,
wherein
   each R² and R³ is —OH or —OCH₃; and X is O or S.

In some embodiments, R² is —OH. In some embodiments, R² is —OCH₃. In some embodiments, R³ is —OH. In some embodiments, R³ is —OCH₃. In some embodiments, X is O. In some embodiments, the trinucleotide cap structure comprises a Cap0 or Cap1 structure. In some embodiments, the trinucleotide cap structure comprises a Cap1 structure. In some embodiments, the trinucleotide cap structure comprises (m^{2′-O})N₁pN₂. In some embodiments, the trinucleotide cap structure is selected from the group consisting of: (m₂^{7,3′-O})Gppp(m^{2′-O})ApG (“CleanCap AG”, “CC413”), (m₂^{7,3′-O})Gppp(m^{2′-O})GpG (“CleanCap GG”), (m⁷)Gppp(m^{2′-O})ApG, and (m₂^{7,3′-O})Gppp(m₂^{6,2′-O})ApG.

In some embodiments, complexes described herein comprise a tetranucleotide cap (e.g., ones described herein). In some embodiments, a tetranucleotide cap structure has a structure: G* N₁pN₂pN₃, wherein
G* comprises a structure of formula (I):

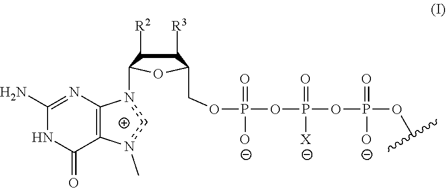

or a salt thereof, wherein each R² and R³ is —OH or —OCH₃; and X is O or S. In some embodiments, a tetranucleotide cap structure comprises (m^{2′-O})N₁pN₂pN₃. In some embodiments, such a tetranucleotide cap is or comprise a cap2 structure. In some embodiments, a tetranucleotide cap structure comprises (m^{2′-O})N₁p(m^{2′-O})N₂pN₃. In some embodiments, a tetranucleotide cap structure comprises (m₂^{7,3′-O})Gppp(m^{2′-O})Cp(m^{2′-O})GpC and (m₂^{7,3′-O})Gppp(m^{2′-O})Gp(m^{2′-O})CpG.

A skilled artisan in the art reading the present disclosure will realize that various caps (e.g., ones described herein) can be selected for use with a certain transcription start site described herein to produce complexes by in vitro transcription reaction (e.g., ones described herein). In some embodiments, a transcription start site is AGA. In some embodiments, a transcription start site is AGC. In some embodiments, a transcription start site is AUA. In some embodiments, a transcription start site is CGC. In some embodiments, a transcription start site is GCG. In some embodiments, a transcription start site is GGG. In some embodiments, a transcription start site is GUG.

### Exemplary Polynucleotides

In some embodiments, an RNA polynucleotide described herein or a composition or medical preparation comprising the same comprises a nucleotide sequence disclosed herein. In some embodiments, an RNA polynucleotide comprises a sequence having at least 80% identity to a nucleotide sequence disclosed herein. In some embodiments, an RNA polynucleotide comprises a sequence encoding a polypeptide having at least 80% identity to a polypeptide sequence disclosed herein. Exemplary nucleotide and polypeptide sequences are provided e.g., in Table 1 or in this section titled “Exemplary polynucleotides” or in Example 1 or 2. In some embodiments, an RNA polynucleotide described herein, or a composition or a medical preparation comprising the same, comprises a nucleotide sequence disclosed herein, wherein the specified cap and cap proximal sequence can be replaced with another combination of a cap and a cap proximal sequence described herein. For example, for illustrative purposes only, the cap (m₂^{7,3′-O}Gppp(m₁^{2′-O})ApG; shown as underlined) and the cap proximal sequence (AGAAU; shown as underlined) of an RNA as set forth in SEQ ID NO: 31 can be replaced with a combination of a different cap (e.g., a dinucleotide, a trinucleotide, or tetranucleotide cap described herein) and/or a different cap proximal sequence comprising a Transcription Start Site described herein (e.g., in some embodiments comprising a GCG transcription start site, or in some embodiments comprising a AUA transcription start site).

In some embodiments, an RNA polynucleotide described herein or a composition or medical preparation comprising the same is transcribed by a DNA template. In some embodiments, a DNA template used to transcribe an RNA polynucleotide described herein comprises a sequence complementary to an RNA polynucleotide.

In some embodiments, a payload described herein is encoded by an RNA polynucleotide described herein comprising a nucleotide sequence disclosed herein, e.g., in Table 1 or in this section titled “Exemplary polynucleotides” or in Example 1 or 2. In some embodiments, an RNA polynucleotide encodes a polypeptide payload having at least 80% o identity to a polypeptide payload sequence disclosed herein. In some embodiments, a payload described herein is encoded by an RNA polynucleotide transcribed by a DNA template comprising a sequence complementary to an RNA polynucleotide.

TABLE 1
Exemplary sequences of RNA constructs disclosed herein

| Sequence | |
|---|---|
| information | Exemplary RNA Sequences |
| Cap proximal | GAGAN₅ or UAGAN₅, or CAGAN₅, or AAGAN₅, AGAN₄N₅, |
| consensus sequence | wherein N₄ and N₅ can be each independently |
| (e.g., nucleotides 1- | any nucleotide (e.g., A, U, G, C) |
| 5 of a 5′ UTR) | |
| comprising AGA | |
| start sequence | |
| Cap proximal | GAGCN₅ or UAGCN₅ or CAGCN₅ or AAGCN₅, AGCN₄N₅, |
| consensus sequence | wherein N₄ and N₅ can be each independently |
| (e.g., nucleotides 1- | any nucleotide (e.g., A, U, G, C) |
| 5 of a 5′ UTR) | |
| comprising AGC | |
| start sequence | |
| Cap proximal | GAUAN₅ or UAUAN₅ or CAUAN₅, or AAUAN₅, AUAN₄N₅, |
| consensus sequence | wherein N₄ and N₅ can be each independently |
| (e.g., nucleotides 1- | any nucleotide (e.g., A, U, G, C) |
| 5 of a 5′ UTR) | |
| comprising AUA | |
| start sequence | |
| Cap proximal | GCGCN₅, UCGCN₅, ACGCN₅, CCGCN₅, CGCN₄N₅, |
| consensus sequence | wherein N₄ and N₅ can be each independently |
| (e.g., nucleotides 1- | any nucleotide (e.g., A, U, G, C) |
| 5 of a 5′ UTR) | |
| comprising CGC | |
| start sequence | |
| Cap proximal | GGCGN₅, UGCGN₅, AGCGN₅, CGCGN₅, GCGN₄N₅, |
| consensus sequence | wherein N₄ and N₅ can be each independently |
| (e.g., nucleotides 1- | any nucleotide (e.g., A, U, G, C) |
| 5 of a 5′ UTR) | |
| comprising GCG | |
| start sequence | |
| Cap proximal | CGGGN₅, UGGGN₅, AGGGN₅, GGGN₄N₅, wherein N₄ and |
| consensus sequence | N₅ can be each independently any nucleotide |
| (e.g., nucleotides 1- | (e.g., A, U, G, C) |
| 5 of a 5′ UTR) | |
| comprising GGG | |
| start sequence | |
| Cap proximal | GGUGN₅, UGUGN₅, AGUGN₅, CGUGN₅, GUGN₄N₅, |
| consensus sequence | wherein N₄ and N₅ can be each independently |
| (e.g., nucleotides 1- | any nucleotide (e.g., A, U, G, C) |
| 5 of a 5′ UTR) | |
| comprising GUG | |
| start sequence | |
| Ligation 3 | GAGUCGCUAGCCGCGUCGCU |
| sequence | |
| S protein PP | MFVFLVLLPLVSSQCVNLTTRTQLPPAYTNSFTRGVYYPDKVFR |
| (amino acid) (SEQ | SSVLHSTQDLFLPFFSNVTWFHAIHVSGTNGTKRFDNPVLPFND |
| ID NO: 9) | GVYFASTEKSNIIRGWIFGTTLDSKTQSLLIVNNATNVVIKVCE |
| | FQFCNDPFLGVYYHKNNKSWMESEFRVYSSANNCTFEYVSQPFL |
| | MDLEGKQGNFKNLREFVFKNIDGYFKIYSKHTPINLVRDLPQGF |
| | SALEPLVDLPIGINITRFQTLLALHRSYLTPGDSSSGWTAGAAA |
| | YYVGYLQPRTFLLKYNENGTITDAVDCALDPLSETKCTLKSFTV |
| | EKGIYQTSNERVQPTESIVRFPNITNLCPFGEVENATRFASVYA |
| | WNRKRISNCVADYSVLYNSASFSTFKCYGVSPTKLNDLCFTNVY |
| | ADSFVIRGDEVRQIAPGQTGKIADYNYKLPDDFTGCVIAWNSNN |
| | LDSKVGGNYNYLYRLERKSNLKPFERDISTEIYQAGSTPCNGVE |
| | GENCYFPLQSYGFQPTNGVGYQPYRVVVLSFELLHAPATVCGPK |
| | KSTNLVKNKCVNFNFNGLTGTGVLTESNKKELPFQQFGRDIADT |
| | TDAVRDPQTLEILDITPCSFGGVSVITPGTNTSNQVAVLYQDVN |
| | CTEVPVAIHADQLTPTWRVYSTGSNVFQTRAGCLIGAEHVNNSY |
| | ECDIPIGAGICASYQTQTNSPRRARSVASQSIIAYTMSLGAENS |
| | VAYSNNSIAIPTNFTISVTTEILPVSMTKTSVDCTMYICGDSTE |
| | CSNLLLQYGSFCTQLNRALTGIAVEQDKNTQEVFAQVKQIYKTP |
| | PIKDFGGFNFSQILPDPSKPSKRSFIEDLLENKVTLADAGFIKQ |
| | YGDCLGDIAARDLICAQKENGLTVLPPLLTDEMIAQYTSALLAG |
| | TITSGWTFGAGAALQIPFAMQMAYRENGIGVTQNVLYENQKLIA |
| | NQFNSAIGKIQDSLSSTASALGKLQDVVNQNAQALNTLVKQLSS |
| | NFGAISSVLNDILSRLDPPEAEVQIDRLITGRLQSLQTYVTQQL |
| | IRAAEIRASANLAATKMSECVLGQSKRVDFCGKGYHLMSFPQSA |
| | PHGVVFLHVTYVPAQEKNFTTAPAICHDGKAHFPREGVFVSNGT |
| | HWFVTQRNFYEPQIITTDNTFVSGNCDVVIGIVNNTVYDPLQPE |
| | LDSFKEELDKYFKNHTSPDVDLGDISGINASVVNIQKEIDRLNE |
| | VAKNLNESLIDLQELGKYEQYIKWPWYIWLGFIAGLIAIVMVTI |
| | MLCCMTSCCSCLKGCCSCGSCCKFDEDDSEPVLKGVKLHYT |
| RBP020.2 | agaauaaacu aguauucuuc ugguccccac agacucagag |
| | agaacccgcc accauguucg |
| | uguuccuggu gcugcugccu cuggugucca gccagugugu |
| | gaaccugacc accagaacac agcugccucc agccuacacc |
| | aacagcuuua ccagaggcgu guacuacccc gacaaggugu |
| | ucagauccag cgugcugcac ucuacccagg accuguuccu |
| | gccuuucuuc agcaacguga ccugguucca cgccauccac |
| | guguccggca ccaauggcac caagagauuc gacaaccccg |
| | ugcugcccuu caacgacggg guguacuuug ccagcaccga |
| | gaaguccaac aucaucagag gcuggaucuu cggcaccaca |
| | cuggacagca agacccagag ccugcugauc gugaacaacg |
| | ccaccaacgu ggucaucaaa gugugcgagu uccaguucug |
| | caacgacccc uuccugggcg ucuacuacca caagaacaac |
| | aagagcugga uggaaagcga guuccgggug uacagcagcg |
| | ccaacaacug caccuucgag uacguguccc agccuuuccu |
| | gauggaccug gaaggcaagc agggcaacuu caagaaccug |
| | cgcgaguucg uguuuaagaa caucgacggc uacuucaaga |
| | ucuacagcaa gcacaccccu aucaaccucg ugcgggaucu |
| | gccucagggc uucucugcuc |
| | uggaaccccu gguggaucug cccaucggca ucaacaucac |
| | ccgguuucag acacugcugg cccugcacag aagcuaccug |
| | acaccuggcg auagcagcag |
| | ccgcuuacua ugugggcuac cugcagccua gaaccuuccu |
| | gcugaaguac aacgagaacg gcaccaucac cgacgccgug |
| | gauugugcuc uggauccucu gagcgagaca aagugcaccc |
| | ugaaguccuu caccguggaa aagggcaucu accagaccag |
| | caacuuccgg gugcagccca |
| | ccgaauccau cgugcgguuc cccaauauca ccaaucugug |
| | ccccuucggc gagguguuca augccaccag auucgccucu |
| | guguacgccu ggaaccggaa gcggaucagc aauugcgugg |
| | ccgacuacuc cgugcuguac aacuccgcca gcuucagcac |
| | cuucaagugc uacggcgugu ccccuaccaa gcugaacgac |
| | cugugcuuca caaacgugua cgccgacagc uucgugaucc |
| | ggggagauga agugcggcag auugccccug gacagacagg |
| | caagaucgcc gacuacaacu |
| | acaagcugcc cgacgacuuc accggcugug ugauugccug |
| | gaacagcaac aaccuggacu ccaaagucgg cggcaacuac |
| | aauuaccugu accggcuguu ccggaagucc aaucugaagc |
| | ccuucgagcg ggacaucucc accgagaucu aucaggccgg |
| | cagcaccccu uguaacggcg uggaaggcuu caacugcuac |
| | uucccacugc aguccuacgg cuuucagccc acaaauggcg |
| | ugggcuauca gcccuacaga gugguggugc ugagcuucga |
| | acugcugcau gccccugcca |
| | cagugugcgg cccuaagaaa agcaccaauc ucgugaagaa |
| | caaaugcgug aacuucaacu |
| | ucaacggccu gaccggcacc ggcgugcuga cagagagcaa |
| | caagaaguuc cugccauucc agcaguuugg ccgggauauc |
| | gccgauacca cagacgccgu uagagauccc cagacacugg |
| | aaauccugga caucaccccu ugcagcuucg gcggaguguc |
| | ugugaucacc ccuggcacca acaccagcaa ucagguggca |
| | gugcuguacc aggacgugaa cuguaccgaa gugcccgugg |
| | ccauucacgc cgaucagcug acaccuacau ggcgggugua |
| | cuccaccggc agcaaugugu |
| | uucagaccag agccggcugu cugaucggag ccgagcacgu |
| | gaacaauagc uacgagugcg acauccccau cggcgcugga |
| | aucugcgcca gcuaccagac acagacaaac agcccucgga |
| | gagccagaag cguggccagc cagagcauca uugccuacac |
| | aaugucucug ggcgccgaga acagcguggc cuacuccaac |
| | aacucuaucg cuauccccac caacuucacc aucagcguga |
| | ccacagagau ccugccugug uccaugacca agaccagcgu |
| | ggacugcacc auguacaucu gcggcgauuc caccgagugc |
| | uccaaccugc ugcugcagua cggcagcuuc ugcacccagc |
| | ugaauagagc ccugacaggg aucgccgugg aacaggacaa |
| | gaacacccaa gagguguucg cccaagugaa gcagaucuac |
| | aagaccccuc cuaucaagga cuucggcggc uucaauuuca |
| | gccagauucu gcccgauccu agcaagccca gcaagcggag |
| | cuucaucgag gaccugcugu |
| | ucaacaaagu gacacuggcc gacgccggcu ucaucaagca |
| | guauggcgau ugucugggcg acauugccgc cagggaucug |
| | auuugcgccc agaaguuuaa cggacugaca gugcugccuc |
| | cucugcugac cgaugagaug aucgcccagu acacaucugc |
| | ccugcuggcc ggcacaauca caagcggcug gacauuugga |
| | gcaggcgccg cucugcagau ccccuuugcu augcagaugg |
| | ccuaccgguu caacggcauc ggagugaccc agaaugugcu |
| | guacgagaac cagaagcuga |
| | ucgccaacca guucaacagc gccaucggca agauccagga |
| | cagccugagc agcacagcaa |
| | gcgcccuggg aaagcugcag gacgugguca accagaaugc |
| | ccaggcacug aacacccugg ucaagcagcu guccuccaac |
| | uucggcgcca ucagcucugu gcugaacgau auccugagca |
| | gacuggaccc uccugaggcc gaggugcaga ucgacagacu |
| | gaucacaggc agacugcaga gccuccagac auacgugacc |
| | cagcagcuga ucagagccgc cgagauuaga gccucugcca |
| | aucuggccgc caccaagaug ucugagugug ugcugggcca |
| | gagcaagaga guggacuuuu gcggcaaggg cuaccaccug |
| | augagcuucc cucagucugc cccucacggc gugguguuuc |
| | ugcacgugac auaugugccc gcucaagaga agaauuucac |
| | caccgcucca gccaucugcc |
| | acgacggcaa agcccacuuu ccuagagaag gcguguucgu |
| | guccaacggc acccauuggu ucgugacaca gcggaacuuc |
| | uacgagcccc agaucaucac caccgacaac accuucgugu |
| | cuggcaacug cgacgucgug aucggcauug ugaacaauac |
| | cguguacgac ccucugcagc ccgagcugga cagcuucaaa |
| | gaggaacugg acaaguacuu uaagaaccac acaagccccg |
| | acguggaccu gggcgauauc agcggaauca augccagcgu |
| | cgugaacauc cagaaagaga ucgaccggcu gaacgaggug |
| | gccaagaauc ugaacgagag ccugaucgac cugcaagaac |
| | uggggaagua cgagcaguac aucaaguggc ccugguacau |
| | cuggcugggc uuuaucgccg gacugauugc caucgugaug |
| | gucacaauca ugcuguguug caugaccagc ugcuguagcu |
| | gccugaaggg cuguuguagc uguggcagcu gcugcaaguu |
| | cgacgaggac gauucugagc ccgugcugaa gggcgugaaa |
| | cugcacuaca caugaugacu cgagcuggua cugcaugcac |
| | gcaaugcuag cugccccuuu cccguccugg guaccccgag |
| | ucucccccga ccucgggucc cagguaugcu cccaccucca |
| | ccugccccac ucaccaccuc ugcuaguucc agacaccucc |
| | caagcacgca gcaaugcagc ucaaaacgcu uagccuagcc |
| | acacccccac gggaaacagc agugauuaac cuuuagcaau |
| | aaacgaaagu uuaacuaagc uauacuaacc ccaggguugg |
| | ucaauuucgu gccagccaca cccuggagcu agcaaaaaaa |
| | aaaaaaaaaa aaaaaaaaaa aaagcauaug acuaaaaaaa |
| | aaaaaaaaaa aaaaaaaaaa aaaaaaaaaa aaaaaaaaaa |
| | aaaaaaaaaa aaaaaaaaaa aaa |
| Human alpha | AGAAUAAACUAGUAUUCUUCUGGUCCCCACAGACUCAGAGAGAA |
| globin 5′ UTR with | CCC |
| (AGAAU first 5 | |
| nucleotides) SEQ | |
| ID NO: 11 | |
| Human alpha | AAACUAGUAUUCUUCUGGUCCCCACAGACUCAGAGAGAACCC |
| globin 5′ UTR | |
| (without first 5 | |
| nucleotides) | |
| SEQ ID NO: 12 | |
| 3′ UTR | CUGGUACUGCAUGCACGCAAUGCUAGCUGCCCCUUUCCCGUCCU |
| SEQ ID NO: 13 | GGGUACCCCGAGUCUCCCCCGACCUCGGGUCCCAGGUAUGCUCC |
| | CACCUCCACCUGCCCCACUCACCACCUCUGCUAGUUCCAGACAC |
| | CUCCCAAGCACGCAGCAAUGCAGCUCAAAACGCUUAGCCUAGCC |
| | ACACCCCCACGGGAAACAGCAGUGAUUAACCUUUAGCAAUAAAC |
| | GAAAGUUUAACUAAGCUAUACUAACCCCAGGGUUGGUCAAUUUC |
| | GUGCCAGCCACACC |
| A30L70 PolyA | AAAAAAAAAAAAAAAAAAAAAAAAAAAAAAGCAUAUGACUAAAA |
| | AAAAAAAAAAAAAAAAAAAAAAAAAAAAAAAAAAAAAAAAAAAA |
| SEQ ID NO: 14 | AAAAAAAAAAAAAAAAAAAAAA |
| Exemplary RNA | AGAAUAAACUAGUAUUCUUCUGGUCCCCACAGACUCAGAGAGAA |
| polynucleotide (vA | CCCGCCACCCUCGAGCUGGUACUGCAUGCACGCAAUGCUAGCUG |
| 3.0.1) without | CCCCUUUCCCGUCCUGGGUACCCCGAGUCUCCCCCGACCUCGGG |
| coding sequence of | UCCCAGGUAUGCUCCCACCUCCACCUGCCCCACUCACCACCUCU |
| a payload | GCUAGUUCCAGACACCUCCCAAGCACGCAGCAAUGCAGCUCAAA |
| Bold = 5′ UTR | ACGCUUAGCCUAGCCACACCCCCACGGGAAACAGCAGUGAUUAA |
| Bold and | CCUUUAGCAAUAAACGAAAGUUUAACUAAGCUAUACUAACCCCA |
| underlined = cap | GGGUUGGUCAAUUUCGUGCCAGCCACACCCUGGAGCUAGCAAAA |
| proximal sequence | AAAAAAAAAAAAAAAAAAAAAAAAAAGCAUAUGACUAAAAAAAA |
| Underlined = | AAAAAAAAAAAAAAAAAAAAAAAAAAAAAAAAAAAAAAAAAAAA |
| Kozak sequence | AAAAAAAAAAAAAAAAAA |
| Italicized and | |
| underlined = 3′ | |
| UTR | |
| Italicized = PolyA | |
| Exemplary RNA | AGAAUAAACUAGUAUUCUUCUGGUCCCCACAGACUCAGAGAGAA |
| polynucleotide (vA | CCCGCCACCAUGUUCGUGUUCCUGGUGCUGCUGCCUCUGGUGUC |
| 3.0.1) with a | CAGCCAGUGUGUGAACCUGACCACCAGAACACAGCUGCCUCCAG |
| payload sequence | CCUACACCAACAGCUUUACCAGAGGCGUGUACUACCCCGACAAG |
| Underline = | GUGUUCAGAUCCAGCGUGCUGCACUCUACCCAGGACCUGUUCCU |
| exemplary payload | GCCUUUCUUCAGCAACGUGACCUGGUUCCACGCCAUCCACGUGU |
| sequence | CCGGCACCAAUGGCACCAAGAGAUUCGACAACCCCGUGCUGCCC |
| (BNT162b2) | UUCAACGACGGGGUGUACUUUGCCAGCACCGAGAAGUCCAACAU |
| | CAUCAGAGGCUGGAUCUUCGGCACCACACUGGACAGCAAGACCC |
| | AGAGCCUGCUGAUCGUGAACAACGCCACCAACGUGGUCAUCAAA |
| | GUGUGCGAGUUCCAGUUCUGCAACGACCCCUUCCUGGGCGUCUA |
| | CUACCACAAGAACAACAAGAGCUGGAUGGAAAGCGAGUUCCGGG |
| | UGUACAGCAGCGCCAACAACUGCACCUUCGAGUACGUGUCCCAG |
| | CCUUUCCUGAUGGACCUGGAAGGCAAGCAGGGCAACUUCAAGAA |
| | CCUGCGCGAGUUCGUGUUUAAGAACAUCGACGGCUACUUCAAGA |
| | UCUACAGCAAGCACACCCCUAUCAACCUCGUGCGGGAUCUGCCU |
| | CAGGGCUUCUCUGCUCUGGAACCCCUGGUGGAUCUGCCCAUCGG |
| | CAUCAACAUCACCCGGUUUCAGACACUGCUGGCCCUGCACAGAA |
| | GCUACCUGACACCUGGCGAUAGCAGCAGCGGAUGGACAGCUGGU |
| | GCCGCCGCUUACUAUGUGGGCUACCUGCAGCCUAGAACCUUCCU |
| | GCUGAAGUACAACGAGAACGGCACCAUCACCGACGCCGUGGAUU |
| | GUGCUCUGGAUCCUCUGAGCGAGACAAAGUGCACCCUGAAGUCC |
| | UUCACCGUGGAAAAGGGCAUCUACCAGACCAGCAACUUCCGGGU |
| | GCAGCCCACCGAAUCCAUCGUGCGGUUCCCCAAUAUCACCAAUC |
| | UGUGCCCCUUCGGCGAGGUGUUCAAUGCCACCAGAUUCGCCUCU |
| | GUGUACGCCUGGAACCGGAAGCGGAUCAGCAAUUGCGUGGCCGA |
| | CUACUCCGUGCUGUACAACUCCGCCAGCUUCAGCACCUUCAAGU |
| | GCUACGGCGUGUCCCCUACCAAGCUGAACGACCUGUGCUUCACA |
| | AACGUGUACGCCGACAGCUUCGUGAUCCGGGGAGAUGAAGUGCG |
| | GCAGAUUGCCCCUGGACAGACAGGCAAGAUCGCCGACUACAACU |
| | ACAAGCUGCCCGACGACUUCACCGGCUGUGUGAUUGCCUGGAAC |
| | AGCAACAACCUGGACUCCAAAGUCGGCGGCAACUACAAUUACCU |
| | GUACCGGCUGUUCCGGAAGUCCAAUCUGAAGCCCUUCGAGCGGG |
| | ACAUCUCCACCGAGAUCUAUCAGGCCGGCAGCACCCCUUGUAAC |
| | GGCGUGGAAGGCUUCAACUGCUACUUCCCACUGCAGUCCUACGG |
| | CUUUCAGCCCACAAAUGGCGUGGGCUAUCAGCCCUACAGAGUGG |
| | UGGUGCUGAGCUUCGAACUGCUGCAUGCCCCUGCCACAGUGUGC |
| | GGCCCUAAGAAAAGCACCAAUCUCGUGAAGAACAAAUGCGUGAA |
| | CUUCAACUUCAACGGCCUGACCGGCACCGGCGUGCUGACAGAGA |
| | GCAACAAGAAGUUCCUGCCAUUCCAGCAGUUUGGCCGGGAUAUC |
| | GCCGAUACCACAGACGCCGUUAGAGAUCCCCAGACACUGGAAAU |
| | CCUGGACAUCACCCCUUGCAGCUUCGGCGGAGUGUCUGUGAUCA |
| | CCCCUGGCACCAACACCAGCAAUCAGGUGGCAGUGCUGUACCAG |
| | GACGUGAACUGUACCGAAGUGCCCGUGGCCAUUCACGCCGAUCA |
| | GCUGACACCUACAUGGCGGGUGUACUCCACCGGCAGCAAUGUGU |
| | UUCAGACCAGAGCCGGCUGUCUGAUCGGAGCCGAGCACGUGAAC |
| | AAUAGCUACGAGUGCGACAUCCCCAUCGGCGCUGGAAUCUGCGC |
| | CAGCUACCAGACACAGACAAACAGCCCUCGGAGAGCCAGAAGCG |
| | UGGCCAGCCAGAGCAUCAUUGCCUACACAAUGUCUCUGGGCGCC |
| | GAGAACAGCGUGGCCUACUCCAACAACUCUAUCGCUAUCCCCAC |
| | CAACUUCACCAUCAGCGUGACCACAGAGAUCCUGCCUGUGUCCA |
| | UGACCAAGACCAGCGUGGACUGCACCAUGUACAUCUGCGGCGAU |
| | UCCACCGAGUGCUCCAACCUGCUGCUGCAGUACGGCAGCUUCUG |
| | CACCCAGCUGAAUAGAGCCCUGACAGGGAUCGCCGUGGAACAGG |
| | ACAAGAACACCCAAGAGGUGUUCGCCCAAGUGAAGCAGAUCUAC |
| | AAGACCCCUCCUAUCAAGGACUUCGGCGGCUUCAAUUUCAGCCA |
| | GAUUCUGCCCGAUCCUAGCAAGCCCAGCAAGCGGAGCUUCAUCG |
| | AGGACCUGCUGUUCAACAAAGUGACACUGGCCGACGCCGGCUUC |
| | AUCAAGCAGUAUGGCGAUUGUCUGGGCGACAUUGCCGCCAGGGA |
| | UCUGAUUUGCGCCCAGAAGUUUAACGGACUGACAGUGCUGCCUC |
| | CUCUGCUGACCGAUGAGAUGAUCGCCCAGUACACAUCUGCCCUG |
| | CUGGCCGGCACAAUCACAAGCGGCUGGACAUUUGGAGCAGGCGC |
| | CGCUCUGCAGAUCCCCUUUGCUAUGCAGAUGGCCUACCGGUUCA |
| | ACGGCAUCGGAGUGACCCAGAAUGUGCUGUACGAGAACCAGAAG |
| | CUGAUCGCCAACCAGUUCAACAGCGCCAUCGGCAAGAUCCAGGA |
| | CAGCCUGAGCAGCACAGCAAGCGCCCUGGGAAAGCUGCAGGACG |
| | UGGUCAACCAGAAUGCCCAGGCACUGAACACCCUGGUCAAGCAG |
| | CUGUCCUCCAACUUCGGCGCCAUCAGCUCUGUGCUGAACGAUAU |
| | CCUGAGCAGACUGGACCCUCCUGAGGCCGAGGUGCAGAUCGACA |
| | GACUGAUCACAGGCAGACUGCAGAGCCUCCAGACAUACGUGACC |
| | CAGCAGCUGAUCAGAGCCGCCGAGAUUAGAGCCUCUGCCAAUCU |
| | GGCCGCCACCAAGAUGUCUGAGUGUGUGCUGGGCCAGAGCAAGA |
| | GAGUGGACUUUUGCGGCAAGGGCUACCACCUGAUGAGCUUCCCU |
| | CAGUCUGCCCCUCACGGCGUGGUGUUUCUGCACGUGACAUAUGU |
| | GCCCGCUCAAGAGAAGAAUUUCACCACCGCUCCAGCCAUCUGCC |
| | ACGACGGCAAAGCCCACUUUCCUAGAGAAGGCGUGUUCGUGUCC |
| | AACGGCACCCAUUGGUUCGUGACACAGCGGAACUUCUACGAGCC |
| | CCAGAUCAUCACCACCGACAACACCUUCGUGUCUGGCAACUGCG |
| | ACGUCGUGAUCGGCAUUGUGAACAAUACCGUGUACGACCCUCUG |
| | CAGCCCGAGCUGGACAGCUUCAAAGAGGAACUGGACAAGUACUU |
| | UAAGAACCACACAAGCCCCGACGUGGACCUGGGCGAUAUCAGCG |
| | GAAUCAAUGCCAGCGUCGUGAACAUCCAGAAAGAGAUCGACCGG |
| | CUGAACGAGGUGGCCAAGAAUCUGAACGAGAGCCUGAUCGACCU |
| | GCAAGAACUGGGGAAGUACGAGCAGUACAUCAAGUGGCCCUGGU |
| | ACAUCUGGCUGGGCUUUAUCGCCGGACUGAUUGCCAUCGUGAUG |
| | GUCACAAUCAUGCUGUGUUGCAUGACCAGCUGCUGUAGCUGCCU |
| | GAAGGGCUGUUGUAGCUGUGGCAGCUGCUGCAAGUUCGACGAGG |
| | ACGAUUCUGAGCCCGUGCUGAAGGGCGUGAAACUGCACUACACA |
| | UGAUGACUCGAGCUGGUACUGCAUGCACGCAAUGCUAGCUGCCC |
| | CUUUCCCGUCCUGGGUACCCCGAGUCUCCCCCGACCUCGGGUCC |
| | CAGGUAUGCUCCCACCUCCACCUGCCCCACUCACCACCUCUGCU |
| | AGUUCCAGACACCUCCCAAGCACGCAGCAAUGCAGCUCAAAACG |
| | CUUAGCCUAGCCACACCCCCACGGGAAACAGCAGUGAUUAACCU |
| | UUAGCAAUAAACGAAAGUUUAACUAAGCUAUACUAACCCCAGGG |
| | UUGGUCAAUUUCGUGCCAGCCACACCCUGGAGCUAGCAAAAAAA |
| | AAAAAAAAAAAAAAAAAAAAAAAGCAUAUGACUAAAAAAAAAAA |
| | AAAAAAAAAAAAAAAAAAAAAAAAAAAAAAAAAAAAAAAAAAAA |
| | AAAAAAAAAAAAAAA |

RBL063.1 (SEQ ID NO: 28 nucleotide; SEQ ID NO: 9 amino acid)

| | |
|---|---|
| Structure | beta-S-ARCA(D1)-hAg-Kozak-S1S2-PP-FI-A30L70 |
| Encoded antigen | Viral spike protein (S1S2 protein) of the SARS-CoV-2 (S1S2 full-length |
| | protein, sequence variant) |

SEQ ID NO: 28

| | |
|---|---|
| gggcgaacua guauucuucu gguccccaca gacucagaga gaacccgcca ccauguuugu | 60 |
| guuucuugug cugcugccuc uugugucuuc ucagugugug aauuugacaa caagaacaca | 120 |
| gcugccacca gcuuauacaa auucuuuuac cagaggagug uauuauccug auaaaguguu | 180 |
| uagaucuucu gugcugcaca gcacacagga ccuguuucug ccauuuuuua gcaaugugac | 240 |
| augguuucau gcaauucaug ugucuggaac aaauggaaca aaaagauuug auaauccugu | 300 |
| gcugccuuuu aaugauggag uguauuuugc uucaacagaa aagucaaaua uuauuagagg | 360 |
| auggauuuuu ggaacaacac uggauucuaa aacacagucu cugcugauug ugaauaaugc | 420 |
| aacaaaugug gugauuaaag ugugugaauu ucaguuuugu aaugauccuu uucugggagu | 480 |
| guauuaucac aaaaauaaua aaucuuggau ggaaucugaa uuuagagugu auuccucugc | 540 |
| aaauaauugu acauuugaau augugucuca gccuuuucug auggaucugg aaggaaaaca | 600 |
| gggcaauuuu aaaaaucuga gagaauuugu guuuaaaaau auugauggau auuuuaaaau | 660 |
| uuauucuaaa cacacaccaa uuaauuuagu gagagaucug ccucagggau uuucugcucu | 720 |
| ggaaccucug guggaucugc caauuggcau uaauauuaca agauuucaga cacugcuggc | 780 |
| ucugcacaga ucuuaucuga caccuggaga uucuucuucu ggauggacag ccggagcugc | 840 |
| agcuuauuau gugggcuauc ugcagccaag aacauuucug cugaaauaua augaaaaugg | 900 |
| aacaauuaca gaugcugugg auugugcucu ggauccucug ucugaaacaa aauguacauu | 960 |
| aaaaucuuuu acaguggaaa aaggcauuua ucagacaucu aauuuuagag ugcagccaac | 1020 |
| agaaucuauu gugagauuuc caaauauuac aaaucugugu ccauuuggag aaguguuuaa | 1080 |
| ugcaacaaga uuugcaucug uguaugcaug gaauagaaaa agaauuucua auuguguggc | 1140 |
| ugauuauucu gugcuguaua auagugcuuc uuuuuccaca uuuaaauguu auggaguguc | 1200 |
| uccaacaaaa uuaaaugauu uauguuuuac aaauguguau gcugauucuu uugugaucag | 1260 |
| aggugaugaa gugagacaga uugcccccgg acagacagga aaaauugcug auuacaauua | 1320 |
| caaacugccu gaugauuuua caggaugugu gauugcuugg aauucuaaua auuuagauuc | 1380 |
| uaaaguggga ggaaauuaca auuaucugua cagacuguuu agaaaaucaa aucugaaacc | 1440 |
| uuuugaaaga gauauuucaa cagaaauuua ucaggcugga ucaacaccuu guaauggagu | 1500 |
| ggaaggauuu aauuguuauu uuccauuaca gagcuaugga uuucagccaa ccaauggugu | 1560 |
| gggauaucag ccauauagag ugguggugcu gucuuuugaa cugcugcaug caccugcaac | 1620 |
| agugugugga ccuaaaaaau cuacaaauuu agugaaaaau aaauguguga auuuuaauuu | 1680 |
| uaauggauua acaggaacag gagugcugac agaaucuaau aaaaaauuuc ugccuuuuca | 1740 |
| gcaguuuggc agagauauug cagauaccac agaugcagug agagauccuc agacauuaga | 1800 |
| aauucuggau auuacaccuu guucuuuugg gggugugucu gugauuacac cuggaacaaa | 1860 |
| uacaucuaau cagguggcug ugcuguauca ggaugugaau uguacagaag ugccaguggc | 1920 |
| aauucaugca gaucagcuga caccaacaug gagaguguau ucuacaggau cuaauguguu | 1980 |
| ucagacaaga gcaggauguc ugauuggagc agaacaugug aauaauucuu augaauguga | 2040 |
| uauuccaauu ggagcaggca uuugugcauc uuaucagaca cagacaaauu ccccaaggag | 2100 |
| agcaagaucu guggcaucuc agucuauuau ugcauacacc augucucugg gagcagaaaa | 2160 |
| uucuguggca uauucuaaua auucuauugc uauuccaaca aauuuuacca uuucugugac | 2220 |
| aacagaaauu uuaccugugu cuaugacaaa aacaucugug gauuguacca uguacauuug | 2280 |
| uggagauucu acagaauguu cuaaucugcu gcugcaguau ggaucuuuuu guacacagcu | 2340 |
| gaauagagcu uuaacaggaa uugcugugga acaggauaaa aauacacagg aaguguuugc | 2400 |
| ucaggugaaa cagauuuaca aaacaccacc aauuaaagau uuuggaggau uuaauuuuag | 2460 |
| ccagauucug ccugauccuu cuaaaccuuc uaaaagaucu uuuauugaag aucugcuguu | 2520 |
| uaauaaagug acacuggcag augcaggauu uauuaaacag uauggagauu gccuggguga | 2580 |
| uauugcugca agagaucuga uuugugcuca gaaauuuaau ggacugacag ugcugccucc | 2640 |
| ucugcugaca gaugaaauga uugcucagua cacaucugcu uuacuggcug gaacaauuac | 2700 |
| aagcggaugg acauuuggag cuggagcugc ucugcagauu ccuuuugcaa ugcagauggc | 2760 |
| uuacagauuu aauggaauug gagugacaca gaauguguua uaugaaaauc agaaacugau | 2820 |
| ugcaaaucag uuuaauucug caauuggcaa aauucaggau ucucugucuu cuacagcuuc | 2880 |
| ugcucuggga aaacugcagg auguggugaa ucagaaugca caggcacuga auacucuggu | 2940 |
| gaaacagcug ucuagcaauu uuggggcaau uucuucugug cugaaugaua uucugucuag | 3000 |
| acuggauccu ccugaagcug aagugcagau ugauagacug aucacaggaa gacugcaguc | 3060 |
| ucugcagacu uaugugacac agcagcugau uagagcugcu gaaauuagag cuucugcuaa | 3120 |
| ucuggcugcu acaaaaaugu cugaaugugu gcugggacag ucaaaaagag uggauuuuug | 3180 |
| uggaaaagga uaucaucuga ugucuuuucc acagucugcu ccacauggag ugguguuuuu | 3240 |
| acaugugaca uaugugccag cacaggaaaa gaauuuuacc acagcaccag caauuuguca | 3300 |
| ugauggaaaa gcacauuuuc caagagaagg aguguuugug ucuaauggaa cacauugguu | 3360 |
| ugugacacag agaaauuuuu augaaccuca gauuauuaca acagauaaua cauuuguguc | 3420 |
| aggaaauugu gaugugguga uuggaauugu gaauaauaca guguaugauc cacugcagcc | 3480 |
| agaacuggau ucuuuuaaag aagaacugga uaaauauuuu aaaaaucaca caucuccuga | 3540 |
| uguggauuua ggagauauuu cuggaaucaa ugcaucugug gugaauauuc agaaagaaau | 3600 |
| ugauagacug aaugaagugg ccaaaaaucu gaaugaaucu cugauugauc ugcaggaacu | 3660 |
| uggaaaauau gaacaguaca uuaaauggcc uugguacauu uggcuuggau uuauugcagg | 3720 |
| auuaauugca auugugaugg ugacaauuau guuauguugu augacaucau guuguucuug | 3780 |
| uuuaaaagga uguuguucuu guggaagcug uuguaaauuu gaugaagaug auucugaacc | 3840 |
| uguguuaaaa ggagugaaau ugcauuacac augaugacuc gagcugguac ugcaugcacg | 3900 |
| caaugcuagc ugccccuuuc ccguccuggg uaccccgagu cucccccgac cucggguccc | 3960 |
| agguaugcuc ccaccuccac cugccccacu caccaccucu gcuaguucca gacaccuccc | 4020 |
| aagcacgcag caaugcagcu caaaacgcuu agccuagcca cacccccacg ggaaacagca | 4080 |
| gugauuaacc uuuagcaaua aacgaaaguu uaacuaagcu auacuaaccc caggguuggu | 4140 |
| caauuucgug ccagccacac ccuggagcua gcaaaaaaaa aaaaaaaaaa aaaaaaaaaa | 4200 |
| aagcauauga cuaaaaaaaa aaaaaaaaaa aaaaaaaaaa aaaaaaaaaa aaaaaaaaaa | 4260 |
| aaaaaaaaaa aaaaaaaaaa aa | 4282 |

RBL063.2 (SEQ ID NO: 29 nucleotide; SEQ ID NO: 9 amino acid)

| | |
|---|---|
| Structure | beta-S-ARCA(D1)-hAg-Kozak-S1S2-PP-FI-A30L70 |
| Encoded antigen | Viral spike protein (S1S2 protein) of the SARS-CoV-2 (S1S2 full-length |
| | protein, sequence variant) |

SEQ ID NO: 29

| | |
|---|---|
| gggcgaacua guauucuucu gguccccaca gacucagaga gaacccgcca ccauguucgu | 60 |
| guuccuggug cugcugccuc ugguguccag ccagugugug aaccugacca ccagaacaca | 120 |
| gcugccucca gccuacacca acagcuuuac cagaggcgug uacuaccccg acaagguguu | 180 |
| cagauccagc gugcugcacu cuacccagga ccuguuccug ccuuucuuca gcaacgugac | 240 |
| cugguuccac gccauccacg uguccggcac caauggcacc aagagauucg acaaccccgu | 300 |
| gcugcccuuc aacgacgggg uguacuuugc cagcaccgag aaguccaaca ucaucagagg | 360 |
| cuggaucuuc ggcaccacac uggacagcaa gacccagagc cugcugaucg ugaacaacgc | 420 |
| caccaacgug gucaucaaag ugugcgaguu ccaguucugc aacgaccccu uccugggcgu | 480 |
| cuacuaccac aagaacaaca agagcuggau ggaaagcgag uuccgggugu acagcagcgc | 540 |
| caacaacugc accuucgagu acguguccca gccuuuccug auggaccugg aaggcaagca | 600 |
| gggcaacuuc aagaaccugc gcgaguucgu guuuaagaac aucgacggcu acuucaagau | 660 |
| cuacagcaag cacaccccua ucaaccucgu gcgggaucug ccucagggcu ucucugcucu | 720 |
| ggaaccccug guggaucugc ccaucggcau caacaucacc cgguuucaga cacugcuggc | 780 |
| ccugcacaga agcuaccuga caccuggcga uagcagcagc ggauggacag cuggugccgc | 840 |
| cgcuuacuau gugggcuacc ugcagccuag aaccuuccug cugaaguaca acgagaacgg | 900 |
| caccaucacc gacgccgugg auugugcucu ggauccucug agcgagacaa agugcacccu | 960 |
| gaaguccuuc accguggaaa agggcaucua ccagaccagc aacuuccggg ugcagcccac | 1020 |
| cgaauccauc gugcgguucc ccaauaucac caaucugugc cccuucggcg agguguucaa | 1080 |
| ugccaccaga uucgccucug uguacgccug gaaccggaag cggaucagca auugcguggc | 1140 |
| cgacuacucc gugcuguaca acuccgccag cuucagcacc uucaagugcu acggcguguc | 1200 |
| cccuaccaag cugaacgacc ugugcuucac aaacguguac gccgacagcu ucgugauccg | 1260 |
| gggagaugaa gugcggcaga uugccccugg acagacaggc aagaucgccg acuacaacua | 1320 |
| caagcugccc gacgacuuca ccggcugugu gauugccugg aacagcaaca accuggacuc | 1380 |
| caaagucggc ggcaacuaca auuaccugua ccggcuguuc cggaagucca aucugaagcc | 1440 |
| cuucgagcgg gacaucucca ccgagaucua ucaggccggc agcaccccuu guaacggcgu | 1500 |
| ggaaggcuuc aacugcuacu ucccacugca guccuacggc uuucagccca caaauggcgu | 1560 |
| gggcuaucag cccuacagag ugguggugcu gagcuucgaa cugcugcaug ccccugccac | 1620 |
| agugugcggc ccuaagaaaa gcaccaaucu cgugaagaac aaaugcguga acuucaacuu | 1680 |
| caacggccug accggcaccg gcgugcugac agagagcaac aagaaguucc ugccauucca | 1740 |
| gcaguuuggc cgggauaucg ccgauaccac agacgccguu agagaucccc agacacugga | 1800 |
| aauccuggac aucaccccuu gcagcuucgg cggagugucu gugaucaccc cuggcaccaa | 1860 |
| caccagcaau cagguggcag ugcuguacca ggacgugaac uguaccgaag ugcccguggc | 1920 |
| cauucacgcc gaucagcuga caccuacaug gcggguguac uccaccggca gcaauguguu | 1980 |
| ucagaccaga gccggcuguc ugaucggagc cgagcacgug aacaauagcu acgagugcga | 2040 |
| cauccccauc ggcgcuggaa ucugcgccag cuaccagaca cagacaaaca gcccucggag | 2100 |
| agccagaagc guggccagcc agagcaucau ugccuacaca augucucugg gcgccgagaa | 2160 |
| cagcguggcc uacuccaaca acucuaucgc uauccccacc aacuucacca ucagcgugac | 2220 |
| cacagagauc cugccugugu ccaugaccaa gaccagcgug gacugcacca uguacaucug | 2280 |
| cggcgauucc accgagugcu ccaaccugcu gcugcaguac ggcagcuucu gcacccagcu | 2340 |
| gaauagagcc cugacaggga ucgccgugga acaggacaag aacacccaag agguguucgc | 2400 |
| ccaagugaag cagaucuaca agaccccucc uaucaaggac uucggcggcu ucaauuucag | 2460 |
| ccagauucug cccgauccua gcaagcccag caagcggagc uucaucgagg accugcuguu | 2520 |
| caacaaagug acacuggccg acgccggcuu caucaagcag uauggcgauu gucugggcga | 2580 |
| cauugccgcc agggaucuga uuugcgccca gaaguuuaac ggacugacag ugcugccucc | 2640 |
| ucugcugacc gaugagauga ucgcccagua cacaucugcc cugcuggccg gcacaaucac | 2700 |
| aagcggcugg acauuuggag caggcgccgc ucugcagauc cccuuugcua ugcagauggc | 2760 |
| cuaccgguuc aacggcaucg gagugaccca gaaugugcug uacgagaacc agaagcugau | 2820 |
| cgccaaccag uucaacagcg ccaucggcaa gauccaggac agccugagca gcacagcaag | 2880 |
| cgcccuggga aagcugcagg acguggucaa ccagaaugcc caggcacuga acacccuggu | 2940 |
| caagcagcug uccuccaacu ucggcgccau cagcucugug cugaacgaua uccugagcag | 3000 |
| acuggacccu ccugaggccg aggugcagau cgacagacug aucacaggca gacugcagag | 3060 |
| ccuccagaca uacgugaccc agcagcugau cagagccgcc gagauuagag ccucugccaa | 3120 |
| ucuggccgcc accaagaugu cugagugugu gcugggccag agcaagagag uggacuuuug | 3180 |
| cggcaagggc uaccaccuga ugagcuuccc ucagucugcc ccucacggcg ugguguuucu | 3240 |
| gcacgugaca uaugugcccg cucaagagaa gaauuucacc accgcuccag ccaucugcca | 3300 |
| cgacggcaaa gcccacuuuc cuagagaagg cguguucgug uccaacggca cccauugguu | 3360 |
| cgugacacag cggaacuucu acgagcccca gaucaucacc accgacaaca ccuucguguc | 3420 |
| uggcaacugc gacgucguga ucggcauugu gaacaauacc guguacgacc cucugcagcc | 3480 |
| cgagcuggac agcuucaaag aggaacugga caaguacuuu aagaaccaca caagccccga | 3540 |
| cguggaccug ggcgauauca gcggaaucaa ugccagcguc gugaacaucc agaaagagau | 3600 |
| cgaccggcug aacgaggugg ccaagaaucu gaacgagagc cugaucgacc ugcaagaacu | 3660 |
| ggggaaguac gagcaguaca ucaaguggcc cugguacauc uggcugggcu uuaucgccgg | 3720 |
| acugauugcc aucgugaugg ucacaaucau gcuguguugc augaccagcu gcuguagcug | 3780 |
| ccugaagggc uguuguagcu guggcagcug cugcaaguuc gacgaggacg auucugagcc | 3840 |
| cgugcugaag ggcgugaaac ugcacuacac augaugacuc gagcugguac ugcaugcacg | 3900 |
| caaugcuagc ugccccuuuc ccguccuggg uaccccgagu cucccccgac cucggguccc | 3960 |
| agguaugcuc ccaccuccac cugccccacu caccaccucu gcuaguucca gacaccuccc | 4020 |
| aagcacgcag caaugcagcu caaaacgcuu agccuagcca cacccccacg ggaaacagca | 4080 |
| gugauuaacc uuuagcaaua aacgaaaguu uaacuaagcu auacuaaccc caggguuggu | 4140 |
| caauuucgug ccagccacac ccuggagcua gcaaaaaaaa aaaaaaaaaa aaaaaaaaaa | 4200 |
| aagcauauga cuaaaaaaaa aaaaaaaaaa aaaaaaaaaa aaaaaaaaaa aaaaaaaaaa | 4260 |
| aaaaaaaaaa aaaaaaaaaa aa | 4282 |

BNT162a1; RBL063.3 (SEQ ID NO: 30 nucleotide; SEQ ID NO: 21 amino acid)

| | |
|---|---|
| Structure | beta-S-ARCA(D1)-hAg-Kozak-RBD-GS-Fibritin-FI-A30L70 |
| Encoded antigen | Viral spike protein (S protein) of the SARS-COV-2 (partial sequence, |
| | Receptor Binding Domain (RBD) of S1S2 protein) |

SEQ ID NO: 30

| | |
|---|---|
| gggcgaacua guauucuucu gguccccaca gacucagaga gaacccgcca ccauguuugu | 60 |
| guuucuugug cugcugccuc uugugucuuc ucagugugug gugagauuuc caaauauuac | 120 |
| aaaucugugu ccauuuggag aaguguuuaa ugcaacaaga uuugcaucug uguaugcaug | 180 |
| gaauagaaaa agaauuucua auuguguggc ugauuauucu gugcuguaua auagugcuuc | 240 |
| uuuuuccaca uuuaaauguu auggaguguc uccaacaaaa uuaaaugauu uauguuuuac | 300 |
| aaauguguau gcugauucuu uugugaucag aggugaugaa gugagacaga uugcccccgg | 360 |
| acagacagga aaaauugcug auuacaauua caaacugccu gaugauuuua caggaugugu | 420 |
| gauugcuugg aauucuaaua auuuagauuc uaaaguggga ggaaauuaca auuaucugua | 480 |
| cagacuguuu agaaaaucaa aucugaaacc uuuugaaaga gauauuucaa cagaaauuua | 540 |
| ucaggcugga ucaacaccuu guaauggagu ggaaggauuu aauuguuauu uuccauuaca | 600 |
| gagcuaugga uuucagccaa ccaauggugu gggauaucag ccauauagag ugguggugcu | 660 |
| gucuuuugaa cugcugcaug caccugcaac agugugugga ccuaaaggcu cccccggcuc | 720 |
| cggcuccgga ucugguuaua uuccugaagc uccaagagau gggcaagcuu acguucguaa | 780 |
| agauggcgaa uggguauuac uuucuaccuu uuuaggccgg ucccuggagg ugcuguucca | 840 |
| gggccccggc ugaugacucg agcugguacu gcaugcacgc aaugcuagcu gccccuuucc | 900 |
| cguccugggu accccgaguc ucccccgacc ucggguccca gguaugcucc caccuccacc | 960 |
| ugccccacuc accaccucug cuaguuccag acaccuccca agcacgcagc aaugcagcuc | 1020 |
| aaaacgcuua gccuagccac acccccacgg gaaacagcag ugauuaaccu uuagcaauaa | 1080 |
| acgaaaguuu aacuaagcua uacuaacccc aggguugguc aauuucgugc cagccacacc | 1140 |
| cuggagcuag caaaaaaaaa aaaaaaaaaa aaaaaaaaaa agcauaugac uaaaaaaaaa | 1200 |
| aaaaaaaaaa aaaaaaaaaa aaaaaaaaaa aaaaaaaaaa aaaaaaaaaa aaaaaaaaaa | 1260 |
| a | 1261 |

BNT162b2; RBP020.1 (SEQ ID NO: 31 nucleotide; SEQ ID NO: 9 amino acid)

| | |
|---|---|
| Structure | m₂^{7,3′-O}Gppp(m₁^{2′-O})ApG-hAg-Kozak-S1S2-PP-FI-A30L70 |
| Encoded antigen | Viral spike protein (S1S2 protein) of the SARS-CoV-2 (S1S2 full-length |
| | protein, sequence variant) |

SEQ ID NO: 31

| | |
|---|---|
| agaauaaacu aguauucuuc ugguccccac agacucagag agaacccgcc accauguuug | 60 |
| uguuucuugu gcugcugccu cuugugucuu cucagugugu gaauuugaca acaagaacac | 120 |
| agcugccacc agcuuauaca aauucuuuua ccagaggagu guauuauccu gauaaagugu | 180 |
| uuagaucuuc ugugcugcac agcacacagg accuguuucu gccauuuuuu agcaauguga | 240 |
| caugguuuca ugcaauucau gugucuggaa caaauggaac aaaaagauuu gauaauccug | 300 |
| ugcugccuuu uaaugaugga guguauuuug cuucaacaga aaagucaaau auuauuagag | 360 |
| gauggauuuu uggaacaaca cuggauucua aaacacaguc ucugcugauu gugaauaaug | 420 |
| caacaaaugu ggugauuaaa gugugugaau uucaguuuug uaaugauccu uuucugggag | 480 |
| uguauuauca caaaaauaau aaaucuugga uggaaucuga auuuagagug uauuccucug | 540 |
| caaauaauug uacauuugaa uaugugucuc agccuuuucu gauggaucug gaaggaaaac | 600 |
| agggcaauuu uaaaaaucug agagaauuug uguuuaaaaa uauugaugga uauuuuaaaa | 660 |
| uuuauucuaa acacacacca auuaauuuag ugagagaucu gccucaggga uuuucugcuc | 720 |
| uggaaccucu gguggaucug ccaauuggca uuaauauuac aagauuucag acacugcugg | 780 |
| cucugcacag aucuuaucug acaccuggag auucuucuuc uggauggaca gccggagcug | 840 |
| cagcuuauua ugugggcuau cugcagccaa gaacauuucu gcugaaauau aaugaaaaug | 900 |
| gaacaauuac agaugcugug gauugugcuc uggauccucu gucugaaaca aaauguacau | 960 |
| uaaaaucuuu uacaguggaa aaaggcauuu aucagacauc uaauuuuaga gugcagccaa | 1020 |
| cagaaucuau ugugagauuu ccaaauauua caaaucugug uccauuugga gaaguguuua | 1080 |
| augcaacaag auuugcaucu guguaugcau ggaauagaaa aagaauuucu aauugugugg | 1140 |
| cugauuauuc ugugcuguau aauagugcuu cuuuuuccac auuuaaaugu uauggagugu | 1200 |
| cuccaacaaa auuaaaugau uuauguuuua caaaugugua ugcugauucu uuugugauca | 1260 |
| gaggugauga agugagacag auugcccccg gacagacagg aaaaauugcu gauuacaauu | 1320 |
| acaaacugcc ugaugauuuu acaggaugug ugauugcuug gaauucuaau aauuuagauu | 1380 |
| cuaaaguggg aggaaauuac aauuaucugu acagacuguu uagaaaauca aaucugaaac | 1440 |
| cuuuugaaag agauauuuca acagaaauuu aucaggcugg aucaacaccu uguaauggag | 1500 |
| uggaaggauu uaauuguuau uuuccauuac agagcuaugg auuucagcca accaauggug | 1560 |
| ugggauauca gccauauaga gugguggugc ugucuuuuga acugcugcau gcaccugcaa | 1620 |
| cagugugugg accuaaaaaa ucuacaaauu uagugaaaaa uaaaugugug aauuuuaauu | 1680 |
| uuaauggauu aacaggaaca ggagugcuga cagaaucuaa uaaaaaauuu cugccuuuuc | 1740 |
| agcaguuugg cagagauauu gcagauacca cagaugcagu gagagauccu cagacauuag | 1800 |
| aaauucugga uauuacaccu uguucuuuug gggguguguc ugugauuaca ccuggaacaa | 1860 |
| auacaucuaa ucagguggcu gugcuguauc aggaugugaa uuguacagaa gugccagugg | 1920 |
| caauucaugc agaucagcug acaccaacau ggagagugua uucuacagga ucuaaugugu | 1980 |
| uucagacaag agcaggaugu cugauuggag cagaacaugu gaauaauucu uaugaaugug | 2040 |
| auauuccaau uggagcaggc auuugugcau cuuaucagac acagacaaau uccccaagga | 2100 |
| gagcaagauc uguggcaucu cagucuauua uugcauacac caugucucug ggagcagaaa | 2160 |
| auucuguggc auauucuaau aauucuauug cuauuccaac aaauuuuacc auuucuguga | 2220 |
| caacagaaau uuuaccugug ucuaugacaa aaacaucugu ggauuguacc auguacauuu | 2280 |
| guggagauuc uacagaaugu ucuaaucugc ugcugcagua uggaucuuuu uguacacagc | 2340 |
| ugaauagagc uuuaacagga auugcugugg aacaggauaa aaauacacag gaaguguuug | 2400 |
| cucaggugaa acagauuuac aaaacaccac caauuaaaga uuuuggagga uuuaauuuua | 2460 |
| gccagauucu gccugauccu ucuaaaccuu cuaaaagauc uuuuauugaa gaucugcugu | 2520 |
| uuaauaaagu gacacuggca gaugcaggau uuauuaaaca guauggagau ugccugggug | 2580 |
| auauugcugc aagagaucug auuugugcuc agaaauuuaa uggacugaca gugcugccuc | 2640 |
| cucugcugac agaugaaaug auugcucagu acacaucugc uuuacuggcu ggaacaauua | 2700 |
| caagcggaug gacauuugga gcuggagcug cucugcagau uccuuuugca augcagaugg | 2760 |
| cuuacagauu uaauggaauu ggagugacac agaauguguu auaugaaaau cagaaacuga | 2820 |
| uugcaaauca guuuaauucu gcaauuggca aaauucagga uucucugucu ucuacagcuu | 2880 |
| cugcucuggg aaaacugcag gaugugguga aucagaaugc acaggcacug aauacucugg | 2940 |
| ugaaacagcu gucuagcaau uuuggggcaa uuucuucugu gcugaaugau auucugucua | 3000 |
| gacuggaucc uccugaagcu gaagugcaga uugauagacu gaucacagga agacugcagu | 3060 |
| cucugcagac uuaugugaca cagcagcuga uuagagcugc ugaaauuaga gcuucugcua | 3120 |
| aucuggcugc uacaaaaaug ucugaaugug ugcugggaca gucaaaaaga guggauuuuu | 3180 |
| guggaaaagg auaucaucug augucuuuuc cacagucugc uccacaugga gugguguuuu | 3240 |
| uacaugugac auaugugcca gcacaggaaa agaauuuuac cacagcacca gcaauuuguc | 3300 |
| augauggaaa agcacauuuu ccaagagaag gaguguuugu gucuaaugga acacauuggu | 3360 |
| uugugacaca gagaaauuuu uaugaaccuc agauuauuac aacagauaau acauuugugu | 3420 |
| caggaaauug ugauguggug auuggaauug ugaauaauac aguguaugau ccacugcagc | 3480 |
| cagaacugga uucuuuuaaa gaagaacugg auaaauauuu uaaaaaucac acaucuccug | 3540 |
| auguggauuu aggagauauu ucuggaauca augcaucugu ggugaauauu cagaaagaaa | 3600 |
| uugauagacu gaaugaagug gccaaaaauc ugaaugaauc ucugauugau cugcaggaac | 3660 |
| uuggaaaaua ugaacaguac auuaaauggc cuugguacau uuggcuugga uuuauugcag | 3720 |
| gauuaauugc aauugugaug gugacaauua uguuauguug uaugacauca uguuguucuu | 3780 |
| guuuaaaagg auguuguucu uguggaagcu guuguaaauu ugaugaagau gauucugaac | 3840 |
| cuguguuaaa aggagugaaa uugcauuaca caugaugacu cgagcuggua cugcaugcac | 3900 |
| gcaaugcuag cugccccuuu cccguccugg guaccccgag ucucccccga ccucgggucc | 3960 |
| cagguaugcu cccaccucca ccugccccac ucaccaccuc ugcuaguucc agacaccucc | 4020 |
| caagcacgca gcaaugcagc ucaaaacgcu uagccuagcc acacccccac gggaaacagc | 4080 |
| agugauuaac cuuuagcaau aaacgaaagu uuaacuaagc uauacuaacc ccaggguugg | 4140 |
| ucaauuucgu gccagccaca cccuggagcu agcaaaaaaa aaaaaaaaaa aaaaaaaaaa | 4200 |
| aaagcauaug acuaaaaaaa aaaaaaaaaa aaaaaaaaaa aaaaaaaaaa aaaaaaaaaa | 4260 |
| aaaaaaaaaa aaaaaaaaaa aaa | 4283 |

RBP020.2 (SEQ ID NO: 10 nucleotide; SEQ ID NO: 9 amino acid) (see Table 1)

| | |
|---|---|
| Structure | m₂^{7,3′-O}Gppp(m₁^{2′-O})ApG-hAg-Kozak-S1S2-PP-FI-A30L70 |
| Encoded antigen | Viral spike protein (S1S2 protein) of the SARS-CoV-2 (S1S2 full-length |
| | protein, sequence variant) |

BNT162b1; RBP020.3 (SEQ ID NO: 32; SEQ ID NO: 21 amino acid)

| | |
|---|---|
| Structure | m₂^{7,3′-O}Gppp(m₁^{2′-O})ApG-hAg-Kozak-RBD-GS-Fibritin-FI-A30L70 |
| Encoded antigen | Viral spike protein (S1S2 protein) of the SARS-CoV-2 (partial sequence, |
| | Receptor Binding Domain (RBD) of S1S2 protein fused to fibritin) |

SEQ ID NO: 32

| | |
|---|---|
| agaauaaacu aguauucuuc ugguccccac agacucagag agaacccgcc accauguuug | 60 |
| uguuucuugu gcugcugccu cuugugucuu cucagugugu ggugagauuu ccaaauauua | 120 |
| caaaucugug uccauuugga gaaguguuua augcaacaag auuugcaucu guguaugcau | 180 |
| ggaauagaaa aagaauuucu aauugugugg cugauuauuc ugugcuguau aauagugcuu | 240 |
| cuuuuuccac auuuaaaugu uauggagugu cuccaacaaa auuaaaugau uuauguuuua | 300 |
| caaaugugua ugcugauucu uuugugauca gaggugauga agugagacag auugcccccg | 360 |
| gacagacagg aaaaauugcu gauuacaauu acaaacugcc ugaugauuuu acaggaugug | 420 |
| ugauugcuug gaauucuaau aauuuagauu cuaaaguggg aggaaauuac aauuaucugu | 480 |
| acagacuguu uagaaaauca aaucugaaac cuuuugaaag agauauuuca acagaaauuu | 540 |
| aucaggcugg aucaacaccu uguaauggag uggaaggauu uaauuguuau uuuccauuac | 600 |
| agagcuaugg auuucagcca accaauggug ugggauauca gccauauaga gugguggugc | 660 |
| ugucuuuuga acugcugcau gcaccugcaa cagugugugg accuaaaggc ucccccggcu | 720 |
| ccggcuccgg aucugguuau auuccugaag cuccaagaga ugggcaagcu uacguucgua | 780 |
| aagauggcga auggguauua cuuucuaccu uuuuaggccg gucccuggag gugcuguucc | 840 |
| agggccccgg cugaugacuc gagcugguac ugcaugcacg caaugcuagc ugccccuuuc | 900 |
| ccguccuggg uaccccgagu cucccccgac cucggguccc agguaugcuc ccaccuccac | 960 |
| cugccccacu caccaccucu gcuaguucca gacaccuccc aagcacgcag caaugcagcu | 1020 |
| caaaacgcuu agccuagcca cacccccacg ggaaacagca gugauuaacc uuuagcaaua | 1080 |
| aacgaaaguu uaacuaagcu auacuaaccc caggguuggu caauuucgug ccagccacac | 1140 |
| ccuggagcua gcaaaaaaaa aaaaaaaaaa aaaaaaaaaa aagcauauga cuaaaaaaaa | 1200 |
| aaaaaaaaaa aaaaaaaaaa aaaaaaaaaa aaaaaaaaaa aaaaaaaaaa aaaaaaaaaa | 1260 |
| aa | 1262 |

RBS004.1 (SEQ ID NO: 33; SEQ ID NO: 9 amino acid)

| | |
|---|---|
| Structure | beta-S-ARCA(D1)-replicase-S1S2-PP-FI-A30L70 |
| Encoded antigen | Viral spike protein (S protein) of the SARS-CoV-2 (S1S2 full-length |
| | protein, sequence variant) |

SEQ ID NO: 33

| | |
|---|---|
| gaugggcggc gcaugagaga agcccagacc aauuaccuac ccaaaaugga gaaaguucac | 60 |
| guugacaucg aggaagacag cccauuccuc agagcuuugc agcggagcuu cccgcaguuu | 120 |
| gagguagaag ccaagcaggu cacugauaau gaccaugcua augccagagc guuuucgcau | 180 |
| cuggcuucaa aacugaucga aacggaggug gacccauccg acacgauccu ugacauugga | 240 |
| agugcgcccg cccgcagaau guauucuaag cacaaguauc auuguaucug uccgaugaga | 300 |
| ugugcggaag auccggacag auuguauaag uaugcaacua agcugaagaa aaacuguaag | 360 |
| gaaauaacug auaaggaauu ggacaagaaa augaaggagc ucgccgccgu caugagcgac | 420 |
| ccugaccugg aaacugagac uaugugccuc cacgacgacg agucgugucg cuacgaaggg | 480 |
| caagucgcug uuuaccagga uguauacgcg guugacggac cgacaagucu cuaucaccaa | 540 |
| gccaauaagg gaguuagagu cgccuacugg auaggcuuug acaccacccc uuuuauguuu | 600 |
| aagaacuugg cuggagcaua uccaucauac ucuaccaacu gggccgacga aaccguguua | 660 |
| acggcucgua acauaggccu augcagcucu gacguuaugg agcggucacg uagagggaug | 720 |
| uccauucuua gaaagaagua uuugaaacca uccaacaaug uucuauucuc uguuggcucg | 780 |
| accaucuacc acgaaaagag ggacuuacug aggagcuggc accugccguc uguauuucac | 840 |
| uuacguggca agcaaaauua cacaugucgg ugugagacua uaguuaguug cgacggguac | 900 |
| gucguuaaaa gaauagcuau caguccaggc cuguauggga agccuucagg cuaugcugcu | 960 |
| acgaugcacc gcgagggauu cuugugcugc aaagugacag acacauugaa cggggagagg | 1020 |
| gucucuuuuc ccgugugcac guaugugcca gcuacauugu gugaccaaau gacuggcaua | 1080 |
| cuggcaacag augucagugc ggacgacgcg caaaaacugc ugguugggcu caaccagcgu | 1140 |
| auagucguca acggucgcac ccagagaaac accaauacca ugaaaaauua ccuuuugccc | 1200 |
| guaguggccc aggcauuugc uaggugggca aaggaauaua aggaagauca agaagaugaa | 1260 |
| aggccacuag gacuacgaga uagacaguua gucauggggu guuguugggc uuuuagaagg | 1320 |
| cacaagauaa caucuauuua uaagcgcccg gauacccaaa ccaucaucaa agugaacagc | 1380 |
| gauuuccacu cauucgugcu gcccaggaua ggcaguaaca cauuggagau cgggcugaga | 1440 |
| acaagaauca ggaaaauguu agaggagcac aaggagccgu caccucucau uaccgccgag | 1500 |
| gacguacaag aagcuaagug cgcagccgau gaggcuaagg aggugcguga agccgaggag | 1560 |
| uugcgcgcag cucuaccacc uuuggcagcu gauguugagg agcccacucu ggaagccgau | 1620 |
| gucgacuuga uguuacaaga ggcuggggcc ggcucagugg agacaccucg uggcuugaua | 1680 |
| aagguuacca gcuacgcugg cgaggacaag aucggcucuu acgcugugcu uucuccgcag | 1740 |
| gcuguacuca agagugaaaa auuaucuugc auccacccuc ucgcugaaca agucauagug | 1800 |
| auaacacacu cuggccgaaa agggcguuau gccguggaac cauaccaugg uaaaguagug | 1860 |
| gugccagagg gacaugcaau acccguccag gacuuucaag cucugaguga aagugccacc | 1920 |
| auuguguaca acgaacguga guucguaaac agguaccugc accauauugc cacacaugga | 1980 |
| ggagcgcuga acacugauga agaauauuac aaaacuguca agcccagcga gcacgacggc | 2040 |
| gaauaccugu acgacaucga caggaaacag ugcgucaaga aagagcuagu cacugggcua | 2100 |
| gggcucacag gcgagcuggu cgauccuccc uuccaugaau ucgccuacga gagucugaga | 2160 |
| acacgaccag ccgcuccuua ccaaguacca accauagggg uguauggcgu gccaggauca | 2220 |
| ggcaagucug gcaucauuaa aagcgcaguc accaaaaaag aucuaguggu gagcgccaag | 2280 |
| aaagaaaacu gugcagaaau uauaagggac gucaagaaaa ugaaagggcu ggacgucaau | 2340 |
| gccagaacug uggacucagu gcucuugaau ggaugcaaac accccguaga gacccuguau | 2400 |
| auugacgagg cuuuugcuug ucaugcaggu acucucagag cgcucauagc cauuauaaga | 2460 |
| ccuaaaaagg cagugcucug cggagauccc aaacagugcg guuuuuuuaa caugaugugc | 2520 |
| cugaaagugc auuuuaacca cgagauuugc acacaagucu uccacaaaag caucucucgc | 2580 |
| cguugcacua aaucugugac uucggucguc ucaaccuugu uuuacgacaa aaaaaugaga | 2640 |
| acgacgaauc cgaaagagac uaagauugug auugacacua ccggcaguac caaaccuaag | 2700 |
| caggacgauc ucauucucac uuguuucaga ggguggguga agcaguugca aauagauuac | 2760 |
| aaaggcaacg aaauaaugac ggcagcugcc ucucaagggc ugacccguaa agguguguau | 2820 |
| gccguucggu acaaggugaa ugaaaauccu cuguacgcac ccaccucaga acaugugaac | 2880 |
| guccuacuga cccgcacgga ggaccgcauc guguggaaaa cacuagccgg cgacccaugg | 2940 |
| auaaaaacac ugacugccaa guacccuggg aauuucacug ccacgauaga ggaguggcaa | 3000 |
| gcagagcaug augccaucau gaggcacauc uuggagagac cggacccuac cgacgucuuc | 3060 |
| cagaauaagg caaacgugug uugggccaag gcuuuagugc cggugcugaa gaccgcuggc | 3120 |
| auagacauga ccacugaaca auggaacacu guggauuauu uugaaacgga caaagcucac | 3180 |
| ucagcagaga uaguauugaa ccaacuaugc gugagguucu uuggacucga ucuggacucc | 3240 |
| ggucuauuuu cugcacccac uguuccguua uccauuagga auaaucacug ggauaacucc | 3300 |
| ccgucgccua acauguacgg gcugaauaaa gaaguggucc gucagcucuc ucgcagguac | 3360 |
| ccacaacugc cucgggcagu ugccacuggu agagucuaug acaugaacac ugguacacug | 3420 |
| cgcaauuaug auccgcgcau aaaccuagua ccuguaaaca gaagacugcc ucaugcuuua | 3480 |
| guccuccacc auaaugaaca cccacagagu gacuuuucuu cauucgucag caaauugaag | 3540 |
| ggcagaacug uccugguggu cggggaaaag uuguccgucc caggcaaaau gguugacugg | 3600 |
| uugucagacc ggccugaggc uaccuucaga gcucggcugg auuuaggcau cccaggugau | 3660 |
| gugcccaaau augacauaau auuuguuaau gugaggaccc cauauaaaua ccaucacuau | 3720 |
| cagcagugug aagaccaugc cauuaagcua agcauguuga ccaagaaagc augucugcau | 3780 |
| cugaaucccg gcggaaccug ugucagcaua gguuaugguu acgcugacag ggccagcgaa | 3840 |
| agcaucauug gugcuauagc gcggcaguuc aaguuuuccc gaguaugcaa accgaaaucc | 3900 |
| ucacuugagg agacggaagu ucuguuugua uucauugggu acgaucgcaa ggcccguacg | 3960 |
| cacaauccuu acaagcuauc aucaaccuug accaacauuu auacagguuc cagacuccac | 4020 |
| gaagccggau gugcacccuc auaucaugug gugcgagggg auauugccac ggccaccgaa | 4080 |
| ggagugauua uaaaugcugc uaacagcaaa ggacaaccug gcggaggggu gugcggagcg | 4140 |
| cuguauaaga aauucccgga aaguuucgau uuacagccga ucgaaguagg aaaagcgcga | 4200 |
| cuggucaaag gugcagcuaa acauaucauu caugccguag gaccaaacuu caacaaaguu | 4260 |
| ucggagguug aaggugacaa acaguuggca gaggcuuaug aguccaucgc uaagauuguc | 4320 |
| aacgauaaca auuacaaguc aguagcgauu ccacuguugu ccaccggcau cuuuuccggg | 4380 |
| aacaaagauc gacuaaccca aucauugaac cauuugcuga cagcuuuaga caccacugau | 4440 |
| gcagauguag ccauauacug cagggacaag aaaugggaaa ugacucucaa ggaagcagug | 4500 |
| gcuaggagag aagcagugga ggagauaugc auauccgacg auucuucagu gacagaaccu | 4560 |
| gaugcagagc uggugagggu gcaucccaag aguucuuugg cuggaaggaa gggcuacagc | 4620 |
| acaagcgaug gcaaaacuuu cucauauuug gaagggacca aguuucacca ggcggccaag | 4680 |
| gauauagcag aaauuaaugc cauguggccc guugcaacgg aggccaauga gcagguaugc | 4740 |
| auguauaucc ucggagaaag caugagcagu auuaggucga aaugccccgu cgaggagucg | 4800 |
| gaagccucca caccaccuag cacgcugccu ugcuugugca uccaugccau gacuccagaa | 4860 |
| agaguacagc gccuaaaagc cucacgucca gaacaaauua cugugugcuc auccuuucca | 4920 |
| uugccgaagu auagaaucac uggugugcag aagauccaau gcucccagcc uauauuguuc | 4980 |
| ucaccgaaag ugccugcgua uauucaucca aggaaguauc ucguggaaac accaccggua | 5040 |
| gacgagacuc cggagccauc ggcagagaac caauccacag aggggacacc ugaacaacca | 5100 |
| ccacuuauaa ccgaggauga gaccaggacu agaacgccug agccgaucau caucgaagaa | 5160 |
| gaagaagaag auagcauaag uuugcuguca gauggcccga cccaccaggu gcugcaaguc | 5220 |
| gaggcagaca uucacgggcc gcccucugua ucuagcucau ccugguccau uccucaugca | 5280 |
| uccgacuuug auguggacag uuuauccaua cuugacaccc uggagggagc uagcgugacc | 5340 |
| agcggggcaa cgucagccga gacuaacucu uacuucgcaa agaguaugga guuucuggcg | 5400 |
| cgaccggugc cugcgccucg aacaguauuc aggaacccuc cacaucccgc uccgcgcaca | 5460 |
| agaacaccgu cacuugcacc cagcagggcc ugcuccagaa ccagccuagu uuccaccccg | 5520 |
| ccaggcguga auagggugau cacuagagag gagcucgaag cgcuuacccc gucacgcacu | 5580 |
| ccuagcaggu cggucuccag aaccagccug gucuccaacc cgccaggcgu aaauagggug | 5640 |
| auuacaagag aggaguuuga ggcguucgua gcacaacaac aaugacgguu ugaugcgggu | 5700 |
| gcauacaucu uuuccuccga caccggucaa gggcauuuac aacaaaaauc aguaaggcaa | 5760 |
| acggugcuau ccgaaguggu guuggagagg accgaauugg agauuucgua ugccccgcgc | 5820 |
| cucgaccaag aaaaagaaga auuacuacgc aagaaauuac aguuaaaucc cacaccugcu | 5880 |
| aacagaagca gauaccaguc caggaaggug gagaacauga aagccauaac agcuagacgu | 5940 |
| auucugcaag gccuagggca uuauuugaag gcagaaggaa aaguggagug cuaccgaacc | 6000 |
| cugcauccug uuccuuugua uucaucuagu gugaaccgug ccuuuucaag ccccaagguc | 6060 |
| gcaguggaag ccuguaacgc cauguugaaa gagaacuuuc cgacuguggc uucuuacugu | 6120 |
| auuauuccag aguacgaugc cuauuuggac augguugacg gagcuucaug cugcuuagac | 6420 |
| acugccaguu uuugcccugc aaagcugcgc agcuuuccaa agaaacacuc cuauuuggaa | 6240 |
| cccacaauac gaucggcagu gccuucagcg auccagaaca cgcuccagaa cguccuggca | 6300 |
| gcugccacaa aaagaaauug caaugucacg caaaugagag aauugcccgu auuggauucg | 6360 |
| gcggccuuua auguggaaug cuucaagaaa uaugcgugua auaaugaaua uugggaaacg | 6420 |
| uuuaaagaaa accccaucag gcuuacugaa gaaaacgugg uaaauuacau uaccaaauua | 6480 |
| aaaggaccaa aagcugcugc ucuuuuugcg aagacacaua auuugaauau guugcaggac | 6540 |
| auaccaaugg acagguuugu aauggacuua aagagagacg ugaaagugac uccaggaaca | 6600 |
| aaacauacug aagaacggcc caagguacag gugauccagg cugccgaucc gcuagcaaca | 6660 |
| gcguaucugu gcggaaucca ccgagagcug guuaggagau uaaaugcggu ccugcuuccg | 6720 |
| aacauucaua cacuguuuga uaugucggcu gaagacuuug acgcuauuau agccgagcac | 6780 |
| uuccagccug gggauugugu ucuggaaacu gacaucgcgu cguuugauaa aagugaggac | 6840 |
| gacgccaugg cucugaccgc guuaaugauu cuggaagacu uaggugugga cgcagagcug | 6900 |
| uugacgcuga uugaggcggc uuucggcgaa auuucaucaa uacauuugcc cacuaaaacu | 6960 |
| aaauuuaaau ucggagccau gaugaaaucu ggaauguucc ucacacuguu ugugaacaca | 7020 |
| gucauuaaca uuguaaucgc aagcagagug uugagagaac ggcuaaccgg aucaccaugu | 7080 |
| gcagcauuca uuggagauga caauaucgug aaaggaguca aaucggacaa auuaauggca | 7140 |
| gacaggugcg ccaccugguu gaauauggaa gucaagauua uagaugcugu ggugggcgag | 7200 |
| aaagcgccuu auuucugugg aggguuuauu uugugugacu ccgugaccgg cacagcgugc | 7260 |
| cguguggcag acccccuaaa aaggcuguuu aagcuaggca aaccucuggc agcagacgau | 7320 |
| gaacaugaug augacaggag aagggcauug caugaggagu caacacgcug gaaccgagug | 7380 |
| gguauucuuu cagagcugug caaggcagua gaaucaaggu augaaaccgu aggaacuucc | 7440 |
| aucauaguua uggccaugac uacucuagcu agcaguguua aaucauucag cuaccugaga | 7500 |
| ggggccccua uaacucucua cggcuaaccu gaauggacua cgacauaguc uaguccgcca | 7560 |
| agacuaguau guuuguguuu cuugugcugc ugccucuugu gucuucucag ugugugaauu | 7620 |
| ugacaacaag aacacagcug ccaccagcuu auacaaauuc uuuuaccaga ggaguguauu | 7680 |
| auccugauaa aguguuuaga ucuucugugc ugcacagcac acaggaccug uuucugccau | 7740 |
| uuuuuagcaa ugugacaugg uuucaugcaa uucauguguc uggaacaaau ggaacaaaaa | 7800 |
| gauuugauaa uccugugcug ccuuuuaaug auggagugua uuuugcuuca acagaaaagu | 7860 |
| caaauauuau uagaggaugg auuuuuggaa caacacugga uucuaaaaca cagucucugc | 7920 |
| ugauugugaa uaaugcaaca aaugugguga uuaaagugug ugaauuucag uuuuguaaug | 7980 |
| auccuuuucu gggaguguau uaucacaaaa auaauaaauc uuggauggaa ucugaauuua | 8040 |
| gaguguauuc cucugcaaau aauuguacau uugaauaugu gucucagccu uuucugaugg | 8100 |
| aucuggaagg aaaacagggc aauuuuaaaa aucugagaga auuuguguuu aaaaauauug | 8160 |
| auggauauuu uaaaauuuau ucuaaacaca caccaauuaa uuuagugaga gaucugccuc | 8220 |
| agggauuuuc ugcucuggaa ccucuggugg aucugccaau uggcauuaau auuacaagau | 8280 |
| uucagacacu gcuggcucug cacagaucuu aucugacacc uggagauucu ucuucuggau | 8340 |
| ggacagccgg agcugcagcu uauuaugugg gcuaucugca gccaagaaca uuucugcuga | 8400 |
| aauauaauga aaauggaaca auuacagaug cuguggauug ugcucuggau ccucugucug | 8460 |
| aaacaaaaug uacauuaaaa ucuuuuacag uggaaaaagg cauuuaucag acaucuaauu | 8520 |
| uuagagugca gccaacagaa ucuauuguga gauuuccaaa uauuacaaau cuguguccau | 8580 |
| uuggagaagu guuuaaugca acaagauuug caucugugua ugcauggaau agaaaaagaa | 8640 |
| uuucuaauug uguggcugau uauucugugc uguauaauag ugcuucuuuu uccacauuua | 8700 |
| aauguuaugg agugucucca acaaaauuaa augauuuaug uuuuacaaau guguaugcug | 8760 |
| auucuuuugu gaucagaggu gaugaaguga gacagauugc ccccggacag acaggaaaaa | 8820 |
| uugcugauua caauuacaaa cugccugaug auuuuacagg augugugauu gcuuggaauu | 8880 |
| cuaauaauuu agauucuaaa gugggaggaa auuacaauua ucuguacaga cuguuuagaa | 8940 |
| aaucaaaucu gaaaccuuuu gaaagagaua uuucaacaga aauuuaucag gcuggaucaa | 9000 |
| caccuuguaa uggaguggaa ggauuuaauu guuauuuucc auuacagagc uauggauuuc | 9060 |
| agccaaccaa ugguguggga uaucagccau auagaguggu ggugcugucu uuugaacugc | 9120 |
| ugcaugcacc ugcaacagug uguggaccua aaaaaucuac aaauuuagug aaaaauaaau | 9180 |
| gugugaauuu uaauuuuaau ggauuaacag gaacaggagu gcugacagaa ucuaauaaaa | 9240 |
| aauuucugcc uuuucagcag uuuggcagag auauugcaga uaccacagau gcagugagag | 9300 |
| auccucagac auuagaaauu cuggauauua caccuuguuc uuuugggggu gugucuguga | 9360 |
| uuacaccugg aacaaauaca ucuaaucagg uggcugugcu guaucaggau gugaauugua | 9420 |
| cagaagugcc aguggcaauu caugcagauc agcugacacc aacauggaga guguauucua | 9480 |
| caggaucuaa uguguuucag acaagagcag gaugucugau uggagcagaa caugugaaua | 9540 |
| auucuuauga augugauauu ccaauuggag caggcauuug ugcaucuuau cagacacaga | 9600 |
| caaauucccc aaggagagca agaucugugg caucucaguc uauuauugca uacaccaugu | 9660 |
| cucugggagc agaaaauucu guggcauauu cuaauaauuc uauugcuauu ccaacaaauu | 9720 |
| uuaccauuuc ugugacaaca gaaauuuuac cugugucuau gacaaaaaca ucuguggauu | 9780 |
| guaccaugua cauuugugga gauucuacag aauguucuaa ucugcugcug caguauggau | 9840 |
| cuuuuuguac acagcugaau agagcuuuaa caggaauugc uguggaacag gauaaaaaua | 9900 |
| cacaggaagu guuugcucag gugaaacaga uuuacaaaac accaccaauu aaagauuuug | 9960 |
| gaggauuuaa uuuuagccag auucugccug auccuucuaa accuucuaaa agaucuuuua | 10020 |
| uugaagaucu gcuguuuaau aaagugacac uggcagaugc aggauuuauu aaacaguaug | 10080 |
| gagauugccu gggugauauu gcugcaagag aucugauuug ugcucagaaa uuuaauggac | 10140 |
| ugacagugcu gccuccucug cugacagaug aaaugauugc ucaguacaca ucugcuuuac | 10200 |
| uggcuggaac aauuacaagc ggauggacau uuggagcugg agcugcucug cagauuccuu | 10260 |
| uugcaaugca gauggcuuac agauuuaaug gaauuggagu gacacagaau guguuauaug | 10320 |
| aaaaucagaa acugauugca aaucaguuua auucugcaau uggcaaaauu caggauucuc | 10380 |
| ugucuucuac agcuucugcu cugggaaaac ugcaggaugu ggugaaucag aaugcacagg | 10440 |
| cacugaauac ucuggugaaa cagcugucua gcaauuuugg ggcaauuucu ucugugcuga | 10500 |
| augauauucu gucuagacug gauccuccug aagcugaagu gcagauugau agacugauca | 10560 |
| caggaagacu gcagucucug cagacuuaug ugacacagca gcugauuaga gcugcugaaa | 10620 |
| uuagagcuuc ugcuaaucug gcugcuacaa aaaugucuga augugugcug ggacagucaa | 10680 |
| aaagagugga uuuuugugga aaaggauauc aucugauguc uuuuccacag ucugcuccac | 10740 |
| auggaguggu guuuuuacau gugacauaug ugccagcaca ggaaaagaau uuuaccacag | 10800 |
| caccagcaau uugucaugau ggaaaagcac auuuuccaag agaaggagug uuugugucua | 10860 |
| auggaacaca uugguuugug acacagagaa auuuuuauga accucagauu auuacaacag | 10920 |
| auaauacauu ugugucagga aauugugaug uggugauugg aauugugaau aauacagugu | 10980 |
| augauccacu gcagccagaa cuggauucuu uuaaagaaga acuggauaaa uauuuuaaaa | 11040 |
| aucacacauc uccugaugug gauuuaggag auauuucugg aaucaaugca ucugugguga | 11100 |
| auauucagaa agaaauugau agacugaaug aaguggccaa aaaucugaau gaaucucuga | 11160 |
| uugaucugca ggaacuugga aaauaugaac aguacauuaa auggccuugg uacauuuggc | 11220 |
| uuggauuuau ugcaggauua auugcaauug ugauggugac aauuauguua uguuguauga | 11280 |
| caucauguug uucuuguuua aaaggauguu guucuugugg aagcuguugu aaauuugaug | 11340 |
| aagaugauuc ugaaccugug uuaaaaggag ugaaauugca uuacacauga ugacucgagc | 11400 |
| ugguacugca ugcacgcaau gcuagcugcc ccuuucccgu ccuggguacc ccgagucucc | 11460 |
| cccgaccucg ggucccaggu augcucccac cuccaccugc cccacucacc accucugcua | 11520 |
| guuccagaca ccucccaagc acgcagcaau gcagcucaaa acgcuuagcc uagccacacc | 11580 |
| cccacgggaa acagcaguga uuaaccuuua gcaauaaacg aaaguuuaac uaagcuauac | 11640 |
| uaaccccagg guuggucaau uucgugccag ccacaccgcg gccgcaugaa uacagcagca | 11700 |
| auuggcaagc ugcuuacaua gaacucgcgg cgauuggcau gccgccuuaa aauuuuuauu | 11760 |
| uuauuuuuuc uuuucuuuuc cgaaucggau uuuguuuuua auauuucaaa aaaaaaaaaa | 11820 |
| aaaaaaaaaa aaaaaaagca uaugacuaaa aaaaaaaaaa aaaaaaaaaa aaaaaaaaaa | 11880 |
| aaaaaaaaaa aaaaaaaaaa aaaaaaaaaa aaaaaaa | 11917 |

RBS004.2 (SEQ ID NO: 34; SEQ ID NO: 9 amino acid)

| | |
|---|---|
| Structure | beta-S-ARCA(D1)-replicase-S1S2-PP-FI-A30L70 |
| Encoded antigen | Viral spike protein (S protein) of the SARS-CoV-2 (S1S2 full-length |
| | protein, sequence variant) |

SEQ ID NO: 34

| | |
|---|---|
| gaugggcggc gcaugagaga agcccagacc aauuaccuac ccaaaaugga gaaaguucac | 60 |
| guugacaucg aggaagacag cccauuccuc agagcuuugc agcggagcuu cccgcaguuu | 120 |
| gagguagaag ccaagcaggu cacugauaau gaccaugcua augccagagc guuuucgcau | 180 |
| cuggcuucaa aacugaucga aacggaggug gacccauccg acacgauccu ugacauugga | 240 |
| agugcgcccg cccgcagaau guauucuaag cacaaguauc auuguaucug uccgaugaga | 300 |
| ugugcggaag auccggacag auuguauaag uaugcaacua agcugaagaa aaacuguaag | 360 |
| gaaauaacug auaaggaauu ggacaagaaa augaaggagc ucgccgccgu caugagcgac | 420 |
| ccugaccugg aaacugagac uaugugccuc cacgacgacg agucgugucg cuacgaaggg | 480 |
| caagucgcug uuuaccagga uguauacgcg guugacggac cgacaagucu cuaucaccaa | 540 |
| gccaauaagg gaguuagagu cgccuacugg auaggcuuug acaccacccc uuuuauguuu | 600 |
| aagaacuugg cuggagcaua uccaucauac ucuaccaacu gggccgacga aaccguguua | 660 |
| acggcucgua acauaggccu augcagcucu gacguuaugg agcggucacg uagagggaug | 720 |
| uccauucuua gaaagaagua uuugaaacca uccaacaaug uucuauucuc uguuggcucg | 780 |
| accaucuacc acgaaaagag ggacuuacug aggagcuggc accugccguc uguauuucac | 840 |
| uuacguggca agcaaaauua cacaugucgg ugugagacua uaguuaguug cgacggguac | 900 |
| gucguuaaaa gaauagcuau caguccaggc cuguauggga agccuucagg cuaugcugcu | 960 |
| acgaugcacc gcgagggauu cuugugcugc aaagugacag acacauugaa cggggagagg | 1020 |
| gucucuuuuc ccgugugcac guaugugcca gcuacauugu gugaccaaau gacuggcaua | 1080 |
| cuggcaacag augucagugc ggacgacgcg caaaaacugc ugguugggcu caaccagcgu | 1140 |
| auagucguca acggucgcac ccagagaaac accaauacca ugaaaaauua ccuuuugccc | 1200 |
| guaguggccc aggcauuugc uaggugggca aaggaauaua aggaagauca agaagaugaa | 1260 |
| aggccacuag gacuacgaga uagacaguua gucauggggu guuguugggc uuuuagaagg | 1320 |
| cacaagauaa caucuauuua uaagcgcccg gauacccaaa ccaucaucaa agugaacagc | 1380 |
| gauuuccacu cauucgugcu gcccaggaua ggcaguaaca cauuggagau cgggcugaga | 1440 |
| acaagaauca ggaaaauguu agaggagcac aaggagccgu caccucucau uaccgccgag | 1500 |
| gacguacaag aagcuaagug cgcagccgau gaggcuaagg aggugcguga agccgaggag | 1560 |
| uugcgcgcag cucuaccacc uuuggcagcu gauguugagg agcccacucu ggaagccgau | 1620 |
| gucgacuuga uguuacaaga ggcuggggcc ggcucagugg agacaccucg uggcuugaua | 1680 |
| aagguuacca gcuacgcugg cgaggacaag aucggcucuu acgcugugcu uucuccgcag | 1740 |
| gcuguacuca agagugaaaa auuaucuugc auccacccuc ucgcugaaca agucauagug | 1800 |
| auaacacacu cuggccgaaa agggcguuau gccguggaac cauaccaugg uaaaguagug | 1860 |
| gugccagagg gacaugcaau acccguccag gacuuucaag cucugaguga aagugccacc | 1920 |
| auuguguaca acgaacguga guucguaaac agguaccugc accauauugc cacacaugga | 1980 |
| ggagcgcuga acacugauga agaauauuac aaaacuguca agcccagcga gcacgacggc | 2040 |
| gaauaccugu acgacaucga caggaaacag ugcgucaaga aagagcuagu cacugggcua | 2100 |
| gggcucacag gcgagcuggu cgauccuccc uuccaugaau ucgccuacga gagucugaga | 2160 |
| acacgaccag ccgcuccuua ccaaguacca accauagggg uguauggcgu gccaggauca | 2220 |
| ggcaagucug gcaucauuaa aagcgcaguc accaaaaaag aucuaguggu gagcgccaag | 2280 |
| aaagaaaacu gugcagaaau uauaagggac gucaagaaaa ugaaagggcu ggacgucaau | 2340 |
| gccagaacug uggacucagu gcucuugaau ggaugcaaac accccguaga gacccuguau | 2400 |
| auugacgagg cuuuugcuug ucaugcaggu acucucagag cgcucauagc cauuauaaga | 2460 |
| ccuaaaaagg cagugcucug cggagauccc aaacagugcg guuuuuuuaa caugaugugc | 2520 |
| cugaaagugc auuuuaacca cgagauuugc acacaagucu uccacaaaag caucucucgc | 2580 |
| cguugcacua aaucugugac uucggucguc ucaaccuugu uuuacgacaa aaaaaugaga | 2640 |
| acgacgaauc cgaaagagac uaagauugug auugacacua ccggcaguac caaaccuaag | 2700 |
| caggacgauc ucauucucac uuguuucaga ggguggguga agcaguugca aauagauuac | 2760 |
| aaaggcaacg aaauaaugac ggcagcugcc ucucaagggc ugacccguaa agguguguau | 2820 |
| gccguucggu acaaggugaa ugaaaauccu cuguacgcac ccaccucaga acaugugaac | 2880 |
| guccuacuga cccgcacgga ggaccgcauc guguggaaaa cacuagccgg cgacccaugg | 2940 |
| auaaaaacac ugacugccaa guacccuggg aauuucacug ccacgauaga ggaguggcaa | 3000 |
| gcagagcaug augccaucau gaggcacauc uuggagagac cggacccuac cgacgucuuc | 3060 |
| cagaauaagg caaacgugug uugggccaag gcuuuagugc cggugcugaa gaccgcuggc | 3120 |
| auagacauga ccacugaaca auggaacacu guggauuauu uugaaacgga caaagcucac | 3180 |
| ucagcagaga uaguauugaa ccaacuaugc gugagguucu uuggacucga ucuggacucc | 3240 |
| ggucuauuuu cugcacccac uguuccguua uccauuagga auaaucacug ggauaacucc | 3300 |
| ccgucgccua acauguacgg gcugaauaaa gaaguggucc gucagcucuc ucgcagguac | 3360 |
| ccacaacugc cucgggcagu ugccacuggu agagucuaug acaugaacac ugguacacug | 3420 |
| cgcaauuaug auccgcgcau aaaccuagua ccuguaaaca gaagacugcc ucaugcuuua | 3480 |
| guccuccacc auaaugaaca cccacagagu gacuuuucuu cauucgucag caaauugaag | 3540 |
| ggcagaacug uccugguggu cggggaaaag uuguccgucc caggcaaaau gguugacugg | 3600 |
| uugucagacc ggccugaggc uaccuucaga gcucggcugg auuuaggcau cccaggugau | 3660 |
| gugcccaaau augacauaau auuuguuaau gugaggaccc cauauaaaua ccaucacuau | 3720 |
| cagcagugug aagaccaugc cauuaagcua agcauguuga ccaagaaagc augucugcau | 3780 |
| cugaaucccg gcggaaccug ugucagcaua gguuaugguu acgcugacag ggccagcgaa | 3840 |
| agcaucauug gugcuauagc gcggcaguuc aaguuuuccc gaguaugcaa accgaaaucc | 3900 |
| ucacuugagg agacggaagu ucuguuugua uucauugggu acgaucgcaa ggcccguacg | 3960 |
| cacaauccuu acaagcuauc aucaaccuug accaacauuu auacagguuc cagacuccac | 4020 |
| gaagccggau gugcacccuc auaucaugug gugcgagggg auauugccac ggccaccgaa | 4080 |
| ggagugauua uaaaugcugc uaacagcaaa ggacaaccug gcggaggggu gugcggagcg | 4140 |
| cuguauaaga aauucccgga aaguuucgau uuacagccga ucgaaguagg aaaagcgcga | 4200 |
| cuggucaaag gugcagcuaa acauaucauu caugccguag gaccaaacuu caacaaaguu | 4260 |
| ucggagguug aaggugacaa acaguuggca gaggcuuaug aguccaucgc uaagauuguc | 4320 |
| aacgauaaca auuacaaguc aguagcgauu ccacuguugu ccaccggcau cuuuuccggg | 4380 |
| aacaaagauc gacuaaccca aucauugaac cauuugcuga cagcuuuaga caccacugau | 4440 |
| gcagauguag ccauauacug cagggacaag aaaugggaaa ugacucucaa ggaagcagug | 4500 |
| gcuaggagag aagcagugga ggagauaugc auauccgacg auucuucagu gacagaaccu | 4560 |
| gaugcagagc uggugagggu gcaucccaag aguucuuugg cuggaaggaa gggcuacagc | 4620 |
| acaagcgaug gcaaaacuuu cucauauuug gaagggacca aguuucacca ggcggccaag | 4680 |
| gauauagcag aaauuaaugc cauguggccc guugcaacgg aggccaauga gcagguaugc | 4740 |
| auguauaucc ucggagaaag caugagcagu auuaggucga aaugccccgu cgaggagucg | 4800 |
| gaagccucca caccaccuag cacgcugccu ugcuugugca uccaugccau gacuccagaa | 4860 |
| agaguacagc gccuaaaagc cucacgucca gaacaaauua cugugugcuc auccuuucca | 4920 |
| uugccgaagu auagaaucac uggugugcag aagauccaau gcucccagcc uauauuguuc | 4980 |
| ucaccgaaag ugccugcgua uauucaucca aggaaguauc ucguggaaac accaccggua | 5040 |
| gacgagacuc cggagccauc ggcagagaac caauccacag aggggacacc ugaacaacca | 5100 |
| ccacuuauaa ccgaggauga gaccaggacu agaacgccug agccgaucau caucgaagaa | 5160 |
| gaagaagaag auagcauaag uuugcuguca gauggcccga cccaccaggu gcugcaaguc | 5220 |
| gaggcagaca uucacgggcc gcccucugua ucuagcucau ccugguccau uccucaugca | 5280 |
| uccgacuuug auguggacag uuuauccaua cuugacaccc uggagggagc uagcgugacc | 5340 |
| agcggggcaa cgucagccga gacuaacucu uacuucgcaa agaguaugga guuucuggcg | 5400 |
| cgaccggugc cugcgccucg aacaguauuc aggaacccuc cacaucccgc uccgcgcaca | 5460 |
| agaacaccgu cacuugcacc cagcagggcc ugcuccagaa ccagccuagu uuccaccccg | 5520 |
| ccaggcguga auagggugau cacuagagag gagcucgaag cgcuuacccc gucacgcacu | 5580 |
| ccuagcaggu cggucuccag aaccagccug gucuccaacc cgccaggcgu aaauagggug | 5640 |
| auuacaagag aggaguuuga ggcguucgua gcacaacaac aaugacgguu ugaugcgggu | 5700 |
| gcauacaucu uuuccuccga caccggucaa gggcauuuac aacaaaaauc aguaaggcaa | 5760 |
| acggugcuau ccgaaguggu guuggagagg accgaauugg agauuucgua ugccccgcgc | 5820 |
| cucgaccaag aaaaagaaga auuacuacgc aagaaauuac aguuaaaucc cacaccugcu | 5880 |
| aacagaagca gauaccaguc caggaaggug gagaacauga aagccauaac agcuagacgu | 5940 |
| auucugcaag gccuagggca uuauuugaag gcagaaggaa aaguggagug cuaccgaacc | 6000 |
| cugcauccug uuccuuugua uucaucuagu gugaaccgug ccuuuucaag ccccaagguc | 6060 |
| gcaguggaag ccuguaacgc cauguugaaa gagaacuuuc cgacuguggc uucuuacugu | 6120 |
| auuauuccag aguacgaugc cuauuuggac augguugacg gagcuucaug cugcuuagac | 6180 |
| acugccaguu uuugcccugc aaagcugcgc agcuuuccaa agaaacacuc cuauuuggaa | 6240 |
| cccacaauac gaucggcagu gccuucagcg auccagaaca cgcuccagaa cguccuggca | 6300 |
| gcugccacaa aaagaaauug caaugucacg caaaugagag aauugcccgu auuggauucg | 6360 |
| gcggccuuua auguggaaug cuucaagaaa uaugcgugua auaaugaaua uugggaaacg | 6420 |
| uuuaaagaaa accccaucag gcuuacugaa gaaaacgugg uaaauuacau uaccaaauua | 6480 |
| aaaggaccaa aagcugcugc ucuuuuugcg aagacacaua auuugaauau guugcaggac | 6540 |
| auaccaaugg acagguuugu aauggacuua aagagagacg ugaaagugac uccaggaaca | 6600 |
| aaacauacug aagaacggcc caagguacag gugauccagg cugccgaucc gcuagcaaca | 6660 |
| gcguaucugu gcggaaucca ccgagagcug guuaggagau uaaaugcggu ccugcuuccg | 6720 |
| aacauucaua cacuguuuga uaugucggcu gaagacuuug acgcuauuau agccgagcac | 6780 |
| uuccagccug gggauugugu ucuggaaacu gacaucgcgu cguuugauaa aagugaggac | 6840 |
| gacgccaugg cucugaccgc guuaaugauu cuggaagacu uaggugugga cgcagagcug | 6900 |
| uugacgcuga uugaggcggc uuucggcgaa auuucaucaa uacauuugcc cacuaaaacu | 6960 |
| aaauuuaaau ucggagccau gaugaaaucu ggaauguucc ucacacuguu ugugaacaca | 7020 |
| gucauuaaca uuguaaucgc aagcagagug uugagagaac ggcuaaccgg aucaccaugu | 7080 |
| gcagcauuca uuggagauga caauaucgug aaaggaguca aaucggacaa auuaauggca | 7140 |
| gacaggugcg ccaccugguu gaauauggaa gucaagauua uagaugcugu ggugggcgag | 7200 |
| aaagcgccuu auuucugugg aggguuuauu uugugugacu ccgugaccgg cacagcgugc | 7260 |
| cguguggcag acccccuaaa aaggcuguuu aagcuaggca aaccucuggc agcagacgau | 7320 |
| gaacaugaug augacaggag aagggcauug caugaggagu caacacgcug gaaccgagug | 7380 |
| gguauucuuu cagagcugug caaggcagua gaaucaaggu augaaaccgu aggaacuucc | 7440 |
| aucauaguua uggccaugac uacucuagcu agcaguguua aaucauucag cuaccugaga | 7500 |
| ggggccccua uaacucucua cggcuaaccu gaauggacua cgacauaguc uaguccgcca | 7560 |
| agacuaguau guucguguuc cuggugcugc ugccucuggu guccagccag ugugugaacc | 7620 |
| ugaccaccag aacacagcug ccuccagccu acaccaacag cuuuaccaga ggcguguacu | 7680 |
| accccgacaa gguguucaga uccagcgugc ugcacucuac ccaggaccug uuccugccuu | 7740 |
| ucuucagcaa cgugaccugg uuccacgcca uccacguguc cggcaccaau ggcaccaaga | 7800 |
| gauucgacaa ccccgugcug cccuucaacg acggggugua cuuugccagc accgagaagu | 7860 |
| ccaacaucau cagaggcugg aucuucggca ccacacugga cagcaagacc cagagccugc | 7920 |
| ugaucgugaa caacgccacc aacgugguca ucaaagugug cgaguuccag uucugcaacg | 7980 |
| accccuuccu gggcgucuac uaccacaaga acaacaagag cuggauggaa agcgaguucc | 8040 |
| ggguguacag cagcgccaac aacugcaccu ucgaguacgu gucccagccu uuccugaugg | 8100 |
| accuggaagg caagcagggc aacuucaaga accugcgcga guucguguuu aagaacaucg | 8160 |
| acggcuacuu caagaucuac agcaagcaca ccccuaucaa ccucgugcgg gaucugccuc | 8220 |
| agggcuucuc ugcucuggaa ccccuggugg aucugcccau cggcaucaac aucacccggu | 8280 |
| uucagacacu gcuggcccug cacagaagcu accugacacc uggcgauagc agcagcggau | 8340 |
| ggacagcugg ugccgccgcu uacuaugugg gcuaccugca gccuagaacc uuccugcuga | 8400 |
| aguacaacga gaacggcacc aucaccgacg ccguggauug ugcucuggau ccucugagcg | 8460 |
| agacaaagug cacccugaag uccuucaccg uggaaaaggg caucuaccag accagcaacu | 8520 |
| uccgggugca gcccaccgaa uccaucgugc gguuccccaa uaucaccaau cugugccccu | 8580 |
| ucggcgaggu guucaaugcc accagauucg ccucugugua cgccuggaac cggaagcgga | 8640 |
| ucagcaauug cguggccgac uacuccgugc uguacaacuc cgccagcuuc agcaccuuca | 8700 |
| agugcuacgg cguguccccu accaagcuga acgaccugug cuucacaaac guguacgccg | 8760 |
| acagcuucgu gauccgggga gaugaagugc ggcagauugc cccuggacag acaggcaaga | 8820 |
| ucgccgacua caacuacaag cugcccgacg acuucaccgg cugugugauu gccuggaaca | 8880 |
| gcaacaaccu ggacuccaaa gucggcggca acuacaauua ccuguaccgg cuguuccgga | 8940 |
| aguccaaucu gaagcccuuc gagcgggaca ucuccaccga gaucuaucag gccggcagca | 9000 |
| ccccuuguaa cggcguggaa ggcuucaacu gcuacuuccc acugcagucc uacggcuuuc | 9060 |
| agcccacaaa uggcgugggc uaucagcccu acagaguggu ggugcugagc uucgaacugc | 9120 |
| ugcaugcccc ugccacagug ugcggcccua agaaaagcac caaucucgug aagaacaaau | 9180 |
| gcgugaacuu caacuucaac ggccugaccg gcaccggcgu gcugacagag agcaacaaga | 9240 |
| aguuccugcc auuccagcag uuuggccggg auaucgccga uaccacagac gccguuagag | 9300 |
| auccccagac acuggaaauc cuggacauca ccccuugcag cuucggcgga gugucuguga | 9360 |
| ucaccccugg caccaacacc agcaaucagg uggcagugcu guaccaggac gugaacugua | 9420 |
| ccgaagugcc cguggccauu cacgccgauc agcugacacc uacauggcgg guguacucca | 9480 |
| ccggcagcaa uguguuucag accagagccg gcugucugau cggagccgag cacgugaaca | 9540 |
| auagcuacga gugcgacauc cccaucggcg cuggaaucug cgccagcuac cagacacaga | 9600 |
| caaacagccc ucggagagcc agaagcgugg ccagccagag caucauugcc uacacaaugu | 9660 |
| cucugggcgc cgagaacagc guggccuacu ccaacaacuc uaucgcuauc cccaccaacu | 9720 |
| ucaccaucag cgugaccaca gagauccugc cuguguccau gaccaagacc agcguggacu | 9780 |
| gcaccaugua caucugcggc gauuccaccg agugcuccaa ccugcugcug caguacggca | 9840 |
| gcuucugcac ccagcugaau agagcccuga cagggaucgc cguggaacag gacaagaaca | 9900 |
| cccaagaggu guucgcccaa gugaagcaga ucuacaagac cccuccuauc aaggacuucg | 9960 |
| gcggcuucaa uuucagccag auucugcccg auccuagcaa gcccagcaag cggagcuuca | 10020 |
| ucgaggaccu gcuguucaac aaagugacac uggccgacgc cggcuucauc aagcaguaug | 10080 |
| gcgauugucu gggcgacauu gccgccaggg aucugauuug cgcccagaag uuuaacggac | 10140 |
| ugacagugcu gccuccucug cugaccgaug agaugaucgc ccaguacaca ucugcccugc | 10200 |
| uggccggcac aaucacaagc ggcuggacau uuggagcagg cgccgcucug cagauccccu | 10260 |
| uugcuaugca gauggccuac cgguucaacg gcaucggagu gacccagaau gugcuguacg | 10320 |
| agaaccagaa gcugaucgcc aaccaguuca acagcgccau cggcaagauc caggacagcc | 10380 |
| ugagcagcac agcaagcgcc cugggaaagc ugcaggacgu ggucaaccag aaugcccagg | 10440 |
| cacugaacac ccuggucaag cagcuguccu ccaacuucgg cgccaucagc ucugugcuga | 10500 |
| acgauauccu gagcagacug gacccuccug aggccgaggu gcagaucgac agacugauca | 10560 |
| caggcagacu gcagagccuc cagacauacg ugacccagca gcugaucaga gccgccgaga | 10620 |
| uuagagccuc ugccaaucug gccgccacca agaugucuga gugugugcug ggccagagca | 10680 |
| agagagugga cuuuugcggc aagggcuacc accugaugag cuucccucag ucugccccuc | 10740 |
| acggcguggu guuucugcac gugacauaug ugcccgcuca agagaagaau uucaccaccg | 10800 |
| cuccagccau cugccacgac ggcaaagccc acuuuccuag agaaggcgug uucgugucca | 10860 |
| acggcaccca uugguucgug acacagcgga acuucuacga gccccagauc aucaccaccg | 10920 |
| acaacaccuu cgugucuggc aacugcgacg ucgugaucgg cauugugaac aauaccgugu | 10980 |
| acgacccucu gcagcccgag cuggacagcu ucaaagagga acuggacaag uacuuuaaga | 11040 |
| accacacaag ccccgacgug gaccugggcg auaucagcgg aaucaaugcc agcgucguga | 11100 |
| acauccagaa agagaucgac cggcugaacg agguggccaa gaaucugaac gagagccuga | 11160 |
| ucgaccugca agaacugggg aaguacgagc aguacaucaa guggcccugg uacaucuggc | 11220 |
| ugggcuuuau cgccggacug auugccaucg ugauggucac aaucaugcug uguugcauga | 11280 |
| ccagcugcug uagcugccug aagggcuguu guagcugugg cagcugcugc aaguucgacg | 11340 |
| aggacgauuc ugagcccgug cugaagggcg ugaaacugca cuacacauga ugacucgagc | 11400 |
| ugguacugca ugcacgcaau gcuagcugcc ccuuucccgu ccuggguacc ccgagucucc | 11460 |
| cccgaccucg ggucccaggu augcucccac cuccaccugc cccacucacc accucugcua | 11520 |
| guuccagaca ccucccaagc acgcagcaau gcagcucaaa acgcuuagcc uagccacacc | 11580 |
| cccacgggaa acagcaguga uuaaccuuua gcaauaaacg aaaguuuaac uaagcuauac | 11640 |
| uaaccccagg guuggucaau uucgugccag ccacaccgcg gccgcaugaa uacagcagca | 11700 |
| auuggcaagc ugcuuacaua gaacucgcgg cgauuggcau gccgccuuaa aauuuuuauu | 11760 |
| uuauuuuuuc uuuucuuuuc cgaaucggau uuuguuuuua auauuucaaa aaaaaaaaaa | 11820 |
| aaaaaaaaaa aaaaaaagca uaugacuaaa aaaaaaaaaa aaaaaaaaaa aaaaaaaaaa | 11880 |
| aaaaaaaaaa aaaaaaaaaa aaaaaaaaaa aaaaaaa | 11917 |

BNT162c1; RBS004.3 (SEQ ID NO: 35; SEQ ID NO: 21 amino acid)

| | |
|---|---|
| Structure | beta-S-ARCA(D1)-replicase-RBD-GS-Fibritin-FI-A30L70 |
| Encoded antigen | Viral spike protein (S protein) of the SARS-CoV-2 (partial sequence, |
| | Receptor Binding Domain (RBD) of S1S2 protein) |

SEQ ID NO: 35

| | |
|---|---|
| gaugggcggc gcaugagaga agcccagacc aauuaccuac ccaaaaugga gaaaguucac | 60 |
| guugacaucg aggaagacag cccauuccuc agagcuuugc agcggagcuu cccgcaguuu | 120 |
| gagguagaag ccaagcaggu cacugauaau gaccaugcua augccagage guuuucgcau | 180 |
| cuggcuucaa aacugaucga aacggaggug gacccauccg acacgauccu ugacauugga | 240 |
| agugcgcccg cccgcagaau guauucuaag cacaaguauc auuguaucug uccgaugaga | 300 |
| ugugeggaag auccggacag auuguauaag uaugcaacua agcugaagaa aaacuguaag | 360 |
| gaaauaacug auaaggaauu ggacaagaaa augaaggage ucgccgccgu caugagcgac | 420 |
| ccugaccugg aaacugagac uaugugccuc cacgacgacg agucgugucg cuacgaaggg | 480 |
| caagucgcug uuuaccagga uguauacgcg guugacggac cgacaagucu cuaucaccaa | 540 |
| gccaauaagg gaguuagagu cgccuacugg auaggcuuug acaccacccc uuuuauguuu | 600 |
| aagaacuugg cuggagcaua uccaucauac ucuaccaacu gggccgacga aaccguguua | 660 |
| acggcucgua acauaggccu augcagcucu gacguuaugg agcggucacg uagagggaug | 720 |
| uccauucuua gaaagaagua uuugaaacca uccaacaaug uucuauucuc uguuggcucg | 780 |
| accaucuacc acgaaaagag ggacuuacug aggagcuggc accugccguc uguauuucac | 840 |
| uuacguggca agcaaaauua cacaugucgg ugugagacua uaguuaguug cgacggguac | 900 |
| gucguuaaaa gaauagcuau caguccaggc cuguauggga agccuucagg cuaugcugcu | 960 |
| acgaugcacc gcgagggauu cuugugcugc aaagugacag acacauugaa cggggagagg | 1020 |
| gucucuuuuc ccgugugcac guaugugcca gcuacauugu gugaccaaau gacuggcaua | 1080 |
| cuggcaacag augucagugc ggacgacgcg caaaaacugc ugguugggcu caaccagcgu | 1140 |
| auagucguca acggucgcac ccagagaaac accaauacca ugaaaaauua ccuuuugccc | 1200 |
| guaguggccc aggcauuugc uaggugggca aaggaauaua aggaagauca agaagaugaa | 1260 |
| aggccacuag gacuacgaga uagacaguua gucauggggu guuguugggc uuuuagaagg | 1320 |
| cacaagauaa caucuauuua uaagcgcccg gauacccaaa ccaucaucaa agugaacagc | 1380 |
| gauuuccacu cauucgugcu gcccaggaua ggcaguaaca cauuggagau cgggcugaga | 1440 |
| acaagaauca ggaaaauguu agaggagcac aaggagccgu caccucucau uaccgccgag | 1500 |
| gacguacaag aagcuaagug cgcagccgau gaggcuaagg aggugcguga agccgaggag | 1560 |
| uugcgcgcag cucuaccacc uuuggcagcu gauguugagg agcccacucu ggaagccgau | 1620 |
| gucgacuuga uguuacaaga ggcuggggcc ggcucagugg agacaccucg uggcuugaua | 1680 |
| aagguuacca gcuacgcugg cgaggacaag aucggcucuu acgcugugcu uucuccgcag | 1740 |
| gcuguacuca agagugaaaa auuaucuugc auccacccuc ucgcugaaca agucauagug | 1800 |
| auaacacacu cuggccgaaa agggcguuau gccguggaac cauaccaugg uaaaguagug | 1860 |
| gugccagagg gacaugcaau acccguccag gacuuucaag cucugaguga aagugccacc | 1920 |
| auuguguaca acgaacguga guucguaaac agguaccugc accauauugc cacacaugga | 1980 |
| ggagcgcuga acacugauga agaauauuac aaaacuguca agcccagcga gcacgacggc | 2040 |
| gaauaccugu acgacaucga caggaaacag ugcgucaaga aagagcuagu cacugggcua | 2100 |
| gggcucacag gcgagcuggu cgauccuccc uuccaugaau ucgccuacga gagucugaga | 2160 |
| acacgaccag ccgcuccuua ccaaguacca accauagggg uguauggcgu gccaggauca | 2220 |
| ggcaagucug gcaucauuaa aagcgcaguc accaaaaaag aucuaguggu gagcgccaag | 2280 |
| aaagaaaacu gugcagaaau uauaagggac gucaagaaaa ugaaagggcu ggacgucaau | 2340 |
| gccagaacug uggacucagu gcucuugaau ggaugcaaac accccguaga gacccuguau | 2400 |
| auugacgagg cuuuugcuug ucaugcaggu acucucagag cgcucauagc cauuauaaga | 2460 |
| ccuaaaaagg cagugcucug cggagauccc aaacagugcg guuuuuuuaa caugaugugc | 2520 |
| cugaaagugc auuuuaacca cgagauuugc acacaagucu uccacaaaag caucucucgc | 2580 |
| cguugcacua aaucugugac uucggucguc ucaaccuugu uuuacgacaa aaaaaugaga | 2640 |
| acgacgaauc cgaaagagac uaagauugug auugacacua ccggcaguac caaaccuaag | 2700 |
| caggacgauc ucauucucac uuguuucaga ggguggguga agcaguugca aauagauuac | 2760 |
| aaaggcaacg aaauaaugac ggcagcugcc ucucaagggc ugacccguaa agguguguau | 2820 |
| gccguucggu acaaggugaa ugaaaauccu cuguacgcac ccaccucaga acaugugaac | 2880 |
| guccuacuga cccgcacgga ggaccgcauc guguggaaaa cacuagccgg cgacccaugg | 2940 |
| auaaaaacac ugacugccaa guacccuggg aauuucacug ccacgauaga ggaguggcaa | 3000 |
| gcagagcaug augccaucau gaggcacauc uuggagagac cggacccuac cgacgucuuc | 3060 |
| cagaauaagg caaacgugug uugggccaag gcuuuagugc cggugcugaa gaccgcuggc | 3120 |
| auagacauga ccacugaaca auggaacacu guggauuauu uugaaacgga caaagcucac | 3180 |
| ucagcagaga uaguauugaa ccaacuaugc gugagguucu uuggacucga ucuggacucc | 3240 |
| ggucuauuuu cugcacccac uguuccguua uccauuagga auaaucacug ggauaacucc | 3300 |
| ccgucgccua acauguacgg gcugaauaaa gaaguggucc gucagcucuc ucgcagguac | 3360 |
| ccacaacugc cucgggcagu ugccacuggu agagucuaug acaugaacac ugguacacug | 3420 |
| cgcaauuaug auccgcgcau aaaccuagua ccuguaaaca gaagacugcc ucaugcuuua | 3480 |
| guccuccacc auaaugaaca cccacagagu gacuuuucuu cauucgucag caaauugaag | 3540 |
| ggcagaacug uccugguggu cggggaaaag uuguccgucc caggcaaaau gguugacugg | 3600 |
| uugucagacc ggccugaggc uaccuucaga gcucggcugg auuuaggcau cccaggugau | 3660 |
| gugcccaaau augacauaau auuuguuaau gugaggaccc cauauaaaua ccaucacuau | 3720 |
| cagcagugug aagaccaugc cauuaagcua agcauguuga ccaagaaagc augucugcau | 3780 |
| cugaaucccg geggaaccug ugucagcaua gguuaugguu acgcugacag ggccagcgaa | 3840 |
| agcaucauug gugcuauagc gcggcaguuc aaguuuuccc gaguaugcaa accgaaaucc | 3900 |
| ucacuugagg agacggaagu ucuguuugua uucauugggu acgaucgcaa ggcccguacg | 3960 |
| cacaauccuu acaagcuauc aucaaccuug accaacauuu auacagguuc cagacuccac | 4020 |
| gaagccggau gugcacccuc auaucaugug gugcgagggg auauugccac ggccaccgaa | 4080 |
| ggagugauua uaaaugcugc uaacagcaaa ggacaaccug gcggaggggu gugcggagcg | 4140 |
| cuguauaaga aauucccgga aaguuucgau uuacagccga ucgaaguagg aaaagcgcga | 4200 |
| cuggucaaag gugcagcuaa acauaucauu caugccguag gaccaaacuu caacaaaguu | 4260 |
| ucggagguug aaggugacaa acaguuggca gaggcuuaug aguccaucgc uaagauuguc | 4320 |
| aacgauaaca auuacaaguc aguagcgauu ccacuguugu ccaccggcau cuuuuccggg | 4380 |
| aacaaagauc gacuaaccca aucauugaac cauuugcuga cagcuuuaga caccacugau | 4440 |
| gcagauguag ccauauacug cagggacaag aaaugggaaa ugacucucaa ggaagcagug | 4500 |
| gcuaggagag aagcagugga ggagauaugc auauccgacg auucuucagu gacagaaccu | 4560 |
| gaugcagagc uggugagggu gcaucccaag aguucuuugg cuggaaggaa gggcuacagc | 4620 |
| acaagcgaug gcaaaacuuu cucauauuug gaagggacca aguuucacca ggcggccaag | 4680 |
| gauauagcag aaauuaaugc cauguggccc guugcaacgg aggccaauga gcagguaugc | 4740 |
| auguauaucc ucggagaaag caugagcagu auuaggucga aaugccccgu cgaggagucg | 4800 |
| gaagccucca caccaccuag cacgcugccu ugcuugugca uccaugccau gacuccagaa | 4860 |
| agaguacagc gccuaaaagc cucacgucca gaacaaauua cugugugcuc auccuuucca | 4920 |
| uugccgaagu auagaaucac uggugugcag aagauccaau gcucccagcc uauauuguuc | 4980 |
| ucaccgaaag ugccugcgua uauucaucca aggaaguauc ucguggaaac accaccggua | 5040 |
| gacgagacuc cggagccauc ggcagagaac caauccacag aggggacacc ugaacaacca | 5100 |
| ccacuuauaa ccgaggauga gaccaggacu agaacgccug agccgaucau caucgaagaa | 5160 |
| gaagaagaag auagcauaag uuugcuguca gauggcccga cccaccaggu gcugcaaguc | 5220 |
| gaggcagaca uucacgggcc gcccucugua ucuagcucau ccugguccau uccucaugca | 5280 |
| uccgacuuug auguggacag uuuauccaua cuugacaccc uggagggagc uagcgugacc | 5340 |
| agcggggcaa cgucagccga gacuaacucu uacuucgcaa agaguaugga guuucuggcg | 5400 |
| cgaccggugc cugcgccucg aacaguauuc aggaacccuc cacaucccgc uccgcgcaca | 5460 |
| agaacaccgu cacuugcacc cagcagggcc ugcuccagaa ccagccuagu uuccaccccg | 5520 |
| ccaggcguga auagggugau cacuagagag gagcucgaag cgcuuacccc gucacgcacu | 5580 |
| ccuagcaggu cggucuccag aaccagccug gucuccaacc cgccaggcgu aaauagggug | 5640 |
| auuacaagag aggaguuuga ggcguucgua gcacaacaac aaugacgguu ugaugcgggu | 5700 |
| gcauacaucu uuuccuccga caccggucaa gggcauuuac aacaaaaauc aguaaggcaa | 5760 |
| acggugcuau ccgaaguggu guuggagagg accgaauugg agauuucgua ugccccgcgc | 5820 |
| cucgaccaag aaaaagaaga auuacuacgc aagaaauuac aguuaaaucc cacaccugcu | 5880 |
| aacagaagca gauaccaguc caggaaggug gagaacauga aagccauaac agcuagacgu | 5940 |
| auucugcaag gccuagggca uuauuugaag gcagaaggaa aaguggagug cuaccgaacc | 6000 |
| cugcauccug uuccuuugua uucaucuagu gugaaccgug ccuuuucaag ccccaagguc | 6060 |
| gcaguggaag ccuguaacgc cauguugaaa gagaacuuuc cgacuguggc uucuuacugu | 6120 |
| auuauuccag aguacgauge cuauuuggac augguugacg gagcuucaug cugcuuagac | 6180 |
| acugccaguu uuugcccugc aaagcugcgc agcuuuccaa agaaacacuc cuauuuggaa | 6240 |
| cccacaauac gaucggcagu gccuucagcg auccagaaca cgcuccagaa cguccuggca | 6300 |
| gcugccacaa aaagaaauug caaugucacg caaaugagag aauugcccgc auuggauucg | 6360 |
| gcggccuuua auguggaaug cuucaagaaa uaugcgugua auaaugaaua uugggaaacg | 6420 |
| uuuaaagaaa accccaucag gcuuacugaa gaaaacgugg uaaauuacau uaccaaauua | 6480 |
| aaaggaccaa aagcugcugc ucuuuuugcg aagacacaua auuugaauau guugcaggac | 6540 |
| auaccaaugg acagguuugu aauggacuua aagagagacg ugaaagugac uccaggaaca | 6600 |
| aaacauacug aagaacggcc caagguacag gugauccagg cugccgaucc gcuagcaaca | 6660 |
| gcguaucugu geggaaucca ccgagagcug guuaggagau uaaaugcggu ccugcuuccg | 6720 |
| aacauucaua cacuguuuga uaugucggcu gaagacuuug acgcuauuau agccgagcac | 6780 |
| uuccagccug gggauugugu ucuggaaacu gacaucgcgu cguuugauaa aagugaggac | 6840 |
| gacgccaugg cucugaccgc guuaaugauu cuggaagacu uaggugugga cgcagagcug | 6900 |
| uugacgcuga uugaggcggc uuucggcgaa auuucaucaa uacauuugcc cacuaaaacu | 6960 |
| aaauuuaaau ucggagccau gaugaaaucu ggaauguucc ucacacuguu ugugaacaca | 7020 |
| gucauuaaca uuguaaucgc aagcagagug uugagagaac ggcuaaccgg aucaccaugu | 7080 |
| gcagcauuca uuggagauga caauaucgug aaaggaguca aaucggacaa auuaauggca | 7140 |
| gacaggugcg ccaccugguu gaauauggaa gucaagauua uagaugcugu ggugggcgag | 7200 |
| aaagcgccuu auuucugugg aggguuuauu uugugugacu ccgugaccgg cacagcgugc | 7260 |
| cguguggcag acccccuaaa aaggcuguuu aagcuaggca aaccucuggc agcagacgau | 7320 |
| gaacaugaug augacaggag aagggcauug caugaggagu caacacgcug gaaccgagug | 7380 |
| gguauucuuu cagagcugug caaggcagua gaaucaaggu augaaaccgu aggaacuucc | 7440 |
| aucauaguua uggccaugac uacucuagcu agcaguguua aaucauucag cuaccugaga | 7500 |
| ggggccccua uaacucucua cggcuaaccu gaauggacua cgacauaguc uaguccgcca | 7560 |
| agacuaguau guuuguguuu cuugugcugc ugccucuugu gucuucucag ugugugguga | 7620 |
| gauuuccaaa uauuacaaau cuguguccau uuggagaagu guuuaaugca acaagauuug | 7680 |
| caucugugua ugcauggaau agaaaaagaa uuucuaauug uguggcugau uauucugugc | 7740 |
| uguauaauag ugcuucuuuu uccacauuua aauguuaugg agugucucca acaaaauuaa | 7800 |
| augauuuaug uuuuacaaau guguaugcug auucuuuugu gaucagaggu gaugaaguga | 7860 |
| gacagauugc ccccggacag acaggaaaaa uugcugauua caauuacaaa cugccugaug | 7920 |
| auuuuacagg augugugauu gcuuggaauu cuaauaauuu agauucuaaa gugggaggaa | 7980 |
| auuacaauua ucuguacaga cuguuuagaa aaucaaaucu gaaaccuuuu gaaagagaua | 8040 |
| uuucaacaga aauuuaucag gcuggaucaa caccuuguaa uggaguggaa ggauuuaauu | 8100 |
| guuauuuucc auuacagage uauggauuuc agccaaccaa ugguguggga uaucagccau | 8160 |
| auagaguggu ggugcugucu uuugaacugc ugcaugcacc ugcaacagug uguggaccua | 8220 |
| aaggcucccc cggcuccggc uccggaucug guuauauucc ugaagcucca agagaugggc | 8280 |
| aagcuuacgu ucguaaagau ggcgaauggg uauuacuuuc uaccuuuuua ggccgguccc | 8340 |
| uggaggugcu guuccagggc cccggcugau gacucgagcu gguacugcau gcacgcaaug | 8400 |
| cuagcugccc cuuucccguc cuggguaccc cgagucuccc ccgaccucgg gucccaggua | 8460 |
| ugcucccacc uccaccugcc ccacucacca ccucugcuag uuccagacac cucccaagca | 8520 |
| cgcagcaaug cagcucaaaa cgcuuagccu agccacaccc ccacgggaaa cagcagugau | 8580 |
| uaaccuuuag caauaaacga aaguuuaacu aagcuauacu aaccccaggg uuggucaauu | 8640 |
| ucgugccagc cacaccgcgg ccgcaugaau acagcagcaa uuggcaagcu gcuuacauag | 8700 |
| aacucgcggc gauuggcaug ccgccuuaaa auuuuuauuu uauuuuuucu aaaaaagcau | 8760 |
| gaaucggauu uuguuuuuaa uauuucaaaa aaaaaaaaaa aaaaaaaaaa aaaaaagcau | 8820 |
| augacuaaaa aaaaaaaaaa aaaaaaaaaa aaaaaaaaaa aaaaaaaaaa aaaaaaaaaa | 8880 |
| aaaaaaaaaa aaaaaa | 8896 |

RBS004.4 (SEQ ID NO: 36; SEQ ID NO: 37)

| | |
|---|---|
| Structure | beta-S-ARCA(D1)-replicase-RBD-GS-Fibritin-TM-FI-A30L70 |
| Encoded antigen | Viral spike protein (S protein) of the SARS-CoV-2 (partial sequence, |
| | Receptor Binding Domain (RBD) of S1S2 protein) |

SEQ ID NO: 36

| | |
|---|---|
| gaugggcggc gcaugagaga agcccagacc aauuaccuac ccaaaaugga gaaaguucac | 60 |
| guugacaucg aggaagacag cccauuccuc agagcuuugc agcggagcuu cccgcaguuu | 120 |
| gagguagaag ccaagcaggu cacugauaau gaccaugcua augccagagc guuuucgcau | 180 |
| cuggcuucaa aacugaucga aacggaggug gacccauccg acacgauccu ugacauugga | 240 |
| agugcgcccg cccgcagaau guauucuaag cacaaguauc auuguaucug uccgaugaga | 300 |
| ugugcggaag auccggacag auuguauaag uaugcaacua agcugaagaa aaacuguaag | 360 |
| gaaauaacug auaaggaauu ggacaagaaa augaaggagc ucgccgccgu caugagcgac | 420 |
| ccugaccugg aaacugagac uaugugccuc cacgacgacg agucgugucg cuacgaaggg | 480 |
| caagucgcug uuuaccagga uguauacgcg guugacggac cgacaagucu cuaucaccaa | 540 |
| gccaauaagg gaguuagagu cgccuacugg auaggcuuug acaccacccc uuuuauguuu | 600 |
| aagaacuugg cuggagcaua uccaucauac ucuaccaacu gggccgacga aaccguguua | 660 |
| acggcucgua acauaggccu augcagcucu gacguuaugg agcggucacg uagagggaug | 720 |
| uccauucuua gaaagaagua uuugaaacca uccaacaaug uucuauucuc uguuggcucg | 780 |
| accaucuacc acgaaaagag ggacuuacug aggagcuggc accugccguc uguauuucac | 840 |
| uuacguggca agcaaaauua cacaugucgg ugugagacua uaguuaguug cgacggguac | 900 |
| gucguuaaaa gaauagcuau caguccaggc cuguauggga agccuucagg cuaugcugcu | 960 |
| acgaugcacc gcgagggauu cuugugcugc aaagugacag acacauugaa cggggagagg | 1020 |
| gucucuuuuc ccgugugcac guaugugcca gcuacauugu gugaccaaau gacuggcaua | 1080 |
| cuggcaacag augucagugc ggacgacgcg caaaaacugc ugguugggcu caaccagcgu | 1140 |
| auagucguca acggucgcac ccagagaaac accaauacca ugaaaaauua ccuuuugccc | 1200 |
| guaguggccc aggcauuugc uaggugggca aaggaauaua aggaagauca agaagaugaa | 1260 |
| aggccacuag gacuacgaga uagacaguua gucauggggu guuguugggc uuuuagaagg | 1320 |
| cacaagauaa caucuauuua uaagcgcccg gauacccaaa ccaucaucaa agugaacagc | 1380 |
| gauuuccacu cauucgugcu gcccaggaua ggcaguaaca cauuggagau cgggcugaga | 1440 |
| acaagaauca ggaaaauguu agaggagcac aaggagccgu caccucucau uaccgccgag | 1500 |
| gacguacaag aagcuaagug cgcagccgau gaggcuaagg aggugcguga agccgaggag | 1560 |
| uugcgcgcag cucuaccacc uuuggcagcu gauguugagg agcccacucu ggaagccgau | 1620 |
| gucgacuuga uguuacaaga ggcuggggcc ggcucagugg agacaccucg uggcuugaua | 1680 |
| aagguuacca gcuacgcugg cgaggacaag aucggcucuu acgcugugcu uucuccgcag | 1740 |
| gcuguacuca agagugaaaa auuaucuugc auccacccuc ucgcugaaca agucauagug | 1800 |
| auaacacacu cuggccgaaa agggcguuau gccguggaac cauaccaugg uaaaguagug | 1860 |
| gugccagagg gacaugcaau acccguccag gacuuucaag cucugaguga aagugccacc | 1920 |
| auuguguaca acgaacguga guucguaaac agguaccugc accauauugc cacacaugga | 1980 |
| ggagcgcuga acacugauga agaauauuac aaaacuguca agcccagcga gcacgacggc | 2040 |
| gaauaccugu acgacaucga caggaaacag ugcgucaaga aagagcuagu cacugggcua | 2100 |
| gggcucacag gcgagcuggu cgauccuccc uuccaugaau ucgccuacga gagucugaga | 2160 |
| acacgaccag ccgcuccuua ccaaguacca accauagggg uguauggcgu gccaggauca | 2220 |
| ggcaagucug gcaucauuaa aagcgcaguc accaaaaaag aucuaguggu gagcgccaag | 2280 |
| aaagaaaacu gugcagaaau uauaagggac gucaagaaaa ugaaagggcu ggacgucaau | 2340 |
| gccagaacug uggacucagu gcucuugaau ggaugcaaac accccguaga gacccuguau | 2400 |
| auugacgagg cuuuugcuug ucaugcaggu acucucagag cgcucauagc cauuauaaga | 2460 |
| ccuaaaaagg cagugcucug cggagauccc aaacagugcg guuuuuuuaa caugaugugc | 2520 |
| cugaaagugc auuuuaacca cgagauuugc acacaagucu uccacaaaag caucucucgc | 2580 |
| cguugcacua aaucugugac uucggucguc ucaaccuugu uuuacgacaa aaaaaugaga | 2640 |
| acgacgaauc cgaaagagac uaagauugug auugacacua ccggcaguac caaaccuaag | 2700 |
| caggacgauc ucauucucac uuguuucaga ggguggguga agcaguugca aauagauuac | 2760 |
| aaaggcaacg aaauaaugac ggcagcugcc ucucaagggc ugacccguaa agguguguau | 2820 |
| gccguucggu acaaggugaa ugaaaauccu cuguacgcac ccaccucaga acaugugaac | 2880 |
| guccuacuga cccgcacgga ggaccgcauc guguggaaaa cacuagccgg cgacccaugg | 2940 |
| auaaaaacac ugacugccaa guacccuggg aauuucacug ccacgauaga ggaguggcaa | 3000 |
| gcagagcaug augccaucau gaggcacauc uuggagagac cggacccuac cgacgucuuc | 3060 |
| cagaauaagg caaacgugug uugggccaag gcuuuagugc cggugcugaa gaccgcuggc | 3120 |
| auagacauga ccacugaaca auggaacacu guggauuauu uugaaacgga caaagcucac | 3180 |
| ucagcagaga uaguauugaa ccaacuaugc gugagguucu uuggacucga ucuggacucc | 3240 |
| ggucuauuuu cugcacccac uguuccguua uccauuagga auaaucacug ggauaacucc | 3300 |
| ccgucgccua acauguacgg gcugaauaaa gaaguggucc gucagcucuc ucgcagguac | 3360 |
| ccacaacugc cucgggcagu ugccacuggu agagucuaug acaugaacac ugguacacug | 3420 |
| cgcaauuaug auccgcgcau aaaccuagua ccuguaaaca gaagacugcc ucaugcuuua | 3480 |
| guccuccacc auaaugaaca cccacagagu gacuuuucuu cauucgucag caaauugaag | 3540 |
| ggcagaacug uccugguggu cggggaaaag uuguccgucc caggcaaaau gguugacugg | 3600 |
| uugucagacc ggccugaggc uaccuucaga gcucggcugg auuuaggcau cccaggugau | 3660 |
| gugcccaaau augacauaau auuuguuaau gugaggaccc cauauaaaua ccaucacuau | 3720 |
| cagcagugug aagaccaugc cauuaagcua agcauguuga ccaagaaagc augucugcau | 3780 |
| cugaaucccg gcggaaccug ugucagcaua gguuaugguu acgcugacag ggccagcgaa | 3840 |
| agcaucauug gugcuauagc gcggcaguuc aaguuuuccc gaguaugcaa accgaaaucc | 3900 |
| ucacuugagg agacggaagu ucuguuugua uucauugggu acgaucgcaa ggcccguacg | 3960 |
| cacaauccuu acaagcuauc aucaaccuug accaacauuu auacagguuc cagacuccac | 4020 |
| gaagccggau gugcacccuc auaucaugug gugcgagggg auauugccac ggccaccgaa | 4080 |
| ggagugauua uaaaugcugc uaacagcaaa ggacaaccug gcggaggggu gugcggagcg | 4140 |
| cuguauaaga aauucccgga aaguuucgau uuacagccga ucgaaguagg aaaagcgcga | 4200 |
| cuggucaaag gugcagcuaa acauaucauu caugccguag gaccaaacuu caacaaaguu | 4260 |
| ucggagguug aaggugacaa acaguuggca gaggcuuaug aguccaucgc uaagauuguc | 4320 |
| aacgauaaca auuacaaguc aguagcgauu ccacuguugu ccaccggcau cuuuuccggg | 4380 |
| aacaaagauc gacuaaccca aucauugaac cauuugcuga cagcuuuaga caccacugau | 4440 |
| gcagauguag ccauauacug cagggacaag aaaugggaaa ugacucucaa ggaagcagug | 4500 |
| gcuaggagag aagcagugga ggagauaugc auauccgacg auucuucagu gacagaaccu | 4560 |
| gaugcagagc uggugagggu gcaucccaag aguucuuugg cuggaaggaa gggcuacagc | 4620 |
| acaagcgaug gcaaaacuuu cucauauuug gaagggacca aguuucacca ggcggccaag | 4680 |
| gauauagcag aaauuaaugc cauguggccc guugcaacgg aggccaauga gcagguaugc | 4740 |
| auguauaucc ucggagaaag caugagcagu auuaggucga aaugccccgu cgaggagucg | 4800 |
| gaagccucca caccaccuag cacgcugccu ugcuugugca uccaugccau gacuccagaa | 4860 |
| agaguacagc gccuaaaagc cucacgucca gaacaaauua cugugugcuc auccuuucca | 4920 |
| uugccgaagu auagaaucac uggugugcag aagauccaau gcucccagcc uauauuguuc | 4980 |
| ucaccgaaag ugccugcgua uauucaucca aggaaguauc ucguggaaac accaccggua | 5040 |
| gacgagacuc cggagccauc ggcagagaac caauccacag aggggacacc ugaacaacca | 5100 |
| ccacuuauaa ccgaggauga gaccaggacu agaacgccug agccgaucau caucgaagaa | 5160 |
| gaagaagaag auagcauaag uuugcuguca gauggcccga cccaccaggu gcugcaaguc | 5220 |
| gaggcagaca uucacgggcc gcccucugua ucuagcucau ccugguccau uccucaugca | 5280 |
| uccgacuuug auguggacag uuuauccaua cuugacaccc uggagggagc uagcgugacc | 5340 |
| agcggggcaa cgucagccga gacuaacucu uacuucgcaa agaguaugga guuucuggcg | 5400 |
| cgaccggugc cugcgccucg aacaguauuc aggaacccuc cacaucccgc uccgcgcaca | 5460 |
| agaacaccgu cacuugcacc cagcagggcc ugcuccagaa ccagccuagu uuccaccccg | 5520 |
| ccaggcguga auagggugau cacuagagag gagcucgaag cgcuuacccc gucacgcacu | 5580 |
| ccuagcaggu cggucuccag aaccagccug gucuccaacc cgccaggcgu aaauagggug | 5640 |
| auuacaagag aggaguuuga ggcguucgua gcacaacaac aaugacgguu ugaugcgggu | 5700 |
| gcauacaucu uuuccuccga caccggucaa gggcauuuac aacaaaaauc aguaaggcaa | 5760 |
| acggugcuau ccgaaguggu guuggagagg accgaauugg agauuucgua ugccccgcgc | 5820 |
| cucgaccaag aaaaagaaga auuacuacgc aagaaauuac aguuaaaucc cacaccugcu | 5880 |
| aacagaagca gauaccaguc caggaaggug gagaacauga aagccauaac agcuagacgu | 5940 |
| auucugcaag gccuagggca uuauuugaag gcagaaggaa aaguggagug cuaccgaacc | 6000 |
| cugcauccug uuccuuugua uucaucuagu gugaaccgug ccuuuucaag ccccaagguc | 6060 |
| gcaguggaag ccuguaacgc cauguugaaa gagaacuuuc cgacuguggc uucuuacugu | 6120 |
| auuauuccag aguacgaugc cuauuuggac augguugacg gagcuucaug cugcuuagac | 6180 |
| acugccaguu uuugcccugc aaagcugcgc agcuuuccaa agaaacacuc cuauuuggaa | 6240 |
| cccacaauac gaucggcagu gccuucagcg auccagaaca cgcuccagaa cguccuggca | 6300 |
| gcugccacaa aaagaaauug caaugucacg caaaugagag aauugcccgu auuggauucg | 6360 |
| gcggccuuua auguggaaug cuucaagaaa uaugcgugua auaaugaaua uugggaaacg | 6420 |
| uuuaaagaaa accccaucag gcuuacugaa gaaaacgugg uaaauuacau uaccaaauua | 6480 |
| aaaggaccaa aagcugcugc ucuuuuugcg aagacacaua auuugaauau guugcaggac | 6540 |
| auaccaaugg acagguuugu aauggacuua aagagagacg ugaaagugac uccaggaaca | 6600 |
| aaacauacug aagaacggcc caagguacag gugauccagg cugccgaucc gcuagcaaca | 6660 |
| gcguaucugu gcggaaucca ccgagagcug guuaggagau uaaaugcggu ccugcuuccg | 6720 |
| aacauucaua cacuguuuga uaugucggcu gaagacuuug acgcuauuau agccgagcac | 6780 |
| uuccagccug gggauugugu ucuggaaacu gacaucgcgu cguuugauaa aagugaggac | 6840 |
| gacgccaugg cucugaccgc guuaaugauu cuggaagacu uaggugugga cgcagagcug | 6900 |
| uugacgcuga uugaggcggc uuucggcgaa auuucaucaa uacauuugcc cacuaaaacu | 6960 |
| aaauuuaaau ucggagccau gaugaaaucu ggaauguucc ucacacuguu ugugaacaca | 7020 |
| gucauuaaca uuguaaucgc aagcagagug uugagagaac ggcuaaccgg aucaccaugu | 7080 |
| gcagcauuca uuggagauga caauaucgug aaaggaguca aaucggacaa auuaauggca | 7140 |
| gacaggugcg ccaccugguu gaauauggaa gucaagauua uagaugcugu ggugggcgag | 7200 |
| aaagcgccuu auuucugugg aggguuuauu uugugugacu ccgugaccgg cacagcgugc | 7260 |
| cguguggcag acccccuaaa aaggcuguuu aagcuaggca aaccucuggc agcagacgau | 7320 |
| gaacaugaug augacaggag aagggcauug caugaggagu caacacgcug gaaccgagug | 7380 |
| gguauucuuu cagagcugug caaggcagua gaaucaaggu augaaaccgu aggaacuucc | 7440 |
| aucauaguua uggccaugac uacucuagcu agcaguguua aaucauucag cuaccugaga | 7500 |
| ggggccccua uaacucucua cggcuaaccu gaauggacua cgacauaguc uaguccgcca | 7560 |
| agacuaguau guuuguguuu cuugugcugc ugccucuugu gucuucucag ugugugguga | 7620 |
| gauuuccaaa uauuacaaau cuguguccau uuggagaagu guuuaaugca acaagauuug | 7680 |
| caucugugua ugcauggaau agaaaaagaa uuucuaauug uguggcugau uauucugugc | 7740 |
| uguauaauag ugcuucuuuu uccacauuua aauguuaugg agugucucca acaaaauuaa | 7800 |
| augauuuaug uuuuacaaau guguaugcug auucuuuugu gaucagaggu gaugaaguga | 7860 |
| gacagauugc ccccggacag acaggaaaaa uugcugauua caauuacaaa cugccugaug | 7920 |
| auuuuacagg augugugauu gcuuggaauu cuaauaauuu agauucuaaa gugggaggaa | 7980 |
| auuacaauua ucuguacaga cuguuuagaa aaucaaaucu gaaaccuuuu gaaagagaua | 8040 |
| uuucaacaga aauuuaucag gcuggaucaa caccuuguaa uggaguggaa ggauuuaauu | 8100 |
| guuauuuucc auuacagagc uauggauuuc agccaaccaa ugguguggga uaucagccau | 8160 |
| auagaguggu ggugcugucu uuugaacugc ugcaugcacc ugcaacagug uguggaccua | 8220 |
| aaggcucccc cggcuccggc uccggaucug guuauauucc ugaagcucca agagaugggc | 8280 |
| aagcuuacgu ucguaaagau ggcgaauggg uauuacuuuc uaccuuuuua ggaagcggca | 8340 |
| gcggaucuga acaguacauu aaauggccuu gguacauuug gcuuggauuu auugcaggau | 8400 |
| uaauugcaau ugugauggug acaauuaugu uauguuguau gacaucaugu uguucuuguu | 8460 |
| uaaaaggaug uuguucuugu ggaagcuguu guaaauuuga ugaagaugau ucugaaccug | 8520 |
| uguuaaaagg agugaaauug cauuacacau gaugacucga gcugguacug caugcacgca | 8580 |
| augcuagcug ccccuuuccc guccugggua ccccgagucu cccccgaccu cgggucccag | 8640 |
| guaugcuccc accuccaccu gccccacuca ccaccucugc uaguuccaga caccucccaa | 8700 |
| gcacgcagca augcagcuca aaacgcuuag ccuagccaca cccccacggg aaacagcagu | 8760 |
| gauuaaccuu uagcaauaaa cgaaaguuua acuaagcuau acuaacccca ggguugguca | 8820 |
| auuucgugcc agccacaccg cggccgcaug aauacagcag caauuggcaa gcugcuuaca | 8880 |
| uagaacucgc ggcgauuggc augccgccuu aaaauuuuua uuuuauuuuu ucuuuucuuu | 8940 |
| uccgaaucgg auuuuguuuu uaauauuuca aaaaaaaaaa aaaaaaaaaa aaaaaaaaag | 9000 |
| cauaugacua aaaaaaaaaa aaaaaaaaaa aaaaaaaaaa aaaaaaaaaa aaaaaaaaaa | 9060 |
| aaaaaaaaaa aaaaaaaaa | 9079 |

```
SEQ ID NO: 37
Met Phe Val Phe Leu Val Leu Leu Pro Leu Val Ser Ser Gln Cys Val
1 5 10 15
Val Arg Phe Pro Asn Ile Thr Asn Leu Cys Pro Phe Gly Glu Val Phe
                        20 25 30
Asn Ala Thr Arg Phe Ala Ser Val Tyr Ala Trp Asn Arg Lys Arg Ile
                35 40 45
Ser Asn Cys Val Ala Asp Tyr Ser Val Leu Tyr Asn Ser Ala Ser Phe
        50 55 60
Ser Thr Phe Lys Cys Tyr Gly Val Ser Pro Thr Lys Leu Asn Asp Leu
65 70 75 80
Cys Phe Thr Asn Val Tyr Ala Asp Ser Phe Val Ile Arg Gly Asp Glu
                                85 90 95
Val Arg Gln Ile Ala Pro Gly Gln Thr Gly Lys Ile Ala Asp Tyr Asn
                        100 105 110
Tyr Lys Leu Pro Asp Asp Phe Thr Gly cys Val Ile Ala Trp Asn Ser
                115 120 125
Asn Asn Leu Asp Ser Lys Val Gly Gly Asn Tyr Asn Tyr Leu Try Arg
        130 135 140
Leu Phe Arg Lys Ser Asn Leu Lys Pro Phe Glu Arg Asp Ile Ser Thr
145 150 155 160
Glu Ile Tyr Gln Ala Gly Ser Thr Pro Cys Asn Gly Val Glu Gly Phe
                                165 170 175
Asn cys Tyr Phe Pro Leu Gln Ser Tyr Gly Phe Gln Pro Thr Asn Gly
                        180 185 190
Val Gly Tyr Gln Pro Tyr Arg Val Val Val Leu Ser Phe Glu Leu Leu
                195 200 205
His Ala Pro Ala Thr Val cys Gly Pro Lys Gly Ser Pro Gly Ser Gly
        210 215 220
Ser Gly Ser Gly Tyr Ile Pro Glu Ala Pro Arg Asp Gly Gln Ala Tyr
225 230 235 240
Val Arg Lys Asp Gly Glu Trp Val Leu Leu Ser Thr Phe Leu Gly Ser
                                245 250 255
Gly Ser Gly Ser Glu Gln Tyr Ile Lys Trp Pro Trp Tyr Ile Trp Leu
                        260 265 270
Gly Phe Ile Ala Gly Leu Ile Ala Ile Val Met Val Thr Ile Met Leu
                275 280 285
Cys Cys Met Thr Ser Cys Cys Ser Cys Leu Lys Gly Cys Cys Ser Cys
        290 295 300
Gly Ser Cys Cys Lys Phe Asp Glu Asp Asp Ser Glu Pro Val Leu Lys
305 310 315 320
Gly Val Lys Leu His Tyr Thr
                                325
BNT162b3c (SEQ ID NO: 38; SEQ ID NO: 39)
```

| | |
|---|---|
| Structure | m₂^{7,3′-O}Gppp(m₁^{2′-O})ApG-hAg-Kozak-RBD-GS-Fibritin-GS-TM-FI-A30L70 |
| Encoded antigen | Viral spike protein (S1S2 protein) of the SARS-CoV-2 (partial sequence, |
| | Receptor Binding Domain (RBD) of S1S2 protein fused to Fibritin fused |
| | to Transmembrane Domain (TM) of S1S2 protein); intrinsic S1S2 protein |
| | secretory signal peptide (aa 1-19) at the N-terminus of the antigen |
| | sequence |

```
SEQ ID NO: 38
Met Phe Val Phe Leu Val Leu Leu Pro Leu Val Ser Ser Gln Cys Val
1 5 10 15
Asn Leu Thr Val Arg Phe Pro Asn Ile Thr Asn Leu cys Pro Phe Gly
                        20 25 30
Glu Val Phe Asn Ala Thr Arg Phe Ala Ser Val Tyr Ala Trp Asn Arg
                35 40 45
Lys Arg Ile Ser Asn Cys Val Ala Asp Tyr Ser Val Leu Tyr Asn Ser
        50 55 60
Ala Ser Phe Ser Thr Phe Lys Cys Tyr Gly Val Ser Pro Thr Lys Leu
65 70 75 80
Asn Asp Leu Cys Phe thr Asn Val Tyr Ala Asp Ser Phe Val Ile Arg
                                85 90 95
Gly Asp Glu Val Arg Gln Ile Ala Pro Gly Gln Thr Gly Lys Ile Ala
                        100 105 110
Asp Tyr Asn Tyr Lys Leu Pro Asp Asp Phe Thr Gly cys Val Ile Ala
                115 120 125
Trp Asn Ser Asn Asn Leu Asp Ser LYs Val Gly Gly Asn Tyr Asn Tyr
        130 135 140
Leu Tyr Arg Leu Phe Arg Lys Ser Asn Leu Lys Pro Phe Glu Arg Asp
145 150 155 160
Ile Ser Thr Glu Ile Tyr Gln Ala Gly Ser Thr Pro Cys Asn Gly Val
                                165 170 175
Glu Gly Phe Asn cys Tyr Phe Pro Leu Gln Ser Tyr Gly Phe Gln Pro
                        180 185 190
Thr Asn Gly Val Gly Tyr Gln Pro Tyr Arg Val Val Val Leu Ser Phe
                195 200 205
Glu Leu Leu His Ala Pro Ala Thr Val Cys Gly Pro Lys Gly Ser Pro
        210 215 220
Gly Ser Gly Ser Gly Ser Gly Tyr Ile Pro Glu Ala Pro Arg Asp Gly
225 230 235 240
Gln Ala Tyr Val Arg Lys Asp Gly Glu Trp Val Leu Leu Ser Thr Phe
                                245 250 255
Leu Gly Ser Gly Ser Gly Ser Glu Gln Tyr Ile Lys Trp Pro Trp Tyr
                        260 265 270
Ile Trp Leu Gly Phe Ile Ala Gly Leu Ile Ala Ile Val Met Val Thr
                275 280 285
Ile Met Leu Cys Cys Met Thr Ser Cys Cys Ser Cys Leu Lys Gly Cys
        290 295 300
Cys Ser Cys Gly Ser Cys Cys
305 310
SEQ ID NO: 39
```

| | |
|---|---|
| agaauaaacu aguauucuuc ugguccccac agacucagag agaacccgcc accauguuug | 60 |
| uguuucuugu gcugcugccu cuugugucuu cucagugugu gaauuugaca gugagauuuc | 120 |
| caaauauuac aaaucugugu ccauuuggag aaguguuuaa ugcaacaaga uuugcaucug | 180 |
| uguaugcaug gaauagaaaa agaauuucua auuguguggc ugauuauucu gugcuguaua | 240 |
| auagugcuuc uuuuuccaca uuuaaauguu auggaguguc uccaacaaaa uuaaaugauu | 300 |
| uauguuuuac aaauguguau gcugauucuu uugugaucag aggugaugaa gugagacaga | 360 |
| uugcccccgg acagacagga aaaauugcug auuacaauua caaacugccu gaugauuuua | 420 |
| caggaugugu gauugcuugg aauucuaaua auuuagauuc uaaaguggga ggaaauuaca | 480 |
| auuaucugua cagacuguuu agaaaaucaa aucugaaacc uuuugaaaga gauauuucaa | 540 |
| cagaaauuua ucaggcugga ucaacaccuu guaauggagu ggaaggauuu aauuguuauu | 600 |
| uuccauuaca gagcuaugga uuucagccaa ccaauggugu gggauaucag ccauauagag | 660 |
| ugguggugcu gucuuuugaa cugcugcaug caccugcaac agugugugga ccuaaaggcu | 720 |
| cccccggcuc cggcuccgga ucugguuaua uuccugaagc uccaagagau gggcaagcuu | 780 |
| acguucguaa agauggcgaa uggguauuac uuucuaccuu uuuaggaagc ggcagcggau | 840 |
| cugaacagua cauuaaaugg ccuugguaca uuuggcuugg auuuauugca ggauuaauug | 900 |
| caauugugau ggugacaauu auguuauguu guaugacauc auguuguucu uguuuaaaag | 960 |
| gauguuguuc uuguggaagc uguuguugau gacucgagcu gguacugcau gcacgcaaug | 1020 |
| cuagcugccc cuuucccguc cuggguaccc cgagucuccc ccgaccucgg gucccaggua | 1080 |
| ugcucccacc uccaccugcc ccacucacca ccucugcuag uuccagacac cucccaagca | 1140 |
| cgcagcaaug cagcucaaaa cgcuuagccu agccacaccc ccacgggaaa cagcagugau | 1200 |
| uaaccuuuag caauaaacga aaguuuaacu aagcuauacu aaccccaggg uuggucaauu | 1260 |
| ucgugccagc cacacccugg agcuagcaaa aaaaaaaaaa aaaaaaaaaa aaaaaaagca | 1320 |
| uaugacuaaa aaaaaaaaaa aaaaaaaaaa aaaaaaaaaa aaaaaaaaaa aaaaaaaaaa | 1380 |
| aaaaaaaaaa aaaaaaa | 1397 |

BNT162b3d (SEQ ID NO: 40; SEQ ID NO: 41)

| | |
|---|---|
| Structure | m₂^{7,3′-O}Gppp(m₁^{2′-O})ApG-hAg-Kozak-RBD-GS-Fibritin-GS-TM-FI-A30L70 |
| Encoded antigen | Viral spike protein (S1S2 protein) of the SARS-CoV-2 (partial sequence, |
| | Receptor Binding Domain (RBD) of S1S2 protein fused to Fibritin fused |
| | to Transmembrane Domain (TM) of S1S2 protein); immunoglobulin secretory |
| | signal peptide (aa 1-22) at the N-terminus of the antigen sequence |

```
SEQ ID NO: 40
Met Asp Trp Ile Trp Arg Ile Leu Phe Leu Val Gly Ala Ala Thr Gly
1 5 10 15
Ala His Ser Gln Met Gln Val Arg Phe Pro Asn Ile Thr Asn Leu Cys
                        20 25 30
Pro Phe Gly Glu Val Phe Asn Ala Thr Arg Phe Ala Ser Val Tyr Ala
                35 40 45
Trp Asn Arg Lys Arg Ile Ser Asn Cys Val Ala Asp Tyr Ser Val Leu
        50 55 60
Tyr Asn Ser Ala Ser Phe Ser Thr Phe Lys Cys Tyr Gly Val Ser Pro
65 70 75 80
Thr Lys Leu Asn Asp Leu Cys Phe Thr Asn Val Tyr Ala Asp Ser Phe
                                85 90 95
Val Ile Arg Gly Asp Glu Val Arg Gln Ile Ala Pro Gly Gln Thr Gly
                        100 105 110
Lys Ile Ala Asp Tyr Asn Tyr Lys Leu Pro Asp Asp Phe Thr Gly Cys
                115 120 125
Val Ile Ala Trp Asn Ser Asn Asn Leu Asp Ser Lys Val Gly Gly Asn
        130 135 140
Tyr Asn Tyr Leu Tyr Arg Leu Phe Arg Lys Ser Asn Leu Lys Pro Phe
145 150 155 160
Glu Arg Asp Ile Ser Thr Glu Ile Tyr Gln Ala Gly Ser Thr Pro Cys
                                165 170 175
Asn Gly Val Glu Gly Phe Asn Cys Tyr Phe Pro Leu Gln Ser Tyr Gly
                        180 185 190
Phe Gln Pro Thr Asn Gly Val Gly Tyr Gln Pro Tyr Arg Val Val Val
                195 200 205
Leu Ser Phe Glu Leu Leu His Ala Pro Ala Thr Val Cys Gly Pro Lys
        210 215 220
Gly Ser Pro Gly Ser Gly Ser Gly Ser Gly Tyr Ile Pro Glu Ala Pro
225 230 235 240
Arg Asp Gly Gln Ala Tyr Val Arg Lys Asp Gly Glu Trp Val Leu Leu
                                245 250 255
Ser Thr Phe Leu Gly Ser Gly Ser Gly Ser Glu Gln Tyr Ile Lys Trp
                        260 265 270
Pro Trp Tyr Ile Trp Leu Gly Phe Ile Ala Gly Leu Ile Ala Ile Val
                275 280 285
Met Val Thr Ile Met Leu Cys Cys Met Thr Ser Cys Cys Ser Cys Leu
        290 295 300
Lys Gly Cys Cys Ser Cys Gly Ser Cys Cys
305 310
SEQ ID NO: 41
```

| | |
|---|---|
| agaauaaacu aguauucuuc ugguccccac agacucagag agaacccgcc accauggauu | 60 |
| ggauuuggag aauccuguuc cucgugggag ccgcuacagg agcccacucc cagaugcagg | 120 |
| ugagauuucc aaauauuaca aaucuguguc cauuuggaga aguguuuaau gcaacaagau | 180 |
| uugcaucugu guaugcaugg aauagaaaaa gaauuucuaa uuguguggcu gauuauucug | 240 |
| ugcuguauaa uagugcuucu uuuuccacau uuaaauguua uggagugucu ccaacaaaau | 300 |
| uaaaugauuu auguuuuaca aauguguaug cugauucuuu ugugaucaga ggugaugaag | 360 |
| ugagacagau ugcccccgga cagacaggaa aaauugcuga uuacaauuac aaacugccug | 420 |
| augauuuuac aggaugugug auugcuugga auucuaauaa uuuagauucu aaagugggag | 480 |
| gaaauuacaa uuaucuguac agacuguuua gaaaaucaaa ucugaaaccu uuugaaagag | 540 |
| auauuucaac agaaauuuau caggcuggau caacaccuug uaauggagug gaaggauuua | 600 |
| auuguuauuu uccauuacag agcuauggau uucagccaac caauggugug ggauaucagc | 660 |
| cauauagagu gguggugcug ucuuuugaac ugcugcaugc accugcaaca guguguggac | 720 |
| cuaaaggcuc ccccggcucc ggcuccggau cugguuauau uccugaagcu ccaagagaug | 780 |
| ggcaagcuua cguucguaaa gauggcgaau ggguauuacu uucuaccuuu uuaggaagcg | 840 |
| gcagcggauc ugaacaguac auuaaauggc cuugguacau uuggcuugga uuuauugcag | 900 |
| gauuaauugc aauugugaug gugacaauua uguuauguug uaugacauca uguuguucuu | 960 |
| guuuaaaagg auguuguucu uguggaagcu guuguugaug acucgagcug guacugcaug | 1020 |
| cacgcaaugc uagcugcccc uuucccgucc uggguacccc gagucucccc cgaccucggg | 1080 |
| ucccagguau gcucccaccu ccaccugccc cacucaccac cucugcuagu uccagacacc | 1140 |
| ucccaagcac gcagcaaugc agcucaaaac gcuuagccua gccacacccc cacgggaaac | 1200 |
| agcagugauu aaccuuuagc aauaaacgaa aguuuaacua agcuauacua accccagggu | 1260 |
| uggucaauuu cgugccagcc acacccugga gcuagcaaaa aaaaaaaaaa aaaaaaaaaa | 1320 |
| aaaaaagcau augacuaaaa aaaaaaaaaa aaaaaaaaaa aaaaaaaaaa aaaaaaaaaa | 1380 |
| aaaaaaaaaa aaaaaaaaaa aaaaaa | 1406 |

### Nucleic Acid Containing Particles

Nucleic acids described herein such as RNA encoding a payload may be administered formulated as particles. In the context of the present disclosure, the term “particle” relates to a structured entity formed by molecules or molecule complexes. In some embodiments, the term “particle” relates to a micro- or nano-sized structure, such as a micro- or nano-sized compact structure dispersed in a medium. In some embodiments, a particle is a nucleic acid containing particle such as a particle comprising DNA, RNA or a mixture thereof. In some embodiments, nucleic acid containing particles include lipid nanoparticles, lipoplex, polyplexes (PLX), lipidated polyplexes (LPLX), liposomes, or polysaccharide nanoparticles. Such particles are known in the art to deliver an active agent. See, e.g., Lächelt, Ulrich, and Ernst Wagner. “Nucleic acid therapeutics using polyplexes: a journey of 50 years (and beyond)” Chemical reviews 115.19 (2015): 11043-11078; Plucinski, Alexander, Zan Lyu, and Bernhard VKJ Schmidt, “Polysaccharide nanoparticles: from fabrication to applications.” Journal of Materials Chemistry B (2021); and Tenchov, Rumiana, et al. “Lipid Nanoparticles—From Liposomes to mRNA Vaccine Delivery, a Landscape of Research Diversity and Advancement,” ACS nano 15.11 (2021): 16982-17015, the contents of each of which are hereby incorporated by reference herein in their entirety.

In some embodiments, electrostatic interactions between positively charged molecules such as polymers and lipids and negatively charged nucleic acid are involved in particle formation. This results in complexation and spontaneous formation of nucleic acid particles. In some embodiments, a nucleic acid particle is a nanoparticle.

As used in the present disclosure, “nanoparticle” refers to a particle having an average diameter suitable for parenteral administration.

A “nucleic acid particle” can be used to deliver nucleic acid to a target site of interest (e.g., cell, tissue, organ, and the like). A nucleic acid particle may be formed from at least one cationic or cationically ionizable lipid or lipid-like material, at least one cationic polymer such as protamine, or a mixture thereof and nucleic acid. Nucleic acid particles include lipid nanoparticle (LNP)-based and lipoplex (LPX)-based formulations.

Without intending to be bound by any theory, it is believed that the cationic or cationically ionizable lipid or lipid-like material and/or the cationic polymer combine together with the nucleic acid to form aggregates, and this aggregation results in colloidally stable particles.

In some embodiments, particles described herein further comprise at least one lipid or lipid-like material other than a cationic or cationically ionizable lipid or lipid-like material, at least one polymer other than a cationic polymer, or a mixture thereof.

In some embodiments, nucleic acid particles comprise more than one type of nucleic acid molecules, where the molecular parameters of the nucleic acid molecules may be similar or different from each other, like with respect to molar mass or fundamental structural elements such as molecular architecture, capping, coding regions or other features. Nucleic acid particles described herein may have an average diameter that in some embodiments ranges from about 30 nm to about 1000 nm, from about 50 nm to about 800 nm, from about 70 nm to about 600 nm, from about 90 nm to about 400 nm, or from about 100 nm to about 300 nm. In some embodiments, nucleic acid particles described herein may have an average diameter ranging from about 50 nm to about 200 nm or from about 50 nm to about 150 nm, or from about 50 nm to about 100 nm.

Nucleic acid particles described herein may exhibit a polydispersity index less than about 0.5, less than about 0.4, less than about 0.3, or about 0.2 or less. By way of example, the nucleic acid particles can exhibit a polydispersity index in a range of about 0.1 to about 0.3 or about 0.2 to about 0.3.

With respect to RNA lipid particles, the N/P ratio gives the ratio of the nitrogen groups in the lipid to the number of phosphate groups in the RNA. It is correlated to the charge ratio, as the nitrogen atoms (depending on the pH) are usually positively charged and the phosphate groups are negatively charged. The N/P ratio, where a charge equilibrium exists, depends on the pH. Lipid formulations are frequently formed at N/P ratios larger than four up to twelve, because positively charged nanoparticles are considered favorable for transfection. In that case, RNA is considered to be completely bound to nanoparticles.

Nucleic acid particles described herein can be prepared using a wide range of methods that may involve obtaining a colloid from at least one cationic or cationically ionizable lipid or lipid-like material and/or at least one cationic polymer and mixing the colloid with nucleic acid to obtain nucleic acid particles.

The term “colloid” as used herein relates to a type of homogeneous mixture in which dispersed particles do not settle out. The insoluble particles in the mixture are microscopic, with particle sizes between 1 and 1000 nanometers. The mixture may be termed a colloid or a colloidal suspension. Sometimes the term “colloid” only refers to the particles in the mixture and not the entire suspension.

For the preparation of colloids comprising at least one cationic or cationically ionizable lipid or lipid-like material and/or at least one cationic polymer methods are applicable herein that are conventionally used for preparing liposomal vesicles and are appropriately adapted. The most commonly used methods for preparing liposomal vesicles share the following fundamental stages: (i) lipids dissolution in organic solvents, (ii) drying of the resultant solution, and (iii) hydration of dried lipid (using various aqueous media).

In the film hydration method, lipids are firstly dissolved in a suitable organic solvent, and dried down to yield a thin film at the bottom of the flask. The obtained lipid film is hydrated using an appropriate aqueous medium to produce a liposomal dispersion. Furthermore, an additional downsizing step may be included.

Reverse phase evaporation is an alternative method to the film hydration for preparing liposomal vesicles that involves formation of a water-in-oil emulsion between an aqueous phase and an organic phase containing lipids. A brief sonication of this mixture is required for system homogenization. The removal of the organic phase under reduced pressure yields a milky gel that turns subsequently into a liposomal suspension.

The term “ethanol injection technique” refers to a process, in which an ethanol solution comprising lipids is rapidly injected into an aqueous solution through a needle. This action disperses the lipids throughout the solution and promotes lipid structure formation, for example lipid vesicle formation such as liposome formation. Generally, the RNA lipoplex particles described herein are obtainable by adding RNA to a colloidal liposome dispersion. Using the ethanol injection technique, such colloidal liposome dispersion is, in some embodiments, formed as follows: an ethanol solution comprising lipids, such as cationic lipids and additional lipids, is injected into an aqueous solution under stirring. In some embodiments, the RNA lipoplex particles described herein are obtainable without a step of extrusion.

The term “extruding” or “extrusion” refers to the creation of particles having a fixed, cross-sectional profile. In particular, it refers to the downsizing of a particle, whereby the particle is forced through filters with defined pores.

Other methods having organic solvent free characteristics may also be used according to the present disclosure for preparing a colloid.

LNPs typically comprise four components: ionizable cationic lipids, neutral lipids such as phospholipids, a steroid such as cholesterol, and a polymer conjugated lipid such as polyethylene glycol (PEG)-lipids. Each component is responsible for payload protection, and enables effective intracellular delivery. LNPs may be prepared by mixing lipids dissolved in ethanol rapidly with nucleic acid in an aqueous buffer.

The term “average diameter” refers to the mean hydrodynamic diameter of particles as measured by dynamic laser light scattering (DLS) with data analysis using the so-called cumulant algorithm, which provides as results the so-called Zaverage with the dimension of a length, and the polydispersity index (PI), which is dimensionless (Koppel, D., J. Chem. Phys. 57, 1972, pp 4814-4820, ISO 13321). Here “average diameter”, “diameter” or “size” for particles is used synonymously with this value of the Zaverage.

The “polydispersity index” is preferably calculated based on dynamic light scattering measurements by the so-called cumulant analysis as mentioned in the definition of the “average diameter”. Under certain prerequisites, it can be taken as a measure of the size distribution of an ensemble of nanoparticles.

Different types of nucleic acid containing particles have been described previously to be suitable for delivery of nucleic acid in particulate form (e.g. Kaczmarek, J. C. et al. 2017, Genome Medicine 9, 60). For non-viral nucleic acid delivery vehicles, nanoparticle encapsulation of nucleic acid physically protects nucleic acid from degradation and, depending on the specific chemistry, can aid in cellular uptake and endosomal escape.

The present disclosure describes particles comprising nucleic acid, at least one cationic or cationically ionizable lipid or lipid-like material, and/or at least one cationic polymer which associate with nucleic acid to form nucleic acid particles and compositions comprising such particles. The nucleic acid particles may comprise nucleic acid which is complexed in different forms by non-covalent interactions to the particle. The particles described herein are not viral particles, in particular infectious viral particles, i.e., they are not able to virally infect cells. Suitable cationic or cationically ionizable lipids or lipid-like materials and cationic polymers are those that form nucleic acid particles and are included by the term “particle forming components” or “particle forming agents”. The term “particle forming components” or “particle forming agents” relates to any components which associate with nucleic acid to form nucleic acid particles. Such components include any component which can be part of nucleic acid particles.

Some embodiments described herein relate to compositions, methods and uses involving more than one, e.g., 2, 3, 4, 5, 6 or even more nucleic acid species such as RNA species, e.g., a) a nucleic acid comprising a first nucleotide sequence encoding an amino acid sequence comprising at least a fragment of a parental virus protein, wherein amino acid positions in the at least a fragment of a parental virus protein are modified to comprise amino acids found in the corresponding amino acid positions of one or more virus protein variants; and b) a nucleic acid comprising a second nucleotide sequence encoding an amino acid sequence comprising at least a fragment of a parental virus protein, wherein amino acid positions in the at least a fragment of a parental virus protein are modified to comprise amino acids found in the corresponding amino acid positions of one or more virus protein variants.

In a particulate formulation, it is possible that each nucleic acid species is separately formulated as an individual particulate formulation. In that case, each individual particulate formulation will comprise one nucleic acid species. The individual particulate formulations may be present as separate entities, e.g. in separate containers. Such formulations are obtainable by providing each nucleic acid species separately (typically each in the form of a nucleic acid-containing solution) together with a particle-forming agent, thereby allowing the formation of particles. Respective particles will contain exclusively the specific nucleic acid species that is being provided when the particles are formed (individual particulate formulations).

In some embodiments, a composition such as a pharmaceutical composition comprises more than one individual particle formulation. Respective pharmaceutical compositions are referred to as mixed particulate formulations. Mixed particulate formulations according to the invention are obtainable by forming, separately, individual particulate formulations, as described above, followed by a step of mixing of the individual particulate formulations. By the step of mixing, a formulation comprising a mixed population of nucleic acid-containing particles is obtainable. Individual particulate populations may be together in one container, comprising a mixed population of individual particulate formulations.

Alternatively, it is possible that different nucleic acid species are formulated together as a combined particulate formulation. Such formulations are obtainable by providing a combined formulation (typically combined solution) of different RNA species together with a particle-forming agent, thereby allowing the formation of particles. As opposed to a mixed particulate formulation, a combined particulate formulation will typically comprise particles which comprise more than one RNA species. In a combined particulate composition different RNA species are typically present together in a single particle.

### Cationic Polymeric Materials (e.g., Polymers)

Given their high degree of chemical flexibility, polymeric materials are commonly used for nanoparticle-based delivery. Typically, cationic materials are used to electrostatically condense the negatively charged nucleic acid into nanoparticles. These positively charged groups often consist of amines that change their state of protonation in the pH range between 5.5 and 7.5, thought to lead to an ion imbalance that results in endosomal rupture. Polymers such as poly-L-lysine, polyamidoamine, protamine and polyethyleneimine, as well as naturally occurring polymers such as chitosan have all been applied to nucleic acid delivery and are suitable as cationic materials useful in some embodiments herein. In addition, some investigators have synthesized polymeric materials specifically for nucleic acid delivery. Poly(O-amino esters), in particular, have gained widespread use in nucleic acid delivery owing to their ease of synthesis and biodegradability. In some embodiments, such synthetic materials may be suitable for use as cationic materials herein.

A “polymeric material”, as used herein, is given its ordinary meaning, i.e., a molecular structure comprising one or more repeat units (monomers), connected by covalent bonds. In some embodiments, such repeat units can all be identical; alternatively, in some cases, there can be more than one type of repeat unit present within the polymeric material. In some cases, a polymeric material is biologically derived, e.g., a biopolymer such as a protein. In some cases, additional moieties can also be present in the polymeric material, for example targeting moieties such as those described herein.

Those skilled in the art are aware that, when more than one type of repeat unit is present within a polymer (or polymeric moiety), then the polymer (or polymeric moiety) is said to be a “copolymer.” In some embodiments, a polymer (or polymeric moiety) utilized in accordance with the present disclosure may be a copolymer. Repeat units forming the copolymer can be arranged in any fashion. For example, in some embodiments, repeat units can be arranged in a random order; alternatively or additionally, in some embodiments, repeat units may be arranged in an alternating order, or as a “block” copolymer, i.e., comprising one or more regions each comprising a first repeat unit (e.g., a first block), and one or more regions each comprising a second repeat unit (e.g., a second block), etc. Block copolymers can have two (a diblock copolymer), three (a triblock copolymer), or more numbers of distinct blocks. In certain embodiments, a polymeric material for use in accordance with the present disclosure is biocompatible. Biocompatible materials are those that typically do not result in significant cell death at moderate concentrations. In certain embodiments, a biocompatible material is biodegradable, i.e., is able to degrade, chemically and/or biologically, within a physiological environment, such as within the body.

In certain embodiments, a polymeric material may be or comprise protamine or polyalkyleneimine, in particular protamine.

As those skilled in the art are aware term “protamine” is often used to refer to any of various strongly basic proteins of relatively low molecular weight that are rich in arginine and are found associated especially with DNA in place of somatic histones in the sperm cells of various animals (as fish). In particular, the term “protamine” is often used to refer to proteins found in fish sperm that are strongly basic, are soluble in water, are not coagulated by heat, and yield chiefly arginine upon hydrolysis. In purified form, they are used in a long-acting formulation of insulin and to neutralize the anticoagulant effects of heparin.

In some embodiments, the term “protamine” as used herein is refers to a protamine amino acid sequence obtained or derived from natural or biological sources, including fragments thereof and/or multimeric forms of said amino acid sequence or fragment thereof, as well as (synthesized) polypeptides which are artificial and specifically designed for specific purposes and cannot be isolated from native or biological sources.

In some embodiments, a polyalkyleneimine comprises polyethylenimine and/or polypropylenimine, preferably polyethyleneimine. In some embodiments, a preferred polyalkyleneimine is polyethyleneimine (PEI). In some embodiments, the average molecular weight of PEI is preferably 0.75-102 to 107 Da, preferably 1000 to 105 Da, more preferably 10000 to 40000 Da, more preferably 15000 to 30000 Da, even more preferably 20000 to 25000 Da.

Preferred according to certain embodiments of the disclosure is linear polyalkyleneimine such as linear polyethyleneimine (PEI).

Cationic materials (e.g., polymeric materials, including polycationic polymers) contemplated for use herein include those which are able to electrostatically bind nucleic acid. In some embodiments, cationic polymeric materials contemplated for use herein include any cationic polymeric materials with which nucleic acid can be associated, e.g. by forming complexes with the nucleic acid or forming vesicles in which the nucleic acid is enclosed or encapsulated.

In some embodiments, particles described herein may comprise polymers other than cationic polymers, e.g., non-cationic polymeric materials and/or anionic polymeric materials.

Collectively, anionic and neutral polymeric materials are referred to herein as non-cationic polymeric materials.

### Lipid and Lipid-Like Material

The terms “lipid” and “lipid-like material” are used herein to refer to molecules which comprise one or more hydrophobic moieties or groups and optionally also one or more hydrophilic moieties or groups. Molecules comprising hydrophobic moieties and hydrophilic moieties are also frequently denoted as amphiphiles. Lipids are usually poorly soluble in water. In an aqueous environment, the amphiphilic nature allows the molecules to self-assemble into organized structures and different phases. One of those phases consists of lipid bilayers, as they are present in vesicles, multilamellar/unilamellar liposomes, or membranes in an aqueous environment. Hydrophobicity can be conferred by the inclusion of apolar groups that include, but are not limited to, long-chain saturated and unsaturated aliphatic hydrocarbon groups and such groups substituted by one or more aromatic, cycloaliphatic, or heterocyclic group(s). In some embodiments, hydrophilic groups may comprise polar and/or charged groups and include carbohydrates, phosphate, carboxylic, sulfate, amino, sulfhydryl, nitro, hydroxyl, and other like groups.

As used herein, the term “amphiphilic” refers to a molecule having both a polar portion and a non-polar portion. Often, an amphiphilic compound has a polar head attached to a long hydrophobic tail. In some embodiments, the polar portion is soluble in water, while the non-polar portion is insoluble in water. In addition, the polar portion may have either a formal positive charge, or a formal negative charge. Alternatively, the polar portion may have both a formal positive and a negative charge, and be a zwitterion or inner salt. For purposes of the disclosure, the amphiphilic compound can be, but is not limited to, one or a plurality of natural or non-natural lipids and lipid-like compounds.

The term “lipid-like material”, “lipid-like compound” or “lipid-like molecule” relates to substances that structurally and/or functionally relate to lipids but may not be considered as lipids in a strict sense. For example, the term includes compounds that are able to form amphiphilic layers as they are present in vesicles, multilamellar/unilamellar liposomes, or membranes in an aqueous environment and includes surfactants, or synthesized compounds with both hydrophilic and hydrophobic moieties. Generally speaking, the term refers to molecules, which comprise hydrophilic and hydrophobic moieties with different structural organization, which may or may not be similar to that of lipids. As used herein, the term “lipid” is to be construed to cover both lipids and lipid-like materials unless otherwise indicated herein or clearly contradicted by context.

Specific examples of amphiphilic compounds that may be included in an amphiphilic layer include, but are not limited to, phospholipids, aminolipids and sphingolipids.

In certain embodiments, the amphiphilic compound is a lipid. The term “lipid” refers to a group of organic compounds that are characterized by being insoluble in water, but soluble in many organic solvents. Generally, lipids may be divided into eight categories: fatty acids, glycerolipids, glycerophospholipids, sphingolipids, saccharolipids, polyketides (derived from condensation of ketoacyl subunits), sterol lipids and prenol lipids (derived from condensation of isoprene subunits). Although the term “lipid” is sometimes used as a synonym for fats, fats are a subgroup of lipids called triglycerides. Lipids also encompass molecules such as fatty acids and their derivatives (including tri-, di-, monoglycerides, and phospholipids), as well as sterol-containing metabolites such as cholesterol.

Fatty acids, or fatty acid residues are a diverse group of molecules made of a hydrocarbon chain that terminates with a carboxylic acid group; this arrangement confers the molecule with a polar, hydrophilic end, and a nonpolar, hydrophobic end that is insoluble in water. The carbon chain, typically between four and 24 carbons long, may be saturated or unsaturated, and may be attached to functional groups containing oxygen, halogens, nitrogen, and sulfur. If a fatty acid contains a double bond, there is the possibility of either a cis or trans geometric isomerism, which significantly affects the molecule's configuration. Cis-double bonds cause the fatty acid chain to bend, an effect that is compounded with more double bonds in the chain. Other major lipid classes in the fatty acid category are the fatty esters and fatty amides.

Glycerolipids are composed of mono-, di-, and tri-substituted glycerols, the best-known being the fatty acid triesters of glycerol, called triglycerides. The word “triacylglycerol” is sometimes used synonymously with “triglyceride”. In these compounds, the three hydroxyl groups of glycerol are each esterified, typically by different fatty acids. Additional subclasses of glycerolipids are represented by glycosylglycerols, which are characterized by the presence of one or more sugar residues attached to glycerol via a glycosidic linkage.

The glycerophospholipids are amphipathic molecules (containing both hydrophobic and hydrophilic regions) that contain a glycerol core linked to two fatty acid-derived “tails” by ester linkages and to one “head” group by a phosphate ester linkage. Examples of glycerophospholipids, usually referred to as phospholipids (though sphingomyelins are also classified as phospholipids) are phosphatidylcholine (also known as PC, GPCho or lecithin), phosphatidylethanolamine (PE or GPEtn) and phosphatidylserine (PS or GPSer).

Sphingolipids are a complex family of compounds that share a common structural feature, a sphingoid base backbone. The major sphingoid base in mammals is commonly referred to as sphingosine. Ceramides (N-acyl-sphingoid bases) are a major subclass of sphingoid base derivatives with an amide-linked fatty acid. The fatty acids are typically saturated or mono-unsaturated with chain lengths from 16 to 26 carbon atoms. The major phosphosphingolipids of mammals are sphingomyelins (ceramide phosphocholines), whereas insects contain mainly ceramide phosphoethanolamines and fungi have phytoceramide phosphoinositols and mannose-containing headgroups. The glycosphingolipids are a diverse family of molecules composed of one or more sugar residues linked via a glycosidic bond to the sphingoid base. Examples of these are the simple and complex glycosphingolipids such as cerebrosides and gangliosides. Sterol lipids, such as cholesterol and its derivatives, or tocopherol and its derivatives, are an important component of membrane lipids, along with the glycerophospholipids and sphingomyelins.

Saccharolipids describe compounds in which fatty acids are linked directly to a sugar backbone, forming structures that are compatible with membrane bilayers. In the saccharolipids, a monosaccharide substitutes for the glycerol backbone present in glycerolipids and glycerophospholipids. The most familiar saccharolipids are the acylated glucosamine precursors of the Lipid A component of the lipopolysaccharides in Gram-negative bacteria. Typical lipid A molecules are disaccharides of glucosamine, which are derivatized with as many as seven fatty-acyl chains. The minimal lipopolysaccharide required for growth in E. coli is Kdo2-Lipid A, a hexa-acylated disaccharide of glucosamine that is glycosylated with two 3-deoxy-D-manno-octulosonic acid (Kdo) residues.

Polyketides are synthesized by polymerization of acetyl and propionyl subunits by classic enzymes as well as iterative and multimodular enzymes that share mechanistic features with the fatty acid synthases. They comprise a large number of secondary metabolites and natural products from animal, plant, bacterial, fungal and marine sources, and have great structural diversity. Many polyketides are cyclic molecules whose backbones are often further modified by glycosylation, methylation, hydroxylation, oxidation, or other processes. According to the disclosure, lipids and lipid-like materials may be cationic, anionic or neutral. Neutral lipids or lipid-like materials exist in an uncharged or neutral zwitterionic form at a selected pH.

### Cationic or Cationically Ionizable Lipids or Lipid-Like Materials

In some embodiments, nucleic acid particles described and/or utilized in accordance with the present disclosure may comprise at least one cationic or cationically ionizable lipid or lipid-like material as particle forming agent. Cationic or cationically ionizable lipids or lipid-like materials contemplated for use herein include any cationic or cationically ionizable lipids or lipid-like materials which are able to electrostatically bind nucleic acid. In some embodiments, cationic or cationically ionizable lipids or lipid-like materials contemplated for use herein can be associated with nucleic acid, e.g. by forming complexes with the nucleic acid or forming vesicles in which the nucleic acid is enclosed or encapsulated.

As used herein, a “cationic lipid” or “cationic lipid-like material” refers to a lipid or lipid-like material having a net positive charge. Cationic lipids or lipid-like materials bind negatively charged nucleic acid by electrostatic interaction. Generally, cationic lipids possess a lipophilic moiety, such as a sterol, an acyl chain, a diacyl or more acyl chains, and the head group of the lipid typically carries the positive charge.

In certain embodiments, a cationic lipid or lipid-like material has a net positive charge only at certain pH, in particular acidic pH, while it has preferably no net positive charge, preferably has no charge, i.e., it is neutral, at a different, preferably higher pH such as physiological pH. This ionizable behavior is thought to enhance efficacy through helping with endosomal escape and reducing toxicity as compared with particles that remain cationic at physiological pH.

For purposes of the present disclosure, such “cationically ionizable” lipids or lipid-like materials are comprised by the term “cationic lipid or lipid-like material” unless contradicted by the circumstances.

In some embodiments, a cationic or cationically ionizable lipid or lipid-like material comprises a head group which includes at least one nitrogen atom (N) which is positive charged or capable of being protonated.

Examples of cationic lipids include, but are not limited to: ((4-hydroxybutyl)azanediyl)bis(hexane-6,1-diyl)bis(2-hexyldecanoate); 1,2-dioleoyl-3-trimethylammonium propane (DOTAP); N,N-dimethyl-2,3-dioleyloxypropylamine (DODMA), 1,2-di-O-octadecenyl-3-trimethylammonium propane (DOTMA), 3-(N—(N′,N′-dimethylaminoethane)-carbamoyl)cholesterol (DC-Chol), dimethyldioctadecylammonium (DDAB); 1,2-dioleoyl-3-dimethylammonium-propane (DODAP); 1,2-diacyloxy-3-dimethylammonium propanes; 1,2-dialkyloxy-3-dimethylammonium propanes; dioctadecyldimethyl ammonium chloride (DODAC), 1,2-distearyloxy-N,N-dimethyl-3-aminopropane (DSDMA), 2,3-di(tetradecoxy)propyl-(2-hydroxyethyl)-dimethylazanium (DMRIE), 1,2-dimyristoyl-sn-glycero-3-ethylphosphocholine (DMEPC), 1,2-dimyristoyl-3-trimethylammonium propane (DMTAP), 1,2-dioleyloxypropyl-3-dimethyl-hydroxyethyl ammonium bromide (DORIE), and 2,3-dioleoyloxy-N-[2(spermine carboxamide)ethyl]-N,N-dimethyl-1-propanamium trifluoroacetate (DOSPA), 1,2-dilinoleyloxy-N,N-dimethylaminopropane (DLinDMA), 1,2-dilinolenyloxy-N,N-dimethylaminopropane (DLenDMA), dioctadecylamidoglycyl spermine (DOGS), 3-dimethylamino-2-(cholest-5-en-3-beta-oxybutan-4-oxy)-1-(cis,cis-9,12-oc-tadecadienoxy)propane (CLinDMA), 2-[5′-(cholest-5-en-3-beta-oxy)-3′-oxapentoxy)-3-dimethyl-1-(cis,cis-9′,12′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 2,3-Dilinoleoyloxy-N,N-dimethylpropylamine (DLinDAP), 1,2-N,N′-Dilinoleylcarbamyl-3-dimethylaminopropane (DLincarbDAP), 1,2-Dilinoleoylcarbamyl-3-dimethylaminopropane (DLinCDAP), 2,2-dilinoleyl-4-dimethylaminomethyl-[1,3]-dioxolane (DLin-K-DMA), 2,2-dilinoleyl-4-dimethylaminoethyl-[1,3]-dioxolane (DLin-K-XTC2-DMA), 2,2-dilinoleyl-4-(2-dimethylaminoethyl)-[1,3]-dioxolane (DLin-KC2-DMA), heptatriaconta-6,9,28,31-tetraen-19-yl-4-(dimethylamino)butanoate (DLin-MC3-DMA), N-(2-Hydroxyethyl)-N,N-dimethyl-2,3-bis(tetradecyloxy)-1-propanaminium bromide (DMRIE), (±)—N-(3-aminopropyl)-N,N-dimethyl-2,3-bis(cis-9-tetradecenyloxy)-1-propanaminium bromide (GAP-DMORIE), (±)—N-(3-aminopropyl)-N,N-dimethyl-2,3-bis(dodecyloxy)-1-propanaminium bromide (GAP-DLRIE), (±)—N-(3-aminopropyl)-N,N-dimethyl-2,3-bis(tetradecyloxy)-1-propanaminium bromide (GAP-DMRIE), N-(2-Aminoethyl)-N,N-dimethyl-2,3-bis(tetradecyloxy)-1-propanaminium bromide (βAE-DMRIE), N-(4-carboxybenzyl)-N,N-dimethyl-2,3-bis(oleoyloxy)propan-1-aminium (DOBAQ), 2-({8-[(33)-cholest-5-en-3-yloxy]octyl}oxy)-N,N-dimethyl-3-[(9Z,12Z)-octadeca-9,12-dien-1-yloxy]propan-1-amine (Octyl-CLinDMA), 1,2-dimyristoyl-3-dimethylammonium-propane (DMDAP), 1,2-dipalmitoyl-3-dimethylammonium-propane (DPDAP), N1-[2-((1S)-1-[(3-aminopropyl)amino]-4-[di(3-amino-propyl)amino]butylcarboxamido)ethyl]-3,4-di[oleyloxy]-benzamide (MVL5), 1,2-dioleoyl-sn-glycero-3-ethylphosphocholine (DOEPC), 2,3-bis(dodecyloxy)-N-(2-hydroxyethyl)-N,N-dimethylpropan-1-ammonium bromide (DLRIE), N-(2-aminoethyl)-N,N-dimethyl-2,3-bis(tetradecyloxy)propan-1-aminium bromide (DMORIE), di((Z)-non-2-en-1-yl) 8,8′-((((2(dimethylamino)ethyl)thio)carbonyl)azanediyl)dioctanoate (ATX), N,N-dimethyl-2,3-bis(dodecyloxy)propan-1-amine (DLDMA), N,N-dimethyl-2,3-bis(tetradecyloxy)propan-1-amine (DMDMA), Di((Z)-non-2-en-1-yl)-9-((4-(dimethylaminobutanoyl)oxy)heptadecanedioate (L319), N-Dodecyl-3-((2-dodecylcarbamoyl-ethyl)-{2-[(2-dodecylcarbamoyl-ethyl)-2-{(2-dodecylcarbamoyl-ethyl)-[2-(2-dodecylcarbamoyl-ethylamino)-ethyl]-amino}-ethylamino)propionamide (lipidoid 98N12-5), 1-[2-[bis(2-hydroxydodecyl)amino]ethyl-[2-[4-[2-[bis(2 hydroxydodecyl)amino]ethyl]piperazin-1-yl]ethyl]amino]dodecan-2-ol (lipidoid C12-200); or heptadecan-9-yl 8-((2-hydroxyethyl) (6-oxo-6-(undecyloxy) hexyl) amino) octanoate (SM-102).

In some embodiments, a cationic lipid is or comprises heptadecan-9-yl 8-((2-hydroxyethyl) (6-oxo-6-(undecyloxy) hexyl) amino) octanoate (SM-102). In some embodiments, a cationic lipid is or comprises a cationic lipid shown in the structure below.

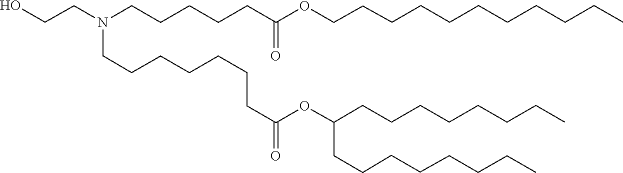 [SMILES: CCCCCCCCCCCOC(=O)CCCCCN(CCO)CCCCCCCC(=O)OC(CCCCCCCC)CCCCCCCC]

In some embodiments, a cationic lipid is or comprises ((4-hydroxybutyl)azanediyl)bis(hexane-6,1-diyl)bis(2-hexyldecanoate) which is also referred to as ALC-0315 herein.

In some embodiments, a cationic lipid may comprise from about 10 mol % to about 100 mol %, about 20 mol % to about 100 mol %, about 30 mol % to about 100 mol %, about 40 mol % to about 100 mol %, or about 50 mol % to about 100 mol % of the total lipid present in the particle.

In some particular embodiments, a particle for use in accordance with the present disclosure includes ALC-0315, for example in a weight percent within a range of about 40-55 mol percent of total lipids.

### Additional Lipids or Lipid-Like Materials

In some embodiments, particles described herein comprise (e.g., in addition to a cationic lipid such as ALC315), one or more lipids or lipid-like materials other than cationic or cationically ionizable lipids or lipid-like materials, e.g., non-cationic lipids or lipid-like materials (including non-cationically ionizable lipids or lipid-like materials). Collectively, anionic and neutral lipids or lipid-like materials are referred to herein as non-cationic lipids or lipid-like materials. Optimizing the formulation of nucleic acid particles by addition of other hydrophobic moieties, such as cholesterol and lipids, in addition to an ionizable/cationic lipid or lipid-like material may enhance particle stability and efficacy of nucleic acid delivery.

An additional lipid or lipid-like material may be incorporated which may or may not affect the overall charge of the nucleic acid particles. In certain embodiments, the additional lipid or lipid-like material is a non-cationic lipid or lipid-like material. The non-cationic lipid may comprise, e.g., one or more anionic lipids and/or neutral lipids. As used herein, an “anionic lipid” refers to any lipid that is negatively charged at a selected pH. As used herein, a “neutral lipid” refers to any of a number of lipid species that exist either in an uncharged or neutral zwitterionic form at a selected pH. In preferred embodiments, the additional lipid comprises one of the following neutral lipid components: (1) a phospholipid, (2) cholesterol or a derivative thereof; or (3) a mixture of a phospholipid and cholesterol or a derivative thereof. Examples of cholesterol derivatives include, but are not limited to, cholestanol, cholestanone, cholestenone, coprostanol, cholesteryl-2′-hydroxyethyl ether, cholesteryl-4′-hydroxybutyl ether, tocopherol and derivatives thereof, and mixtures thereof.

Specific phospholipids that can be used include, but are not limited to, phosphatidylcholines, phosphatidylethanolamines, phosphatidylglycerols, phosphatidic acids, phosphatidylserines or sphingomyelin. Such phospholipids include in particular diacylphosphatidylcholines, such as distearoylphosphatidylcholine (DSPC), dioleoylphosphatidylcholine (DOPC), dimyristoylphosphatidylcholine (DMPC), dipentadecanoylphosphatidylcholine, dilauroylphosphatidylcholine, dipalmitoylphosphatidylcholine (DPPC), diarachidoylphosphatidylcholine (DAPC), dibehenoylphosphatidylcholine (DBPC), ditricosanoylphosphatidylcholine (DTPC), dilignoceroylphatidylcholine (DLPC), palmitoyloleoyl-phosphatidylcholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC) and phosphatidylethanolamines, in particular diacylphosphatidylethanolamines, such as dioleoylphosphatidylethanolamine (DOPE), distearoyl-phosphatidylethanolamine (DSPE), dipalmitoyl-phosphatidylethanolamine (DPPE), dimyristoyl-phosphatidylethanolamine (DMPE), dilauroyl-phosphatidylethanolamine (DLPE), diphytanoyl-phosphatidylethanolamine (DPyPE), and further phosphatidylethanolamine lipids with different hydrophobic chains.

In certain preferred embodiments, the additional lipid is DSPC or DSPC and cholesterol. In certain embodiments, the nucleic acid particles include both a cationic lipid and an additional lipid.

In some embodiments, particles described herein include a polymer conjugated lipid such as a pegylated lipid. The term “pegylated lipid” refers to a molecule comprising both a lipid portion and a polyethylene glycol portion. Pegylated lipids are known in the art. In some embodiments, a pegylated lipid is ALC-0159, also referred to herein as (2-[(polyethylene glycol)-2000]—N,N-ditetradecylacetamide).

Without wishing to be bound by theory, the amount of the at least one cationic lipid compared to the amount of the at least one additional lipid may affect important nucleic acid particle characteristics, such as charge, particle size, stability, tissue selectivity, and bioactivity of the nucleic acid. Accordingly, in some embodiments, the molar ratio of the at least one cationic lipid to the at least one additional lipid is from about 10:0 to about 1:9, about 4:1 to about 1:2, or about 3:1 to about 1:1.

In some embodiments, the non-cationic lipid, in particular neutral lipid, (e.g., one or more phospholipids and/or cholesterol) may comprise from about 0 mol % to about 90 mol %, from about 0 mol % to about 80 mol %, from about 0 mol % to about 70 mol %, from about 0 mol % to about 60 mol %, or from about 0 mol % to about 50 mol %, of the total lipid present in the particle.

In some embodiments, particles for use in accordance with the present disclosure may include, for example, ALC-0315, DSPC, CHOL, and ALC-0159, for example, wherein ALC-0315 is at about 40 to 55 mol percent; DSPC is at about 5 to 15 mol percent; CHOL is at about 30 to 50 mol percent; and ALC-0159 is at about 1 to 10 mol percent.

### Lipoplex Particles

In certain embodiments of the present disclosure, an RNA may be present in RNA lipoplex particles.

In the context of the present disclosure, the term “RNA lipoplex particle” relates to a particle that contains lipid, in particular cationic lipid, and RNA. Electrostatic interactions between positively charged liposomes and negatively charged RNA results in complexation and spontaneous formation of RNA lipoplex particles. Positively charged liposomes may be generally synthesized using a cationic lipid, such as DOTMA, and additional lipids, such as DOPE. In some embodiments, a RNA lipoplex particle is a nanoparticle.

In certain embodiments, the RNA lipoplex particles include both a cationic lipid and an additional lipid. In an exemplary embodiment, the cationic lipid is DOTMA and the additional lipid is DOPE.

In some embodiments, the molar ratio of the at least one cationic lipid to the at least one additional lipid is from about 10:0 to about 1:9, about 4:1 to about 1:2, or about 3:1 to about 1:1. In specific embodiments, the molar ratio may be about 3:1, about 2.75:1, about 2.5:1, about 2.25:1, about 2:1, about 1.75:1, about 1.5:1, about 1.25:1, or about 1:1. In an exemplary embodiment, the molar ratio of the at least one cationic lipid to the at least one additional lipid is about 2:1.

RNA lipoplex particles described herein have an average diameter that in some embodiments ranges from about 200 nm to about 1000 nm, from about 200 nm to about 800 nm, from about 250 to about 700 nm, from about 400 to about 600 nm, from about 300 nm to about 500 nm, or from about 350 nm to about 400 nm. In specific embodiments, the RNA lipoplex particles have an average diameter of about 200 nm, about 225 nm, about 250 nm, about 275 nm, about 300 nm, about 325 nm, about 350 nm, about 375 nm, about 400 nm, about 425 nm, about 450 nm, about 475 nm, about 500 nm, about 525 nm, about 550 nm, about 575 nm, about 600 nm, about 625 nm, about 650 nm, about 700 nm, about 725 nm, about 750 nm, about 775 nm, about 800 nm, about 825 nm, about 850 nm, about 875 nm, about 900 nm, about 925 nm, about 950 nm, about 975 nm, or about 1000 nm. In an embodiment, the RNA lipoplex particles have an average diameter that ranges from about 250 nm to about 700 nm. In another embodiment, the RNA lipoplex particles have an average diameter that ranges from about 300 nm to about 500 nm. In an exemplary embodiment, the RNA lipoplex particles have an average diameter of about 400 nm.

In some embodiments, RNA lipoplex particles and/or compositions comprising RNA lipoplex particles described herein are useful for delivery of RNA to a target tissue after parenteral administration, in particular after intravenous administration. In some embodiments, RNA lipoplex particles may be prepared using liposomes that may be obtained by injecting a solution of the lipids in ethanol into water or a suitable aqueous phase. In some embodiments, the aqueous phase has an acidic pH. In some embodiments, the aqueous phase comprises acetic acid, e.g., in an amount of about 5 mM. Liposomes may be used for preparing RNA lipoplex particles by mixing the liposomes with RNA. In some embodiments, the liposomes and RNA lipoplex particles comprise at least one cationic lipid and at least one additional lipid. In some embodiments, the at least one cationic lipid comprises 1,2-di-O-octadecenyl-3-trimethylammonium propane (DOTMA) and/or 1,2-dioleoyl-3-trimethylammonium-propane (DOTAP). In some embodiments, the at least one additional lipid comprises 1,2-di-(9Z-octadecenoyl)-sn-glycero-3-phosphoethanolamine (DOPE), cholesterol (Chol) and/or 1,2-dioleoyl-sn-glycero-3-phosphocholine (DOPC). In some embodiments, the at least one cationic lipid comprises 1,2-di-O-octadecenyl-3-trimethylammonium propane (DOTMA) and the at least one additional lipid comprises 1,2-di-(9Z-octadecenoyl)-sn-glycero-3-phosphoethanolamine (DOPE). In some embodiments, the liposomes and RNA lipoplex particles comprise 1,2-di-O-octadecenyl-3-trimethylammonium propane (DOTMA) and 1,2-di-(9Z-octadecenoyl)-sn-glycero-3-phosphoethanolamine (DOPE).

Spleen targeting RNA lipoplex particles are described in WO 2013/143683, herein incorporated by reference. It has been found that RNA lipoplex particles having a net negative charge may be used to preferentially target spleen tissue or spleen cells such as antigen-presenting cells, in particular dendritic cells. Accordingly, following administration of the RNA lipoplex particles, RNA accumulation and/or RNA expression in the spleen occurs. Thus, RNA lipoplex particles of the disclosure may be used for expressing RNA in the spleen. In an embodiment, after administration of the RNA lipoplex particles, no or essentially no RNA accumulation and/or RNA expression in the lung and/or liver occurs. In some embodiments, after administration of the RNA lipoplex particles, RNA accumulation and/or RNA expression in antigen presenting cells, such as professional antigen presenting cells in the spleen occurs. Thus, RNA lipoplex particles of the disclosure may be used for expressing RNA in such antigen presenting cells. In some embodiments, the antigen presenting cells are dendritic cells and/or macrophages.

### Lipid Nanoparticles (LNPs)

In some embodiments, nucleic acid such as RNA described herein is administered in the form of lipid nanoparticles (LNPs). The LNP may comprise any lipid capable of forming a particle to which the one or more nucleic acid molecules are attached, or in which the one or more nucleic acid molecules are encapsulated.

In some embodiments, the LNP comprises one or more cationic lipids, and one or more stabilizing lipids. Stabilizing lipids include neutral lipids and pegylated lipids.

In some embodiments, the LNP comprises a cationic lipid, a neutral lipid, a steroid, a polymer conjugated lipid; and the RNA, encapsulated within or associated with the lipid nanoparticle.

In some embodiments, an LNP comprises from 40 to 55 mol percent, from 40 to 50 mol percent, from 41 to 49 mol percent, from 41 to 48 mol percent, from 42 to 48 mol percent, from 43 to 48 mol percent, from 44 to 48 mol percent, from 45 to 48 mol percent, from 46 to 48 mol percent, from 47 to 48 mol percent, or from 47.2 to 47.8 mol percent of the cationic lipid. In some embodiments, the LNP comprises about 47.0, 47.1, 47.2, 47.3, 47.4, 47.5, 47.6, 47.7, 47.8, 47.9 or 48.0 mol percent of the cationic lipid.

In some embodiments, the neutral lipid is present in a concentration ranging from 5 to 15 mol percent, from 7 to 13 mol percent, or from 9 to 11 mol percent. In some embodiments, the neutral lipid is present in a concentration of about 9.5, 10 or 10.5 mol percent.

In some embodiments, the steroid is present in a concentration ranging from 30 to 50 mol percent, from 35 to 45 mol percent or from 38 to 43 mol percent. In some embodiments, the steroid is present in a concentration of about 40, 41, 42, 43, 44, 45 or 46 mol percent.

In some embodiments, the LNP comprises from 1 to 10 mol percent, from 1 to 5 mol percent, or from 1 to 2.5 mol percent of the polymer conjugated lipid.

In some embodiments, the LNP comprises from 40 to 50 mol percent a cationic lipid; from 5 to 15 mol percent of a neutral lipid; from 35 to 45 mol percent of a steroid; from 1 to 10 mol percent of a polymer conjugated lipid; and the RNA, encapsulated within or associated with the lipid nanoparticle.

In some embodiments, the mol percent is determined based on total mol of lipid present in the lipid nanoparticle.

In some embodiments, the neutral lipid is selected from the group consisting of DSPC, DPPC, DMPC, DOPC, POPC, DOPE, DOPG, DPPG, POPE, DPPE, DMPE, DSPE, and SM. In some embodiments, the neutral lipid is selected from the group consisting of DSPC, DPPC, DMPC, DOPC, POPC, DOPE and SM. In some embodiments, the neutral lipid is DSPC.

In some embodiments, the steroid is cholesterol.

In some embodiments, the polymer conjugated lipid is a pegylated lipid. In some embodiments, the pegylated lipid has the following structure:

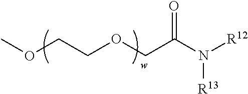

or a pharmaceutically acceptable salt, tautomer or stereoisomer thereof, wherein: R¹² and R¹³ are each independently a straight or branched, saturated or unsaturated alkyl chain containing from 10 to 30 carbon atoms, wherein the alkyl chain is optionally interrupted by one or more ester bonds; and w has a mean value ranging from 30 to 60. In some embodiments, R¹² and R¹³ are each independently straight, saturated alkyl chains containing from 12 to 16 carbon atoms. In some embodiments, w has a mean value ranging from 40 to 55. In some embodiments, the average w is about 45. In some embodiments, R¹² and R¹³ are each independently a straight, saturated alkyl chain containing about 14 carbon atoms, and w has a mean value of about 45.

In some embodiments, the pegylated lipid is DMG-PEG 2000, e.g., having the following structure:

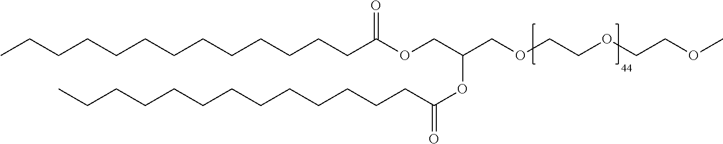

In some embodiments, the cationic lipid component of the LNPs has the structure of Formula (III):

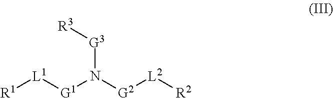

or a pharmaceutically acceptable salt, tautomer, prodrug or stereoisomer thereof, wherein:
one of L¹ or L² is —O(C═O)—, —(C═O)O—, —C(═O)—, —O—, —S(O)ₓ—, —S—S—, —C(═O)S—, SC(═O)—, —NR^{a}C(═O)—, —C(═O)NR^{a}—, NR^{a}C(═O)NR^{a}—, —OC(═O)NR^{a}— or —NR^{a}C(═O)O—, and the other of L¹ or L² is —O(C═O)—, —(C═O)O—, —C(═O)—, —O—, —S(O)ₓ—, —S—S—, —C(═O)S—, SC(═O)—, —NR^{a}C(═O)—, —C(═O)NR^{a}—, —NR^{a}C(═O)NR^{a}—, —OC(═O)NR^{a}— or —NR^{a}C(═O)O— or a direct bond;
G¹ and G² are each independently unsubstituted C₁-C₁₂ alkylene or C₁-C₁₂ alkenylene;
G³ is C₁-C₂₄ alkylene, C₁-C₂₄ alkenylene, C₃-C₈ cycloalkylene, C₃-C₈ cycloalkenylene;
R^{a} is H or C₁-C₁₂ alkyl;
R¹ and R² are each independently C₆-C₂₄ alkyl or C₆-C₂₄ alkenyl;
R³ is H, OR⁵, CN, —C(═O)OR⁴, —OC(═O)R⁴ or —NR⁵C(═O)R⁴;
R⁴ is C₁-C₁₂ alkyl;
R⁵ is H or C₁-C₆ alkyl; and
x is 0, 1 or 2.

In some of the foregoing embodiments of Formula (III), the lipid has one of the following structures (IIIA) or (IIIB):

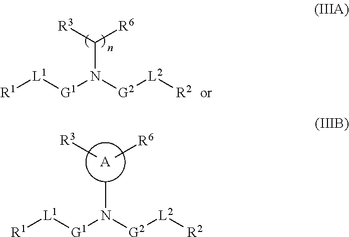

wherein:
A is a 3 to 8-membered cycloalkyl or cycloalkylene ring;
R⁶ is, at each occurrence, independently H, OH or C₁-C₂₄ alkyl;
n is an integer ranging from 1 to 15.

In some of the foregoing embodiments of Formula (III), the lipid has structure (IIIA), and in other embodiments, the lipid has structure (IIIB).

In other embodiments of Formula (III), the lipid has one of the following structures (IIIC) or (IIID):

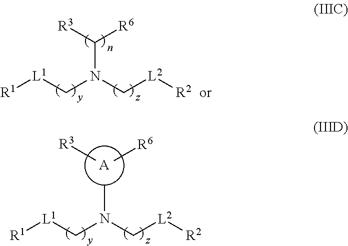

wherein y and z are each independently integers ranging from 1 to 12.

In any of the foregoing embodiments of Formula (III), one of L¹ or L² is —O(C═O)—. For example, in some embodiments each of L¹ and L² are —O(C═O)—. In some different embodiments of any of the foregoing, L¹ and L² are each independently —(C═O)O— or —O(C═O)—. For example, in some embodiments each of L¹ and L² is —(C═O)O—.

In some different embodiments of Formula (III), the lipid has one of the following structures (IIIE) or (IIIF):

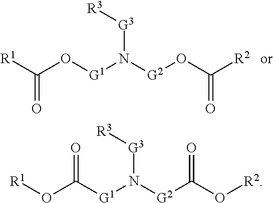

In some of the foregoing embodiments of Formula (III), the lipid has one of the following structures (IIIG), (IIIH), (IIII), or (IIIJ):

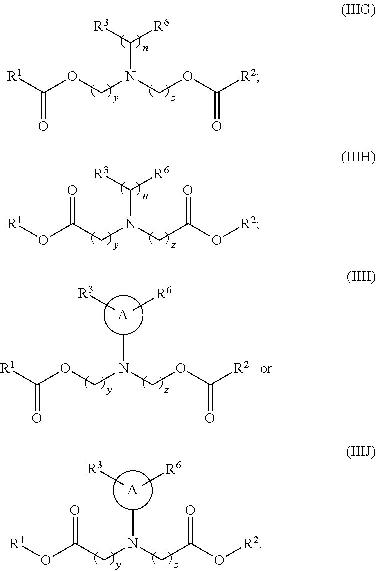

In some of the foregoing embodiments of Formula (III), n is an integer ranging from 2 to 12, for example from 2 to 8 or from 2 to 4. For example, in some embodiments, n is 3, 4, 5 or 6. In some embodiments, n is 3. In some embodiments, n is 4. In some embodiments, n is 5. In some embodiments, n is 6.

In some other of the foregoing embodiments of Formula (III), y and z are each independently an integer ranging from 2 to 10. For example, in some embodiments, y and z are each independently an integer ranging from 4 to 9 or from 4 to 6.

In some of the foregoing embodiments of Formula (III), R⁶ is H. In other of the foregoing embodiments, R⁶ is C₁-C₂₄ alkyl. In other embodiments, R⁶ is OH.

In some embodiments of Formula (III), G³ is unsubstituted. In other embodiments, G³ is substituted. In various different embodiments, G³ is linear C1-C₂₄ alkylene or linear C1-C₂₄ alkenylene.

In some other foregoing embodiments of Formula (III), R¹ or R², or both, is C₆-C₂₄ alkenyl. For example, in some embodiments, R¹ and R² each, independently have the following structure:

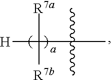

wherein:
R^{7a} and R^{7b} are, at each occurrence, independently H or C₁-C₁₂ alkyl; and
a is an integer from 2 to 12,
wherein R^{7a}, R^{7b} and a are each selected such that R¹ and R² each independently comprise from 6 to 20 carbon atoms. For example, in some embodiments a is an integer ranging from 5 to 9 or from 8 to 12.

In some of the foregoing embodiments of Formula (III), at least one occurrence of R^{7a} is H. For example, in some embodiments, R^{7a} is H at each occurrence. In other different embodiments of the foregoing, at least one occurrence of R^{7b} is C₁-C₈ alkyl. For example, in some embodiments, C₁-C₈ alkyl is methyl, ethyl, n-propyl, iso-propyl, n-butyl, iso-butyl, tert-butyl, n-hexyl or n-octyl.

In different embodiments of Formula (III), R¹ or R², or both, has one of the following structures:

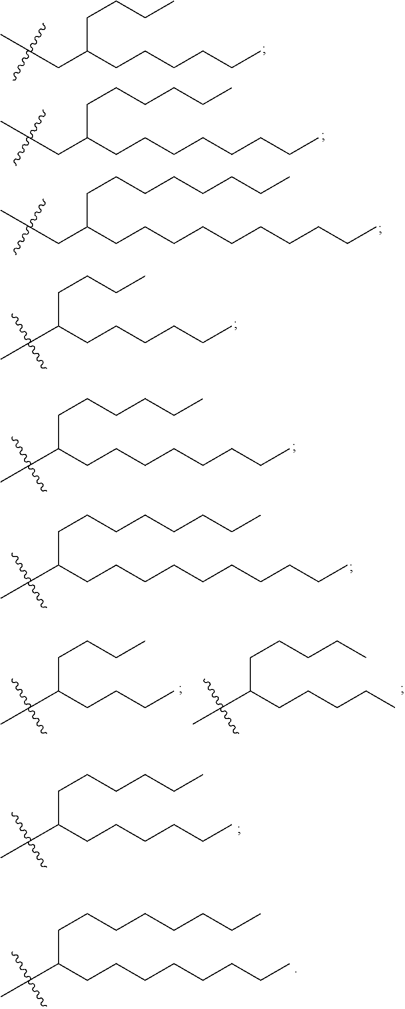

In some of the foregoing embodiments of Formula (III), R³ is OH, CN, —C(═O)OR⁴, —OC(═O)R⁴ or —NHC(═O)R⁴. In some embodiments, R⁴ is methyl or ethyl.

In various different embodiments, the cationic lipid of Formula (III) has one of the structures set forth in the table below.

### Representative Compounds of Formula (III).

| No. | Structure |
|---|---|
| III-1 | 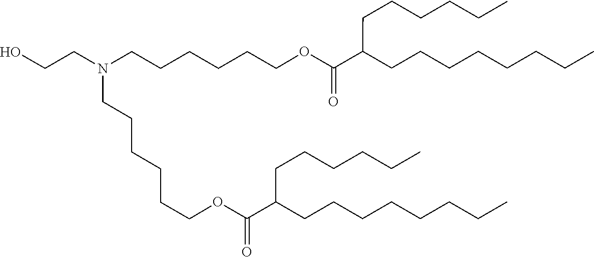 [SMILES: CCCCCCCCC(CCCCCC)C(=O)OCCCCCCN(CCO)CCCCCCOC(=O)C(CCCCCC)CCCCCCCC] |
| III-2 | 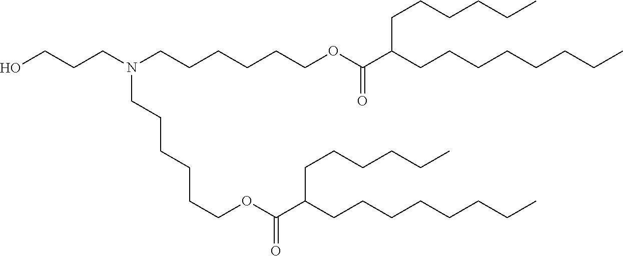 [SMILES: CCCCCCCCC(CCCCCC)C(=O)OCCCCCCN(CCCO)CCCCCCOC(=O)C(CCCCCC)CCCCCCCC] |
| III-3 | 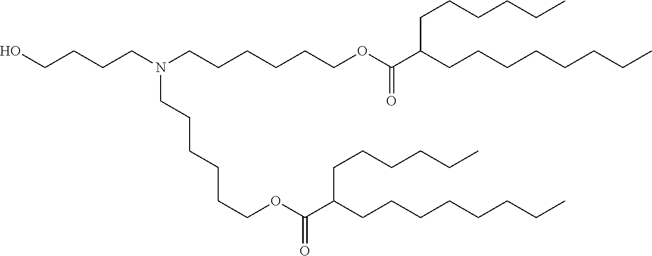 [SMILES: CCCCCCCCC(CCCCCC)C(=O)OCCCCCCN(CCCCO)CCCCCCOC(=O)C(CCCCCC)CCCCCCCC] |
| III-4 | 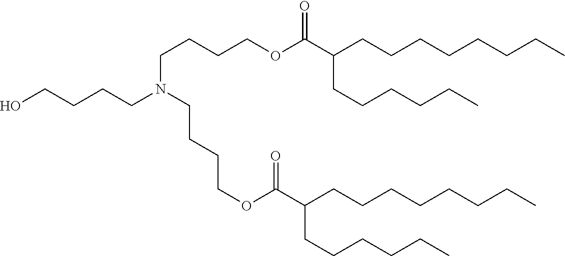 [SMILES: CCCCCCCCC(CCCCCC)C(=O)OCCCCN(CCCCO)CCCCOC(=O)C(CCCCCC)CCCCCCCC] |
| III-5 | 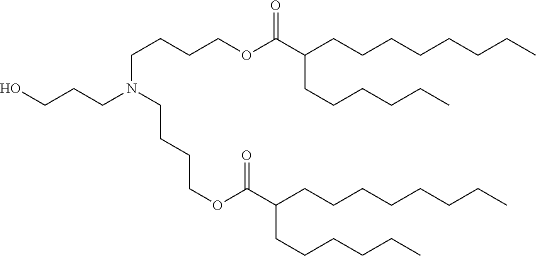 [SMILES: CCCCCCCCC(CCCCCC)C(=O)OCCCCN(CCCO)CCCCOC(=O)C(CCCCCC)CCCCCCCC] |
| III-6 | 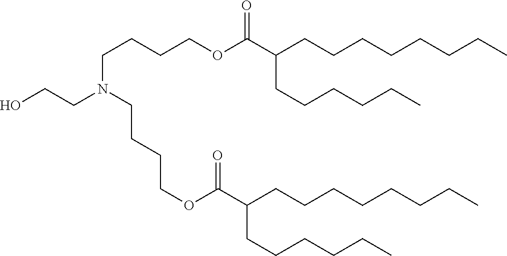 [SMILES: CCCCCCCCC(CCCCCC)C(=O)OCCCCN(CCO)CCCCOC(=O)C(CCCCCC)CCCCCCCC] |
| III-7 | 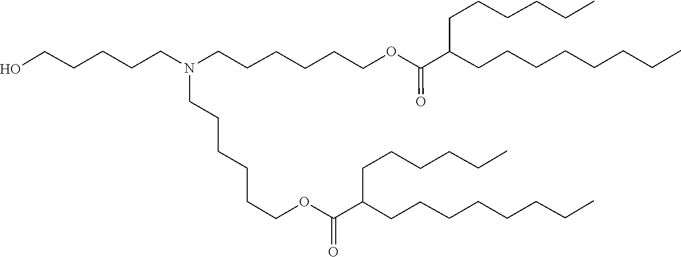 [SMILES: CCCCCCCCC(CCCCCC)C(=O)OCCCCCCN(CCCCCO)CCCCCCOC(=O)C(CCCCCC)CCCCCCCC] |
| III-8 | 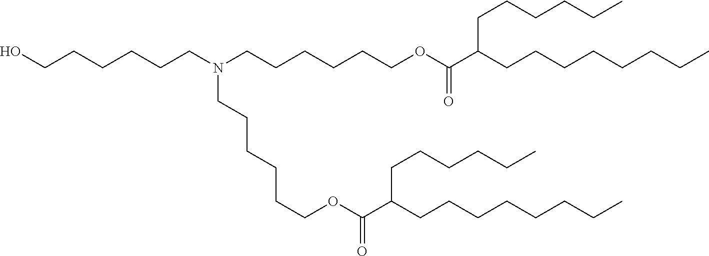 [SMILES: CCCCCCCCC(CCCCCC)C(=O)OCCCCCCN(CCCCCCO)CCCCCCOC(=O)C(CCCCCC)CCCCCCCC] |
| III-9 | 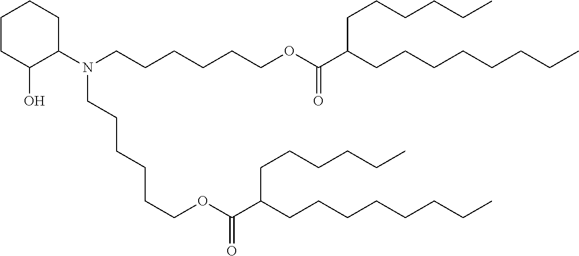 [SMILES: CCCCCCCCC(CCCCCC)C(=O)OCCCCCCN(CCCCCCOC(=O)C(CCCCCC)CCCCCCCC)C1CCCCC1O] |
| III-10 | 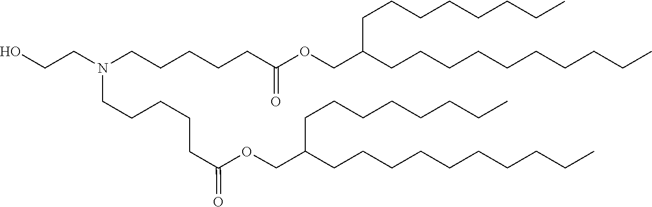 [SMILES: CCCCCCCCCCC(CCCCCCCC)COC(=O)CCCCCN(CCO)CCCCCC(=O)OCC(CCCCCCCC)CCCCCCCCCC] |
| III-11 | 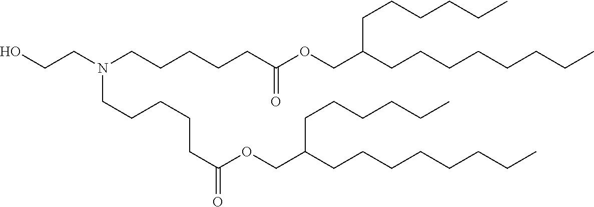 [SMILES: CCCCCCCCC(CCCCCC)COC(=O)CCCCCN(CCO)CCCCCC(=O)OCC(CCCCCC)CCCCCCCC] |
| III-12 | 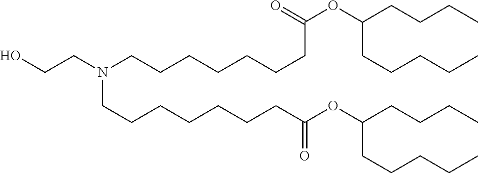 [SMILES: CCCCCC(CCCC)OC(=O)CCCCCCCN(CCO)CCCCCCCC(=O)OC(CCCC)CCCCC] |
| III-13 | 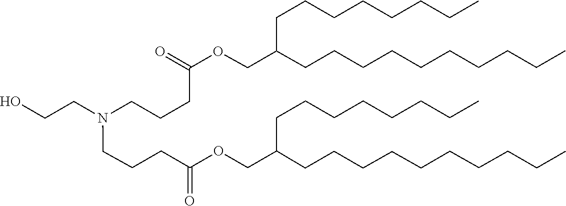 [SMILES: CCCCCCCCCCC(CCCCCCCC)COC(=O)CCCN(CCO)CCCC(=O)OCC(CCCCCCCC)CCCCCCCCCC] |
| III-14 | 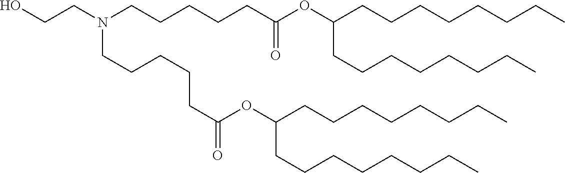 [SMILES: CCCCCCCCC(CCCCCCCC)OC(=O)CCCCCN(CCO)CCCCCC(=O)OC(CCCCCCCC)CCCCCCCC] |
| III-15 | 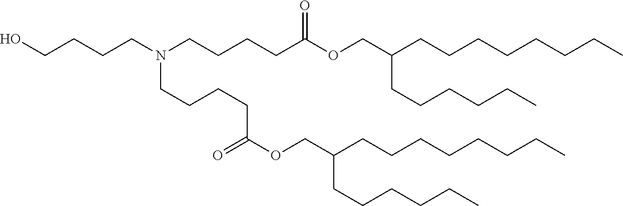 [SMILES: CCCCCCCCC(CCCCCC)COC(=O)CCCCN(CCCCO)CCCCC(=O)OCC(CCCCCC)CCCCCCCC] |
| III-16 | 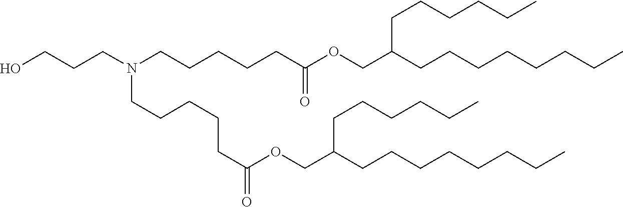 [SMILES: CCCCCCCCC(CCCCCC)COC(=O)CCCCCN(CCCO)CCCCCC(=O)OCC(CCCCCC)CCCCCCCC] |
| III-17 | 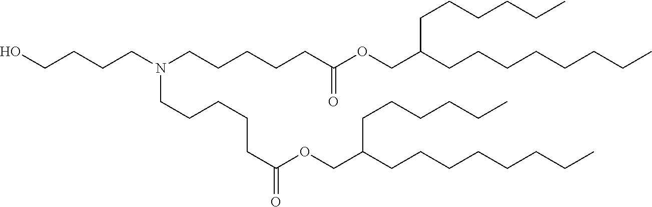 [SMILES: CCCCCCCCC(CCCCCC)COC(=O)CCCCCN(CCCCO)CCCCCC(=O)OCC(CCCCCC)CCCCCCCC] |
| III-18 | 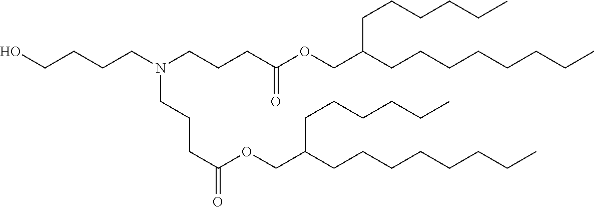 [SMILES: CCCCCCCCC(CCCCCC)COC(=O)CCCN(CCCCO)CCCC(=O)OCC(CCCCCC)CCCCCCCC] |
| III-19 | 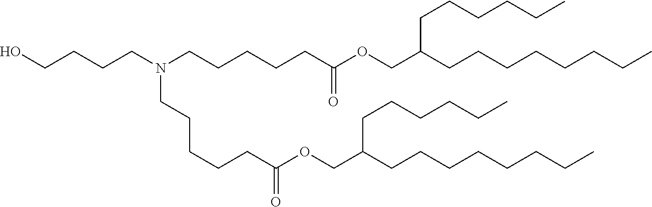 [SMILES: CCCCCCCCC(CCCCCC)COC(=O)CCCCCN(CCCCO)CCCCCC(=O)OCC(CCCCCC)CCCCCCCC] |
| III-20 | 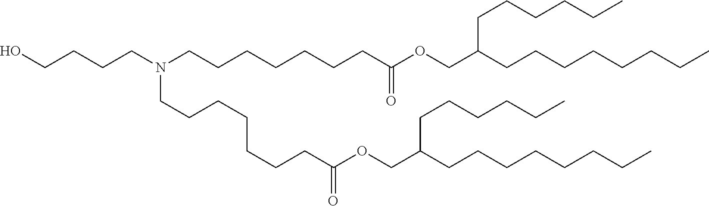 [SMILES: CCCCCCCCC(CCCCCC)COC(=O)CCCCCCCN(CCCCO)CCCCCCCC(=O)OCC(CCCCCC)CCCCCCCC] |
| III-21 | 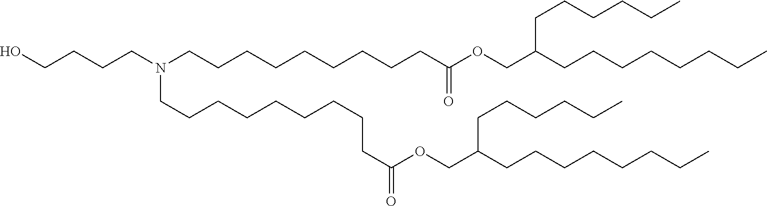 [SMILES: CCCCCCCCC(CCCCCC)COC(=O)CCCCCCCCCN(CCCCO)CCCCCCCCCC(=O)OCC(CCCCCC)CCCCCCCC] |
| III-22 | 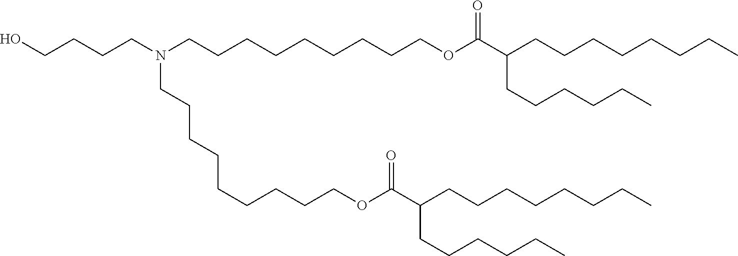 [SMILES: CCCCCCCCC(CCCCCC)C(=O)OCCCCCCCCCN(CCCCO)CCCCCCCCCOC(=O)C(CCCCCC)CCCCCCCC] |
| III-23 | 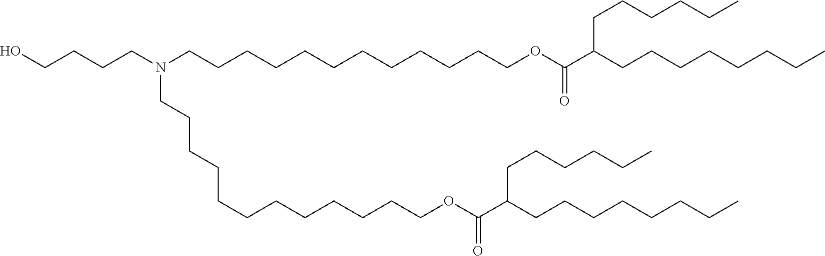 [SMILES: CCCCCCCCC(CCCCCC)C(=O)OCCCCCCCCCCCCN(CCCCO)CCCCCCCCCCCCOC(=O)C(CCCCCC)CCCCCCCC] |
| III-24 | 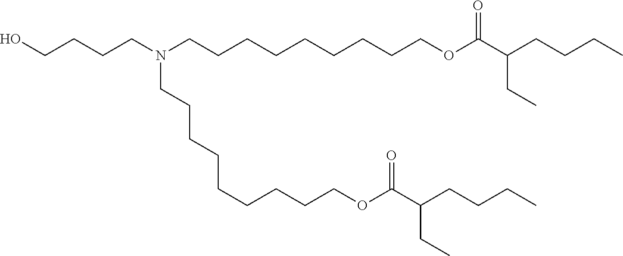 [SMILES: CCCCC(CC)C(=O)OCCCCCCCCCN(CCCCO)CCCCCCCCCOC(=O)C(CC)CCCC] |
| III-25 | 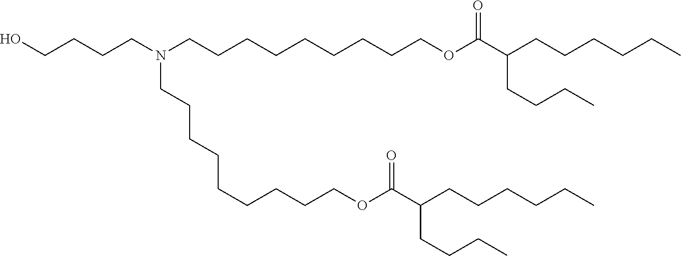 [SMILES: CCCCCCC(CCCC)C(=O)OCCCCCCCCCN(CCCCO)CCCCCCCCCOC(=O)C(CCCC)CCCCCC] |
| III-26 | 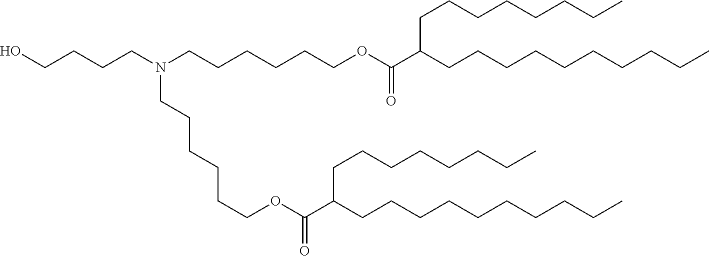 [SMILES: CCCCCCCCCCC(CCCCCCCC)C(=O)OCCCCCCN(CCCCO)CCCCCCOC(=O)C(CCCCCCCC)CCCCCCCCCC] |
| III-27 | 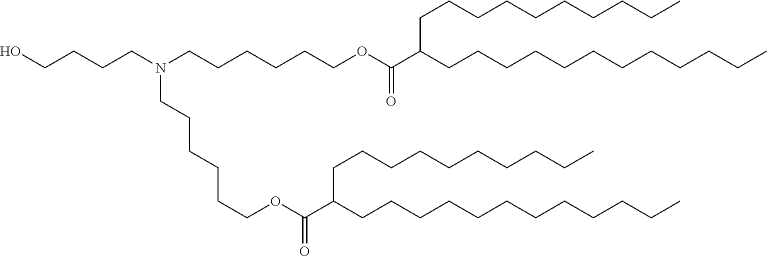 [SMILES: CCCCCCCCCCCCC(CCCCCCCCCC)C(=O)OCCCCCCN(CCCCO)CCCCCCOC(=O)C(CCCCCCCCCC)CCCCCCCCCCCC] |
| III-28 | 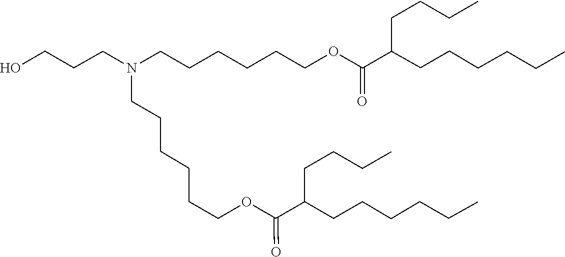 [SMILES: CCCCCCC(CCCC)C(=O)OCCCCCCN(CCCO)CCCCCCOC(=O)C(CCCC)CCCCCC] |
| III-29 | 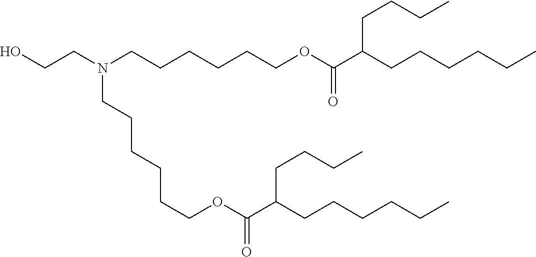 [SMILES: CCCCCCC(CCCC)C(=O)OCCCCCCN(CCO)CCCCCCOC(=O)C(CCCC)CCCCCC] |
| III-30 | 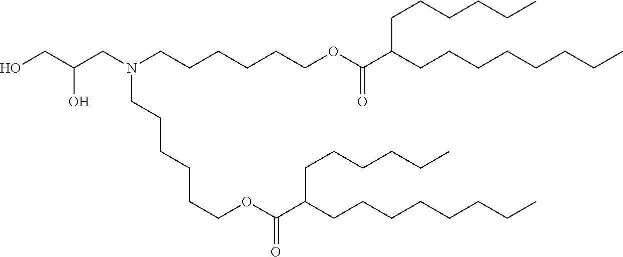 [SMILES: CCCCCCCCC(CCCCCC)C(=O)OCCCCCCN(CCCCCCOC(=O)C(CCCCCC)CCCCCCCC)CC(O)CO] |
| III-31 | 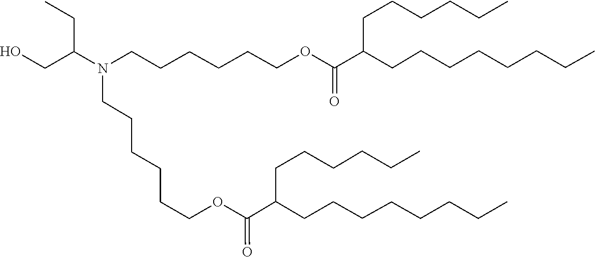 [SMILES: CCCCCCCCC(CCCCCC)C(=O)OCCCCCCN(CCCCCCOC(=O)C(CCCCCC)CCCCCCCC)C(CC)CO] |
| III-32 | 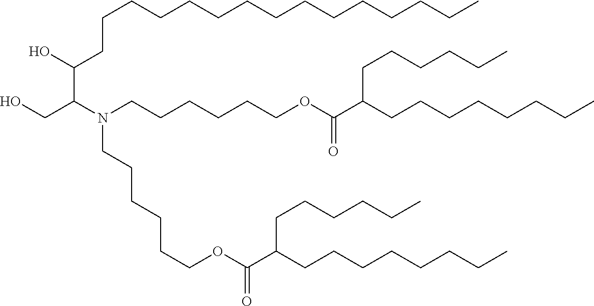 [SMILES: CCCCCCCCCCCCCCCC(O)C(CO)N(CCCCCCOC(=O)C(CCCCCC)CCCCCCCC)CCCCCCOC(=O)C(CCCCCC)CCCCCCCC] |
| III-33 | 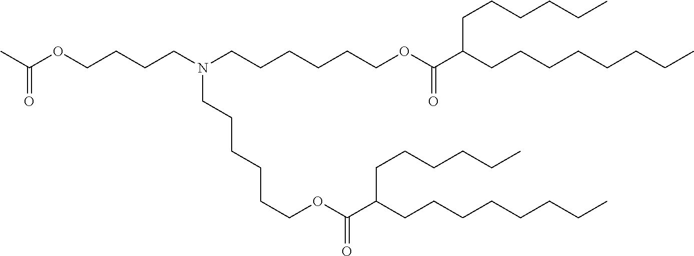 [SMILES: CCCCCCCCC(CCCCCC)C(=O)OCCCCCCN(CCCCCCOC(=O)C(CCCCCC)CCCCCCCC)CCCCOC(C)=O] |
| III-34 | 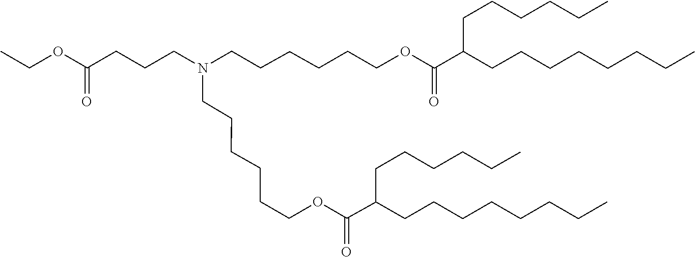 [SMILES: CCCCCCCCC(CCCCCC)C(=O)OCCCCCCN(CCCCCCOC(=O)C(CCCCCC)CCCCCCCC)CCCC(=O)OCC] |
| III-35 | 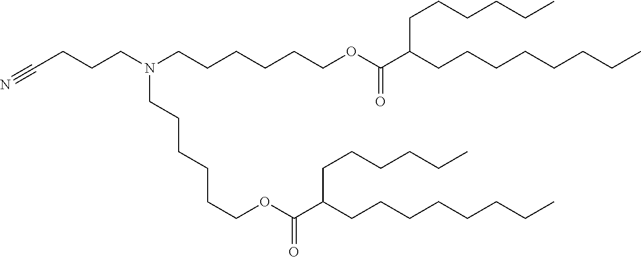 [SMILES: CCCCCCCCC(CCCCCC)C(=O)OCCCCCCN(CCCC#N)CCCCCCOC(=O)C(CCCCCC)CCCCCCCC] |
| III-36 | 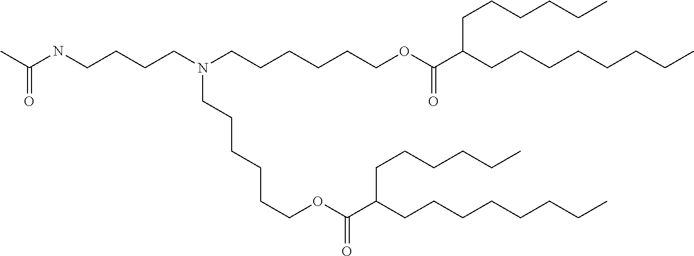 |

In some embodiments, an LNP comprises a lipid of Formula (III), RNA, a neutral lipid, a steroid and a pegylated lipid. In some embodiments, a lipid of Formula (III) is compound III-3. In some embodiments, a neutral lipid is DSPC. In some embodiments, a steroid is cholesterol. In some embodiments, a pegylated lipid is ALC-0159.

In some embodiments, the cationic lipid is present in the LNP in an amount from about 40 to about 50 mole percent. In some embodiments, the neutral lipid is present in the LNP in an amount from about 5 to about 15 mole percent. In some embodiments, the steroid is present in the LNP in an amount from about 35 to about 45 mole percent. In some embodiments, the pegylated lipid is present in the LNP in an amount from about 1 to about 10 mole percent.

In some embodiments, the LNP comprises compound III-3 in an amount from about 40 to about 50 mole percent, DSPC in an amount from about 5 to about 15 mole percent, cholesterol in an amount from about 35 to about 45 mole percent, and ALC-0159 in an amount from about 1 to about 10 mole percent.

In some embodiments, the LNP comprises compound III-3 in an amount of about 47.5 mole percent, DSPC in an amount of about 10 mole percent, cholesterol in an amount of about 40.7 mole percent, and ALC-0159 in an amount of about 1.8 mole percent.

In various different embodiments, the cationic lipid has one of the structures set forth in the table below.

| No. | Structure |
|---|---|
| A | 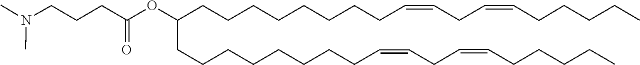 [SMILES: CCCCC/C=C\C/C=C\CCCCCCCCC(CCCCCCCC/C=C\C/C=C\CCCCC)OC(=O)CCCN(C)C] |
| B | 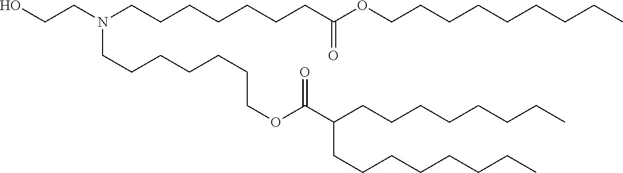 [SMILES: CCCCCCCCCOC(=O)CCCCCCCN(CCO)CCCCCCCOC(=O)C(CCCCCCCC)CCCCCCCC] |
| C | 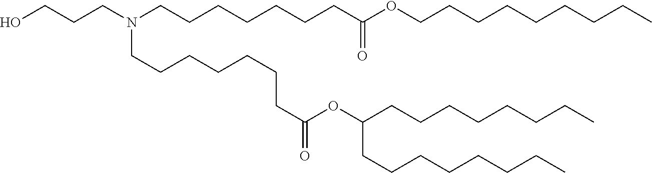 [SMILES: CCCCCCCCCOC(=O)CCCCCCCN(CCCO)CCCCCCCC(=O)OC(CCCCCCCC)CCCCCCCC] |
| D | 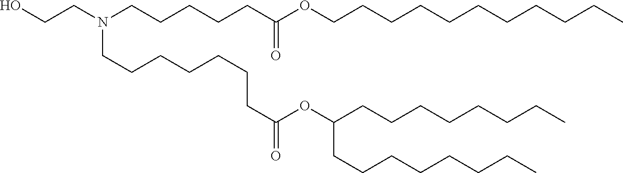 [SMILES: CCCCCCCCCCCOC(=O)CCCCCN(CCO)CCCCCCCC(=O)OC(CCCCCCCC)CCCCCCCC] |
| E | 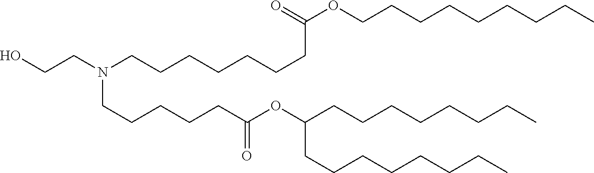 [SMILES: CCCCCCCCCOC(=O)CCCCCCCN(CCO)CCCCCC(=O)OC(CCCCCCCC)CCCCCCCC] |
| F | 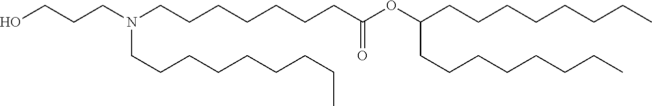 [SMILES: CCCCCCCCCN(CCCO)CCCCCCCC(=O)OC(CCCCCCCC)CCCCCCCC] |

In some embodiments, the LNP comprises a cationic lipid shown in the above table, e.g., a cationic lipid of Formula (B) or Formula (D), in particular a cationic lipid of Formula (D), RNA, a neutral lipid, a steroid and a pegylated lipid. In some embodiments, the neutral lipid is DSPC. In some embodiments, the steroid is cholesterol. In some embodiments, the pegylated lipid is DMG-PEG 2000.

In some embodiments, the LNP comprises a cationic lipid that is an ionizable lipid-like material (lipidoid). In some embodiments, the cationic lipid has the following structure:

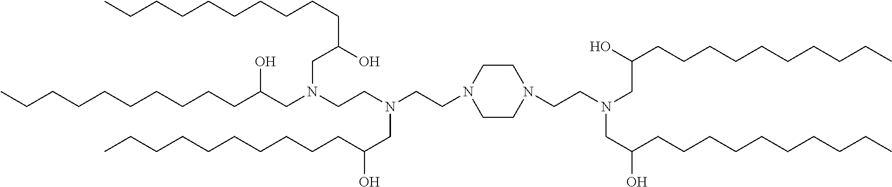 [SMILES: CCCCCCCCCCC(O)CN(CCN1CCN(CCN(CC(O)CCCCCCCCCC)CC(O)CCCCCCCCCC)CC1)CCN(CC(O)CCCCCCCCCC)CC(O)CCCCCCCCCC]

The N/P value is preferably at least about 4. In some embodiments, the N/P value ranges from 4 to 20, 4 to 12, 4 to 10, 4 to 8, or 5 to 7. In some embodiments, the N/P value is about 6.

LNP described herein may have an average diameter that in some embodiments ranges from about 30 nm to about 200 nm, or from about 60 nm to about 120 nm.

### Pharmaceutical Compositions

In some embodiments, a pharmaceutical composition comprises an RNA polynucleotide disclosed herein formulated as a particle. In some embodiments, a particle is or comprises a lipid nanoparticle (LNP) or a lipoplex (LPX) particle.

In some embodiments, an RNA polynucleotide disclosed herein may be administered in a pharmaceutical composition or a medicament and may be administered in the form of any suitable pharmaceutical composition.

In some embodiments, a pharmaceutical composition described herein is an immunogenic composition for inducing an immune response. For example, in some embodiments, an immunogenic composition is a vaccine.

In some embodiments, an RNA polynucleotide disclosed herein may be administered in a pharmaceutical composition which may comprise a pharmaceutically acceptable carrier and may optionally comprise one or more adjuvants, stabilizers etc. In some embodiments, a pharmaceutical composition is for therapeutic or prophylactic treatments.

The term “adjuvant” relates to a compound which prolongs, enhances or accelerates an immune response. Adjuvants comprise a heterogeneous group of compounds such as oil emulsions (e.g., Freund's adjuvants), mineral compounds (such as alum), bacterial products (such as Bordetella pertussis toxin), or immune-stimulating complexes. Examples of adjuvants include, without limitation, LPS, GP96, CpG oligodeoxynucleotides, growth factors, and cytokines, such as monokines, lymphokines, interleukins, chemokines. The cytokines may be IL1, IL2, IL3, IL4, IL5, IL6, IL7, IL8, IL9, IL10, IL12, IFNα, IFNγ, GM-CSF, LT-a. Further known adjuvants are aluminium hydroxide, Freund's adjuvant or oil such as Montanide® ISA51. Other suitable adjuvants for use in the present disclosure include lipopeptides, such as Pam3Cys.
The pharmaceutical compositions according to the present disclosure are generally applied in a “pharmaceutically effective amount” and in “a pharmaceutically acceptable preparation”.
The term “pharmaceutically acceptable” refers to the non-toxicity of a material which does not interact with the action of the active component of the pharmaceutical composition.
The term “pharmaceutically effective amount” or “therapeutically effective amount” refers to the amount which achieves a desired reaction or a desired effect alone or together with further doses. In the case of the treatment of a particular disease, the desired reaction preferably relates to inhibition of the course of the disease. This comprises slowing down the progress of the disease and, in particular, interrupting or reversing the progress of the disease. The desired reaction in a treatment of a disease may also be delay of the onset or a prevention of the onset of said disease or said condition. An effective amount of the compositions described herein will depend on the condition to be treated, the severeness of the disease, the individual parameters of the patient, including age, physiological condition, size and weight, the duration of treatment, the type of an accompanying therapy (if present), the specific route of administration and similar factors. Accordingly, the doses administered of the compositions described herein may depend on various of such parameters. In the case that a reaction in a patient is insufficient with an initial dose, higher doses (or effectively higher doses achieved by a different, more localized route of administration) may be used.

In some embodiments, a pharmaceutical composition disclosed herein may contain salts, buffers, preservatives, and optionally other therapeutic agents. In some embodiments, a pharmaceutical composition disclosed herein comprises one or more pharmaceutically acceptable carriers, diluents and/or excipients.

Suitable preservatives for use in a pharmaceutical compositions of the present disclosure include, without limitation, benzalkonium chloride, chlorobutanol, paraben and thimerosal.

The term “excipient” as used herein refers to a substance which may be present in a pharmaceutical composition of the present disclosure but is not an active ingredient. Examples of excipients, include without limitation, carriers, binders, diluents, lubricants, thickeners, surface active agents, preservatives, stabilizers, emulsifiers, buffers, flavoring agents, or colorants.

The term “diluent” relates a diluting and/or thinning agent. Moreover, the term “diluent” includes any one or more of fluid, liquid or solid suspension and/or mixing media. Examples of suitable diluents include ethanol, glycerol and water.

The term “carrier” refers to a component which may be natural, synthetic, organic, inorganic in which the active component is combined in order to facilitate, enhance or enable administration of the pharmaceutical composition. A carrier as used herein may be one or more compatible solid or liquid fillers, diluents or encapsulating substances, which are suitable for administration to subject. Suitable carrier include, without limitation, sterile water, Ringer, Ringer lactate, sterile sodium chloride solution, isotonic saline, polyalkylene glycols, hydrogenated naphthalenes and, in particular, biocompatible lactide polymers, lactide/glycolide copolymers or polyoxyethylene/polyoxy-propylene copolymers. In some embodiments, the pharmaceutical composition of the present disclosure includes isotonic saline.

Pharmaceutically acceptable carriers, excipients or diluents for therapeutic use are well known in the pharmaceutical art, and are described, for example, in Remington's Pharmaceutical Sciences, Mack Publishing Co. (A. R Gennaro edit. 1985).

Pharmaceutical carriers, excipients or diluents can be selected with regard to the intended route of administration and standard pharmaceutical practice.

In some embodiments, a pharmaceutical composition described herein may be administered intravenously, intraarterially, subcutaneously, intradermally or intramuscularly. In certain embodiments, the pharmaceutical composition is formulated for local administration or systemic administration. Systemic administration may include enteral administration, which involves absorption through the gastrointestinal tract, or parenteral administration. As used herein, “parenteral administration” refers to the administration in any manner other than through the gastrointestinal tract, such as by intravenous injection. In a preferred embodiment, the pharmaceutical composition is formulated for intramuscular administration. In another embodiment, the pharmaceutical composition is formulated for systemic administration, e.g., for intravenous administration.

### Characterization

In some embodiments, an RNA polynucleotide disclosed herein is characterized in that, when assessed in an organism administered a composition or medical preparation comprising an RNA polynucleotide, elevated expression of a payload is observed relative to an appropriate reference comparator.

In some embodiments, an RNA polynucleotide disclosed herein is characterized in that, when assessed in an organism administered a composition or medical preparation comprising an RNA polynucleotide, increased duration of expression (e.g., prolonged expression) of a payload is observed relative to an appropriate reference comparator.

In some embodiments, an RNA polynucleotide disclosed herein is characterized in that, when assessed in an organism administered a composition or medical preparation comprising an RNA polynucleotide, decreased interaction with IFIT1 of an RNA polynucleotide is observed relative to an appropriate reference comparator.

In some embodiments, an RNA polynucleotide disclosed herein is characterized in that, when assessed in an organism administered a composition or medical preparation comprising an RNA polynucleotide, increased translation an RNA polynucleotide is observed relative to an appropriate reference comparator.

In some embodiments, a reference comparator comprises an organism administered an otherwise similar RNA polynucleotide without a cap described herein. In some embodiments, a reference comparator comprises an organism administered an otherwise similar RNA polynucleotide without a cap proximal sequence disclosed herein. In some embodiments, a reference comparator comprises an organism administered an otherwise similar RNA polynucleotide with a self-hybridizing sequence.

In some embodiments, an RNA polynucleotide disclosed herein is characterized in that, when assessed in an organism administered a composition or medical preparation comprising an RNA polynucleotide, elevated expression and increased duration of expression (e.g., prolonged expression) of a payload is observed relative to an appropriate reference comparator.

In some embodiments, elevated expression is determined at least 24 hours, at least 48 hours at least 72 hours, at least 96 hours or at least 120 hours after administration of a composition or medical preparation comprising an RNA polynucleotide. In some embodiments, elevated expression is determined at least 24 hours after administration of a composition or medical preparation comprising an RNA polynucleotide. In some embodiments, elevated expression is determined at least 48 hours after administration of a composition or medical preparation comprising an RNA polynucleotide. In some embodiments, elevated expression is determined at least 72 hours after administration of a composition or medical preparation comprising an RNA polynucleotide. In some embodiments, elevated expression is determined at least 96 hours after administration of a composition or medical preparation comprising an RNA polynucleotide. In some embodiments, elevated expression is determined at least 120 hours after administration of a composition or medical preparation comprising an RNA polynucleotide.

In some embodiments, elevated expression is determined at about 24-120 hours after administration of a composition or medical preparation comprising an RNA polynucleotide. In some embodiments, elevated expression is determined at about 24-110 hours, about 24-100 hours, about 24-90 hours, about 24-80 hours, about 24-70 hours, about 24-60 hours, about 24-50 hours, about 24-40 hours, about 24-30 hours, about 30-120 hours, about 40-120 hours, about 50-120 hours, about 60-120 hours, about 70-120 hours, about 80-120 hours, about 90-120 hours, about 100-120 hours, or about 110-120 hours after administration of a composition or medical preparation comprising an RNA polynucleotide.

In some embodiments, elevated expression of a payload is at least 2-fold to at least 10-fold. In some embodiments, elevated expression of a payload is at least 2-fold. In some embodiments, elevated expression of a payload is at least 3-fold. In some embodiments, elevated expression of a payload is at least 4-fold. In some embodiments, elevated expression of a payload is at least 6-fold. In some embodiments, elevated expression of a payload is at least 8-fold. In some embodiments, elevated expression of a payload is at least 10-fold.

In some embodiments, elevated expression of a payload is about 2-fold to about 50-fold. In some embodiments, elevated expression of a payload is about 2-fold to about 45-fold, about 2-fold to about 40-fold, about 2-fold to about 30-fold, about 2-fold to about 25-fold, about 2-fold to about 20-fold, about 2-fold to about 15-fold, about 2-fold to about 10-fold, about 2-fold to about 8-fold, about 2-fold to about 5-fold, about 5-fold to about 50-fold, about 10-fold to about 50-fold, about 15-fold to about 50-fold, about 20-fold to about 50-fold, about 25-fold to about 50-fold, about 30-fold to about 50-fold, about 40-fold to about 50-fold, or about 45-fold to about 50-fold.

In some embodiments, elevated expression (e.g., increased duration of expression) of a payload persists for at least 24 hours, at least 48 hours, at least 72 hours, at least 96 hours, or at least 120 hours after administration of a composition or a medical preparation comprising an RNA polynucleotide. In some embodiments, elevated expression of a payload persists for at least 24 hours after administration. In some embodiments, elevated expression of a payload persists for at least 48 hours after administration. In some embodiments, elevated expression of a payload persists for at least 72 hours after administration. In some embodiments, elevated expression of a payload persists for at least 96 hours after administration. In some embodiments, elevated expression of a payload persists for at least 120 hours after administration of a composition or medical preparation comprising an RNA polynucleotide.

In some embodiments, elevated expression of a payload persists for at about 24-120 hours after administration of a composition or medical preparation comprising an RNA polynucleotide. In some embodiments, elevated expression persists for about 24-110 hours, about 24-100 hours, about 24-90 hours, about 24-80 hours, about 24-70 hours, about 24-60 hours, about 24-50 hours, about 24-40 hours, about 24-30 hours, about 30-120 hours, about 40-120 hours, about 50-120 hours, about 60-120 hours, about 70-120 hours, about 80-120 hours, about 90-120 hours, about 100-120 hours, or about 110-120 hours after administration of a composition or medical preparation comprising an RNA polynucleotide.

### Uses

Disclosed herein, among other things, are methods of making and methods of using an RNA polynucleotide comprising a 5′cap; a 5′ UTR comprising a cap proximal structure; and a sequence encoding a payload.

In some embodiments, disclosed herein is a method of producing a polypeptide comprising a step of: providing an RNA polynucleotide that comprises a 5′ cap (e.g., as described herein), a cap proximal sequence that comprises positions +1, +2, +3, +4, and +5 of an RNA polynucleotide, and a sequence encoding a payload; wherein an RNA polynucleotide is characterized in that when assessed in an organism administered an RNA polynucleotide or a composition comprising the same, elevated expression and/or increased duration of expression of an payload is observed relative to an appropriate reference comparator.

In some embodiments, disclosed herein is a method comprising: administering to a subject, a pharmaceutical composition comprising an RNA polynucleotide formulated in a particle described herein (e.g., in some embodiments a lipid nanoparticle (LNP) or a lipoplex (LPX) particle disclosed herein).

In some embodiments, disclosed herein is a method of inducing an immune response in a subject, comprising administering to a subject, a pharmaceutical composition comprising an RNA polynucleotide formulated in particle described herein (e.g., in some embodiments a lipid nanoparticle (LNP) or a lipoplex (LPX) particle disclosed herein).

In some embodiments, disclosed herein is a method of vaccination of a subject, comprising administering to a subject, a pharmaceutical composition comprising an RNA polynucleotide formulated in a particle described herein (e.g., in some embodiments a lipid nanoparticle (LNP) or a lipoplex (LPX) particle disclosed herein).

In some embodiments, provided herein is a method of decreasing interaction with IFIT1 of an RNA polynucleotide that comprises a 5′ cap and a cap proximal sequence comprising positions +1, +2, +3, +4, and +5 of the RNA polynucleotide, the method comprising a step of: providing a variant of an RNA polynucleotide that differs from a parental RNA polynucleotide by substitution of one or more residues within the cap proximal sequence, and determining that interaction of a variant with IFIT1 is decreased relative to that of a parental RNA polynucleotide. In some embodiments, determining comprises administering the RNA polynucleotide or a composition comprising the same to a cell or an organism.

In some embodiments, disclosed herein is a method of increasing translatability of an RNA polynucleotide that comprises a 5′ cap, a cap proximal sequence that comprises positions +1, +2, +3, +4, and +5 of the RNA polynucleotide and a sequence encoding a payload, the method comprising a step of: providing a variant of an RNA polynucleotide that differs from a parental RNA polynucleotide by substitution of one or more residues within a cap proximal sequence; and determining that expression of a variant is increased relative to that of a parental RNA polynucleotide. In some embodiments, determining comprises administering the RNA polynucleotide or a composition comprising the same to a cell or an organism. In some embodiments, increased translatability is assessed by increased expression and/or a persistence of expression of the payload. In some embodiments, increased expression is determined at least 6 hours, at least 24 hours, at least 48 hours at least 72 hours, at least 96 hours or at least 120 hours after administering. In some embodiments, increase in expression is at least 2-fold to 10-fold. In some embodiments, increase in expression is about 2-fold to 50-fold. In some embodiments, elevated expression persists for at least 24 hours, at least 48 hours, at least 72 hours, at least 96 hours, or at least 120 hours after administration.

In some embodiments of any of the methods disclosed herein, an immune response is induced in a subject. In some embodiments of any of the methods disclosed herein, an immune response is a prophylactic immune response or a therapeutic immune response.

In some embodiments of any of the methods disclosed herein, a subject is a mammal.

In some embodiments of any of the methods disclosed herein, a subject is a human.

In some embodiments of any of the methods disclosed herein, a subject has a disease or disorder disclosed herein.

In some embodiments of any of the methods disclosed herein, vaccination generates an immune response to an agent. In some embodiments, an immune response is a prophylactic immune response.

In some embodiments of any of the methods disclosed herein, a subject has a disease or disorder disclosed herein.

In some embodiments of any of the methods disclosed herein, one dose of a pharmaceutical composition is administered.

In some embodiments of any of the methods disclosed herein, a plurality of doses of a pharmaceutical composition is administered.

In some embodiments of any of the methods disclosed herein, the method further comprises administration of one or more therapeutic agents. In some embodiments, one or more therapeutic agents are administered before, after, or concurrently with administration of a pharmaceutical composition comprising an RNA polynucleotide.

Also provided herein is a method of improving capping efficiency (e.g., percentage of capped transcripts in an in vitro transcription reaction) of RNA transcripts, the improvement that comprises including a pyrimidine at +2 position of a transcription start site in a DNA template for in vitro transcription. Exemplary pyrimidines include, e.g., C or U. In some embodiments, the +1 position of a transcription start site is G. In some embodiments, the +3 position of a transcription start site is a pyrimidine or a purine. In some embodiments, the +3 position of a transcription start site is G. In some embodiments, a transcription start site may be GCG, GUG, or GCA. In some embodiments, such improvements can be observed independent of the identity of a 5′ UTR, capping method (e.g., enzymatic capping vs. co-transcriptional capping), cap structures (e.g., Cap0, Cap1, or Cap2), coding sequences, types of ribonucleotides (e.g., modified nucleotides vs. non-modified nucleotides), formulation (e.g., lipoplex vs. lipid nanoparticles) or combinations thereof.

Also provided herein is a method of improving quality of an RNA preparation (e.g., of an in vitro transcribed RNA, e.g., the amount of short polynucleotide byproducts produced), the improvement that comprises including a pyrimidine at +2 position of a transcription start site in a DNA template for in vitro transcription. Exemplary pyrimidines include, e.g., C or U. In some embodiments, the +1 position of a transcription start site is G. In some embodiments, the +3 position of a transcription start site is a pyrimidine or a purine. In some embodiments, the +3 position of a transcription start site is G. In some embodiments, a transcription start site may be GCG, GUG, or GCA. In some embodiments, such improvements can be observed independent of the identity of a 5′ UTR, capping method (e.g., enzymatic capping vs. co-transcriptional capping), cap structures (e.g., Cap0, Cap1, or Cap2), coding sequences, types of ribonucleotides (e.g., modified nucleotides vs. non-modified nucleotides), formulation (e.g., lipoplex vs. lipid nanoparticles), or combinations thereof.

Also provided herein is a method of improving translation efficiency of an RNA encoding a payload, and/or expression of a polypeptide payload encoded by an RNA, the improvement that comprises including a pyrimidine at +2 position of a transcription start site in a DNA template for in vitro transcription. Exemplary pyrimidines include, e.g., C or U. In some embodiments, the +1 position of a transcription start site is G. In some embodiments, the +3 position of a transcription start site is a pyrimidine or a purine. In some embodiments, the +3 position of a transcription start site is G. In some embodiments, a transcription start site may be GCG, GUG, or GCA. In some embodiments, such improvements can be observed independent of the identity of a 5′ UTR, capping method (e.g., enzymatic capping vs. co-transcriptional capping), cap structures (e.g., Cap0, Cap1, or Cap2), coding sequences, types of ribonucleotides (e.g., modified nucleotides vs. non-modified nucleotides), formulation (e.g., lipoplex vs. lipid nanoparticles), or combinations thereof.

Also provided herein in some embodiments, is a method of providing a framework for an RNA polynucleotide that comprises a 5′ cap, a cap proximal sequence, and a payload sequence, the method comprising a step of:
assessing at least two variants of an RNA polynucleotide, wherein:
each variant includes a same 5′ cap and payload sequence; and the variants differ from one another at one or more specific residues of a cap proximal sequence;
wherein the assessing comprises determining expression levels and/or duration of expression of a payload; and
selecting at least one combination of 5′ cap and a cap proximal sequence that displays elevated expression relative to at least one other combination.

In some embodiments, assessing comprises administering an RNA construct or a composition comprising the same to a cell or an organism:

In some embodiments, elevated expression of a payload is detected at a time point at least 6 hours, at least 24 hours, at least 48 hours, at least 72 hours, at least 96 hours, or at least 120 hours after administering. In some embodiments, elevated expression is at least 2-fold to 10-fold. In some embodiments, elevated expression is about 2-fold to about 50-fold.

In some embodiments, elevated expression of a payload persists for at least 24 hours, at least 48 hours, at least 72 hours, at least 96 hours, or at least 120 hours after administering.

In some embodiments of any of the methods disclosed herein, an RNA polynucleotide comprises one or more features of an RNA polynucleotide provided herein.

In some embodiments of any of the methods disclosed herein, a composition comprising an RNA polynucleotide comprises a pharmaceutical composition provided herein.

### Exemplary Enumerated Embodiments

1. A composition or medical preparation comprising an RNA polynucleotide comprising:
   a 5′ cap; a cap proximal sequence comprising positions +1, +2, +3, +4, and +5 of the RNA polynucleotide; and a sequence encoding a payload, wherein:
      (i) the 5′ cap is a trinucleotide cap structure comprising N₁pN₂, wherein N₁ is position +1 of the RNA polynucleotide, and N₂ is position +2 of the RNA polynucleotide, and wherein N₁ and N₂ are selected from one of the following combinations: (a) N₁ is G and N₂ is G; (b) N₁ is U and N₂ is G; (c) N₁ is A and N₂ is G; or (d) N₁ is C and N₂ is G; and
      (ii) the cap proximal sequence comprises:
         N₁ and N₂ of the trinucleotide cap structure and a sequence comprising N₃N₄N₅ at positions +3, +4, and +5 respectively of the RNA polynucleotide, wherein N₃ is C, N₄ is G, and N₅ is selected from: A, C, G, and U.
2. The composition or medical preparation of embodiment 1, wherein N₁ is G and N₂ is G.
3. The composition or medical preparation of embodiment 1, wherein N₁ is U and N₂ is G.
4. The composition or medical preparation of embodiment 1, wherein N₁ is A and N₂ is G.
5. The composition or medical preparation of embodiment 1, wherein N₁ is C and N₂ is G.
6. A composition or medical preparation comprising an RNA polynucleotide comprising:
   a 5′ cap; a cap proximal sequence comprising positions +1, +2, +3, +4, and +5 of the RNA polynucleotide; and a sequence encoding a payload, wherein:
      (i) the 5′ cap is a trinucleotide cap structure comprising N₁pN₂, wherein N₁ is position +1 of the RNA polynucleotide, and N₂ is position +2 of the RNA polynucleotide, and wherein N₁ is G and N₂ is C; and
      (ii) the cap proximal sequence comprises:
         N₁ and N₂ of the trinucleotide cap structure and a sequence comprising N₃N₄N₅ at positions +3, +4, and +5 respectively of the RNA polynucleotide, wherein N₃ is G, and each N₄ and N₅ is selected from: A, C, G, and U.
7. A composition or medical preparation comprising an RNA polynucleotide comprising:
   a 5′ cap; a cap proximal sequence comprising positions +1, +2, +3, +4, and +5 of the RNA polynucleotide; and a sequence encoding a payload, wherein:
      (i) the 5′ cap is a trinucleotide cap structure comprising N₁pN₂, wherein N₁ is position +1 of the RNA polynucleotide, and N₂ is position +2 of the RNA polynucleotide, and wherein N₁ is C and N₂ is G; and
      (ii) the cap proximal sequence comprises:
         N₁ and N₂ of the trinucleotide cap structure and a sequence comprising N₃N₄N₅ at positions +3, +4, and +5 respectively of the RNA polynucleotide, wherein N₃ is C, and each N₄ and N₅ is selected from: A, C, G, and U.
8. A composition or medical preparation comprising an RNA polynucleotide comprising:
   a 5′ cap; a cap proximal sequence comprising positions +1, +2, +3, +4, and +5 of the RNA polynucleotide; and a sequence encoding a payload, wherein:
      (i) the 5′ cap is a trinucleotide cap structure comprising N₁pN₂, wherein N₁ is position +1 of the RNA polynucleotide, and N₂ is position +2 of the RNA polynucleotide, and wherein N₁ and N₂ are selected from one of the following combinations: (a) N₁ is C and N₂ is G; (b) N₁ is U and N₂ is G; and (c) N₁ is A and N₂ is G; and
      (ii) the cap proximal sequence comprises:
         N₁ and N₂ of the trinucleotide cap structure and a sequence comprising N₃N₄N₅ at positions +3, +4, and +5 respectively of the RNA polynucleotide, wherein N₃ and N₄ are G, and N₅ is selected from: A, C, G, and U.
9. The composition or medical preparation of embodiment 8, wherein N₁ is C and N₂ is G.
10. The composition or medical preparation of embodiment 8, wherein N₁ is U and N₂ is G.
11. The composition or medical preparation of embodiment 8, wherein N₁ is A and N₂ is G.
12. A composition or medical preparation comprising an RNA polynucleotide comprising:
   a 5′ cap; a cap proximal sequence comprising positions +1, +2, +3, +4, and +5 of the RNA polynucleotide; and a sequence encoding a payload, wherein:
      (i) the 5′ cap is a trinucleotide cap structure comprising N₁pN₂, wherein N₁ is position +1 of the RNA polynucleotide, and N₂ is position +2 of the RNA polynucleotide, and wherein N₁ and N₂ are each G; and
      (ii) the cap proximal sequence comprises:
         N₁ and N₂ of the trinucleotide cap structure and a sequence comprising N₃N₄N₅ at positions +3, +4, and +5 respectively of the RNA polynucleotide, wherein N₃ is G, and each N₄ and N₅ is selected from: A, C, G, and U.
13. The composition or medical preparation of any one of embodiments 1-12, wherein the trinucleotide cap structure has a structure: G*N¹pN₂, wherein
   G* comprises a structure of formula (I):

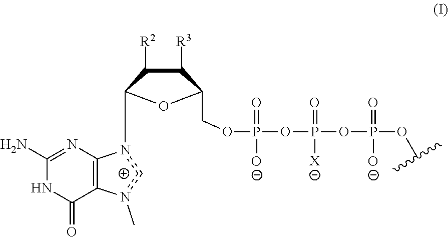

or a salt thereof,
wherein
   each R² and R³ is —OH or —OCH₃; and
   X is O or S.
14. The composition or medical preparation of embodiment 13, wherein R² is —OH.
15. The composition or medical preparation of embodiment 13, wherein R² is —OCH₃.
16. The composition or medical preparation of any one of embodiments 13-15, wherein R³ is —OH.
17. The composition or medical preparation of any one of embodiments 13-15, wherein R³ is —OCH₃.
18. The composition or medical preparation of any one of embodiments 13-17, wherein X is O.
19. The composition or medical preparation of any one of embodiments 1-18, wherein the trinucleotide cap structure comprises a Cap0 or Cap1 structure.
20. The composition or medical preparation of any one of embodiments 1-19, wherein the trinucleotide cap structure comprises a Cap1 structure.
21. The composition or medical preparation of any one of embodiments 1-19, wherein the trinucleotide cap structure comprises (m^{2′-O})N₁pN₂.
22. The composition or medical preparation of any one of embodiments 1-21, wherein the trinucleotide cap structure is selected from the group consisting of: (m₂^{7,3′-O})Gppp(m^{2′-O})ApG (“CleanCap AG”, “CC413”), (m₂^{7,3′-O})Gppp(m^{2′-O})GpG (“CleanCap GG”), (m⁷)Gppp(m^{2′-O})ApG, and (m₂^{7,3′-O})Gppp(m₂^{6,2′-O})ApG.
23. A composition or medical preparation comprising an RNA polynucleotide comprising:
   a 5′ cap; a cap proximal sequence comprising positions +1, +2, +3, +4, and +5 of the RNA polynucleotide; and a sequence encoding a payload, wherein:
      (i) the 5′ cap is a dinucleotide cap structure comprising N₁, wherein N₁ is position +1 of the RNA polynucleotide, and wherein N₁ is G; and
      (ii) the cap proximal sequence comprises:
         N₁ of the dinucleotide cap structure and a sequence comprising N₂N₃N₄N₅ at positions +2, +3, +4, and +5 respectively of the RNA polynucleotide, wherein each N₂ and N₃ is G, and each N₄ and N₅ is selected from: A, C, G, and U.
24. A composition or medical preparation comprising an RNA polynucleotide comprising:
   a 5′ cap; a cap proximal sequence comprising positions +1, +2, +3, +4, and +5 of the RNA polynucleotide; and a sequence encoding a payload, wherein:
      (i) the 5′ cap is a dinucleotide cap structure comprising N₁, wherein N₁ is position +1 of the RNA polynucleotide, and wherein N₁ is G; and
      (ii) the cap proximal sequence comprises:
         N₁ of the dinucleotide cap structure and a sequence comprising N₂N₃N₄N₅ at positions +2, +3, +4, and +5 respectively of the RNA polynucleotide, wherein N₂ is C, N₃ is G, and each N₄ and N₅ is selected from: A, C, G, and U.
25. The composition or medical preparation of any one of embodiments 23-24, wherein the dinucleotide cap structure is: G*N₁, wherein
   G* comprises a structure of formula (I):

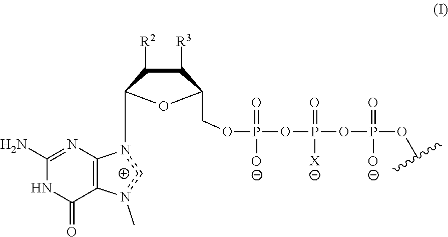

or a salt thereof,
wherein
   each R² and R³ is —OH or —OCH₃; and
   X is O or S.
26. The composition or medical preparation of embodiment 25, wherein R² is —OH.
27. The composition or medical preparation of embodiment 25, wherein R² is —OCH₃.
28. The composition or medical preparation of any one of embodiments 25-27, wherein R³ is —OH.
29. The composition or medical preparation of any one of embodiments 25-27, wherein R³ is —OCH₃.
30. The composition or medical preparation of any one of embodiments 25-29, wherein X is O.
31. The composition or medical preparation of any one of embodiments 25-29, wherein X is S.
32. The composition or medical preparation of any one of embodiments 23-31, wherein the dinucleotide cap structure comprises a Cap0 or Cap1 structure.
33. The composition or medical preparation of any one of embodiments 23-32, wherein the dinucleotide cap structure comprises a Cap0 structure.
34. The composition or medical preparation of any one of embodiments 23-32, wherein the dinucleotide cap structure comprises a Cap1 structure.
35. The composition or medical preparation of any one of embodiments 23-32 or 34, wherein the dinucleotide cap structure comprises (m^{2′-O})N₁.
36. The composition or medical preparation of any one of embodiments 23-35, wherein the dinucleotide cap structure is selected from the group consisting of (m⁷)GpppG (“Ecap0”), (m⁷)Gppp(^{2′-O})G (“Ecap1”), (m₂^{7,3′-O})GpppG (“ARCA” or “D1”), and (m₂^{7,2′-O})GppSpG (“beta-S-ARCA”).
37. A composition or medical preparation comprising an RNA polynucleotide comprising:
   a 5′ cap; a cap proximal sequence comprising positions +1, +2, +3, +4, and +5 of the RNA polynucleotide; and a sequence encoding a payload, wherein:
      (i) the 5′ cap is a tetranucleotide cap structure comprising N₁pN₂pN₃, wherein N₁ is position +1 of the RNA polynucleotide, N₂ is position +2 of the RNA polynucleotide, and N₃ is position +3 of the polynucleotide, and wherein N₁, N₂, and N₃ are selected from one of the following combinations: (a) N₁ is C, N₂ is G, and N₃ is C; (b) N₁ is U, N₂ is G, and N₃ is C; or (c) N₁ is A, N₂ is G, and N₃ is C; and
      (ii) the cap proximal sequence comprises:
         N₁, N₂, and N₃ of the tetranucleotide cap structure and a sequence comprising N₄N₅ at positions +4 and +5 respectively of the RNA polynucleotide, wherein N₄ is G, and N₅ is selected from: A, C, G, and U.
38. The composition or medical preparation of embodiment 37, wherein N₁ is C, N₂ is G, and N₃ is C.
39. The composition or medical preparation of embodiment 37, wherein N₁ is U, N₂ is G, and N₃ is C.
40. The composition or medical preparation of embodiment 37, wherein N₁ is A, N₂ is G, and N₃ is C.
41. A composition or medical preparation comprising an RNA polynucleotide comprising:
   a 5′ cap; a cap proximal sequence comprising positions +1, +2, +3, +4, and +5 of the RNA polynucleotide; and a sequence encoding a payload, wherein:
      (i) the 5′ cap is a tetranucleotide cap structure comprising N₁pN₂pN₃, wherein N₁ is position +1 of the RNA polynucleotide, N₂ is position +2 of the RNA polynucleotide, and N₃ is position +3 of the polynucleotide, and wherein N₁ is G, N₂ is C, and N₃ is G; and
      (ii) the cap proximal sequence comprises:
         N₁, N₂, and N₃ of the tetranucleotide cap structure and a sequence comprising N₄N₅ at positions +4 and +5 respectively of the RNA polynucleotide, wherein each N₄ and N₅ is selected from: A, C, G, and U.
42. The composition or medical preparation of any one of embodiments 37-41, wherein the tetranucleotide cap structure is has a structure: G* N₁pN₂pN₃, wherein
   G* comprises a structure of formula (I):

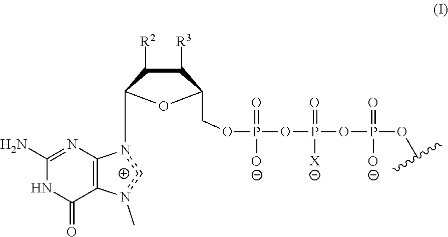

or a salt thereof,
wherein
   each R² and R³ is —OH or —OCH₃; and
   X is O or S.
43. The composition or medical preparation of embodiment 42, wherein R² is —OH.
44. The composition or medical preparation of embodiment 42, wherein R² is —OCH₃.
45. The composition or medical preparation of any one of embodiments 42-44, wherein R³ is —OH.
46. The composition or medical preparation of any one of embodiments 42-44, wherein R³ is —OCH₃.
47. The composition or medical preparation of any one of embodiments 42-46, wherein X is O.
48. The composition or medical preparation of any one of embodiments 37-47, wherein the tetranucleotide cap structure comprises a Cap0, Cap1, or Cap2 structure.
49. The composition or medical preparation of any one of embodiments 37-48, wherein the tetranucleotide cap structure comprises a Cap1 structure.
50. The composition or medical preparation of any one of embodiments 37-48, wherein the tetranucleotide cap structure comprises (m^{2′-O})N₁pN₂pN₃.
51. The composition or medical preparation of any one of embodiments 37-48, wherein the tetranucleotide cap structure comprises a Cap2 structure.
52. The composition or medical preparation of any one of embodiments 37-48, wherein the tetranucleotide cap structure comprises (m^{2′-O})N₁p(m^{2′-O})N₂pN₃.
53. The composition or medical preparation of any one of embodiments 37-52, wherein the tetranucleotide cap structure is selected from the group consisting of: (m₂^{7,3′-O})Gppp(m^{2′-O})Cp(m^{2′-O})GpC and (m₂^{7,3′-O})Gppp(m^{2′-O})Gp(m^{2′-O})CpG.
54. A composition or medical preparation comprising an RNA polynucleotide comprising:
   a 5′ cap; a cap proximal sequence comprising positions +1, +2, +3, +4, and +5 of the RNA polynucleotide; and a sequence encoding a payload, wherein:
      (i) the 5′ cap is m₂^{(7,3′O)}Gppp^{(m2′O)}A₁pG₂, wherein A₁ is position +1 of the RNA polynucleotide, and G₂ is position +2 of the RNA polynucleotide; and
      (ii) the cap proximal sequence comprises:
         A₁ and G₂ of the 5′ cap and a sequence comprising N₃N₄N₅ at positions +3, +4, and +5 respectively of the RNA polynucleotide, wherein N₃ is A, and N₄ and N₅ are selected from: A, C, G, and U.
55. A composition or medical preparation comprising an RNA polynucleotide comprising:
   a 5′ cap; a cap proximal sequence comprising positions +1, +2, +3, +4, and +5 of the RNA polynucleotide; and a sequence encoding a payload, wherein:
      (i) the 5′ cap is m₂^{(7,3′O)}Gppp^{(m2′O)}A₁pG₂, wherein A₁ is position +1 of the RNA polynucleotide, and G₂ is position +2 of the RNA polynucleotide; and
      (ii) the cap proximal sequence comprises:
         A₁ and G₂ of the 5′ cap and a sequence comprising N₃N₄N₅ at positions +3, +4, and +5 respectively of the RNA polynucleotide, wherein N₃ and N₄ are G, and N₅ is selected from: A, C, G, and U.
56. A composition or medical preparation comprising an RNA polynucleotide comprising:
   a 5′ cap; a cap proximal sequence comprising positions +1, +2, +3, +4, and +5 of the RNA polynucleotide; and a sequence encoding a payload, wherein:
      (i) the 5′ cap is m₂^{(7,3′O)}Gppp^{(m2′O)}A₁pG₂, wherein A₁ is position +1 of the RNA polynucleotide, and G₂ is position +2 of the RNA polynucleotide; and
      (ii) the cap proximal sequence comprises:
         A₁ and G₂ of the 5′ cap and a sequence comprising N₃N₄N₅ at positions +3, +4, and +5 respectively of the RNA polynucleotide, wherein N₃ is C, N₄ is G, and N₅ is selected from: A, C, G, and U.
57. The composition or medical preparation of any one of embodiments 6-7, 12-36, or 41-54, wherein N₄ is A.
58. The composition or medical preparation of any one of embodiments 6-7, 12-36, or 41-54, wherein N₄ is C.
59. The composition or medical preparation of any one of embodiments 6-7, 12-36, or 41-54, wherein N₄ is G.
60. The composition or medical preparation of any one of embodiments 6-7, 12-36, or 41-54, wherein N₄ is U.
61. The composition or medical preparation of any one of embodiments 1-60, wherein N₅ is A.
62. The composition or medical preparation of any one of embodiments 1-60, wherein N₅ is C.
63. The composition or medical preparation of any one of embodiments 1-60, wherein N₅ is G.
64. The composition or medical preparation of any one of embodiments 1-60, wherein N₅ is U.
65. An in vitro transcription reaction comprising:
   (i) a template DNA strand comprising a polynucleotide sequence complementary to an RNA polynucleotide sequence provided in any one of claims 1-64, wherein the template DNA strand comprises a sequence that is complementary to a GGG transcription start site;
   (ii) a polymerase;
   (iii) ribonucleotides; and
   (iv) a 5′ cap comprising N₁pN₂;
   wherein N₁ is selected from A, C, U, and G and N₂ is G
   wherein the sequence in the template strand that is complementary to GGG is the start site of transcription by an RNA polymerase.
66. The reaction of embodiment 65, wherein N₁ is C and N₂ is G.
67. The reaction of embodiment 65, wherein N₁ is U and N₂ is G
68. The reaction of embodiment 65, wherein N₁ is A and N₂ is G
69. An in vitro transcription reaction comprising:
   (i) a template DNA strand comprising a polynucleotide sequence complementary to an RNA polynucleotide sequence provided in any one of claims 1-64, wherein the template DNA strand comprises a sequence that is complementary to a GCG transcription start site;
   (ii) a polymerase;
   (iii) ribonucleotides; and
   (iv) a 5′ cap comprising N₁pN₂;
   wherein N₁ is A, C, G, or U, and N₂ is G; or
   wherein N₁ is G and N₂ is C; and
   wherein the sequence in the template strand that is complementary to GCG is the start site of transcription by an RNA polymerase.
70. The reaction of embodiment 69, wherein N₁ is G and N₂ is G.
71. The reaction of embodiment 69, wherein N₁ is U and N₂ is G.
72. The reaction of embodiment 69, wherein N₁ is A and N₂ is G.
73. The reaction of embodiment 69, wherein N₁ is C and N₂ is G.
74. An in vitro transcription reaction comprising:
   (i) a template DNA strand comprising a polynucleotide sequence complementary to an RNA polynucleotide sequence provided in any one of claims 1-64, wherein the template DNA strand comprises a sequence that is complementary to a CGC transcription start site;
   (ii) a polymerase;
   (iii) ribonucleotides; and
   (iv) a 5′ cap comprising N₁pN₂;
   wherein N₁ is C and N₂ is G; or
   wherein N₁ is A, C, G, or U, and N₂ is C and
wherein the sequence in the template DNA strand that is complementary to CGC is the start site of transcription by an RNA polymerase.
75. The reaction of embodiment 74, wherein N₁ is G and N₂ is C.
76. The reaction of embodiment 74, wherein N₁ is U and N₂ is C.
77. The reaction of embodiment 74, wherein N₁ is A and N₂ is C.
78. The reaction of embodiment 74, wherein N₁ is C and N₂ is C.
79. An in vitro transcription reaction comprising:
   (i) a template DNA strand comprising a polynucleotide sequence complementary to an RNA polynucleotide sequence provided in any one of claims 1-64, wherein the template DNA strand comprises a sequence that is complementary to a transcription start site comprising GCG, GCC, GCA, GCU, GUG, GUC, GUA, or GUU;
   (ii) a polymerase;
   (iii) ribonucleotides; and
   (iv) a 5′ dinucleotide cap;
   wherein the sequence in the template DNA strand that is complementary to the transcription start site is the start site of transcription by an RNA polymerase.
80. The in vitro transcription reaction of embodiment 79, wherein the template DNA strand comprises a sequence that is complementary to a transcription start site comprising GCG, GUG, or GCA.
81. The in vitro transcription reaction of embodiment 79, wherein the template DNA strand comprises a sequence that is complementary to a transcription start site comprising GCG or GUG.
82. An in vitro transcription reaction comprising:
   (i) a template DNA strand comprising a polynucleotide sequence complementary to an RNA polynucleotide sequence provided in any one of claims 1-64, wherein the template DNA strand comprises a sequence that is complementary to a GGG transcription start site;
   (ii) a polymerase;
   (iii) ribonucleotides; and
   (iv) a 5′ cap comprising N₁pN₂pN₃;
   wherein N₁ is C, A, or U, and N₂ and N₃ are G; and
   wherein the sequence in the template strand complementary to GGG is the start site of transcription by an RNA polymerase.
83. An in vitro transcription reaction comprising:
   (i) a template DNA strand comprising a polynucleotide sequence complementary to an RNA polynucleotide sequence provided in any one of claims 1-64, wherein the template DNA strand comprises a sequence that is complementary to a GGG transcription start site;
   (ii) a polymerase;
   (iii) ribonucleotides; and
   (iv) a 5′ cap comprising N₁pN₂pN₃;
   wherein N₁, N₂, and N₃ are G; and
   wherein the sequence in the template strand complementary to GGG is the start site of transcription by an RNA polymerase.
84. An in vitro transcription reaction comprising:
   (i) a template DNA strand comprising a polynucleotide sequence complementary to an RNA polynucleotide sequence provided in any one of claims 1-64, wherein the template DNA strand comprises a sequence that is complementary to a GCG transcription start site;
   (ii) a polymerase;
   (iii) ribonucleotides; and
   (iv) a 5′ cap comprising N₁pN₂pN₃;
   wherein N₁ is G, U, C, or A, N₂ is G and N₃ is C; and
   wherein the sequence in the template strand complementary to GCG is the start site of transcription by an RNA polymerase.
85. An in vitro transcription reaction comprising:
   (i) a template DNA strand comprising a polynucleotide sequence complementary to an RNA polynucleotide sequence provided in any one of claims 1-64, wherein the template DNA strand comprises a sequence that is complementary to a GCG transcription start site;
   (ii) a polymerase;
   (iii) ribonucleotides; and
   (iv) a 5′ cap comprising N₁pN₂pN₃;
   wherein N₁ is G, N₂ is C and N₃ is G; and
   wherein the sequence in the template strand complementary to GCG is the start site of transcription by an RNA polymerase.
86. An in vitro transcription reaction comprising:
   (i) a template DNA strand comprising a polynucleotide sequence complementary to an RNA polynucleotide sequence provided in any one of claims 1-64, wherein the template DNA strand comprises a sequence that is complementary to a CGC transcription start site;
   (ii) a polymerase;
   (iii) ribonucleotides; and
   (iv) a 5′ cap comprising N₁pN₂pN₃;
   wherein N₁ is G, C, U, or A, N₂ is C and N₃ is G; and
   wherein the sequence in the template strand complementary to CGC is the transcription start site of transcription by an RNA polymerase.
87. An in vitro transcription reaction comprising:
   (i) a template DNA strand comprising a polynucleotide sequence complementary to an RNA polynucleotide sequence provided in any one of claims 1-64, wherein the template DNA strand comprises a sequence that is complementary to a CGC transcription start site;
   (ii) a polymerase;
   (iii) ribonucleotides; and
   (iv) a 5′ cap comprising N₁pN₂pN₃;
   wherein N₁ is C, N₂ is G and N₃ is C; and
   wherein the sequence in the template DNA strand that is complementary to CGC is the start site of transcription by an RNA polymerase
88. The in vitro transcription reaction of any one of embodiments 65-87, wherein the RNA polymerase promoter is or comprises a T7 polymerase promoter.
89. The in vitro transcription reaction of any one of embodiments 65-88, wherein the template DNA strand comprises: a sequence encoding a 5′ UTR, a sequence encoding a payload, a sequence encoding a 3′ UTR, and a sequence encoding a polyA sequence.
90. An RNA polynucleotide produced from an in vitro transcription reaction provided in any one of embodiments 65-89.
91. A method of making a capped RNA polynucleotide comprising a 5′ cap comprising N₁pN₂; a cap proximal sequence comprising positions +1, +2, +3, +4, and +5 of the RNA polynucleotide; and a sequence encoding a payload, wherein:
   the cap proximal sequence comprises N₁ and N₂ of the 5′ cap, and N₃, N₄, and N₅, wherein N₁ to N₅ correspond to positions +1, +2, +3, +4, and +5 of the RNA polynucleotide, wherein N₃, N₄, and N₅ are each independently chosen from: A, C, G, and U; and
   wherein the method comprises transcribing a template DNA strand in the presence of the 5′ cap and an RNA polymerase, wherein the template DNA strand comprises an RNA polymerase promoter sequence and a sequence that is complementary to a transcription start site, wherein the sequence that is complementary to the transcription start site is the start site of the RNA polymerase promoter.
92. The method of embodiment 91, wherein N₁ is complementary to position +1 of the template DNA strand (corresponding to the first nucleotide of the transcription start site), and N₂ is complementary to position +2 of the template DNA strand (corresponding to the second nucleotide of the transcription start site).
93. The method of embodiment 91, wherein N₂is complementary to position +1 of the template DNA strand (corresponding to the first nucleotide of the transcription start site), and N₁ is not complementary to position +1 of the template DNA strand.
94. The method of any one of embodiments 91-93, wherein the RNA polymerase is T7 RNA polymerase.
95. A method of making a capped RNA polynucleotide comprising:
   transcribing a template DNA strand in the presence of a 5′ cap, wherein the 5′ cap comprises the structure N₁pG₂,
   wherein the template DNA strand comprises an RNA polymerase promoter sequence and a sequence that is complementary to a GGG transcription start site;
   wherein N₁ is A, C, G, or U; and
   wherein:
      (a) when N₁ is G, N₁ is complementary to position +1 of the template DNA strand position and position +1 of the template DNA strand (corresponding to the first nucleotide in the transcription start site) is C, position +2 of the template DNA strand (corresponding to the second nucleotide in the transcription start site) is C; position +3 of the template DNA strand is C; and positions +4 and +5 of the template DNA strand are each independently any nucleotide; or
      (b) when N₁ is not G, G₂ is complementary to position +1 of the template DNA strand and position +1 of the template DNA strand is C, position +2 of the template DNA strand is C; position +3 of the template DNA strand is C; and positions +4 and +5 of the template DNA strand are each independently any nucleotide.
96. A method of making a capped RNA polynucleotide comprising:
   (a) transcribing a template DNA strand in the presence of a cap structure comprising G₁pC₂, wherein the template DNA strand comprises an RNA polymerase promoter sequence and a sequence that is complementary to a GCG transcription start site, and wherein:
      G₁ is complementary to position +1 of the template DNA strand (corresponding to the first nucleotide in the transcription start site) and position +1 of the template DNA strand is C, C₂ is complementary to position +2 of the template DNA strand (corresponding to the second nucleotide of the transcription start site) and position +2 of the template DNA strand is G; position +3 of the template DNA strand (corresponding to the third nucleotide of the transcription start site) is C; and positions +4 and +5 of the template DNA strand are each independently any nucleotide; or
   (b) transcribing a template DNA strand in the presence of a cap structure comprising N₁pG₂, wherein N₁ is A, C, or U, and wherein:
      G₂ is complementary to position +1 of the template DNA strand and position +1 of the template DNA strand is C, position +2 of the template DNA strand is G; position +3 of the template DNA strand is C; and positions +4 and +5 of the template DNA strand are each independently any nucleotide.
97. A method of making a capped RNA polynucleotide comprising:
   (a) transcribing a template DNA strand in the presence of a cap structure comprising C₁pG₂, wherein the template DNA strand comprises an RNA polymerase promoter sequence and a sequence that is complementary to a CGC transcription start site, and wherein:
      C₁ is complementary to position +1 of the template DNA strand (corresponding to the first nucleotide in the transcription start site) and position +1 of the template DNA strand is G, G₂ is complementary to position +2 of the template DNA strand (corresponding to the second nucleotide of the transcription start site) and position +2 of the template DNA strand is C; position +3 of the template DNA strand (corresponding to the third nucleotide of the transcription start site) is G; and positions +4 and +5 of the template DNA strand are each independently any nucleotide; or
   (b) transcribing a template DNA strand in the presence of a cap structure comprising N₁pC₂, wherein N₁ is A, C, G, or U, and wherein:
      C2 is complementary to position +1 of the template DNA strand and position +1 of the template DNA strand is G, position +2 of the template DNA strand is C; position +3 of the template DNA strand is G; and positions +4 and +5 of the template DNA strand are each independently any nucleotide.
98. A method of making a capped RNA polynucleotide comprising a 5′ dinucleotide cap; a cap proximal sequence comprising positions +1, +2, +3, +4, and +5 of the RNA polynucleotide; and a sequence encoding a payload, wherein:
   the cap proximal sequence comprises N₁ of the 5′ cap, and N₂, N₃, N₄, and N₅, wherein N₁ to N₅ correspond to positions +1, +2, +3, +4, and +5 of the RNA polynucleotide, wherein N₁ is G, N₂ is U or C, and N₃, N₄, and N₅ are each independently chosen from: A, C, G, and U;
   wherein the method comprises transcribing a template DNA strand in the presence of the 5′ cap and an RNA polymerase.
99. A complex comprising a template DNA strand and a 5′ cap comprising a structure of N₁pN₂, wherein the template DNA strand comprises an RNA polymerase promoter sequence and a sequence that is complementary to a transcription start site;
   wherein N₁ and N₂ are each independently chosen from: A, C, G, and U;
   wherein N₂ interacts with the +1 position of the template DNA strand (corresponding to the first nucleotide of the transcription start site) and N₁ does not interact with the +1 position of the template DNA strand; and
   wherein the sequence in the template strand that is complementary to the transcription start site is the start site of transcription by an RNA polymerase.
100. The complex of embodiment 99, wherein N₁ is A and N₂ is G, and wherein the +1 position of the sequence that is complementary to the transcription start site is C.
101. The complex of embodiment 99, wherein N₁ is U and N₂ is G, and wherein the +1 position of the sequence that is complementary to the transcription start site is C.
102. The complex of embodiment 99, wherein N₁ is C and N₂ is G, and wherein the +1 position of the sequence that is complementary to the transcription start site is C.
103. A complex comprising a template DNA strand and a 5′ cap comprising a structure of N₁pN₂, wherein the template DNA strand comprises an RNA polymerase promoter sequence and a sequence that is complementary to a transcription start site;
   wherein N₁ and N₂ are each independently chosen from: A, C, G, and U;
   wherein N₁ interacts with the +1 position of the template DNA strand (corresponding to the first nucleotide of the transcription start site) and N₂ interacts with the +2 position of the template DNA strand (corresponding to the second nucleotide of the transcription start site); and
   wherein the sequence in the template strand that is complementary to the transcription start site is the start site of transcription by an RNA polymerase.
104. The complex of embodiment 103, wherein N₂ is U or C, and the +2 position of the template DNA strand is A or G.
105. The complex of embodiment 103 or 104, wherein N₃ is A or G, and the +3 position of the template DNA strand is T or C.
106. The complex of embodiment 103, wherein N₁ is A and N₂ is G, and position +1 of the template DNA strand is T and position +2 of the template DNA strand is C.
107. The complex of embodiment 103, wherein N₁ is G and N₂ is C, and position +1 and position +2 of the template DNA strand are C and G, respectively.
108. The complex of embodiment 103, wherein N₁ is A and N₂ is U, and position +1 and position +2 of the template DNA strand is T and A, respectively.
109. A complex comprising a template DNA strand and a 5′ cap comprising a structure of N₁pN₂N₃, wherein the template DNA strand comprises an RNA polymerase promoter sequence and a sequence that is complementary to a transcription start site;
   wherein N₁, N₂, and N₃ are each independently chosen from: A, C, G, and U; and
   wherein N₁, N₂, and N₃ interact with the +1, +2, and +3 positions of the template DNA strand (corresponding to the first, second, and third nucleotides, respectively of the transcription start site); and
   wherein the sequence in the template strand that is complementary to the transcription start site is the start site of transcription by an RNA polymerase.
110. The complex of embodiment 109, wherein N₂ is C or U and the +2 position of the template DNA strand is G or A.
111. The complex of embodiment 109, wherein N₂ is C or U, and N₃ is G or A and the +2 position of the template DNA strand is G or A and the +3 position of the template DNA strand is C or T.
112. The complex of embodiment 109, wherein N₁ is G, N₂ is C and N₃ is G and the +1, +2, and +3 positions of the template DNA strand are C, G, and C, respectively.
113. The complex of embodiment 109, wherein N₁ is A, N₂ is G, and N₃ is C and the +1, +2, and +3 positions of the template DNA strand are T, C, and G, respectively.
114. The complex of embodiment 109, wherein N₁ is A, N₂ is G, and N₃ is A and the +1, +2, and +3 positions of the template DNA strand are T, C, and T, respectively.
115. The complex of embodiment 109, wherein N₁ is A, N₂ is U, and N₃ is A and the +1, +2, and +3 positions of the template DNA strand are T, A, and T, respectively.
116. A complex comprising a template DNA strand and a 5′ cap comprising a structure of N₁, wherein the template DNA strand comprises an RNA polymerase promoter sequence and a sequence that is complementary to a transcription start site;
   wherein N₁ is G; and
   wherein N₁ interacts with the +1 position of the template DNA strand (corresponding to the first nucleotide of the transcription start site);
   wherein the +2 position of the template DNA strand (corresponding to the second nucleotide of the transcription start site) is G or A; and
   wherein the sequence in the template strand that is complementary to the transcription start site is the start site of transcription by an RNA polymerase.
117. The complex of embodiment 116, wherein the +3 position of the template DNA strand is T or C.
118. The complex of embodiment 116 or 117, wherein the +2 position of the template DNA strand is G.
119. The complex of any one of embodiments 116 to 118, wherein the +1 position of the template DNA strand is C, the +2 position of the template DNA strand is G, and the +3 position of the template DNA strand is C.
120. The complex of any one of embodiments 99 to 119, wherein the nucleotides of the cap interact with the nucleotides of the template DNA strand via canonical Watson-Crick base pairing.
121. The complex of any one of embodiments 99 to 120, wherein the RNA polymerase promoter sequence is a T7 RNA polymerase promoter sequence.
122. The complex of any one of embodiments 99 to 121, wherein the complex further comprises an RNA polymerase (e.g., a T7 RNA polymerase).
123. A method of formulating a pharmaceutical composition, the method comprising combining a preparation comprising an RNA polynucleotide of any one of embodiments 1 to 64 with a preparation comprising lipids.
124. The method of embodiment 123, wherein the method comprises combining the preparation comprising RNA polynucleotide with the preparation comprising lipids to form lipid nanoparticles that encapsulate the RNA polynucleotide.
125. The method of embodiment 123, wherein the method comprises combining the preparation comprising RNA polynucleotide with the preparation comprising lipids to form RNA lipoplexes.
126. A method comprising: administering to a subject, a pharmaceutical composition comprising an RNA polynucleotide of any one of embodiments 1-64.
127. The method of embodiment 126, wherein the RNA polynucleotide is formulated in a lipid nanoparticle (LNP) or a lipoplex (LPX) particle.
128. The method of embodiment 126 or 127, wherein the pharmaceutical composition is administered to the subject by injection.
129. The method of embodiment 128, wherein the pharmaceutical composition is administered to the subject by intravenous injection.
130. The method of embodiment 128, wherein the pharmaceutical composition is administered to the subject by intramuscular injection.
131. The method of embodiment 128, wherein the pharmaceutical composition is administered to the subject by intradermal injection.

### EXEMPLIFICATION

### Example 1—Assessment of Effects of Cap Structures and/or Transcription Site on Certain Characteristics of In Vitro-Transcribed RNA

Enzymatic capping is the most commonly used method for producing in vitro transcribed messenger RNA (IVT mRNA), but this procedure is time-consuming and high-cost compared to co-transcriptional caps. However, mRNA research is performed with mRNAs capped with less effective dinucleotide cap reagents.

The present Example describes a trinucleotide cap structure (e.g., a CleanCap AG 3′OMe co-transcriptional cap1 cap (CC413—m₂^{(7,3′O)}Gppp^{(m2′O)}ApG)) with appropriate starting triplet can be used to generate a functional 5′ capped mRNA and provides more effective and less immunogenic mRNA than those with traditional 5′ end cap structures. In particular embodiments, the present Example demonstrates that a CleanCap AG 3′OMe co-transcriptional cap1 cap (CC413) can be used in combination with an AGA transcription start site or a GGG transcription start site to produce a functional capped mRNA. In particular, CC413 with an AGA transcription start site produced more effective and/or less immunogenic mRNA, with improved expression level of an encoded payload and/or increased duration of expression of an encoded payload in vivo.

As described below, to test the performance of mRNA capped with CC413 in the context of capping preparations, mRNAs encoding model proteins (e.g., firefly luciferase and murine erythropoietin (EPO mRNA)) were generated. It is demonstrated that CC413 improved capping efficiency of cap1 mRNA and reduced levels of proinflammatory cytokines, including, e.g., TNFα, IL-1β and/or IL-6 compared to those made by enzymatic capping preparations or dinucleotide caps. In vivo evaluation demonstrated that CC413-capped mRNAs starting with AGA were better translated than enzymatically capped mRNA starting with GGG at each time point following administration. EPO mRNA capped with CC413 was functional because it increased the hematocrit from 41 to 59%, which remained at high level for two weeks.

### Results

CleanCap AG-Capped mRNA Shows Superior Characterisitics

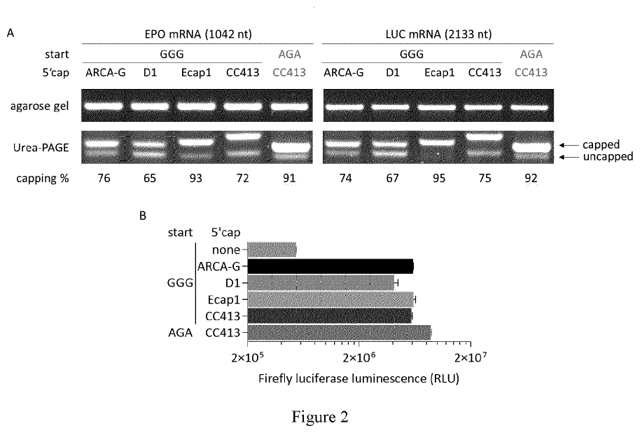

Each cap used in this Example belongs to an anti-reverse cap analog family that cannot be incorporated in the reverse orientation into the mRNA due to a modification at the C2′ or C3′ position of 7-methylguanosine (FIG. 1). Unlike the commonly used dinucleotide cap reagents including anti-reverse cap analog (ARCA-G) and β-S-ARCA (D1), CleanCap AG 3′OMe trinucleotide cap1 cap (CC413) contains a methyl group at the 2′hydroxy-group (2′OH) position of the first nucleotide creating a nature-like cap1 structure (FIG. 1). In order to determine whether CC413 results in equivalent yield and capping efficiency compared to enzymatically capped (Ecap1) mRNA, in vitro transcription reaction was performed. All caps were added directly to the IVT mixture while enzymatic capping was performed post-transcriptionally using Vaccinia virus-derived capping enzymes. Spectrophotometric and gel electrophoretic analysis of mRNAs showed that CC413 could offer high-yielding and intact IVT mRNA encoding murine erythropoietin (EPO mRNA) or firefly luciferase (LUC mRNA) as well as enzymatically prepared or those capped with ARCA-G and D1 dinucleotide caps (FIG. 2A).

Ribozyme assay followed by denaturing urea polyacrylamide gel electrophoresis (PAGE) demonstrated that the use of CC413 for mRNA capping leads to the same highly capped material (>90%) as those made with enzymatic capping system regardless of the coding sequence. Nevertheless, the CC413-capped mRNA starting with AGA displayed an average of 18% higher capping efficiency without transcriptional stuttering compared to those that exhibit GGG as initial triplet (FIG. 2A). Using HILIC assay, nearly 100% capping efficiency was calculated after enzymatic preparation. Based on this data, the second slight band shown on urea gel related to Ecap1 mRNAs corresponds to a capped minor product resulting from the slippage of T7 polymerase on DNA strand, which is in accordance with earlier report used Ribozyme assay for IVT mRNA starting with G (FIG. 2A). The lowest values were observed with mRNAs transcribed in the presence ARCA and D1 dinucleotide caps also shown by HILIC analysis (FIG. 2A). These results indicated that the combination of CC413 with AGA starting site is suitable for making cap1 structure at the 5′end of mRNA while high yield and capping efficiency of mRNA are also achieved.

In order to test whether IVT mRNA capped with CC413, ARCA-G, D1 or enzymatic preparation is translatable, in vitro protein synthesis from mRNA encoding luciferase was determined using in vitro translation assay. Luciferase activity was measured by luciferase assay and compared with the performance of uncapped LUC mRNA. High level of luciferase expression could be detected for all capped mRNAs compared to the uncapped control (none) indicating the presence of 5′cap at their 5′end and that the selected transcripts encode the appropriate protein (FIG. 2B).

Efficient Translation of CleanCap AG-Capped mRNA Encoding Luciferase in Mice

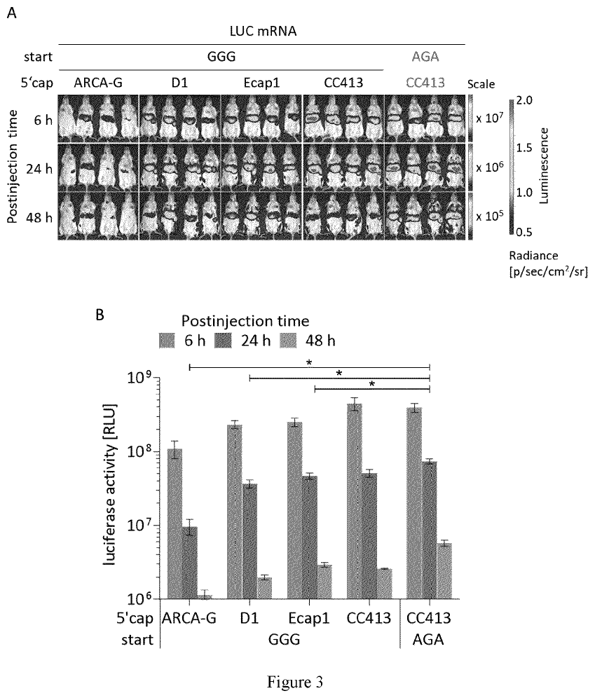

The results of Rabbit Reticulocyte Lysate Translation System show that luciferase was translated well from each LUC mRNA containing N1-methylpseudouridine (m1Ψ) nucleoside modifications. For further investigation whether the duration and tissue distribution of luciferase encoded by CC413-capped mRNA is comparable to Ecap1 mRNA in vivo, 3.0 μg of LUC mRNAs used in the in vitro assay were complexed with TransIT mRNA reagent and immediately administered i.v. in mice. The expression of the encoded protein was determined 6, 24 and 48 hours after mRNA injection by in vivo imaging. The greatest portion of bioluminescent signal obtained by each TransIT-complexed LUC mRNAs was observed in the liver, but translation occurred to a minor extent in the spleen as well (FIG. 3A). All LUC mRNAs except those capped with ARCA-G provided very high translation 6 hours post mRNA injection, but the protein production decreased significantly at later time points due to the rapid turnover of functional mRNA in the liver (26). In general, CC413-capped LUC mRNA starting with AGA induced either as effective or higher luciferase activity at each time point as those starting with GGG regardless of the 5′cap (FIG. 3B). Quantification of IVIS measurement showed at least two and four-fold more luciferase activity in mice that received CC413-capped mRNA compared to those injected with mRNA synthesized with dinucleotide caps (D1 and ARCA-G) at 6 and 24 hours after injection, respectively (FIG. 3B). These results indicate that CleanCap AG trinucleotide cap acts as nature-like cap1 structure on the 5′end of an mRNA molecule.

Injection of EPO mRNA Capped with CleanCap AG Ensures the Long-Term Maintenance of the Encoded Protein and Hematocrit

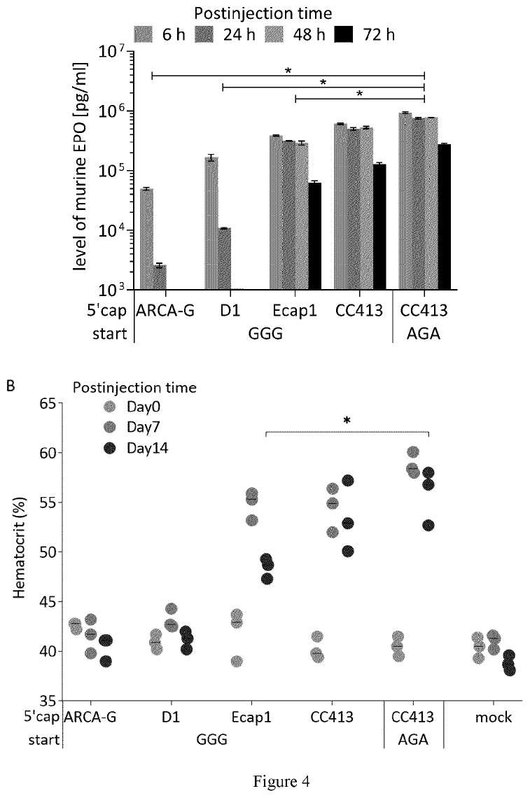

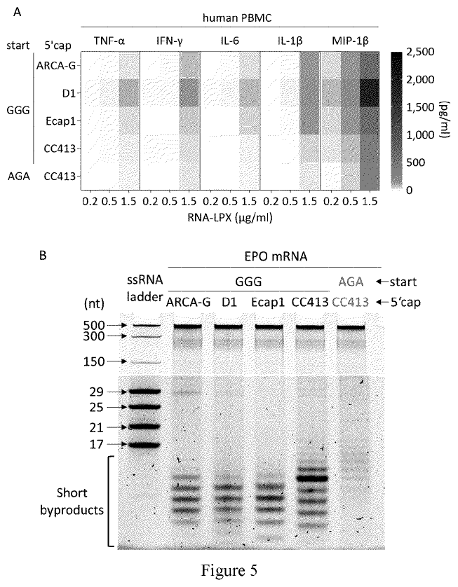

Not only LUC mRNAs but also EPO mRNAs were prepared and investigated, because the erythropoietin assay is more reliable and more suitable for quantitative measurement of the protein of interest than substrate-dependent luminescent-based assay. Moreover, a functional test can also be performed by detection of hematocrit level. Accordingly, mice were injected with 0.15 mg/kg dose of TransIT-complexed EPO mRNAs by IV administration as same as for LUC mRNAs. In line with results obtained from luciferase experiment, CC413-capped EPO mRNA starting with AGA performed the best. At 6 and 24 hours after IV injection of EPO mRNA, 2.4, 5.6, 18.7 and 2.4, 69.5 and 290.6 more protein production was detected in mice that received CC413-capped mRNA compared to those injected with Ecap1, D1- and ARCA-G-capped mRNA, respectively (FIG. 4A). This effect was more pronounced at subsequent time points. Importantly, translational capacity of both CC413-capped mRNAs starting with AGA was higher at each time point than those carrying GGG at position of the first three transcribed nucleotides most likely due to the less capping efficiency (FIGS. 3B and 4A). Plasma EPO levels started to decline markedly at 3 days after cap1 mRNA administration contrast to ARCA-G- and D1-capped mRNA, which showed at least a 10-fold decrease already on the first day compared to values obtained from 6 hours of data (FIG. 4A). These outcomes show the importance of cap1 structure for the efficient translation of IVT mRNA.

In order to determine if CC413-capped mRNA administered in vivo can produce sufficient amounts of functional protein, hematocrits were measured immediately, 7 and 14 days after intravenous injection of these set of EPO mRNAs. The highest hematocrit (59±0.7%) was observed in mice at 7 days after single administration of CC413-capped mRNA starting with AGA, however, those that contain a G triplet as initial sequence were also able to enhance the hematocrit from 40% to 54% (FIG. 4B). The hematocrit remained still elevated after 2 weeks following injection of CC413-capped mRNA starting with AGA (56±1.6%) which was found to be 7-8% higher than that obtained with a single administration of enzymatically capped mRNA initiated with GGG (48±0.6%). In accordance with plasma EPO levels, injection of ARCA-G and D1-capped mRNA or TransIT reagent itself did not lead to a significant increase in the hematocrit at any time point (FIG. 4B). These data demonstrate that co-transcriptional CC413 cap1 cap is suitable for generating a functional cap1 structure on the 5′end of mRNA.

CleanCap AG Cap1 Cap Combined with AGA Start Site Diminishes the Immunogenicity of In Vitro Transcribed mRNA

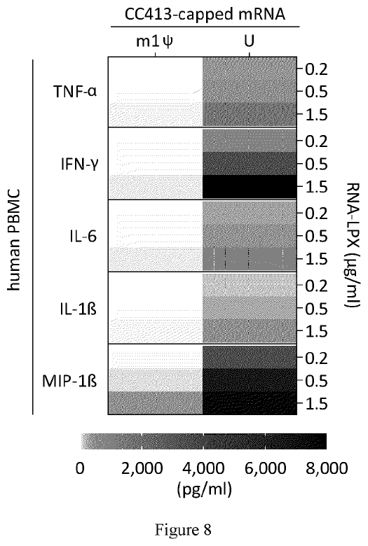

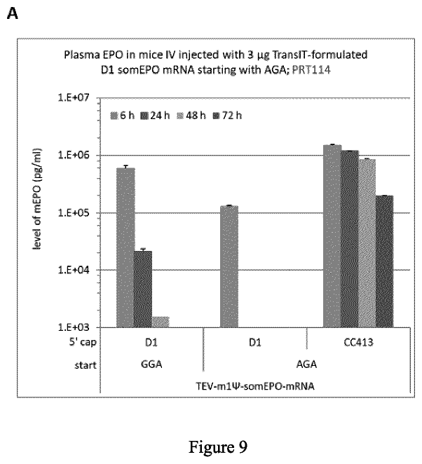

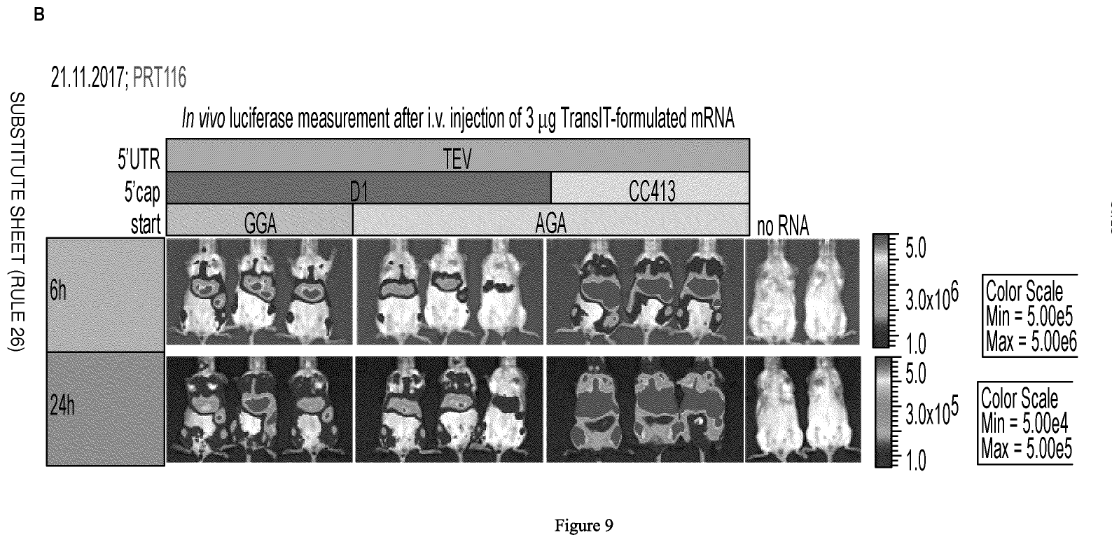

Considering that the quality of five-prime cap and the interaction of the RNA polymerase and transcriptional start site has effects on immunogenicity of mRNA, the level of potentially expressed proinflammatory cytokines and chemokines was measured. First, supernatants of human peripheral blood mononuclear cells (PBMCs) transfected with capped mRNAs formulated with cationic lipoplex (RNA-LPX) were collected 24 hours after transfection and then the levels of TNF-α (Tumor necrosis factor alpha), IFN-γ (Interferon gamma), IL-6 (Interleukin 6), IL-1β (Interleukin 1 beta) and MIP-1β (Macrophage inflammatory protein 1 beta) were measured by using the MSD (Meso Scale Discovery) platform. All these analytes showed no or moderate increase in each sample. Without wishing to be bound by a particular theory, such low immunogenicity can be explained by nucleoside modifications incorporated into the mRNA, as cells treated with lipid-complexed U-containing mRNA capped with CC413, showed significant changes even at the lowest dose (0.2 μg/ml) compared to those transfected with N1-methylpseudouridine-modified (m1Ψ) mRNA (FIG. 8). The comparison of the cytokine/chemokine production induced by transfection of RNA-LPX for capped mRNAs containing m1Ψ modifications applied at different doses is presented in a heatmap (FIG. 5A). After transfection of β-S-ARCA (D1)-capped mRNA, the highest increase of each analyte was observed regardless of the dose used (FIG. 5A). MIP-1β showed a moderate but the highest upregulation in human PBMC for each mRNA sample. A slight increase in levels of other analytes including TNF-α, IFN-γ, IL-6 and IL-1β was also detected after the highest RNA-LPX dose (FIG. 5A). No significant differences were observed between the response for IFN-γ induced by transfection of CC413-capped mRNA and Ecap1 or ARCA-G-capped mRNA. TNF-α, IL-6, IL-1β and MIP-1β showed elevated levels in supernatants of human PBMC treated with Ecap1, ARCA-G and D1-capped mRNA that were significantly higher than those subjected to CC413-capped mRNA regardless of starting site. For all tested cytokines/chemokines levels after transfection of CC413-capped mRNA starting with AGA were comparable to those starting with GGG indicating that the use of CleanCap AG cap1 cap is sufficient to reduce the immunogenicity of IVT mRNA.

The slippage of RNA polymerases frequently occurred on template DNA strand that contains a G triplet as a transcriptional start site resulting in short abortive byproducts (see e.g., Imburgio, et al. (2000) Biochemistry 39:10419-10430), which may contribute to the regulation of gene expression and increased immunogenicity of the mRNA. Accordingly, the pattern of these short contaminants synthesized during in vitro transcription of mRNAs used in this study was analyzed using self-made denaturing urea PAGE. A significant amount of short abortive byproducts in the range of 5-15 nt length was detected in each mRNA starting with GGG regardless of the 5′cap (FIG. 5B). Interestingly, no or negligible short contaminants were observed for CC413 capped mRNA which contains AGA as initial sequence and most of these abortive IVT byproducts were in the range longer than 13 nt (FIG. 5B).

Translational Capacity of mRNA Capped with ARCA CleanCap AG is Better than with Non-ARCA Version

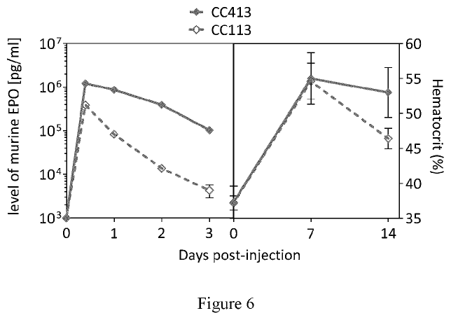

To determine if 3′O-methyl modification of the 7-methylguanosine has any beneficial effect on translation and biological activity of IVT mRNA. Therefore, murine erythropoietin-encoding mRNA (EPO mRNA) was synthesized and capped co-transcriptionally either with anti-reverse CleanCap AG (CC413) cap1 cap or standard CleanCap AG (CC 113), which is not a member of the ARCA cap family. Three microgram of EPO mRNAs were injected intravenously into BALB/c mice and then plasma EPO levels were determined at 6, 24, 48 and 72 h after injection. The results of EPO ELISA showed that ARCA CC413-capped mRNA translated significantly better compared to those capped with non-ARCA CC113 cap1 capat each time point (FIG. 6). Murine EPO translated from both ARCA- and non-ARCA cap1 mRNA resulted in a significant increase of hematocrits at 7 and 14 days after mRNA administration. Interestingly, hematocrit level was comparable in mice injected with non-ARCA cap1 (54.5±2.5%) and ARCA-cap1 EPO RNA (55.0±2.5%) at day 7 after injection despite the fact that there is a significant difference in the EPO level for ARCA CC413. However, at 14 days after mRNA injection hematocrit decreased from 54.5±2.5% to 46.5±1.5% in mice injected with non-ARCA CC113-capped mRNA while after i.v. administration of ARCA CC413-capped mRNA it remained at high level (53.0±3.0%) (FIG. 6). These findings are in good agreement with an earlier study from Hederson et al. (2021) Current Protocols 1:e39 showing that incorporation of any cap1 cap (non-ARCA CC113 or ARCA CC413) lead to generation of functional mRNA, but these results indicate the use of ARCA CC413 for production of Cap1-mRNA due to its long-term beneficial effect on translation and biological activity of IVT mRNA.

### Discussion

One aspect presented herein relates to use of an anti-reverse trinucleotide cap1 cap, called CleanCap AG 3′OMe (CC413) to obtain a functional mRNA with cap1 structure at the 5′end. It is demonstrated that appropriate combination of initial sequence and a cap structure (e.g., a trinucleotide cap, e.g., such as CC413) not only contributes to attenuation of proinflammatory cytokine/chemokine levels, but leads to a superior mRNA that significantly surpasses the translational capacity and biological activity of IVT mRNA capped with the dinucleotide caps or by enzymatic capping procedure.

The same basic structural elements have been incorporated into each mRNA used in this Example. All respective mRNAs contained N1-methylpseudouridine (m1Ψ) nucleoside modification which increases its biological stability and thereby the durability of the encoded protein can be monitored longer compared to unmodified RNAs, as reported in Karikó et al. (2008) Molecular Therapy: the Journal of the American Society of Gene Therapy. In addition, sequence elements have been applied for 5′UTR and 3′UTR which have been published to improve protein production from the corresponding mRNAs (see e.g., Babendure, et al. (2006) RNA 12:851-861); Schrom, et al. (2017) Molecular Therapy: Nucleic Acids 7:350-365; and Orlandini von Niessen, et al (2019) Molecular Therapy: the Journal of the American Society of Gene Therapy 27:824-836). The differences in the properties of the mRNAs presented in the study are mainly due to the non-identical five prime cap (5′cap). There are two approaches that can ensure the essential 5′ cap at the 5′ end of IVT mRNA. One is the enzymatic capping reaction which is still used for mRNA capping, e.g., as in Corbett et al. (2020) The New England Journal of Medicine 383:1544-1555. This method requires not only high costs, but also a second post-transcriptional enzymatic step. Accordingly, the use of various co-transcriptional caps for generating 5′cap structure has come to the fore. These caps have undergone significant development in the past decade, and in the current study the performance of the most recent trinucleotide cap1 cap (CC413) from CleanCap AG family was compared to the commonly used anti-reverse and modified anti-reverse dinucleotide caps (ARCA-G and β-S-ARCA) and enzymatic capping procedure.

Adding of CC413 directly to in vitro transcription mixture resulted in high RNA yields and nearly 100% capping efficiency of mRNA that is comparable with enzymatic capping reaction (FIG. 2A). Both methods led to markedly higher capping efficiencies of mRNA compared to regular dinucleotide ARCA caps (FIG. 2A), which was also strongly manifested at level and durability of proteins measured in mice injected with IVT mRNA encoding firefly luciferase and murine EPO (FIGS. 3 and 4). Interestingly, the translational capability as well as the biological activity of CC413-capped mRNAs outperformed those ones capped by Vaccinia enzymatic method (FIG. 4). Without wishing to be bound by a particular theory, such divergence can be explained by small differences in the quality of 5′cap, even though that the values obtained from capping analysis are almost identical (FIG. 2A). Firstly, the 5′cap of enzymatically capped mRNA is not modified at C2′ or C3′ position and ARCA-G-capped mRNA has a higher translation efficiency compared to conventional caps (see e.g., Stepinski, et al. (2001) RNA 7:1486-1495). CC413 (FIG. 1) has also a variant that does not employ modification at the C3′ position of 7-methylguanosine, best mimicking the natural cap1 structure. The results presented herein showed that the ARCA version (CC413) of the same CleanCap AG cap1 cap provides mRNA property improvement in terms of translational efficiency and biological activity compared to the non-ARCA version (CC113) (FIG. 6).

On the other hand, it is hypothesized that enzymatic capping reaction produces significant amounts of capped but not methylated products due to incomplete methylation of 2′-OH on the first ribose sugar. These products correspond to cap0 mRNA, which has a strong affinity for decapping enzymes and interferon-induced proteins with tetratricopeptide repeats (IFITs) that inhibits cap0-dependent translation in contrast to cap1 mRNA. Diamond et al. (2014) Cytokine & growth Factor Reviews 25:543-550; and Fleith et al. (2018)Nucleic Acids Research 46:5269-5285. In terms of CC413, cap0 mRNA is not generated indicating that the ribozyme assay is not suitable for separating cap0 and cap1 mRNA products and more importantly, the use of CC413 leads to a much higher proportion of mRNA molecules whose 5′end is equivalent to cap1 mRNA compared to those completed by enzymatic capping reaction. Taken together, the results presented herein indicate that CC413 provides superior characteristics to IVT mRNA and it can act as nature-like cap1 structure on the 5′end of an mRNA molecule.

Nevertheless, not only the quality of five-prime cap but the interaction of the RNA polymerase and transcriptional start site have effects on mRNA properties such as stability, translational efficiency as well as immunogenicity. Most of the DNA-dependent RNA polymerases including the extensively studied bacteriophage T7 RNA polymerase primarily displays requirement for initiation with GTP as shown in Krupp et al. (1988) Gene 72:75-89 and Conrad, et al. (2020) Communications Biology 3:439. Accordingly, in mRNA research the most widely used initial sequence at positions +1 to +3 is a guanine triplet (GGG) which is one of the most optimal for RNA polymerase to achieve high transcriptional activity, as shown in Conrad et al. (2020) Communications Biology 3:439. CC413-capped mRNA starting with GGG performed equally to the enzymatically capped mRNA even despite lower capping efficiency. However, comparative examinations as described herein showed that the use of the AG start for a CC413 cap is a better combination. Without wishing to be bound by a particular theory, the slippage of RNA polymerases frequently occurred on the template DNA strand that contains a G triplet (but not AGA) as a transcriptional start site and resulted in a significant amount of short abortive byproducts (see e.g., Imburgio et al. (2000) Biochemistry 39:10419-10430). However, as shown in this Example, use of an AGA as a transcriptional start site can reduce RNA polymerase slippage and thus reduced short abortive byproducts as observed with denaturing urea PAGE (FIG. 5B). These short contaminants cannot be eliminated by facile purification methods and may contribute to the regulation of gene expression and increased immunogenicity of the mRNA. Hence, such outcomes are particularly undesirable in mRNA therapeutic fields that requires a high safety level and the avoidance of immune responses as discussed in Goldman et al. (2009) Science 324:927-928; Mu, et al. (2018) Nucleic Acids Research 46:5239-5249; and van Hoecke et al. (2019) Journal of Translational Medicine 17:54. It is demonstrated that the CC413-capped mRNA which carries methylated adenosine as the first transcribed nucleotide resulted in the lowest amount of short abortive byproducts (FIG. 5B) and slight activation of immune response compared to those starting with GGG, as indicated by the higher level of all tested proinflammatory cytokines and chemokines (FIG. 5A). Despite the fact that several proceedings are available to reduce the immunogenicity of mRNA, including incorporation of nucleoside modifications into mRNA (FIG. 8) as shown in Karikó et al. (2008) Molecular Therapy: the Journal of the American Society of Gene Therapy 16:1833-1840, and the advanced mimicking of natural 5′cap (FIG. 5A), different type of immune responses are still elicited narrowing the use of IVT mRNA for non-immunotherapy applications (see e.g., van Hoecke et al. (2019) Journal of Translational Medicine 17:54; and Devoldere, et al. (2016) Drug Discovery Today (21)11-25).

The results described in this Example demonstrate clearly that a trinucleotide cap structure (e.g., a CleanCap AG 3′OMe co-transcriptional cap1 cap(CC413)) with appropriate starting triplet can be used to generate a functional cap1 structure on the 5′end of mRNA and provides more efficient, less immunogenic and cost-effective mRNA than those made with traditional 5′end structures. Such CC413 capping strategy can be useful for mRNA therapeutic applications including, e.g., vaccines (see, e.g., Sahin, et al. (2021) Nature 595:572-577), protein replacement and cell therapy as well as gene editing.

Exemplary Methods and Materials Used in this Example
In Vitro Transcription of mRNA

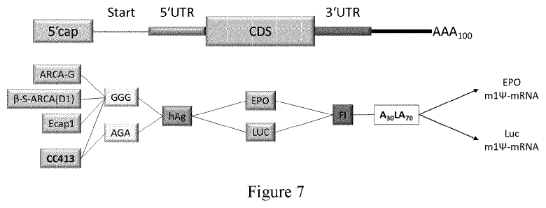

For templates, linearized plasmid encoding codon-optimized murine erythropoietin (EPO) and firefly luciferase (LUC) were used. The mRNAs starting with GGG or AGA were designed to contain 5′ untranslated region (5′UTR) sequences of human α-globin (hAg) mRNA and interrupted 100 nt-long 3′ poly(A) tail (FIG. 7) as flanking region of the coding sequence. The MEGAscript T7 RNA polymerase kit (Thermo Fisher Scientific, Cat #AMB1334-5) was used for transcription, and UTP was replaced with N1-methylpseudouridine (m1Ψ) triphosphate (TriLink Cat #N-1081). Capping of IVT mRNAs was performed post-transcriptionally using m7G capping enzyme and 2′-O-methyltransferase according to the manufacturer (Vaccinia Capping System) or co-transcriptionally using 6 mM of dinucleotide caps (e.g., ARCA-G, TriLink, #N-7003; β-S-ARCA(D1) synthesized as previously described in Kowalska, et al. (2008) RNA 14:119-1131) and 3 mM of trinucleotide conventional cap1 cap (e.g., CleanCap AG, TriLink, #N-7113) and anti-reverse cap1 cap (e.g., CleanCap AG 3′OMe, TriLink, #N-7413). To obtain the desired transcripts generated with caps, the initial GTP concentration in a transcription reaction was reduced from 7.5 mM to 1.5 mM. For removal of template DNA, Turbo DNase (Thermo Fisher Scientific, Cat #AM1907) was added to the reaction mix. The synthesized mRNA was isolated from the reaction mix by precipitation with half of a reaction volume of 8 M LiCl solution. After dissolving in nuclease free water, the mRNA concentration and quality were measured on a NanoDrop2000C spectrophotometer (Thermo Fisher Scientific, Cat #ND-2000c). Small aliquots of mRNA samples were stored in siliconized tubes at −20° C. All mRNAs were cellulose-purified to remove double-stranded RNA contaminants as described in Baiersdörfer, et al. (2019) Mol. Ther. Nucleic Acids 15(15):26-35.

Analysis of mRNA Capping Efficiency

Ribozyme cleavage reaction contains so much molar mass (g/mol) of mRNA that the amount of short cleaved product should be equal to 150 ng and a 3-fold molar excess of ribozyme over mRNA substrate in 30 mM HEPES and 150 mM NaCl. The ribozyme cleavage reaction was performed on a PCR machine utilizing the following program: 95° C. for 2 min, chill the mixture up to 37° C. by ramping rate of 0.1° C./sec, 37° C. for 5 min, after adding 30 mM MgCl₂ solution to each sample, the mixtures were maintained at 37° C. for 60 min followed by stopping the annealing at 80° C. for 2 min and keep on ice for 5 min. After then the short RNA fragments were separated from the long ones using the RNA Clean & Concentrator-5 kit (Zymo Research Europe) according to the manufacturer's instructions. In this Example the following custom-designed hammerhead ribozyme specific for hAg 5′UTR was used: 5′-UGU GGG CUG AUG AGG CCG UGA GGC CGA AAC CAG AAG AAU-3′ and was synthesized by Metabion. For detection of short cleaved products, the samples (30 ng) were resolved on a 21% (vol/vol) 19:1 acrylamide:bisacrylamide denaturing gel supplemented with 8 M urea (Merck). Before loading the samples denatured by incubation at 75° C. for 5 minutes in the presence of 2×RNA loading buffer (New England Biolabs), the gel was pre-run at 180 V for 60 minutes. When the pre-run was finished, the pockets were rinsed with 1×TBE buffer. Immediately afterwards the samples were applied, and the gel was run at 200 V constantly until the dye front has reached the end of the gel. For identification of the short products, the gel was incubated with 1×TBE buffer containing 0.01% SYBR Gold nucleic acid stain (Thermo Fisher Scientific) and the fluorescent signals were captured using a Gel Doc EZ Imager (Bio-Rad).

Validation of ribozyme assay was performed using hydrophilic interaction liquid chromatography (HILIC) coupled with tandem mass spectrometry (MS/MS) following P1 nuclease (Merck) digestion. The mRNAs were filtrated (Amicon Ultra 0.5 ml MWCO 30 kDa), incubated with nuclease P1 on a thermomixer (Eppendorf) for 3 h at 37° C. with 450 rpm and afterwards lyophilized. After reconstitution in 40% acetonitrile, the cleaved 5′ mRNA fragments (the dinucleotide resulting from digestion of the 5′ cap, GTP and ATP which form the 5′ end of the mRNA if no cap is integrated) were analyzed with liquid chromatography on a Prominence HPLC (Shimadzu) connected to an electrospray tandem mass spectrometer (LC-MS/MS; Shimadzu 8050) operating in positive multiple reaction monitoring (MRM) mode using isotope labeled internal standards (Merck). The standards for calibration were obtained from Jena Bioscience (ARCA) and Merck (ATP, GTP). Mobile phase A was 100 mM ammonium carbonate buffer pH 8.9 and mobile phase B was 100% acetonitrile. A Waters ACQUITY PREMIER BEH Amide VanGuard FIT Column, 1.7 μm, 2.1 mm×50 mm heated to 55° C. with a flow rate of 300 L/min was used for all analyses. The gradient profile for elution started at 25% A and was linearly ramped to 55% A over 0.8 minutes. A 0.8-min-rinse step at 55% A was followed by a return to 25% A at 1.7 min. Total run time was 3 minutes.

The ratio of the dinucleotide from the capping unit, GTP and ATP, describes the degree to which RNA is capped during IVT and is calculated using the following formula:

$$Capping Efficiency ( \% ) = \frac{Conc . ( Dinucl . )}{Sum ( Conc . ( Dinucl . ) + Conc . ( GTP ) + Conc . ( ATP ) )} * 100$$

### Visualization of Short Byproducts

For detection of short abortive byproducts, IVT mRNAs (1.5-2 μg) were resolved on a 21% (vol/vol) 19:1 acrylamide:bisacrylamide denaturing gel supplemented with 8 M urea (Merck). Before loading the mRNA samples denatured by incubation at 75° C. for 10 minutes in the presence of 2×RNA loading buffer (New England Biolabs), the gel was pre-run at 180 V for 60 minutes. When the pre-run was finished, the pockets were rinsed with 1×TBE buffer. Immediately afterwards the samples were applied, and the gel was run at 200 V constantly until the dye front has reached the end of the gel. For identification of the short byproducts, the gel was incubated with 1×TBE buffer containing 0.01% SYBR Gold nucleic acid stain (Thermo Fisher Scientific) and the fluorescent signals were captured using a Gel Doc EZ Imager (Bio-Rad).

PBMC Preparation, Culture and Transfection with LPC-mRNAs

Human buffy coats from healthy individuals were obtained and used to isolate peripheral blood mononuclear cells (PBMCs) by Ficoll-Paque™ PLUS (Cytiva) density gradient. For mRNA transfection, cryopreserved PBMCs were thawed and seeded into 96-well plates at a density of 5×10⁵ cells per well in 190 μL RPMI supplemented with 1% non-essential amino acids (NEAA), 1% sodium pyruvate and 10% Fetal Bovine Serum (Merck). Cells were maintained at 37° C. with 5% CO₂ until transfection with 40, 100 and 300 ng lipoplex-formulated EPO mRNA (LPX-RNA) in a final volume of 10 μl of transfection mixture, respectively, as described in Kranz, et al (2016) Nature 534(7607):396-401. Immediately after that, the complexed RNA was added to each well in triplicates and supernatants were collected for measurement of cytokine/chemokine profile at 24 hours after transfection.

### Cytokine/Chemokine Production Assay

To determine the production of selected cytokines/chemokines, supernatants from human PBMCs transfected with LPX-RNA were subjected to cytokine/chemokine profile analysis using the Meso Scale Discovery V-PLEX Custom Human Biomarkers Proinflammatory and Chemokine Panel (Meso Scale Diagnostics) according to the manufacturer's instructions. A sample dilution of 1:5=supernatant: MSD diluent was used in each experiment. The levels of TNF-α (Tumor necrosis factor alpha), IFN-γ (Interferon gamma), IL-6 (Interleukin 6), IL-1β (Interleukin 1 beta) and MIP-1β (Macrophage inflammatory protein 1 beta) were quantified 24 hours after the mRNA transfection.

### In Vitro Translation

In vitro protein synthesis from the uncapped and capped mRNAs encoding firefly luciferase was performed using Rabbit Reticulocyte Lysate (RRL) System (Promega) according to the manufacturer's instructions. Reactions were terminated by incubation on ice followed by addition of 50 μl 1× luciferase cell lysis buffer (Promega). For detection of the firefly luciferase activity, the mixture of 10 μl RRL and lysis buffer was distributed in triplicates to wells of a 96-well white plate (Thermo Fisher Scientific) and subsequently 50 μl of luciferase substrate solution (Promega) were added to each well. Immediately, photon luminescence emission was measured using a Tecan Infinite 200 Pro (Tecan Trading AG). Luciferase data was normalized to the diluent buffer-only control for each sample.

### In Vivo Studies

For in vivo studies, BALB/c female mice from Jackson Laboratory at the age of eight to ten weeks were used in accordance with federal policies on animal research. Mice (4 mice/group) were injected intravenously (i.v.) with 3 μg TransIT-complexed (Mirus Bio) mRNA encoding firefly luciferase or murine EPO in a final volume of 200 μL Dulbecco's modified Eagle's medium. Control mice were injected with TransIT-reagent diluted in DMEM without RNA. In vivo imaging of luciferase expression was performed at 6, 24 and 48 hours post-delivery of LUC mRNA using the IVIS Spectrum In Vivo Imaging System (PerkinElmer).

For measurement of individual hematocrits and EPO levels in animals that were injected with mEPO mRNA complexed with TransIT®-mRNA, eighteen microliters of blood were collected at the indicated times and centrifuged in Drummond microcaps glass capillary tubes (20 μl volume, Merck) as described in Mahiny, et al. (2016) Methods in Molecular Biology 1428:297-306. After determination of the hematocrit, capillary tubes were snapped, and the plasma was collected for the measurement of mouse EPO levels and analyzed for mouse Erythropoietin DuoSet ELISA kit (R&D Systems) according to the manufacturer.

### Statistical Analysis

Statistical analysis for comparing the performance of differently capped IVT mRNAs was performed using GraphPad Prism 9 software. All data is reported as mean±SEM. A p<0.05 was considered statistically significant.

### Example 2—Evaluating the Effect of Starting Triplet on mRNA Expression and Activity

To investigate the effect produced by incorporating a pyrimidine base at the +2 position of the transcription start site, mRNA polynucleotides encoding murine EPO (mEPO), comprising N1-methylpseudouridine (m1Ψ) in place of uridine, a start sequence (GGG, GAG, GGA, GGC, GGU, GCA, GUG or GCG), an anti-reverse dinucleotide cap containing phosphorothioate group (β-S-ARCA D1), and a TEV 5′UTR were synthesized in vitro.

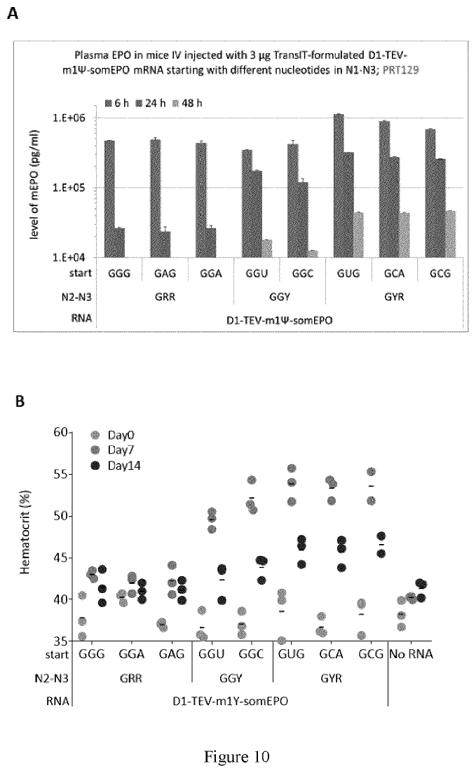

3 μg of each mRNA transcript was TransIT-formulated and injected IV into mice, and mEPO serum expression was measured 6, 24, and 48 hours post injection (results shown in FIG. 10A; each dot represents one mouse). Templates containing a pyrimidine base (C or U) at the +2 position downstream from the T7 promoter translated more efficiently and produced higher serum concentrations of mEPO than transcripts containing a purine base (A or G) at the +2 position. Furthermore, a long-term beneficial effect on translation in mice injected with D1-capped EPO mRNA containing the m1Ψ modification and starting with GCA, GUG and GCG was also observed (FIG. 10A) as serum concentrations of EPO were found to remain elevated for a longer period of time in mice injected with these transcripts as compared to mice injected with transcripts comprising a purine at the +2 position. Hematocrit levels were also found to be elevated in mice injected with D1-capped EPO m1Ψ-mRNA containing a pyrimidine base in the first three transcribed nucleotides, and remained elevated 14 days after injection (FIG. 10B).

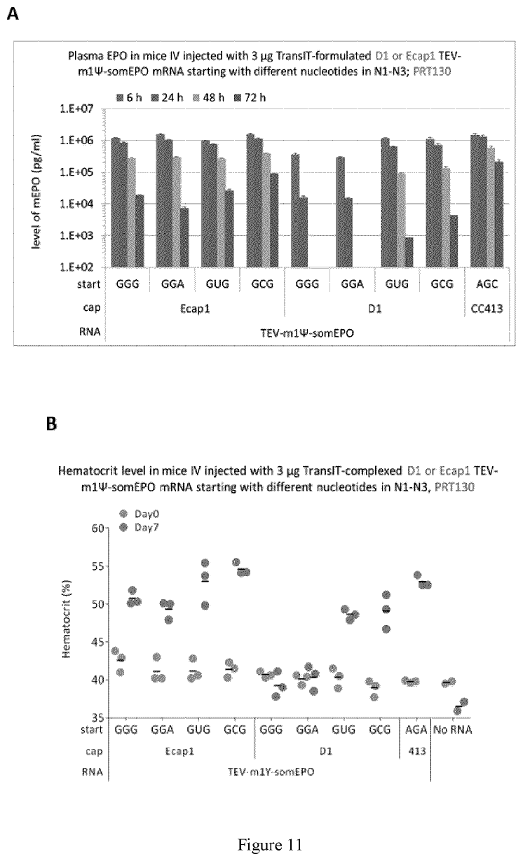

The effect of the first three nucleotides on enzymatically capped RNA was also investigated. RNA encoding murine EPO, and comprising a modified nucleoside (m1Ψ) and a start sequence (GGG, GGA, GUG, or GCG) were synthesized in vitro and enzymatically labeled (Ecap 1 samples in FIG. 11). Similar transcripts were produced using the D1 cap and an RNA polynucleotide comprising an AGC start sequence and the CleanCap AG 3′OMe (CC413) cap was used as a control for comparison. Each of the capped mRNA's was administered by IV to mice, and serum EPO levels were measured 6, 24, 48, and 72 hours post injection (FIG. 11A). Like transcripts comprising the D1 cap, enzymatically labelled transcripts comprising a pyrimidine nucleotide at the +2 position displayed significantly improved expression (FIG. 11A) and biological activity (FIG. 11B) as compared to transcripts comprising a purine nucleotide at the +2 position, although the difference was smaller than that observed in transcripts comprising D1. Importantly, D1-capped mRNA starting with GCG produced EPO concentrations as high as those produced by enzymatically capped mRNA starting with GGG.

### Example 3—Evaluating the Effect of Uridine and 5′UTR on Transcription Efficiency

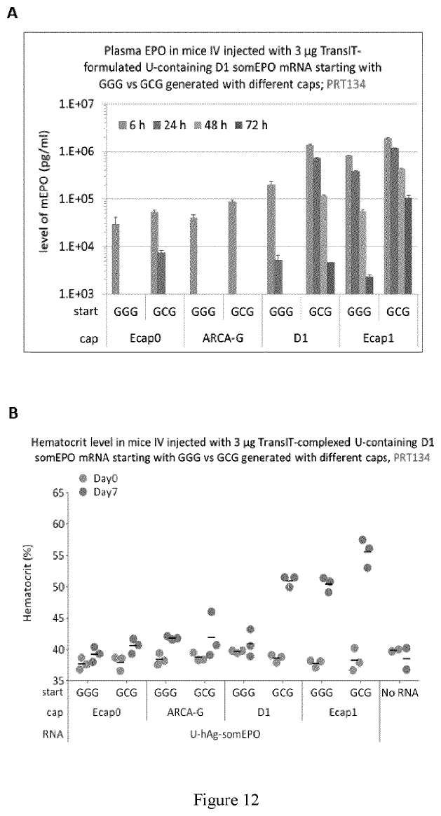

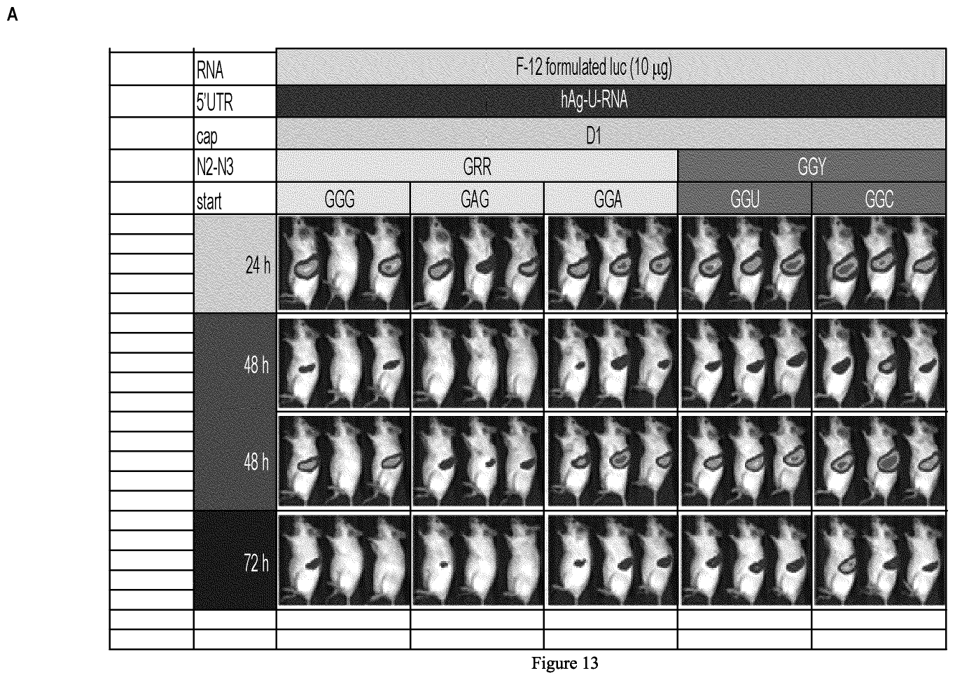

To determine whether the improved translation produced by transcripts comprising a pyrimidine nucleotide at the +2 position was affected by the identity of the 5′ cap or the use of m1Ψ rather than uridine, RNA polynucleotides comprising a start sequence (GGG or GCG), a 5′ cap (Ecap0 (formed by Vaccinia capping system without 2′-O-methyltransferase (NEB)), ARCA-G, D1, or Ecap1) and uridine were synthesized in vitro. Again, 3 μg of each transcript was injected into mice, and mEPO serum concentrations were measured 6, 24, 48, and 72 hours after injection (FIG. 12A) and hematocrit levels were measured 0 and 7 days after injection (FIG. 12b). Improved EPO expression was observed for the GCG start sequence as compared to the GGG start sequence for each 5′ cap tested. Accordingly, the improvement in translation provided by the GCG start sequence as compared to the GGG start sequence appears to be independent of 5′ cap. Expression of EPO in mice injected with a transcript comprising D1 and a GCG start sequence was again found to be similar to that of mice injected with an Ecap1 construct and a GGG start sequence, even though uridine was used rather than m1Ψ. Hematocrit levels were also found to be similar in mice administered a construct comprising D1 cap and a GCG start sequence as compared to mice administered a transcript comprising Ecap1 and a GGG start sequence (FIG. 12B).

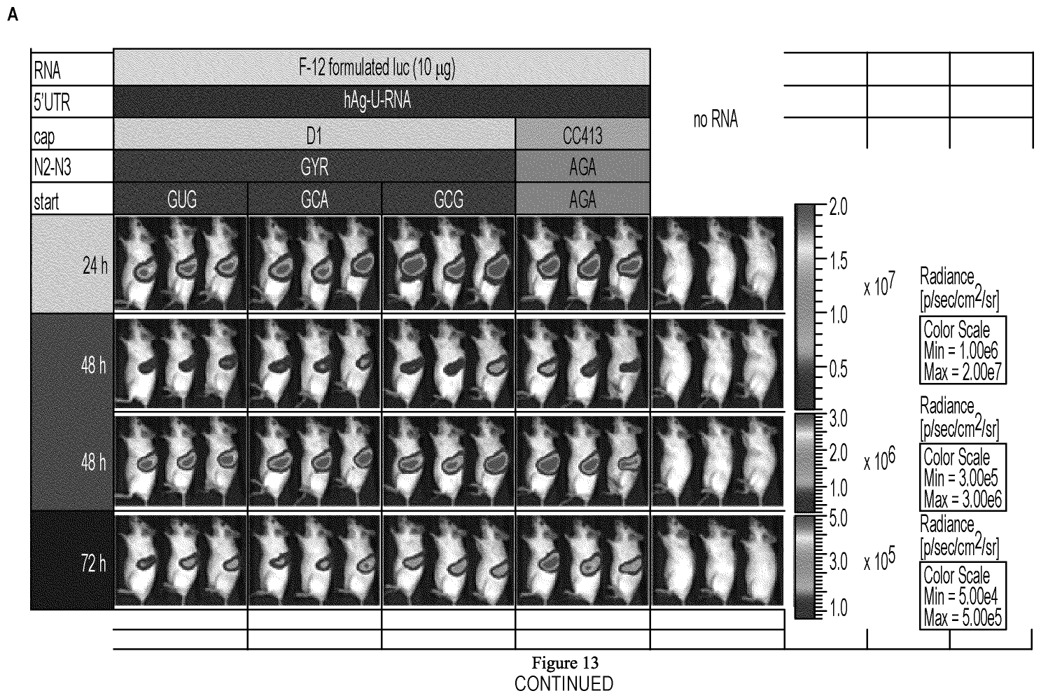

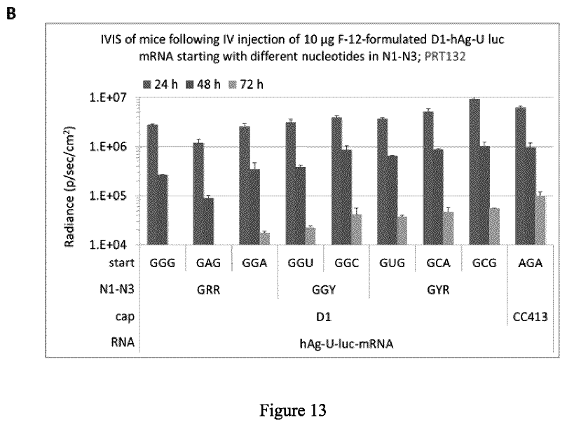

To determine whether the improved translation provided by pyrimidine containing start sequences was affected by the 5′UTR, the encoded protein, or the lipid formulation used to administer the RNA construct, RNA transcripts comprising D1, uridine, and a start sequence (GGG, GAG, GGA, GGU, GGC, GUG, GCA, or GCG) were synthesized. Each construct was formulated with F-12 lipoplex (Kranz, et al (2016) Nature 534(7607):396-401), and 10 μg of the formulated RNA was injected into mice (FIGS. 13A and 13B). Luciferase signal was measured 24, 48, and 72 hours after injection. Constructs comprising a pyridine nucleotide in the first three nucleotides were again found to provide improved translation as compared to constructs comprising a purine in their first three nucleotides. Thus, the GYR effect is independent of nucleoside modification, 5′UTR, coding sequence and formulation.

### Example 4—Confirming the Effect of the Starting Triplet on Translation Efficiency of mRNA Transcripts

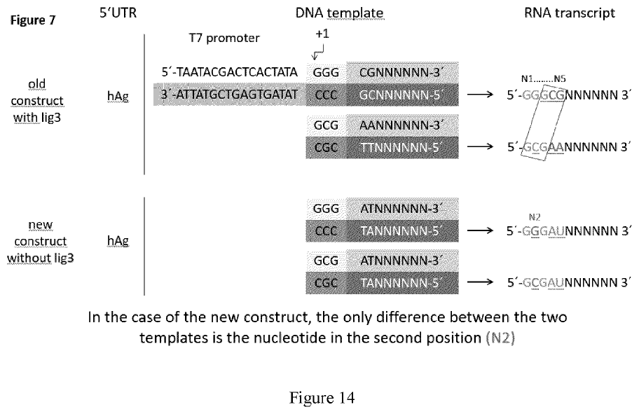

The constructs comprising hAg 5′UTR in the previous experiments differed not only in the nucleotide at the +2 position, but also at positions +4 and +5 (GGGCG vs GCGAA) (FIG. 14). Moreover, these constructs contain a Lig3 self-hybridization sequence in the 3′ UTR, which impacts translation. Because these differences may affect expression independent of the start sequence, we designed constructs that were identical except at position +2 downstream from the T7 promoter (GGGAT vs GCGAT) (FIG. 14). The properties of the mRNAs transcribed from these templates was characterized.

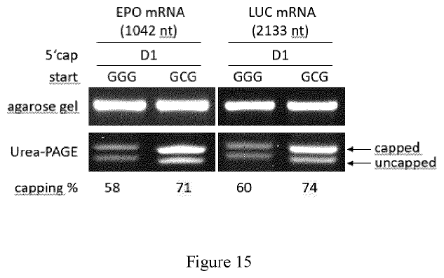

Transcripts encoding EPO or luciferase, and comprising a GGGAU start sequence or a GCGAU start sequence, m1Ψ, a 5′ cap (ARCA-G, D1, or Ecap1), and an hAg 5′UTR were synthesized in vitro. After the transcription reaction was complete, the reaction mixtures were run on an agarose gel and a urea-PAGE gel (FIG. 15). Capping efficiency was estimated by comparing the intensity of the top band (capped) with that of the lower band (uncapped) in the urea-PAGE gel. D1-capped mRNA starting with GCGAU was found to have a significantly higher capping efficiency as compared to D1-capped mRNA starting with GGGAU (FIG. 15).

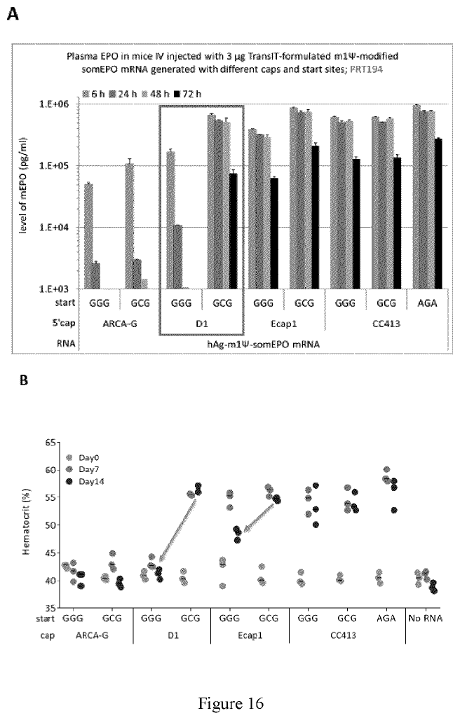

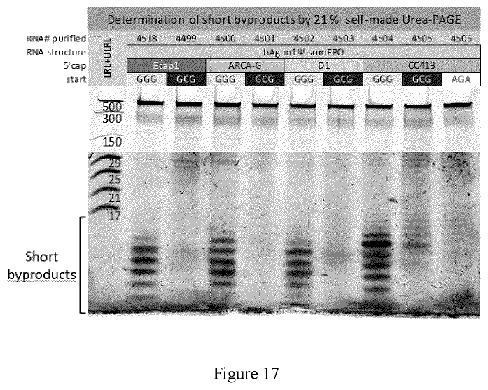

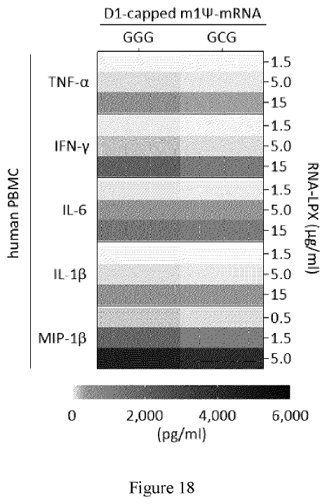

The translational capacity and biological activity of transcripts comprising the GGGAU and GCGAU start sequence was also assessed. Transcripts comprising an hAg 5′ UTR, a 5′ cap (ARCA-G, D1, Ecap1, or CC413), m1Ψ, encoding EPO, and comprising a start sequence of GGGAU or GCGAU were synthesized in vitro and then complexed with TransIT mRNA formulation agent. 3 μg of the TransIT-complexed RNA was then injected into mice by IV. Serum concentrations of mEPO was then measured 6, 24, 48, and 72 hours after injection and hemacrit levels were measured 0, 7, and 14 days after administration (FIG. 16). As seen previously, mice injected with RNA comprising the GCG start sequence and the D1 cap exhibited significantly higher serum concentrations of EPO than mice injected with RNA comprising the GGG start sequence and the D1 cap. This improvement was also observed in transcripts comprising ARCA-G or Ecap1, although the difference was less pronounced. CC413 was the only cap for which no improvement in translation was observed between the GGG and GCG start sequences). These higher mEPO concentrations were also shown to lead to higher hematocrit levels (FIG. 16B). The transcription reaction mixtures were also run on a urea-PAGE gel (FIG. 17). The GCGAU start sequence was found to eliminate short abortive byproducts (FIG. 17) and attenuate levels of proinflammatory cytokines/chemokines compared to transcripts comprising a GGGAU sequence for each cap tested. Transcripts comprising the D1 cap and the GCG start sequence were also found to elicit a reduced cytokine response from PBMCs as compared to transcripts comprising the D1 cap and the GGG start sequence (FIG. 18).

In conclusion, these findings indicate that the use of β-S-ARCA D1 cap with appropriate starting triplet provides more efficient, less immunogenic, and cost-effective mRNA than those starting with standard G triplet and can substitute the natural 5′cap.

### EQUIVALENTS

Those skilled in the art will recognize, or be able to ascertain using no more than routine experimentation, many equivalents to the specific embodiments of the invention described herein. It is to be understood that the invention encompasses all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. Further, it should also be understood that any embodiment or aspect of the invention can be explicitly excluded from the claims, regardless of whether the specific exclusion is recited in the specification. The scope of the present invention is not intended to be limited to the above Description, but rather is as set forth in the claims that follow.

What is claimed is:

1. A composition or medical preparation comprising an RNA polynucleotide comprising:
a 5′ cap; a cap proximal sequence comprising positions +1, +2, +3, +4, and +5 of the RNA polynucleotide; and a sequence encoding a payload, wherein:
(i) the 5′ cap is a trinucleotide cap structure comprising N₁pN₂, wherein N₁ is position +1 of the RNA polynucleotide, and N₂ is position +2 of the RNA polynucleotide, and wherein N₁ is A and N₂ is U; and
(ii) the cap proximal sequence comprises:
N₁ and N₂ of the trinucleotide cap structure and a sequence comprising N₃N₄N₅ at positions +3, +4, and +5 respectively of the RNA polynucleotide, wherein N₃ is A, and each N₄ and N₅ is selected from: A, C, G, and U.

2. A composition or medical preparation comprising an RNA polynucleotide comprising:
a 5′ cap; a cap proximal sequence comprising positions +1, +2, +3, +4, and +5 of the RNA polynucleotide; and a sequence encoding a payload, wherein:
(i) the 5′ cap is a trinucleotide cap structure comprising N₁pN₂, wherein N₁ is position +1 of the RNA polynucleotide, and N₂ is position +2 of the RNA polynucleotide, and wherein N₁ and N₂ are selected from one of the following combinations: (a) N₁ is G and N₂ is G; (b) N₁ is U and N₂ is G; (c) N₁ is A and N₂ is G; or (d) N₁ is C and N₂ is G; and
(ii) the cap proximal sequence comprises:
N₁ and N₂ of the trinucleotide cap structure and a sequence comprising N₃N₄N₅ at positions +3, +4, and +5 respectively of the RNA polynucleotide, wherein N₃ is C, N₄ is G, and N₅ is selected from: A, C, G, and U.

3. A composition or medical preparation comprising an RNA polynucleotide comprising:
a 5′ cap; a cap proximal sequence comprising positions +1, +2, +3, +4, and +5 of the RNA polynucleotide; and a sequence encoding a payload, wherein:
(i) the 5′ cap is a trinucleotide cap structure comprising N₁pN₂, wherein N₁ is position +1 of the RNA polynucleotide, and N₂ is position +2 of the RNA polynucleotide, and wherein N₁ is G and N₂ is C; and
(ii) the cap proximal sequence comprises:
N₁ and N₂ of the trinucleotide cap structure and a sequence comprising N₃N₄N₅ at positions +3, +4, and +5 respectively of the RNA polynucleotide, wherein N₃ is G, and each N₄ and N₅ is selected from: A, C, G, and U.

4. The composition or medical preparation of any one of claims 1-3, wherein the trinucleotide cap structure has a structure: G*N¹pN₂, wherein
G* comprises a structure of formula (I): 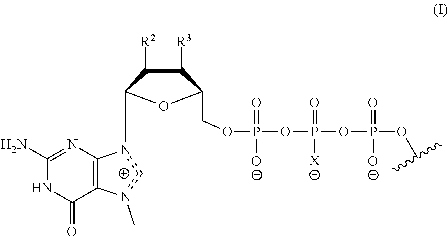
or a salt thereof,
wherein
each R² and R³ is —OH or —OCH₃; and
X is O or S.

5. The composition or medical preparation of claim 4, wherein R² is —OH.

6. The composition or medical preparation of claim 4, wherein R² is —OCH₃.

7. The composition or medical preparation of any one of claims 4-6, wherein R³ is —OH.

8. The composition or medical preparation of any one of claims 4-7, wherein R³ is —OCH₃.

9. The composition or medical preparation of any one of claims 4-8, wherein X is O.

10. The composition or medical preparation of any one of claims 1-9, wherein the trinucleotide cap structure comprises a Cap0 or Cap1 structure.

11. The composition or medical preparation of any one of claims 1-9, wherein the trinucleotide cap structure comprises (m^{2′-O})N₁pN₂.

12. The composition or medical preparation of claim 1, wherein the trinucleotide cap structure is (m⁷)Gppp(m^{2′-O})ApU.

13. The composition or medical preparation of claim 2 or 3, wherein the trinucleotide cap structure is selected from the group consisting of: (m₂^{7,3′-O})Gppp(m^{2′-O})ApG (“CleanCap AG”, “CC413”), (m₂^{7,3′-O})Gppp(m^{2′-O})GpG (“CleanCap GG”), (m⁷)Gppp(m^{2′-O})ApG, and (m₂^{7,3′-O})Gppp(m₂^{6,2′-O})ApG.

14. A composition or medical preparation comprising an RNA polynucleotide comprising:
a 5′ cap; a cap proximal sequence comprising positions +1, +2, +3, +4, and +5 of the RNA polynucleotide; and a sequence encoding a payload, wherein:
(i) the 5′ cap is a dinucleotide cap structure comprising N₁, wherein N₁ is position +1 of the RNA polynucleotide, and wherein N₁ is G; and
(ii) the cap proximal sequence comprises:
N₁ of the dinucleotide cap structure and a sequence comprising N₂N₃N₄N₅ at positions +2, +3, +4, and +5 respectively of the RNA polynucleotide, wherein N₂ is C, N₃ is G, and each N₄ and N₅ is selected from: A, C, G, and U.

15. The composition or medical preparation of claim 14, wherein the dinucleotide cap structure is: G*N₁, wherein
G* comprises a structure of formula (I): 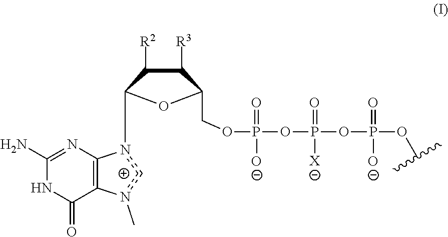
or a salt thereof,
wherein
each R² and R³ is —OH or —OCH₃; and
X is O or S.

15. The composition or medical preparation of claim 14, wherein R² is —OH.

16. The composition or medical preparation of claim 14, wherein R² is —OCH₃.

17. The composition or medical preparation of any one of claims 14-16, wherein R³ is —OH.

18. The composition or medical preparation of any one of claims 14-16, wherein R³ is —OCH₃.

19. The composition or medical preparation of any one of claims 14-18, wherein X is O.

20. The composition or medical preparation of any one of claims 14-18, wherein X is S.

21. The composition or medical preparation of any one of claims 14-20, wherein the dinucleotide cap structure comprises a Cap0 or Cap1 structure.

22. The composition or medical preparation of any one of claims 14-21, wherein the dinucleotide cap structure is selected from the group consisting of (m⁷)GpppG (“Ecap0”), (m⁷)Gppp(^{2′-O})G (“Ecap1”), (m₂^{7,3′-O})GpppG (“ARCA” or “D1”), and (m₂^{7,2′-O})GppSpG (“beta-S-ARCA”).

23. A composition or medical preparation comprising an RNA polynucleotide comprising:
a 5′ cap; a cap proximal sequence comprising positions +1, +2, +3, +4, and +5 of the RNA polynucleotide; and a sequence encoding a payload, wherein:
(i) the 5′ cap is m₂^{(7,3′O)}Gppp^{(m2′O)}A₁pG₂, wherein A₁ is position +1 of the RNA polynucleotide, and G₂ is position +2 of the RNA polynucleotide; and
(ii) the cap proximal sequence comprises:
A₁ and G₂ of the 5′ cap and a sequence comprising N₃N₄N₅ at positions +3, +4, and +5 respectively of the RNA polynucleotide, wherein N₃ is C, N₄ is G, and N₅ is selected from: A, C, G, and U.

24. The composition or medical preparation of any one of claims 1-22, wherein N₄ is A.

25. The composition or medical preparation of any one of claims 1-22, wherein N₄ is C.

26. The composition or medical preparation of any one of claims 1-22, wherein N₄ is G.

27. The composition or medical preparation of any one of claims 1-22, wherein N₄ is U.

28. The composition or medical preparation of any one of claims 1-27, wherein N₅ is A.

29. The composition or medical preparation of any one of claims 1-27, wherein N₅ is C.

30. The composition or medical preparation of any one of claims 1-27, wherein N₅ is G.

31. The composition or medical preparation of any one of claims 1-27, wherein N₅ is U.

32. An in vitro transcription reaction comprising:
(i) a template DNA strand comprising a polynucleotide sequence complementary to an RNA polynucleotide sequence provided in claim 1 or 12, wherein the template DNA strand comprises a sequence that is complementary to a AUA transcription start site;
(ii) a polymerase;
(iii) ribonucleotides; and
(iv) a 5′ cap comprising N₁pN₂;
wherein N₁ is A and N₂ is U.

33. An in vitro transcription reaction comprising:
(i) a template DNA strand comprising a polynucleotide sequence complementary to an RNA polynucleotide sequence provided in any one of claims 2-3 or 13, wherein the template DNA strand comprises a sequence that is complementary to a GCG transcription start site;
(ii) a polymerase;
(iii) ribonucleotides; and
(iv) a 5′ cap comprising N₁pN₂;
wherein N₁ is A, C, G, or U, and N₂ is G; or
wherein N₁ is G and N₂ is C; and
wherein the sequence in the template strand that is complementary to GCG is the start site of transcription by an RNA polymerase.

34. The in vitro transcription reaction of claim 32 or 33, wherein the 5′ cap structure has a structure: G*N¹pN₂, wherein
G* comprises a structure of formula (I): 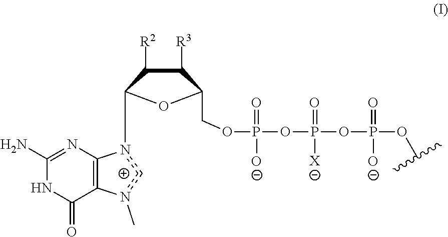
or a salt thereof,
wherein
each R² and R³ is —OH or —OCH₃; and
X is O or S.

35. The in vitro transcription reaction of claim 34, wherein R² is —OH.

36. The in vitro transcription reaction of claim 34, wherein R² is —OCH₃.

37. The in vitro transcription reaction of any one of claims 34-36, wherein R³ is —OH.

38. The in vitro transcription reaction of any one of claims 34-37, wherein R³ is —OCH₃.

39. The in vitro transcription reaction of any one of claims 34-38, wherein X is O.

40. The in vitro transcription reaction of any one of claims 32-39, wherein the trinucleotide cap structure comprises a Cap0 or Cap1 structure.

41. The in vitro transcription reaction of any one of claims 32-39, wherein the trinucleotide cap structure comprises (m^{2′-O})N₁pN₂.

42. The in vitro transcription reaction of claim 32, wherein the trinucleotide cap structure is (m⁷)Gppp(m^{2′-O})ApU.

43. The in vitro transcription reaction of claim 33, wherein the trinucleotide cap structure is selected from the group consisting of: (m₂^{7,3′-O})Gppp(m^{2′-O})ApG (“CleanCap AG”, “CC413”), (m₂^{7,3′-O})Gppp(m^{2′-O})GpG (“CleanCap GG”), (m⁷)Gppp(m^{2′-O})ApG, and (m₂^{7,3′-O})Gppp(m₂^{6,2′-O})ApG.

44. An in vitro transcription reaction comprising:
(i) a template DNA strand comprising a polynucleotide sequence complementary to an RNA polynucleotide sequence provided in any one of claims 14-21, wherein the template DNA strand comprises a sequence that is complementary to a transcription start site comprising GCG;
(ii) a polymerase;
(iii) ribonucleotides; and
(iv) a 5′ dinucleotide cap;
wherein the sequence in the template DNA strand that is complementary to the transcription start site is the start site of transcription by an RNA polymerase.

45. An RNA polynucleotide produced from an in vitro transcription reaction provided in any one of claims 32-44.

46. A method of making a capped RNA polynucleotide comprising:
transcribing a template DNA strand in the presence of ribonucleotides and a cap comprising a structure of A₁pU₂, wherein the template DNA strand comprises an RNA polymerase promoter sequence and a sequence comprising positions +1, +2, +3, +4, and +5 (in the 3′ to 5′ direction), wherein the positions +1, +2, +3 of the template DNA strand are complementary to a AUA transcription start site (i.e., position +1 of the template DNA strand is T, position +2 of the template DNA strand is A; and position +3 of the template DNA strand is T); and wherein positions +4 and +5 of the template DNA strand are each independently any nucleotide, thereby producing a capped RNA polynucleotide.

47. A method of making a capped RNA polynucleotide comprising:
transcribing a template DNA strand in the presence of ribonucleotides and a cap comprising a structure of G₁pC₂, wherein the template DNA strand comprises an RNA polymerase promoter sequence and a sequence comprising positions +1, +2, +3, +4, and +5 (in the 3′ to 5′ direction), wherein the positions +1, +2, +3 of the template DNA strand are complementary to a GCG transcription start site (i.e., position +1 of the template DNA strand is C, position +2 of the template DNA strand is G, and position +3 of the template DNA strand is C), and wherein positions +4 and +5 of the template DNA strand are each independently any nucleotide, thereby producing a capped RNA polynucleotide.

48. A method of making a capped RNA polynucleotide comprising:
a 5′ dinucleotide cap structure comprising N₁; a cap proximal sequence comprising positions +1, +2, +3, +4, and +5 of the RNA polynucleotide; and a sequence encoding a payload, wherein:
the cap proximal sequence comprises N₁ of the 5′ cap, and N₂, N₃, N₄, and N₅, wherein N₁ to N₅ correspond to positions +1, +2, +3, +4, and +5 of the RNA polynucleotide, wherein N₁ is G, N₂ is U or C, and N₃, N₄, and N₅ are each independently chosen from: A, C, G, and U;
wherein the method comprises transcribing a template DNA strand in the presence of the 5′ cap and an RNA polymerase.

49. The method of any one of claims 46-48, wherein positions +4 and +5 of the template DNA strand are each independently A, C, G, or T.

50. The method of any one of claims 46-49, further comprising purifying the capped RNA polynucleotide.

51. A complex comprising a template DNA strand and a 5′ cap comprising a structure of N₁pN₂, wherein the DNA template strand comprises an RNA polymerase promoter sequence and a sequence comprising positions +1, +2, +3, +4, and +5 (in the 3′ to 5′ direction), wherein the positions +1, +2, and +3 of the template DNA strand are complementary to a transcription start site of a coding DNA strand;
wherein N₁ and N₂ are each independently chosen from: A, C, G, and U;
wherein N₂ interacts with the +1 position of the template DNA strand (which is complementary to the first nucleotide of the transcription start site) and N₁ does not interact with the +1 position of the template DNA strand;
wherein the +4, and +5 positions of the template DNA strand are each independently chosen from: A, C, G, and U, and
wherein the sequence in the template DNA strand that is complementary to the transcription start site (i.e., positions +1, +2, +3 of the template DNA strand) is the start site of transcription by an RNA polymerase.

52. The complex of claim 51, wherein:
N₁ is A and N₂ is G, and wherein the +1 position of the template DNA strand is C;
N₁ is U and N₂ is G, and wherein the +1 position of the template DNA strand is C; or
N₁ is C and N₂ is G, and wherein the +1 position of the template DNA strand is C.

53. A complex comprising a template DNA strand and a 5′ cap comprising a structure of N₁pN₂, wherein the DNA template strand comprises an RNA polymerase promoter sequence and a sequence comprising positions +1, +2, +3, +4, and +5 (in the 3′ to 5′ direction), wherein the positions +1, +2, and +3 of the template DNA strand are complementary to a transcription start site of a coding DNA strand;
wherein N₁ and N₂ are each independently chosen from: A, C, G, and U;
wherein N₁ interacts with the +1 position of the template DNA strand (which is complementary to the first nucleotide of the transcription start site) and N₂ interacts with the +2 position of the template DNA strand (which is complementary to the second nucleotide of the transcription start site);
wherein the +4, and +5 positions of the template DNA strand are each independently chosen from: A, C, G, and U, and
wherein the sequence in the template DNA strand that is complementary to the transcription start site (i.e., positions +1, +2, +3 of the template DNA strand) is the start site of transcription by an RNA polymerase.

54. The complex of claim 53, wherein N₂ is U or C, and the +2 position of the template DNA strand is A or G.

55. The complex of claim 53 or 54, wherein N₃ is A or G, and the +3 position of the template DNA strand is T or C.

56. The complex of claim 53, wherein:
N₁ is A and N₂ is G, and position +1 of the template DNA strand is T and position +2 of the template DNA strand is C;
N₁ is G and N₂ is C, and position +1 and position +2 of the template DNA strand are C and G, respectively; or
N₁ is A and N₂ is U, and position +1 and position +2 of the template DNA strand is T and A, respectively.

57. A complex comprising a template DNA strand and a 5′ cap comprising a structure of N₁, wherein the template DNA strand comprises an RNA polymerase promoter sequence and a sequence comprising positions +1, +2, +3, +4, and +5 (in the 3′ to 5′ direction), wherein the positions +1, +2, and +3 of the template DNA strand are complementary to a transcription start site of a coding DNA strand;
wherein N₁ is G; and
wherein N₁ interacts with the +1 position of the template DNA strand (which is complementary to the first nucleotide of the transcription start site);
wherein the +2 position of the template DNA strand (which is complementary to the second nucleotide of the transcription start site) is G or A;
wherein the +4, and +5 positions of the template DNA strand are each independently chosen from: A, C, G, and U, and
wherein the sequence in the template DNA strand that is complementary to the transcription start site is the start site of transcription by an RNA polymerase.

58. The complex of claim 57, wherein the +1 position of the template DNA strand is C, the +2 position of the template DNA strand is G, and the +3 position of the template DNA strand is C.

59. A method of formulating a pharmaceutical composition, the method comprising combining a preparation comprising an RNA polynucleotide of any one of claims 1 to 31 and 45 with a preparation comprising lipids.

60. A method comprising: administering to a subject, a pharmaceutical composition comprising an RNA polynucleotide of any one of claims 1-31 and 45.
